(19) United States
(12) Patent Application Publication
(10) Pub. No.: US 2026/0001876 A1
(43) Pub. Date: Jan. 1, 2026

(54) Compounds and Their Use as PDE4 Activators

(71) Applicant: Mironid Limited, Glasgow (GB)

(72) Inventors: Julia Mary Adam, Glasgow (GB); David Roger Adams, Glasgow (GB); Rutger Folmer, Nijmegen (NL); Koen Hekking, Nijmegen (NL); Yorik Bruseker, Nijmegen (NL)

(73) Assignee: Mironid Limited, Glasgow (GB)

(21) Appl. No.: 19/103,747

(22) PCT Filed: Aug. 17, 2023
(86) PCT No.: PCT/EP2023/072651

§ 371 Date: Feb. 13, 2025

(30) Foreign Application Priority Data
Aug. 17, 2022 (GB) 2212000.0

(51) Int. Cl. C07D 471/04; A61K 31/444; A61K 31/4545; A61K 31/496; A61K 31/4985; A61K 31/4995; A61K 31/519; A61P 5/20; A61P 13/12; A61P 35/00; C07D 519/00

(52) U.S. Cl. CPC C07D 471/04; A61K 31/444; A61K 31/4545; A61K 31/496; A61K 31/4985; A61K 31/4995; A61K 31/519; A61P 5/20; A61P 13/12; A61P 35/00; C07D 519/00

(57) ABSTRACT

The present invention relates to compounds of Formulas I to V and Ib to Vb, their use as activators of long form cyclic nucleotide phosphodiesterase-4 (PDE4) enzymes (isoforms) and to these compounds for use in a method for the treatment or prevention of disorders requiring a reduction of second messenger responses mediated by cyclic 3′,5′-adenosine monophosphate (cAMP).

### FIELD OF THE INVENTION

The present invention relates to compounds as defined herein, their use as activators of long form cyclic nucleotide phosphodiesterase-4 (PDE4) enzymes (isoforms) and to therapies using these compounds. In particular, the invention relates to these compounds for use in a method for the treatment or prevention of disorders requiring a reduction of second messenger responses mediated by cyclic 3′,5′-adenosine monophosphate (CAMP).

### BACKGROUND TO THE INVENTION

Cyclic 3′,5′-adenosine monophosphate—“CAMP”—is a critical intracellular biochemical messenger that is involved in the transduction of the cellular effects of a variety of hormones, neurotransmitters, and other extracellular biological factors in most animal and human cells. The intracellular concentration of CAMP is controlled by the relative balance between its rate of production and degradation. CAMP is generated by biosynthetic enzymes of the adenylyl cyclase superfamily and degraded by members of the cyclic nucleotide phosphodiesterase (PDE) superfamily. Certain members of the PDE superfamily, such as PDE4, specifically degrade cAMP, while others either specifically degrade cyclic guanosine monophosphate (cGMP) or degrade both cAMP and cGMP. PDE4 enzymes inactivate cAMP, thereby terminating its signalling, by hydrolysing cAMP to 5′-AMP (Lugnier, C. Pharmacol Ther. 109:366-398, 2006).

Four PDE4 genes (PDE4A, PDE4B, PDE4C and PDE4D) have been identified, each of which encodes a number of different enzyme isoforms through the use of alternative promoters and mRNA splicing. On the basis of their primary structures, the catalytically active PDE4 splice variants can be classified as “long”, “short” or “super-short” forms (Houslay, M. D. Prog Nucleic Acid Res Mol Biol. 69:249-315, 2001). A “dead short” form also exists, which is not catalytically active (Houslay, M. D., Baillie, G. S. and Maurice, D. H. Circ Res. 100:950-66, 2007). PDE4 long forms have two regulatory regions, called upstream conserved regions 1 and 2 (UCR1 and UCR2), located between their isoform-specific N-terminal portion and the catalytic domain. The UCR1 domain is absent in short forms, whereas the super-short forms not only lack UCR1, but also have a truncated UCR2 domain (Houslay, M. D., Schafer, P. and Zhang, K. Drug Discovery Today 10:1503-1519, 2005).

PDE4 long forms, but not short forms, associate into dimers within cells (Richter, W and Conti, M. J. Biol. Chem. 277:40212-40221, 2002; Bolger, G. B. et al., Cell. Signal. 27:756-769, 2015). A proposed negative allosteric modulation of PDE4 long forms by small molecules has been reported (Burgin A. B. et al., Nat. Biotechnol. 28:63-70, 2010; Gurney M. E. et al., Handb. Exp. Pharmacol. 204:167-192, 2011).

It is known in the art that PDE4 long forms may be activated by endogenous cellular mechanisms, such as phosphorylation (Mackenzie, S. J. et al., Br. J. Pharmacol. 136:421-433, 2002) and phosphatidic acid (Grange et al., J. Biol. Chem. 275:33379-33387, 2000). Activation of PDE4 long forms by ectopic expression of a 57 amino acid protein (called ‘UCR1C’) whose precise sequence reflects part of that of the upstream conserved region 1 of PDE4D (′UCR1C′ sequence reflects that of amino acids 80-136 while UCR is amino acids 17-136: numbering based on the PDE4D3 long isoform) has been reported (Wang, L. et al., Cell. Signal. 27:908-922, 2015: “UCR1C is a novel activator of phosphodiesterase 4 (PDE4) long isoforms and attenuates cardiomyocyte hypertrophy”). The authors hypothesised that PDE4 activation might be used as a potential therapeutic strategy for preventing cardiac hypertrophy.

The first small molecules that act as activators of PDE4 long forms were recently disclosed in WO2016/151300, WO2018/060704 and WO2019/193342. A small molecule activator of PDE4 long forms was recently evaluated in cell-based models of Autosomal Dominant Polycystic Kidney Disease (ADPKD) (Omar et al., PNAS 116:13320-13329, 2019). No small molecule activators of PDE4 long forms have yet been reported in clinical development. There remains a need for further, structurally distinct small molecule activators of PDE4 long forms for potential development as therapeutic agents.

It is amongst the objects of the present invention to provide new small molecule activators of at least one of the long forms of PDE4 for use in a method of therapy, as well as specific disease treatment or prevention.

### SUMMARY OF THE INVENTION

In a first aspect of the present invention, there is provided a compound of Formula I:

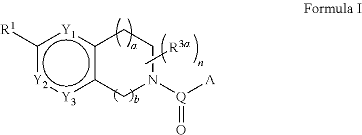

or a pharmaceutically acceptable salt or derivative thereof, wherein:
one or two of Y₁, Y₂ and Y₃ are N and the other(s) are CR^{3b};
Q is C or S(O);
R¹ is a 4- to 10-membered non-aromatic ring that may be monocyclic, bridged or bicyclic containing at least 1 ring N heteroatom and optionally a ring O heteroatom, and wherein R¹ is optionally substituted with 1 or more R⁴;
A is R^{2c}, NR^{2a}R^{2b} or OR^{2f},
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or 6-membered aromatic or heteroaromatic ring that contains 0, 1 or 2 ring N; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered carbocyclic ring or heterocyclic ring containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1 and b is 1 or 2, wherein when b is 2, a is 0;

for use in the treatment or prevention of a disease or disorder that can be ameliorated by activation of long isoforms of PDE4 or a disease or disorder mediated by excessive intracellular cyclic AMP signalling.

In a second aspect of the present invention, there is provided a compound of Formula II:

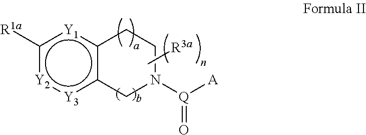

or a pharmaceutically acceptable salt or derivative thereof, wherein:
one or two of Y₁, Y₂ and Y₃ are N and the others are each CR^{3b}; Q is C or S(O);
R^{1a} is a 7- to 9-membered saturated, bridged ring system containing 2 ring N heteroatoms, and wherein R^{1a} is optionally substituted with 1 or more R⁴;
A is R^{2c}, or NR^{2a}R^{2b} or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered carbocylic ring or heterocyclic ring containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1 and b is 1 or 2, wherein when b is 2, a is 0.

In a third aspect of the present invention, there is provided a compound of Formula III:

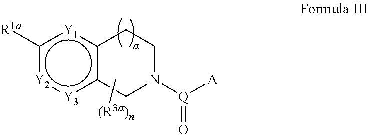

or a pharmaceutically acceptable salt or derivative thereof, wherein:
one of Y₁, Y₂ and Y₃ is N and the others are each CR^{3b};
Q is C or S(O);
R^{1b} is a 4- to 10-membered non-aromatic ring that may be monocyclic, bridged or bicyclic containing at least 1 ring N heteroatom and optionally a ring O heteroatom, wherein at least 1 ring N heteroatom is not at the point of attachment of R^{1b}, and wherein R^{1b} is optionally substituted with 1 or more R⁴;
A is R^{2c}, or NR^{2a}R^{2b}, optionally A is R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered ring carbocylic ring or heterocyclic ring containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

In a fourth aspect of the present invention, there is provided a compound of Formula IV:

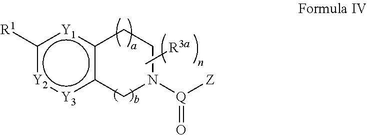

or a pharmaceutically acceptable salt or derivative thereof, wherein:
one or two of Y₁, Y₂ and Y₃ are N and the others are each CR^{3b};
Q is C or S(O);
R¹ is a 4- to 10-membered non-aromatic ring that may be monocyclic, bridged or bicyclic containing at least 1 ring N heteroatom and optionally a ring O heteroatom, and wherein R¹ is optionally substituted with 1 or more R⁴;
Z is R^{2e} or NR^{2d}R^{2b};
R^{2b} is H or (C1-6)alkyl, and wherein (C1-6)alkyl is optionally substituted with 1 or more R⁵; and
R^{2d} is
   a) a (C5-10)alkyl group comprising a cyclic moeity; and wherein R^{2d} is optionally substituted with 1 or more R⁵; or
   b) a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2d} is substituted with 1 or more R⁵ (optionally wherein R⁵ is halogen);
R^{2e} is
   a) a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms; and wherein R^{2e} is optionally substituted with 1 or more R⁵; or
   b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a (C3-10)alkyl group that may be straight chain or branched, wherein a C atom in straight chain portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; and wherein R^{2e} is substituted with 1 or more R⁵ (optionally wherein R⁵ is halogen);
each R^{3a} is independently (C1-6)alkyl, optionally substituted by 1 or more halogen; or
two R^{3a} attached to the same or adjacent carbon atoms may be joined together with the atoms to which they are attached to form a 3- to 6-membered carbocylic ring or heterocyclic ring containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1 and b is 1 or 2, wherein when b is 2, a is 0.

In a fifth aspect of the present invention, there is provided a compound of of Formula V:

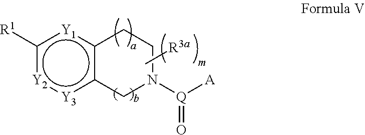

or a pharmaceutically acceptable salt or derivative thereof, wherein:
one or two of Y₁, Y₂ and Y₃ are N and the other(s) are CR^{3b};
Q is C or S(O);
R¹ is a 4- to 10-membered non-aromatic ring that may be monocyclic, bridged or bicyclic containing at least 1 ring N heteroatom and optionally a ring O heteroatom, and wherein R¹ is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered carbocylic ring or heterocyclic ring containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
m is 1, 2, 3 or 4; and
a is 0 or 1 and b is 1 or 2, wherein when b is 2, a is 0.

Compounds described herein are shown in the Examples to activate PDE4 long form enzymes.

In a further aspect, the present invention provides a pharmaceutical composition comprising a compound or a pharmaceutically acceptable salt or derivative as described herein, and a pharmaceutically acceptable excipient.

In a further aspect, the present invention provides a compound or pharmaceutical composition described herein for use in therapy. The therapy may be the treatment or prevention of any disease or disorder as described herein. The therapy may be the treatment or prevention of a disease or disorder that can be ameliorated by activation of long isoforms of PDE4. The therapy may be the treatment or prevention of a disease or disorder mediated by excessive intracellular cAMP signalling. In these diseases, a reduction of second messenger responses mediated by cyclic 3′,5′-adenosine monophosphate (CAMP) should provide a therapeutic benefit.

Also provided is a method of treating or preventing a disease or disorder that can be ameliorated by activation of long isoforms of PDE4, comprising the step of administering an effective amount of a compound or pharmaceutical composition described herein to a patient in need thereof. Also provided is a method of treating or preventing a disease or disorder mediated by excessive intracellular cAMP signalling, comprising the step of administering an effective amount of a compound or pharmaceutical composition described herein to a patient in need thereof.

Also provided is the use of a compound or pharmaceutical composition described herein in the manufacture of a medicament for treating or preventing a disease or disorder that can be ameliorated by activation of long isoforms of PDE4. Also provided is the use of a compound or pharmaceutical composition described herein in the manufacture of a medicament for treating or preventing a disease or disorder mediated by excessive intracellular cAMP signalling.

In certain embodiments of the foregoing aspects, the compounds of the invention are provided for the treatment or prevention of cancer. In certain embodiments of the foregoing aspects, the compounds of the invention are provided for the treatment or prevention of a disease or disorder selected from hyperthyroidism, Jansens's metaphyseal chondrodysplasia, hyperparathyroidism, familial male-limited precocious puberty, pituitary adenomas, Cushing's disease, polycystic kidney disease, polycystic liver disease, McCune-Albright syndrome, cholera, whooping cough, anthrax, tuberculosis, HIV, AIDS, Common Variable Immunodeficiency (CVID), melanoma, pancreatic cancer, leukaemia, prostate cancer, adrenocortical tumours, testicular cancer, primary pigmented nodular adrenocortical diseases (PPNAD), Carney Complex, autosomal dominant polycystic kidney disease (ADPKD), autosomal recessive polycystic kidney disease (ARPKD), maturity onset diabetes of young type 5 (MODY5), or cardiac hypertrophy.

### DETAILED DESCRIPTION

The invention is based on the surprising identification of new compounds that are able to activate long isoforms of PDE4 enzymes. The compounds are small molecules and so are expected to be easier and cheaper to make and formulate into pharmaceuticals than large biological molecules such as polypeptides, proteins or antibodies. The compounds can be chemically synthesized, as demonstrated in the Examples.

The Examples demonstrate that a number of compounds of Formula I to V and Ib to Vb are able to activate long isoforms of PDE4. The Examples go on to demonstrate that certain tested compounds of the invention do not activate a short form of PDE4, thereby demonstrating selectivity for activation of PDE4 long forms over PDE4 short forms. The Examples further demonstrate that PDE4 long form activators of the present invention reduce cAMP-driven cyst formation in an in vitro model of ADPKD.

Various aspects and embodiments are disclosed herein. It will be recognised that features specified in each embodiment may be combined with other specified features to provide further embodiments.

Described herein are compounds of Formula I to V, or pharmaceutically acceptable salts or derivatives thereof, as set out above. Formula I to V are illustrated herein. Compounds of Formula I to V, or pharmaceutically acceptable salts or derivatives thereof, may be provided for use in the treatment or prevention of a disease or disorder that can be ameliorated by activation of long isoforms of PDE4. Compounds of Formula I to V, or pharmaceutically acceptable salts or derivatives thereof, may be provided for use in the treatment or prevention of a disease or disorder mediated by excessive intracellular cAMP signalling.

Also described herein are compounds of Formula Ib to Vb, or pharmaceutically acceptable salts or derivatives thereof, as set out below. Compounds of Formula Ib to Vb, or pharmaceutically acceptable salts or derivatives thereof, may be provided for use in the treatment or prevention of a disease or disorder that can be ameliorated by activation of long isoforms of PDE4. Compounds of Formula Ib to Vb, or pharmaceutically acceptable salts or derivatives thereof, may be provided for use in the treatment or prevention of a disease or disorder mediated by excessive intracellular cAMP signalling.

Described herein is a compound of Formula Ib:

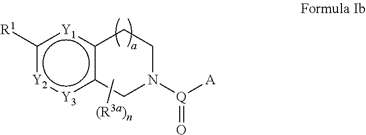

or a pharmaceutically acceptable salt or derivative thereof, wherein:
one or two of Y₁, Y₂ and Y₃ are N and the other(s) are CR^{3b};
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

Also described is a compound of Formula IIb:

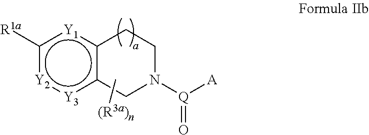

or a pharmaceutically acceptable salt or derivative thereof, wherein:
one or two of Y₁, Y₂ and Y₃ are N and the others are each CR^{3b};
Q is C or S(O);
R^{1a} is a 7- to 9-membered saturated, bridged ring system containing 2 ring N heteroatoms, and wherein R^{1a} is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered ring, optionally additionally containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

Also described is a compound of Formula IIIb:

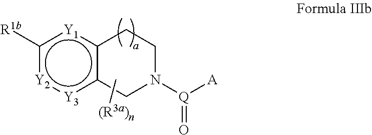

or a pharmaceutically acceptable salt or derivative thereof, wherein:
one or two of Y₁, Y₂ and Y₃ are N and the others are each CR^{3b};
Q is C or S(O);
R^{1b} is a 4- to 10-membered non-aromatic ring that may monocyclic, bridged or bicyclic containing at least 1 ring N heteroatom and optionally a ring O heteroatom, wherein at least 1 ring N heteroatom is not at the point of attachment of R^{1b}, and wherein R^{1b} is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered ring, optionally additionally containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1;
wherein when one of X₁ and X₂ is N and the other is S and X₃ is C, R^{1b} is not a monocyclic piperazine ring.

Also described is a Formula IVb:

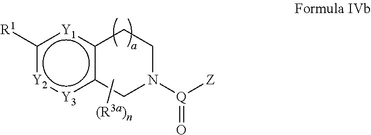

or a pharmaceutically acceptable salt or derivative thereof, wherein:
one or two of Y₁, Y₂ and Y₃ are N and the others are each CR^{3b};
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
Z is NR^{2d}R^{2b} or R^{2e};
R^{2b} is H or (C1-6)alkyl, and wherein (C1-6)alkyl is optionally substituted with 1 or more R⁵; and
R^{2d} is
   a) a (C5-10)alkyl group comprising a cyclic moeity; and wherein R^{2d} is optionally substituted with 1 or more R⁵; or
   b) a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2d} is substituted with 1 or more R⁵ (optionally wherein R⁵ is halogen);
R^{2e} is
   a) a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms; and wherein R^{2e} is optionally substituted with 1 or more R⁵; or
   b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; and wherein R^{2e} is substituted with 1 or more R⁵ (optionally wherein R⁵ is halogen);
each R^{3a} is independently (C1-6)alkyl, optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

Also described is a compound of of Formula Vb:

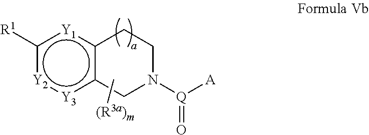

or a pharmaceutically acceptable salt or derivative thereof, wherein:
one or two of Y₁, Y₂ and Y₃ are N and the other(s) are CR^{3b};
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
m is 1, 2, 3 or 4; and
a is 0 or 1.

In the compounds of Formula I, R¹ is a 4- to 10-membered non-aromatic ring that may be monocyclic, bridged or bicyclic containing at least 1 ring N heteroatom and optionally a ring O heteroatom, and wherein R¹ is optionally substituted with 1 or more R⁴. The monocyclic, bridged or bicyclic ring may be saturated, or partially saturated, or in the case of a bicyclic ring, a combination thereof. It will be appreciated that the ring N atom in a saturated or partially saturated ring, when unsubstituted, may be NH (as valency allows). It will also be appreciated that no further ring heteroatoms are present other than the “at least 1 ring N heteroatom” (i.e. 1 or more ring N heteroatoms) and the optional “ring O heteroatom”.

In the compounds of Formula Ib, R¹ is a 4- to 10-membered monocyclic, bridged or bicyclic ring containing at least 1 ring N heteroatom and optionally a ring O heteroatom, and wherein R¹ is optionally substituted with 1 or more R⁴. The monocyclic, bridged or bicyclic ring may be saturated, partially saturated or aromatic, or in the case of a bicyclic ring, a combination thereof. It will be appreciated that the ring N atom in a saturated or partially saturated ring, when unsubstituted, may be NH (as valency allows). It will also be appreciated that no further ring heteroatoms are present other than the “at least 1 ring N heteroatom” (i.e. 1 or more ring N heteroatoms) and the optional “ring O heteroatom”.

In embodiment (1) of Formula I or Ib, R¹ comprises at least 1 ring N heteroatom not at the point of attachment of R¹ (i.e. a ring N atom must be present at a position that is not the point of attachment of R¹ to the ring containing Y₁, Y₂ and Y₃). The remaining moieties may be as defined for Formula I or Ib or any of embodiments (6)-(31) of Formula I or Ib described herein, mutatis mutandis.

In embodiment (2) of Formula I or Ib, R¹ is a 4- to 10-membered ring that may be monocyclic, bridged or bicyclic containing 1 ring N heteroatom, 2 ring N heteroatoms or 1 ring N heteroatom and 1 ring O heteroatom, and wherein R¹ is optionally substituted with 1 or more R⁴. R¹ may comprise at least 1 ring N heteroatom not at the point of attachment of R¹. The remaining moieties may be as defined for Formula I or Ib or any of embodiments (6)-(31) of Formula I or Ib described herein, mutatis mutandis.

In embodiment (3) of Formula I, R¹ is a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom or a 7- to 8-membered saturated, bridged ring containing 1 or 2 ring N heteroatoms; a 9-membered saturated, bridged ring system containing 2 ring N heteroatoms and a ring O-heteroatom; or a 7- to 10-membered saturated, fused or spiro ring system system containing 1 or 2 ring N heteroatoms, optionally 2 ring N heteroatoms; and R¹ is optionally substituted with 1 or more R⁴, optionally wherein R¹ is optionally substituted with 1, 2 or 3 R⁴. R¹ may comprise at least 1 ring N heteroatom not at the point of attachment of R¹. The remaining moieties may be as defined for Formula I or any of embodiments (6)-(31) of Formula I described herein, mutatis mutandis.

In embodiment (3) of Formula Ib, R¹ is a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); a 5- to 6-membered aromatic, monocyclic ring containing 1 or 2 ring N heteroatoms; or a 7- to 8-membered saturated, bridged ring containing 1 or 2 ring N heteroatoms; a 9-membered saturated, bridged ring system containing 2 ring N heteroatoms and a ring O-heteroatom; or a 7- to 10-membered saturated, fused or spiro ring system system containing 1 or 2 ring N heteroatoms, optionally 2 ring N heteroatoms; and R¹ is optionally substituted with 1 or more R⁴, optionally wherein R¹ is optionally substituted with 1, 2 or 3 R⁴. R¹ may comprise at least 1 ring N heteroatom not at the point of attachment of R¹. The remaining moieties may be as defined for Formula Ib or any of embodiments (6)-(31) of Formula Ib described herein, mutatis mutandis.

In Formula I or any of the options for embodiments (1), (2) or (3), R¹ may be a 4- to 10-membered monocyclic, bridged or bicyclic ring containing at least 1 ring N heteroatom (i.e. with no ring O heteroatom). R¹ may be a 6-membered saturated monocyclic ring containing 1 or 2 ring N heteroatoms, or a 7- to 8-membered saturated, bridged ring system containing 1 or 2 ring N heteroatoms, and R¹ is optionally substituted with 1 or more R⁴. R¹ may be a 6-membered saturated monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and R¹ is optionally substituted with 1 or more R⁴. R¹ may be a 6-membered saturated monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and wherein R¹ is optionally substituted with 1 R⁴. R¹ may be a 6-membered saturated monocyclic ring containing 2 ring N heteroatoms; or a 7- to 8-membered saturated, bridged ring system containing 2 ring N heteroatoms, wherein R¹ is optionally substituted with 1 R⁴. R¹ may be a 7- to 8-membered saturated, bridged ring system containing 2 ring N heteroatoms, wherein R¹ is optionally substituted with 1 R⁴, R¹ may be a 7- to 8-membered saturated, bridged ring system containing 2 ring N heteroatoms, for example a bridged piperazine, such as 3,8-diazabicyclo[3.2.1]octanyl, wherein R¹ is optionally substituted with 1 R⁴. The remaining moieties may be as defined for Formula I or any of embodiments (6)-(31) of Formula I described herein, mutatis mutandis.

In Formula Ib or any of the options for embodiments (1), (2) or (3), R¹ may be a 4- to 10-membered monocyclic, bridged or bicyclic ring containing at least 1 ring N heteroatom (i.e. with no ring O heteroatom). R¹ may be a 6-membered saturated or aromatic monocyclic ring containing 1 or 2 ring N heteroatoms, or a 7- to 8-membered saturated, bridged ring system containing 1 or 2 ring N heteroatoms, and R¹ is optionally substituted with 1 or more R⁴. R¹ may be a 6-membered saturated or aromatic monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and R¹ is optionally substituted with 1 or more R⁴. R¹ may be a 6-membered saturated or aromatic monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and wherein R¹ is optionally substituted with 1 R⁴. R¹ may be a 6-membered saturated or aromatic monocyclic ring containing 2 ring N heteroatoms; or a 7- to 8-membered saturated, bridged ring system containing 2 ring N heteroatoms, wherein R¹ is optionally substituted with 1 R⁴. R¹ may be a 7- to 8-membered saturated, bridged ring system containing 2 ring N heteroatoms, wherein R¹ is optionally substituted with 1 R⁴, R¹ may be a 7- to 8-membered saturated, bridged ring system containing 2 ring N heteroatoms, for example a bridged piperazine, such as 3,8-diazabicyclo[3.2.1]octanyl, wherein R¹ is optionally substituted with 1 R⁴. The remaining moieties may be as defined for Formula Ib or any of embodiments (6)-(31) of Formula Ib described herein, mutatis mutandis.

In Formula I or any of the options for embodiments (1), (2) or (3), R¹ may be piperidinyl, piperazinyl, morpholinyl, pyrrolidinyl, azetidinyl, 2,5-diazabicyclo[2.2.1]heptanyl, 2,5-diazabicyclo[2.2.2]octanyl or 3,8-diazabicyclo[3.2.1]octanyl, 3,9-diazabicyclo[3.3.1]nonanyl, 3,6-diazabicyclo[3.1.1]heptanyl, 4,7-diazaspiro[2.5]octanyl, 2,6-diazaspiro[3.3]heptanyl, 2,6-diazaspiro[3.4]octanyl, 2,7-diazaspiro[3.5]nonanyl, octahydro-4H-pyrrolo[3,2-b]pyridinyl, octahydro-5H-pyrrolo[3,2-c]pyridinyl or hexahydropyrrolo[3,4-c]pyrrol-(1H)-yl, each of which is optionally substituted with 1 or more R⁴, preferably optionally substituted with 1-3 R⁴, preferably optionally substituted with 1 R⁴. R¹ may be a group of structure:

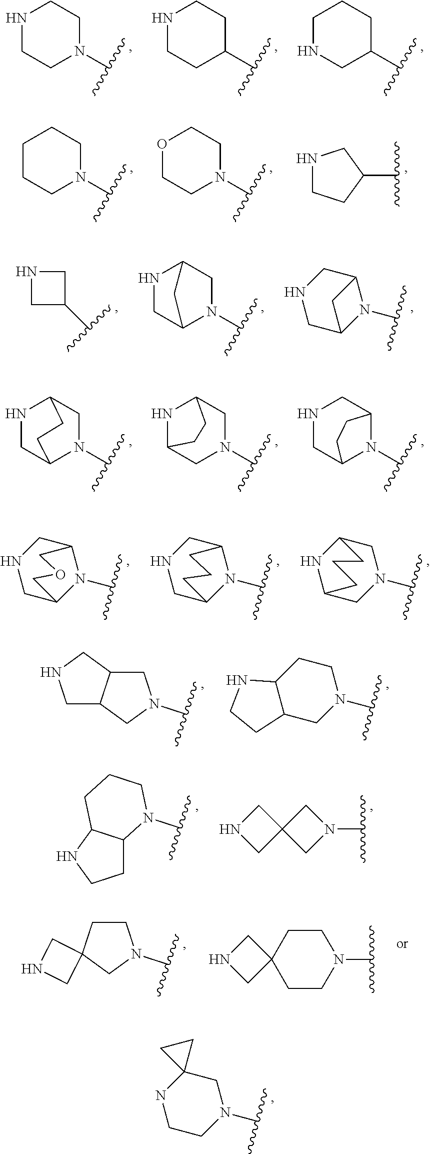

and wherein R¹ is optionally substituted with 1 or more R⁴, optionally wherein R¹ is optionally substituted with 1-3 R⁴. R¹ may be piperidinyl, piperazinyl, pyrrolidinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R¹ may be piperidinyl or piperazinyl, each of which is optionally substituted with 1 or more R⁴, preferably optionally substituted with 1-3 R⁴, preferably optionally substituted with 1 R⁴. R¹ may be: a group of structure

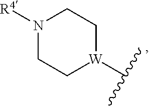

wherein W is CH or N and R⁴ is H or R⁴. R¹ may be a 7- to 8-membered saturated, bridged ring system containing 2 ring N heteroatoms, for example a bridged piperazine such as

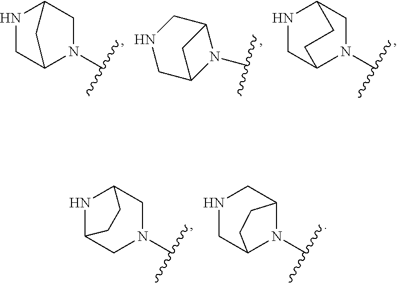

The remaining moieties may be as defined for Formula I or any of embodiments (6)-(31) of Formula I described herein, mutatis mutandis.

In Formula Ib or any of the options for embodiments (1), (2) or (3), R¹ may be piperidinyl, piperazinyl, morpholinyl, pyrrolidinyl, pyrazolyl, imidazolyl, pyridinyl, azetidinyl, 2,5-diazabicyclo[2.2.1]heptanyl, 2,5-diazabicyclo[2.2.2]octanyl or 3,8-diazabicyclo[3.2.1]octanyl, 3,6-diazabicyclo[3.1.1]heptanyl, 4,7-diazaspiro[2.5]octanyl, 2,6-diazaspiro[3.3]heptanyl, 2,6-diazaspiro[3.4]octanyl, 2,7-diazaspiro[3.5]nonanyl, octahydro-4H-pyrrolo[3,2-b]pyridinyl, octahydro-5H-pyrrolo[3,2-c]pyridinyl or hexahydropyrrolo[3,4-c]pyrrol-(1H)-yl, each of which is optionally substituted with 1 or more R⁴, preferably optionally substituted with 1-3 R⁴, preferably optionally substituted with 1 R⁴. R¹ may be a group of structure:

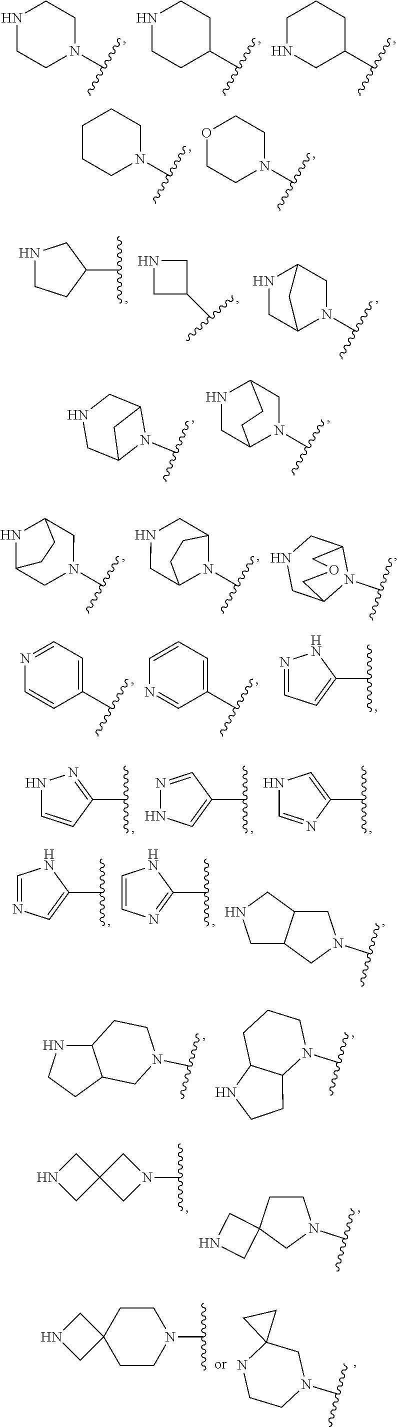

and wherein R¹ is optionally substituted with 1 or more R⁴, optionally wherein R¹ is optionally substituted with 1-3 R⁴. R¹ may be piperidinyl, piperazinyl, pyrrolidinyl, pyrazolyl, imidazolyl, pyridinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R¹ may be piperidinyl, piperazinyl or pyridinyl, each of which is optionally substituted with 1 or more R⁴, preferably optionally substituted with 1-3 R⁴, preferably optionally substituted with 1 R⁴. R¹ may be: a group of structure

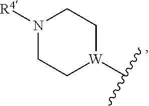

wherein Wis C H or N and R^{4′} is H or R⁴; or pyridyl (optionally 3-pyridyl) optionally substituted with 1 R⁴. R¹ may be a 7- to 8-membered saturated, bridged ring system containing 2 ring N heteroatoms, for example a bridged piperazine such as

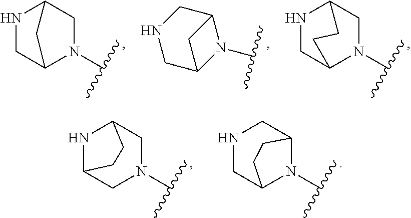 [SMILES: *N1CC2CC1CN2 ; *N1C2CNCC1C2 ; *N1CC2CCC1CN2 ; *N1CC2CCC(C1)N2 ; *N1C2CCC1CNC2]

The remaining moieties may be as defined for Formula Ib or any of embodiments (6)-(31) of Formula Ib described herein, mutatis mutandis.

In embodiment (4) of Formula I or Ib, R¹ is a 5- to 6-membered saturated, monocyclic ring containing at least 1 ring N heteroatom and optionally a ring O heteroatom; or a 7- to 8-membered saturated, bridged ring containing 1 or 2 ring N heteroatoms, and wherein R¹ is optionally substituted with 1, 2 or 3 R⁴. In any of the options for embodiment (4), R¹ may comprise at least 1 ring N heteroatom not at the point of attachment of R¹. R¹ may be a 6-membered saturated monocyclic ring containing 1 or 2 ring N heteroatoms, or a 7- to 8-membered saturated, bridged ring system containing 1 or 2 ring N heteroatoms. R¹ may be a 6-membered saturated monocyclic ring containing 1 or 2 ring N heteroatoms, or a 7- to 8-membered saturated, bridged ring system containing 1 or 2 ring N heteroatoms, wherein R¹ is optionally substituted with 1 R⁴. The remaining moieties may be as defined for Formula I or Ib or any of embodiments (6)-(31) of Formula I or Ib described herein, mutatis mutandis.

In embodiment (5) of Formula I or Ib, R¹ is a 7- to 9-membered saturated, bridged ring system containing 2 ring N heteroatoms, and wherein R¹ is optionally substituted with 1 or more R⁴. R¹ may be a 7- to 9-membered saturated, bridged ring system containing 2 ring N heteroatoms, and wherein R^{1a} is optionally substituted with 1, 2 or 3 R⁴. R¹ may be an optionally substituted 7- to 8-membered saturated, bridged ring system containing 2 ring N heteroatoms. The remaining moieties may be as defined for Formula I or Ib or any of embodiments (6)-(31) of Formula I or Ib described herein, mutatis mutandis.

In Formula I or any of the options for embodiments (1)-(5), R¹ may be substituted with 1 or more R⁴. Where R¹ contains a substitutable ring N atom, R¹ may be substituted on a substitutable ring N atom. R¹ may be substituted by 1 R⁴, preferably on a ring N atom. In embodiments where R¹ is saturated ring, R¹ may be substituted by 1 R⁴, preferably on a ring N atom. In embodiments where R¹ is a 6-membered ring, R¹ may be substituted by 1 R⁴. In embodiments where R¹ is a 5-membered ring, R¹ may be substituted by 1, 2 or 3 R⁴.

In Formula Ib or any of the options for embodiments (1)-(5), R¹ may be substituted with 1 or more R⁴. Where R¹ contains a substitutable ring N atom, R¹ may be substituted on a substitutable ring N atom. In embodiments where R¹ is saturated ring, R¹ may be substituted by 1 R⁴, preferably on a ring N atom. In embodiments where R¹ is an aromatic ring, R¹ may be substituted by 1, 2 or 3 R⁴. In embodiments where R¹ is a 6-membered ring, R¹ may be substituted by 1 R⁴. In embodiments where R¹ is a 5-membered ring, R¹ may be substituted by 1, 2 or 3 R⁴.

In the compounds of Formula I and Ib, each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and (C1-6)alkylene-(C1-6)alkoxy being optionally substituted with 1 or more substituents independently selected from halogen, OH and (C1-6)alkoxy. Each R⁴ may, independently, represent a substituent on a carbon atom or a substitutable N atom.

In embodiment (6) of Formula I or Ib, each R⁴ is independently halogen, OH, CN, (C1-4)alkyl, (C1-3)alkoxy, (C3-6)cycloalkyl or —(C1-3)alkylene-(C1-3)alkoxy, the (C1-3)alkyl, (C1-3)alkoxy, (C3-6)cycloalkyl and —(C1-3)alkylene-(C1-3)alkoxy being optionally substituted with 1 or more substituents independently selected from halogen, OH and (C1-3)alkoxy. Each R⁴ may independently be F, Cl, OH, CN, (C1-4)alkyl, methoxy, ethoxy, cyclopropyl or —(CH₂)₂—O—(CH₂)₂—O—CH₃, the (C1-4)alkyl being optionally substituted with 1 or more substituents independently selected from halogen and OH. The remaining moieties may be as defined for Formula I or Ib or any of embodiments (1)-(5) or (9)-(31) of Formula I or Ib described herein, mutatis mutandis.

In embodiment (7) of Formula I or Ib, each R⁴ is independently halogen, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy. Each R⁴ may independently be halogen, OH, (C1-4)alkyl, (C1-3)alkoxy, (C3-6)cycloalkyl or —(C1-3)alkylene-(C1-3)alkoxy, the (C1-3)alkyl, (C1-3)alkoxy, (C3-6)cycloalkyl and —(C1-3)alkylene-(C1-3)alkoxy being optionally substituted with 1 or more substituents independently selected from halogen, OH and (C1-3)alkoxy. Each R⁴ may independently be F, Cl, OH, (C1-4)alkyl, methoxy, ethoxy, cyclopropyl or —(CH₂)₂—O—(CH₂)₂—O—CH₃, the (C1-4)alkyl being optionally substituted with 1 or more substituents independently selected from halogen and OH. The remaining moieties may be as defined for Formula I or Ib or any of embodiments (1)-(5) or (9)-(31) of Formula I or Ib described herein, mutatis mutandis.

In embodiment (8) of Formula I or Ib, each R⁴ is independently halogen, CN, OH, (C1-2)alkyl, (C1-6)alkoxy, or —(C1-6)alkylene-(C1-6)alkoxy, the (C1-2)alkyl, (C1-6)alkoxy and —(C1-6)alkylene-(C1-6)alkoxy being optionally substituted with 1 or more substituents independently selected from halogen, OH and (C1-6)alkoxy. Each R⁴ may independently be F, Cl, OH, (C1-2)alkyl, methoxy, ethoxy or —(CH₂)₂—O—(CH₂)₂—O—CH₃, the (C1-2)alkyl being optionally substituted with 1 or more substituents independently selected from halogen and OH. The remaining moieties may be as defined for Formula I or Ib or any of embodiments (1)-(5) or (9)-(31) of Formula I or Ib described herein, mutatis mutandis.

In Formula I or Ib or any of the options for embodiments (6)-(8), when attached to a ring N atom, R⁴ may independently be any of the options identified herein for R⁴, except for halogen, CN, OH, and —(C1-6)alkoxy.

In the compounds of Formula I, A is NR^{2a}R^{2b}, R^{2c}, or OR^{2f}, wherein
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or 6-membered aromatic or heteroaromatic ring that contains 0, 1 or 2 ring N atoms;
or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵.

In the compounds of Formula Ib, A is NR^{2a}R^{2b} or R^{2c}, wherein
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof;
(C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵.

In the compounds of Formula I and Ib, each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH.

In embodiment (9) of Formula I or Ib, each R⁵ is independently halogen, OH, CN, (C1-4)alkyl, or (C1-4)alkoxy, the (C1-4)alkyl and (C1-4)alkoxy group being optionally substituted with 1 or more halogen or OH, preferably optionally substituted with 1 or more fluoro or 1 OH. When substituted on an aliphatic group, each R⁵ may independently be halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH; and when substituted on an aromatic group, each R⁵ may independently be halogen, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH. Each R⁵ may independently be halogen, CN, (C1-4)alkyl, or (C1-4)alkoxy, the (C1-4)alkyl and (C1-4)alkoxy group being optionally substituted with 1 or more halogen or OH, preferably optionally substituted with 1 or more fluoro or 1 OH. Each R⁵ may independently be halogen, CN or (C1-4)alkyl, the (C1-4)alkyl group being optionally substituted with 1 or more halogen, preferably optionally substituted with 1 or more fluoro. Each R⁵ may independently be halogen (preferably fluoro), CN or CF₃. Each R⁵ may independently be halogen (preferably fluoro). The remaining moieties may be as defined for Formula I or Ib or any of embodiments (1)-(8) or (10)-(31) of Formula I or Ib described herein, mutatis mutandis.

In embodiment (10) of Formula I or Ib, R^{2a} is a 6-membered aromatic or heteroaromatic ring that contains 0, 1 or 2 ring N atoms, wherein R^{2a} is optionally substituted with 1 or more R⁵. R^{2a} may be substituted with 0, 1 or 2 R⁵, preferably 0 or 1 R⁵. R^{2a} may be substituted with halogen, CN or (C1-4)alkyl, the (C1-4)alkyl group being optionally substituted with 1 or more halogen, preferably optionally substituted with 1 or more fluoro. R^{2a} may be substituted with halogen (preferably fluoro), CN or CF₃. The remaining moieties may be as defined for Formula I or Ib or any of embodiments (1)-(9), (13) or (21)-(31) of Formula I or Ib described herein, mutatis mutandis.

In embodiment (11) of Formula I or Ib, R^{2a} is a CH₂-[6-membered aromatic or heteroaromatic ring that contains 0, 1 or 2 ring N atoms], wherein R^{2a} is optionally substituted with 1 or more R⁵. It will be appreciated that substitution by R⁵ is possible on the —CH₂— linker or aromatic or heteroaromatic ring of R^{2a}. R^{2a} is a CH₂-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃. The remaining moieties may be as defined for Formula I or Ib or any of embodiments (1)-(9), (13) or (21)-(31) of Formula I or Ib described herein, mutatis mutandis.

In embodiment (12) of Formula I or Ib, R^{2a} is a (C2-10)alkyl group that may be straight chain, branched or cyclic, or a combination thereof, wherein R^{2a} is optionally substituted with 1 or more R⁵. R^{2a} may be substituted with 0, 1 or 2 R⁵, preferably 0 or 1 R⁵. R^{2a} may be a (C3-10)alkyl group that may be straight chain, branched or cyclic, or a combination thereof, wherein R^{2a} is optionally substituted with R⁵. R^{2a} may be a (C4-10)alkyl group that may be straight chain, branched or cyclic, or a combination thereof, wherein R^{2a} is optionally substituted with R⁵. R^{2a} may be a (C5-10)alkyl group that may be straight chain, branched or cyclic, or a combination thereof, wherein R^{2a} is optionally substituted with R⁵. R^{2a} may be a (C5-10)alkyl group comprising a cyclic moiety, wherein R^{2a} is optionally substituted with R⁵. The remaining moieties may be as defined for Formula I or Ib or any of embodiments (1)-(9), (13) or (21)-(31) of Formula I or Ib described herein, mutatis mutandis.

In any of embodiments (10)-(12) of Formula I or Ib, R^{2a} may be substituted with 1 or more R⁵. R⁵ may preferably be substituted by halogen (for example, fluoro).

In embodiment (13) of Formula I or Ib, R^{2b} is H or (C1-3)alkyl, and wherein (C1-3)alkyl is optionally substituted with 1 or more R⁵. R^{2b} may be H, CH₃ or CH₂CH₃. The remaining moieties may be as defined for Formula I or Ib or any of embodiments (1)-(12) or (21)-(31) of Formula I or Ib described herein, mutatis mutandis.

In embodiment (14) of Formula I or Ib, R^{2a} and R^{2b}, together with the N atom to which they are attached, form a 5- to 7-membered non-aromatic heterocycle, optionally containing 1 further heteroatom selected from O, and optionally substituted with 1 or more R⁵. R^{2a} and R^{2b}, together with the N atom to which they are attached, may form a 5- to 7-membered non-aromatic heterocycle, optionally substituted with 1 or more R⁵. R^{2a} and R^{2b}, together with the N atom to which they are attached, may form a 5-membered non-aromatic heterocycle, optionally substituted with 1 or more R⁵. A ring formed by R^{2a} and R^{2b} together may be substituted with 0 or 1 R⁵. The remaining moieties may be as defined for Formula I or Ib or any of embodiments (1)-(9) or (21)-(31) of Formula I or Ib described herein, mutatis mutandis.

In embodiment (15) of Formula I or Ib, A is R^{2c}. Preferably, Q is also C.

In embodiment (16) of Formula I or Ib, R^{2c} is CH₂-[6-membered aromatic or heteroaromatic ring that contains 0, 1 or 2 ring N atoms], wherein R^{2c} is optionally substituted with 1 or more R⁵. It will be appreciated that substitution by R⁵ is possible on the —CH₂— linker or aromatic or heteroaromatic ring of R^{2c}. R^{2c} may be a CH₂-[6-membered aromatic or heteroaromatic ring that contains 0, 1 or 2 ring N atoms], wherein the CH₂ is optionally substituted with 1 or 2 halogen (preferably fluoro, to form —CHF— or —CF₂—) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃. The remaining moieties may be as defined for Formula I or Ib or any of embodiments (1)-(9), (15) or (21)-(31) of Formula I or Ib described herein, mutatis mutandis.

In embodiment (17) of Formula I or Ib, R^{2c} is CH₂—O-[6-membered aromatic or heteroaromatic ring that contains 0, 1 or 2 ring N atoms], wherein R^{2c} is optionally substituted with 1 or more R⁵. It will be appreciated that substitution by R⁵ is possible on the —CH₂— linker or aromatic or heteroaromatic ring of R^{2c}. R^{2c} may be a CH₂—O-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃. The remaining moieties may be as defined for Formula I or Ib or any of embodiments (1)-(9), (15) or (21)-(31) of Formula I or Ib described herein, mutatis mutandis.

In embodiment (18) of Formula I or Ib, R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; wherein R^{2c} is optionally substituted with 1 or more R⁵ (for example, 1 or 2 R⁵, wherein R⁵ is fluoro or OH, preferably fluoro). R^{2c} may be a (C3-10)alkyl group comprising a cyclic moeity, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms. and wherein R^{2c} is optionally substituted with 1 or more R⁵ (for example, 1 or 2 R⁵, wherein R⁵ is fluoro or OH, preferably fluoro). R^{2c} may be a (C3-10)alkyl group comprising a cyclic moeity, wherein a C atom in straight chain or cyclic portion of said (C3-10)alkyl group may be optionally replaced by 1 —O— other than at the point of attachment of R^{2c} and wherein R^{2c} is optionally substituted with 1 or more R⁵ (for example, 1 or 2 R⁵, wherein R⁵ is fluoro or OH, preferably fluoro). R^{2c} may be a (C3-10)alkyl group comprising a cyclic moeity, wherein R^{2c} is optionally substituted with 1 or more R⁵ (for example, 1 or 2 R⁵, wherein R⁵ is fluoro or OH, preferably fluoro). R^{2c} may preferably be substituted on a cyclic moiety, for example by 2 R⁵ optionally on the same carbon atom. The remaining moieties may be as defined for Formula I or Ib or any of (1)-(9), (15) or (21)-(31) of Formula I or Ib described herein, mutatis mutandis.

In embodiment (19) of Formula I or Ib, R^{2c} is 6-membered aromatic or heteroaromatic ring that contains 0, 1 or 2 ring N atoms, wherein R^{2c} is optionally substituted with 1 or more R⁵. R^{2c} may be a 6-membered aromatic or heteroaromatic ring that contains 0, 1 or 2 ring N atoms, wherein R^{2c} is optionally substituted with 1 or 2 R⁵, for example halogen (preferably fluoro), (C1-4)alkoxy, or (C1-4)alkyl, the (C1-4)alkyl group being optionally substituted with 1 or more halogen (preferably optionally substituted with 1 or more fluoro). The remaining moieties may be as defined for Formula I or Ib or any of (1)-(9), (15) or (21)-(31) of Formula I or Ib described herein, mutatis mutandis.

In any of embodiments (16)-(19) of Formula I or Ib, R^{2c} may be substituted with 1 or more R⁵. R⁵ may be halogen, for example fluoro.

In embodiment (20) of Formula I, R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; and wherein R^{2f} is optionally substituted with 1 or more R⁵. It will be appreciated that substitution by R⁵ is possible on the —CH₂— linker or aromatic or heteroaromatic ring of R^{2f}. R^{2f} may be CH₂-[6-membered aromatic or heteroaromatic ring that contains 0, 1 or 2 ring N atoms]; or a (C2-10)alkyl group that may be straight chain, branched or cyclic, or a combination thereof; and wherein R^{2f} is optionally substituted with 1 or more R⁵. The remaining moieties may be as defined for Formula I or any of embodiments (1)-(9) or (21)-(31) of Formula I described herein, mutatis mutandis.

In Formula I or Ib or any of embodiments (10)-(20) of Formula I or Ib, when substituted on a 6-membered aromatic or heteroaromatic ring that contains 0, 1 or 2 ring N atoms, R⁵ may independently be halogen, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH.

In the compounds of Formula I and Ib, each R^{3a} is independently (C1-6)alkyl or fluoro, the (C1-6)alkyl being optionally substituted by 1 or more halogen; or two R^{3a} attached to the same or adjacent carbon atoms may be joined together with the atoms to which they are attached to form a 3- to 6-membered carbocyclic ring or heterocyclic ring containing an O heteroatom, wherein said ring is optionally substituted by 1 or more halogen; and each R^{3b} is independently H or (C1-6)alkyl.

In embodiment (21) of Formula I or Ib, each R^{3a} is independently (C1-3)alkyl or fluoro, the (C1-3)alkyl being optionally substituted by 1 or more halogen and/or each R^{3b} is independently H or (C1-3)alkyl. Each R^{3a} may be —CH₃ or F. Each R^{3b} may be —CH₃ or H. The remaining moieties may be as defined for Formula I or Ib or any of embodiments (1)-(20) or (23)-(31) of Formula I or Ib described herein, mutatis mutandis.

In embodiment (22) of Formula I or Ib, two R^{3a} attached to the same or adjacent carbon atoms may be joined together with the atoms to which they are attached to form a 3- to 6-membered ring, wherein said ring is optionally substituted by 1 or more halogen. Two R^{3a} attached to the same or adjacent carbon atoms may be joined together with the atoms to which they are attached to form a 3- to 6-membered ring. It will be appreciated that when two R^{3a} are attached to the same carbon atom, a spiro ring will be formed and when two R^{3a} are attached to adjacent carbon atoms, a fused ring will be formed. Two R^{3a} attached to the same carbon may be joined together with the atom to which they are attached to form a cyclopropyl ring. The remaining moieties may be as defined for Formula I or Ib or any of embodiments (1)-(20) or (23)-(31) of Formula I or Ib described herein, mutatis mutandis.

In embodiment (23) of Formula I or Ib, each R^{3b} may independently be H or (C1-3)alkyl. Each R^{3b} may be —CH₃ or H. Preferably, each R^{3b} is H. The remaining moieties may be as defined for Formula I or Ib or any of embodiments (1)-(22) or (24)-(31) of Formula I or Ib described herein, mutatis mutandis.

In the compounds of Formula I and Ib, n is 0, 1, 2, 3 or 4.

In embodiment (24) of Formula I or Ib, n is 0. The remaining moieties may be as defined for Formula I or Ib or any of embodiments (1)-(23) or (26)-(31) of Formula I or Ib described herein, mutatis mutandis.

In embodiment (25) of Formula I or Ib, n is 0, 1, 2 or 3. Preferably, n is 0, 1 or 2. The remaining moieties may be as defined for Formula I or Ib or any of embodiments (1)-(23) or (26)-(31) of Formula I or Ib described herein, mutatis mutandis.

In the compounds of Formula I and Ib, Q is C or S(O).

In embodiment (26) of Formula I or Ib, Q is C. The remaining moieties may be as defined for Formula I or Ib or any of embodiments (1)-(25) or (27)-(31) of Formula I or Ib described herein, mutatis mutandis.

In the compounds of Formula I and Ib, one or two of Y₁, Y₂ and Y₃ are N and the other(s) are CR^{3b}.

In embodiment (27) of Formula I or Ib, one of Y₁, Y₂ and Y₃ is N and the others are each CR^{3b}. The remaining moieties may be as defined for Formula I or Ib or any of embodiments (1)-(26) or (29)-(31) of Formula I or Ib described herein, mutatis mutandis.

In embodiment (28), of Formula I or Ib, Y₁, is N and Y₂ and Y₃ are CR^{3b}. Accordingly, the compound of Formula I or Ib may be a compound of structure:

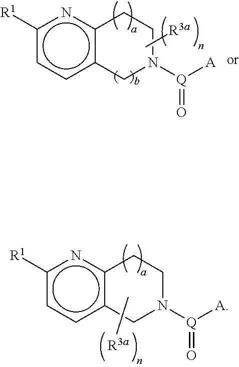

wherein R¹, Q, A, R^{3a}, n, a and b are as defined for Formula I or Ib or any of embodiments (1)-(26) or (29)-(31) of Formula I or Ib described herein.

In the compounds of Formula I, a is 0 or 1 and b is 1 or 2, wherein when b is 2, a is 0. In the compounds of Formula Ib, a is 0 or 1.

In embodiment (29) of Formula I or Ib, a is 0. The compound of Formula I or Ib may be:

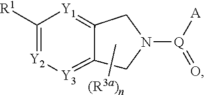

or a pharmaceutically acceptable salt or derivative thereof. The compound may be of formula:

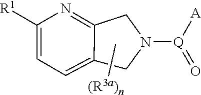

or a pharmaceutically acceptable salt or derivative thereof.

The remaining moieties may be as defined for Formula I or Ib or any of embodiments (1)-(28) of Formula I or Ib described herein, mutatis mutandis.

In embodiment (30) of Formula I or Ib, a is 1 and the compound of Formula I or Ib may be:

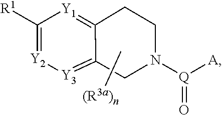

or a pharmaceutically acceptable salt or derivative thereof. The compound may be of formula:

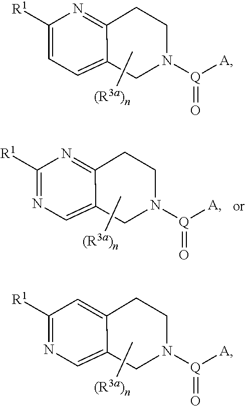

optionally

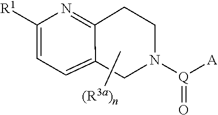

or a pharmaceutically acceptable salt or derivative thereof. The remaining moieties may be as defined for Formula I or Ib or any of embodiments (1)-(28) of Formula I or Ib described herein, mutatis mutandis.

In embodiment (31) of Formula I, a is 0 and b is 2 and the compound is:

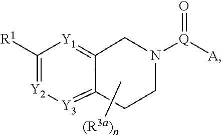

or a pharmaceutically acceptable salt or derivative thereof, optionally

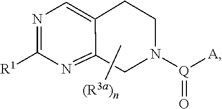

or a pharmaceutically acceptable salt or derivative thereof. The remaining moieties may be as defined for Formula I or any of embodiments (1)-(28) of Formula I described herein, mutatis mutandis.

In embodiment (32) of Formula I or Ib:
Q is C;
R¹ is a a 7- to 9-membered saturated, bridged ring system containing 2 ring N heteroatoms, wherein R¹ is optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2; and optionally
b is 1. The remaining moieties may be as defined for Formula I or Ib or any of embodiments (1)-(31) of Formula I or Ib described herein, mutatis mutandis.

In embodiment (33) of Formula I or Ib, the compound is a compound of structure:

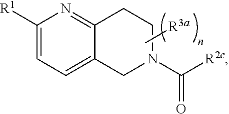

or a pharmaceutically acceptable salt or derivative thereof, wherein:
wherein:
R¹ is a 6-membered saturated monocyclic ring containing 2 ring N heteroatoms or a 7- to 9-membered saturated, bridged ring system containing 2 ring N heteroatoms, optionally substituted with 1 or more R⁴; and/or
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵. The remaining moieties may be as defined for Formula I or Ib or any of embodiments (1)-(31) of Formula I or Ib described herein, mutatis mutandis. For example, n may be 0, R^{2c} may be as defined according to embodiment (18) of Formula I or Ib.

Compounds of Formula I include compounds of Formulas II to V. Compounds of Formula Ib include compounds of Formulas IIb to Vb. Embodiments (1)-(24) of Formula I or Ib may apply mutatis mutandis to each of Formulas II to V or IIb to Vb.

Described herein is a compound of Formula II:

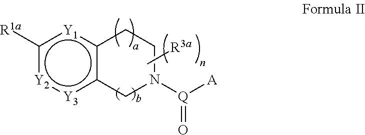

or a pharmaceutically acceptable salt or derivative thereof, wherein:
R^{1a} is a 7- to 9-membered saturated, bridged ring system containing 2 ring N heteroatoms, and wherein R^{1a} is optionally substituted with 1 or more R⁴;
and Y₁, Y₂, Y₃, R^{2a}, R^{2b}, R^{2c}, R^{2f}, R^{3a}, R^{3b}, A, Q, R⁴, R⁵, a, b and n are as defined for Formula I or any of embodiments (6)-(31) of Formula I above.

Also described herein is a compound of Formula IIb:

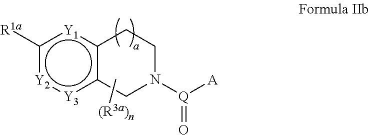

or a pharmaceutically acceptable salt or derivative thereof, wherein:
R^{1a} is a 7- to 9-membered saturated, bridged ring system containing 2 ring N heteroatoms, and wherein R^{1a} is optionally substituted with 1 or more R⁴;
and Y₁, Y₂, Y₃, R^{2a}, R^{2b}, R^{2c}, R^{3a}, R^{3b}, A, Q, R⁴, R⁵, a and n are as defined for Formula Ib or any of embodiments (6)-(31) of Formula Ib above.

In embodiment (1) of Formula II or IIb, R^{1a} is a 7- to 8-membered saturated, bridged ring system containing 2 ring N heteroatoms, wherein R¹ is optionally substituted with 1 or more R⁴, e.g. optionally substituted with 1, 2 or 3 R⁴. R^{1a} may be a 7- to 8-membered saturated, bridged ring system containing 2 ring N heteroatoms, wherein R¹ is optionally substituted with 1 R⁴. R^{1a} may be a bridged piperazine, such as 3,8-diazabicyclo[3.2.1]octanyl, wherein R^{1a} is optionally substituted with 1 R⁴. R^{1a} may be a bridged piperazine such as

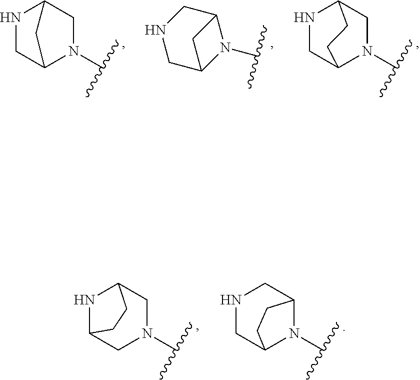 [SMILES: *N1CC2CC1CN2 ; *N1C2CNCC1C2 ; *N1CC2CCC1CN2 ; *N1CC2CCC(C1)N2 ; *N1C2CCC1CNC2]

In Formula II or IIb or any of the embodiments of Formula II or IIb, R^{1a} may be optionally substituted with 1 or more R⁴. Where R^{1a} contains a substitutable ring N atom, R^{1a} may preferably be substituted on a substitutable ring N atom. R^{1a} may be substituted by 1 R⁴, preferably on a ring N atom.

In embodiment (2) of Formula II or IIb, the compound is a compound of structure:

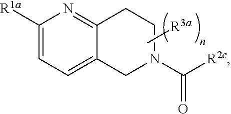

or a pharmaceutically acceptable salt or derivative thereof, wherein:
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵. The remaining moieties may be as defined for Formula I, Ib, II or IIb or any embodiments of Formula I, Ib, II or IIb described herein, mutatis mutandis. For example, n may be 0, R^{2c} may be as defined according to embodiment (18) of Formula I or Ib.

Described herein is a compound of Formula III:

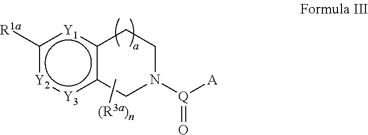

or a pharmaceutically acceptable salt or derivative thereof, wherein:
R^{1b} is a 4- to 10-membered non-aromatic ring that may be monocyclic, bridged or bicyclic containing at least 1 ring N heteroatom and optionally a ring O heteroatom, wherein at least 1 ring N heteroatom is not at the point of attachment of R^{1a}, and wherein R^{1a} is optionally substituted with 1 or more R⁴;
and Y₁, Y₂, Y₃, R^{2a}, R^{2b}, R^{2c}, R^{2f}, R^{3a}, R^{3b}, A, Q, R⁴, R⁵, a and n are as defined for Formula I or any of embodiments (6)-(31) of Formula I above.

Also described herein is a compound of Formula IIIb:

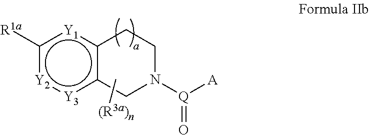

or a pharmaceutically acceptable salt or derivative thereof, wherein:
R^{1b} is a 4- to 10-membered non-aromatic ring that may be monocyclic, bridged or bicyclic containing at least 1 ring N heteroatom and optionally a ring O heteroatom, wherein at least 1 ring N heteroatom is not at the point of attachment of R^{1a}, and wherein R^{1a} is optionally substituted with 1 or more R⁴;
and Y₁, Y₂, Y₃, R^{2a}, R^{2b}, R^{2c}, R^{3a}, R^{3b}, A, Q, R⁴, R⁵, a and n are as defined for Formula Ib or any of embodiments (6)-(31) of Formula Ib above.

In a compound of Formula III or IIIb, R^{1b} comprises at least one ring N heteroatom not at the point of attachment to R^{1b}, i.e. a ring N atom must be present at a position that is not the point of attachment of R^{1b} to the ring containing Y₁, Y₂ and Y₃.

In embodiment (1) of Formula III or IIIb, R^{1b} is a 4- to 10-membered non-aromatic ring that may be monocyclic, bridged or bicyclic containing 1 ring N heteroatom, 2 ring N heteroatoms or 1 ring N heteroatom and 1 ring O heteroatom, and wherein R¹ is optionally substituted with 1 or more R⁴. R^{1b} may be a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); a 7- to 8-membered saturated, bridged ring system containing 1 or 2 ring N heteroatoms; or a 9-membered saturated, bridged ring system containing 2 ring N heteroatoms and a ring O-heteroatom; or a 7- to 10-membered saturated, fused or spiro ring system containing 1 or 2 ring N heteroatoms, and wherein R^{1b} is optionally substituted with 1 or more R⁴, optionally 1, 2 or 3 R⁴.

In embodiment (2) of Formula III or IIIb, R^{1b} is a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); or a 7- to 8-membered saturated, bridged ring containing 1 or 2 ring N heteroatoms, and wherein R^{1b} is optionally substituted with 1 or more R⁴, optionally 1, 2 or 3 R⁴. R^{1b} may be a 6-membered saturated monocyclic ring containing 1 or 2 ring N heteroatoms, optionally wherein at least 1 ring N heteroatom is not at the point of attachment of R^{1b}. R^{1b} may be a 6-membered saturated monocyclic ring containing 1 or 2 ring N heteroatoms, wherein R^{1b} is optionally substituted with 1 R⁴. R^{1b} may be a 7- to 8-membered saturated, bridged ring system containing 1 or 2 ring N heteroatoms, and wherein R^{1b} is optionally substituted with 1 or more R⁴, optionally 1, 2 or 3 R⁴. R^{1b} may be a 7- to 8-membered saturated, bridged ring system containing 2 ring N heteroatoms, for example a bridged piperazine, such as 3,8-diazabicyclo[3.2.1]octanyl, wherein R^{1b} is optionally substituted with 1 R⁴.

In Formula III or IIIb or any of the embodiments of Formula III or IIIb, R^{1b} may be piperidinyl, piperazinyl, morpholinyl, pyrrolidinyl, 2,5-diazabicyclo[2.2.1]heptanyl, 2,5-diazabicyclo[2.2.2]octanyl, 3,8-diazabicyclo[3.2.1]octanyl or 3,9-diazabicyclo[3.3.1]nonanyl, each of which is optionally substituted with 1 or more R⁴, preferably optionally substituted with 1-3 R⁴, preferably optionally substituted with 1 R⁴. R^{1b} may be group of structure:

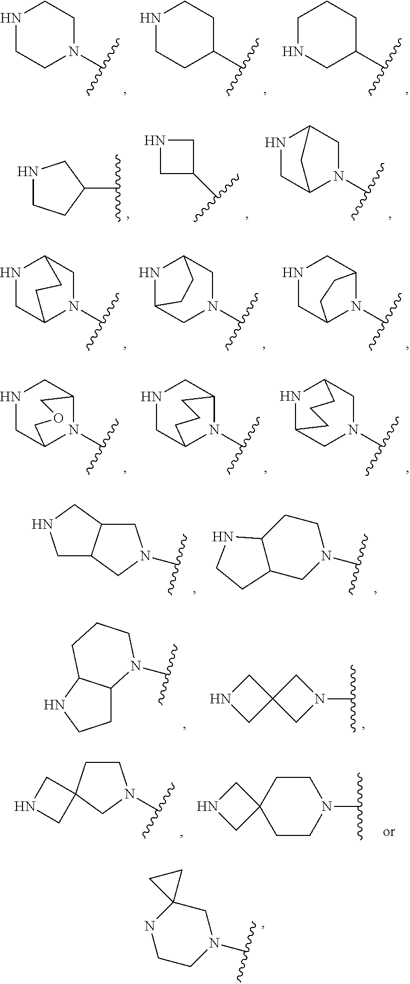

and wherein R^{1b} is optionally substituted with 1 or more R⁴, optionally wherein R^{1b} is optionally substituted with 1-3 R⁴, preferably optionally substituted with 1 R⁴. R^{1b} may be piperidinyl, piperazinyl, pyrrolidinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R^{1b} may be piperidinyl or piperazinyl, each of which is optionally substituted with 1 or more R⁴, preferably optionally substituted with 1-3 R⁴, preferably optionally substituted with 1 R⁴. R^{1b} may be a group of structure

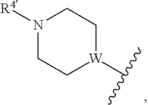

wherein W is CH or N and R^{4′} is H or R⁴. R^{1b} may be a 7- to 8-membered saturated, bridged ring system containing 2 ring N heteroatoms, for example a bridged piperazine such as

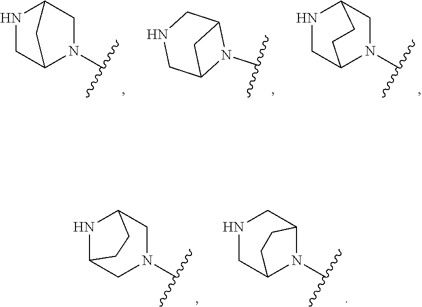 [SMILES: *N1CC2CC1CN2 ; *N1C2CNCC1C2 ; *N1CC2CCC1CN2 ; *N1CC2CCC(C1)N2 ; *N1C2CCC1CNC2]

In Formula III or IIIb or any of the embodiments of Formula III or IIIb, R^{1b} may be optionally substituted with 1 or more R⁴. Where R^{1b} contains a substitutable ring N atom R^{1b} may preferably be substituted on a substitutable ring N atom. R^{1b} may be substituted by 1 R⁴, preferably on a ring N atom.

In Formula III or IIIb or any of the embodiments of Formula or III or IIIb, R^{1b} may be a 4- to 10-membered non-aromatic ring that may be monocyclic, bridged or bicyclic containing at least 1 ring N heteroatom (i.e. with no ring O heteroatom). R^{1b} may be a 6-membered saturated monocyclic ring containing 1 or 2 ring N heteroatoms, or a 7- to 8-membered saturated, bridged ring system containing 1 or 2 ring N heteroatoms, and R^{1b} is optionally substituted with 1 or more R⁴.

In embodiment (3) of Formula III or IIIb:
Q is C;
R^{1a} is a a 7- to 9-membered saturated, bridged ring system containing 2 ring N heteroatoms, wherein R¹ is optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2; and optionally b is 1.

In embodiment (4) of Formula III or IIIb, the compound is a compound of structure:

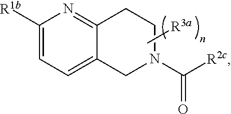

or a pharmaceutically acceptable salt or derivative thereof, wherein:
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may optionally be substituted with a 6-membered aromatic or heteroaromatic ring that contains 0, 1 or 2 ring N atoms; and wherein R^{2c} is optionally substituted with 1 or more R⁵. The remaining moieties may be as defined for Formula I, Ib, III or IIIb or any embodiments of Formula I, Ib, III or IIIb described herein, mutatis mutandis. For example, R^{1b} may be a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom), wherein R^{1b} may be optionally substituted with 1 or more R⁴. R^{1b} may be a 6-membered saturated, monocyclic ring containing at least 1 ring N heteroatom (for example, 1 ring N heteroatom or 2 ring N heteroatoms), wherein R^{1b} may be optionally substituted with 1 or more R⁴. R^{1b} may be a 6-membered saturated, monocyclic ring containing at least 1 ring N heteroatom (for example, 1 ring N heteroatom or 2 ring N heteroatoms), wherein R^{1b} may be optionally substituted with 1 R⁴. R⁴, where present, may be (C1-6)alkyl optionally substituted with OH, optionally (C1-2)alkyl optionally substituted with OH. n may be 0. R^{2c} may be as defined according to embodiment (18) of Formula I or Ib.

In any embodiment of Formula III or IIIb described herein, A is preferably R^{2c} or NR^{2a}R^{2b}, preferably R^{2c}.

Described herein is a compound of Formula IV:

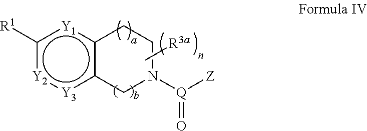

or a pharmaceutically acceptable salt or derivative thereof, wherein:
Z is NR^{2d}R^{2b} or R^{2e};
R^{2b} is H or (C1-6)alkyl, and wherein (C1-6)alkyl is optionally substituted with 1 or more R⁵; and R^{2d} is
   a) a (C5-10)alkyl group comprising a cyclic moeity; and wherein R^{2d} is optionally substituted with 1 or more R⁵; or
   b) a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2d} is substituted with 1 or more R⁵ (optionally wherein R⁵ is halogen);
R^{2e} is
   a) a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms; and wherein R^{2e} is optionally substituted with 1 or more R⁵; or
   b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a (C3-10)alkyl group that may be straight chain or branched, wherein a C atom in straight chain portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; and wherein R^{2e} is substituted with 1 or more R⁵ (optionally wherein R⁵ is halogen);
each R^{3a} is independently (C1-6)alkyl, optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen.

and Y₁, Y₂, Y₃, R¹, R^{3a}, R^{3b}, Q, R⁴, R⁵, a, b and n are as defined for Formula I or any of embodiments (1)-(9) and (21)-(31) of Formula I above.

Also described herein is a compound of Formula IVb:

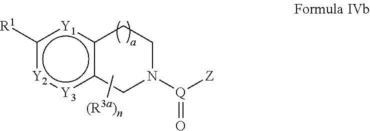

or a pharmaceutically acceptable salt or derivative thereof, wherein:
Z is NR^{2d}R^{2b} or R^{2e};
R^{2b} is H or (C1-6)alkyl, and wherein (C1-6)alkyl is optionally substituted with 1 or more R⁵; and
R^{2d} is
   a) a (C5-10)alkyl group comprising a cyclic moeity; and wherein R^{2d} is optionally substituted with 1 or more R⁵; or
   b) a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2d} is substituted with 1 or more R⁵ (optionally wherein R⁵ is halogen);
R^{2e} is
   a) a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms; and wherein R^{2e} is optionally substituted with 1 or more R⁵; or
   b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; and wherein R^{2e} is substituted with 1 or more R⁵ (optionally wherein R⁵ is halogen);
each R^{3a} is independently (C1-6)alkyl, optionally substituted by 1 or more halogen; or
   two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen.

and Y₁, Y₂, Y₃, R¹, R^{3a}, R^{3b}, Q, R⁴, R⁵, a and n are as defined for Formula Ib or any of embodiments (1)-(9) and (21)-(31) of Formula Ib above.

In embodiment (1) of Formula IV or IVb, R^{2d} is a 6-membered aromatic or heteroaromatic ring that contains 0, 1 or 2 ring N atoms, wherein R^{2d} is substituted with 1 or more R⁵. R^{2d} may be substituted with 1 or 2 R⁵, preferably 1 R⁵. R^{2d} may be substituted with halogen, CN or (C1-4)alkyl, the (C1-4)alkyl group being optionally substituted with 1 or more halogen, preferably optionally substituted with 1 or more fluoro. R^{2d} may be substituted with halogen (preferably fluoro), CN or CF₃.

In embodiment (2) of Formula IV or IVb, R^{2d} is a CH₂-[6-membered aromatic or heteroaromatic ring that contains 0, 1 or 2 ring N atoms], wherein R^{2d} is substituted with 1 or more R⁵. It will be appreciated that substitution by R⁵ is possible on the —CH₂— linker or aromatic or heteroaromatic ring of R^{2d}. R^{2d} is a CH₂-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃.

In embodiment (3) of Formula IV or IVb, R^{2d} is a (C2-10)alkyl group that may be straight chain, branched or cyclic, or a combination thereof, wherein R^{2d} is substituted with 1 or more R⁵. R^{2d} may be substituted with 1 or 2 R⁵, preferably 1 R⁵. R^{2d} may be a (C3-10)alkyl group that may be straight chain, branched or cyclic, or a combination thereof, wherein R^{2d} is substituted with R⁵. R^{2d} may be a (C4-10)alkyl group that may be straight chain, branched or cyclic, or a combination thereof, wherein R^{2d} is substituted with R⁵. R^{2d} may be a (C5-10)alkyl group that may be straight chain, branched or cyclic, or a combination thereof, wherein R^{2d} is substituted with R⁵. R^{2d} may be a (C5-10)alkyl group comprising a cyclic moiety, wherein R^{2d} is substituted with R⁵. The remaining moieties may be as defined for Formula I or Ib or any of embodiments (1)-(9), (13) or (19)-(26) of Formula I or Ib described herein, mutatis mutandis.

In embodiment (4) of Formula IV or IVb, R^{2d} is a (C5-10)alkyl group comprising a cyclic moeity; and wherein R^{2d} is optionally substituted with 1 or more R⁵, R^{2d} may be a (C5-8)cycloalkyl group or CH₂—[(C5-6)cycloalkyl group]. R^{2d} may be substituted with 1 or 2 R⁵, R⁵ may preferably by halogen (for example, fluoro). A cyclic moiety in R^{2d} may be substituted with 2 R⁵ (for example, fluoro) at the same carbon atom.

In embodiment (5) of Formula IV or IVb, R^{2b} is H or (C1-3)alkyl, and wherein (C1-3)alkyl is optionally substituted with 1 or more R⁵. R^{2b} may be H, CH₃ or CH₂CH₃.

In embodiment (6) of Formula IV or IVb, Z is R^{2e}. Preferably, Q is also C.

In embodiment (7) of Formula IV or IVb, R^{2e} is CH₂-[6-membered aromatic or heteroaromatic ring that contains 0, 1 or 2 ring N atoms], wherein R^{2e} is substituted with 1 or more R⁵. It will be appreciated that substitution by R⁵ is possible on the —CH₂— linker or aromatic or heteroaromatic ring of R^{2e}. R^{2e} may be a CH₂-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃.

In embodiment (8) of Formula IV or IVb, R^{2e} is CH₂—O-[6-membered aromatic or heteroaromatic ring that contains 0, 1 or 2 ring N atoms], wherein R^{2e} is substituted with 1 or more R⁵. It will be appreciated that substitution by R⁵ is possible on the —CH₂— linker or aromatic or heteroaromatic ring of R^{2e}. R^{2e} may be a CH₂—O-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃.

In embodiment (9) of Formula IV or IVb, R^{2e} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms; wherein R^{2e} is substituted with 1 or more R⁵ (for example, 1 or 2 R⁵, wherein R⁵ is fluoro or OH, preferably fluoro). R^{2e} may be a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms. and wherein R^{2e} is substituted with 1 or more R⁵ (for example, 1 or 2 R⁵, wherein R⁵ is fluoro or OH, preferably fluoro). R^{2e} may be a (C3-10)alkyl group comprising a cyclic moeity, wherein a C atom in straight chain or cyclic portion of said (C3-10)alkyl group may be optionally replaced by 1 —O— other than at the point of attachment of R^{2e} and wherein R^{2e} is substituted with 1 or more R⁵ (for example, 1 or 2 R⁵, wherein R⁵ is fluoro or OH, preferably fluoro).

In embodiment (10) of Formula IV or IVb, R^{2e} is a (C5-10)alkyl group comprising a cyclic moeity; and wherein R^{2e} is optionally substituted with 1 or more R⁵. R^{2e} may be a (C5-6)cycloalkyl group or (C1-2)alkylene-[(C4-6)cycloalkyl group]. R^{2e} may be substituted with 1 or 2 R⁵, R⁵ may preferably be halogen (for example, fluoro). A cyclic moiety in R^{2e} may be substituted with 2 R⁵ (for example, fluoro) at the same carbon atom.

In embodiment (11) of Formula IV or IVb, R^{2e} is 6-membered aromatic or heteroaromatic ring that contains 0, 1 or 2 ring N atoms, wherein R^{2e} is optionally substituted with 1 or more R⁵. R^{2e} may be a 6-membered aromatic or heteroaromatic ring that contains 0, 1 or 2 ring N atoms, wherein R^{2e} is optionally substituted with 1 or 2 R⁵, for example halogen (preferably fluoro), (C1-4)alkoxy, or (C1-4)alkyl, the (C1-4)alkyl group being optionally substituted with 1 or more halogen (preferably optionally substituted with 1 or more fluoro).

In embodiment (12) of Formula IV or IVb, the compound is a compound of structure:

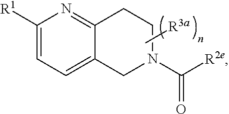

or a pharmaceutically acceptable salt or derivative thereof, wherein:
R^{2e} is a (C5-10)alkyl group comprising a cyclic moeity; and wherein R^{2e} is optionally substituted with 1 or more R⁵. The remaining moieties may be as defined for Formula I, Ib, IV or IVb or any embodiments of Formula I, Ib, IV or IVb described herein, mutatis mutandis. For example, R¹ may be a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom), wherein R¹ may be optionally substituted with 1 or more R⁴. R¹ may be a 6-membered saturated, monocyclic ring containing at least 1 ring N heteroatom (for example, 1 ring N heteroatom or 2 ring N heteroatoms), wherein R¹ may be optionally substituted with 1 or more R⁴. R¹ may be a 6-membered saturated, monocyclic ring containing at least 1 ring N heteroatom (for example, 1 ring N heteroatom or 2 ring N heteroatoms), wherein R¹ may be optionally substituted with 1 R⁴. R⁴, where present, may be (C1-6)alkyl optionally substituted with OH, optionally (C1-2)alkyl optionally substituted with OH. n may be 0. R^{2e} may be as defined according to embodiment (10) of Formula IV or IVb.

Described herein is a compound of Formula V:

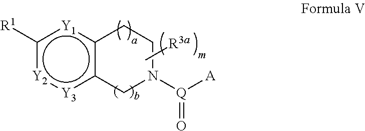

or a pharmaceutically acceptable salt or derivative thereof, wherein:
m is 1, 2, 3 or 4;
and Y₁, Y₂, Y₃, R¹, R^{2a}, R^{2b}, R^{2c}, Raf, R^{3a}, R^{3b}, A, Q, R⁴, R⁵, a and b are as defined for Formula I or any of embodiments (1)-(23) and (26)-(31) of Formula I above.

Also described herein is a compound of Formula Vb:

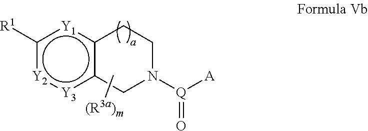

or a pharmaceutically acceptable salt or derivative thereof, wherein:
m is 1, 2, 3 or 4;
and Y₁, Y₂, Y₃, R¹, R^{2a}, R^{2b}, R^{2c}, R^{3a}, R^{3b}, A, Q, R⁴, R⁵ and a are as defined for Formula Ib or any of embodiments (1)-(23) and (26)-(31) of Formula Ib above.

In embodiment (1) of Formula V or Vb, m is 1 or 2.

In a further embodiment of a compound of Formula I to V or Ib to Vb or a pharmaceutically acceptable salt thereof, including any of the embodiments thereof described above, one or more hydrogen atoms are replaced by 2H. The remaining moieties may be as defined for any aspect or embodiment of Formula I to V or Ib to Vb described herein, mutatis mutandis.

Compounds of Formula I to V, including any embodiments thereof, may be preferred.

In an embodiment, the compound of Formula I or Ib is selected from:
1-(2-(3,8-diazabicyclo[3.2.1]octan-8-yl)-7,8-dihydro-1,6-naphthyridin-6(5H)-yl)-2-cyclopentylethan-1-one;
1-(2-(3,8-diazabicyclo[3.2.1]octan-8-yl)-7,8-dihydro-1,6-naphthyridin-6(5H)-yl)-2-(4,4-difluorocyclohexyl)ethan-1-one;
1-(2-(3,8-diazabicyclo[3.2.1]octan-8-yl)-7,8-dihydro-1,6-naphthyridin-6(5H)-yl)-2-phenoxyethan-1-one;
1-(2-(3,8-diazabicyclo[3.2.1]octan-8-yl)-7,8-dihydro-1,6-naphthyridin-6(5H)-yl)-3,3-dimethylbutan-1-one;
1-(2-(3,8-diazabicyclo[3.2.1]octan-8-yl)-7,8-dihydro-1,6-naphthyridin-6(5H)-yl)-3-methylbutan-1-one;
1-(2-(3,8-diazabicyclo[3.2.1]octan-8-yl)-7,8-dihydro-1,6-naphthyridin-6(5H)-yl)-2,3-dimethylbutan-1-one;
1-(2-(3,8-diazabicyclo[3.2.1]octan-8-yl)-7,8-dihydro-1,6-naphthyridin-6(5H)-yl)-2-phenylethan-1-one;
1-(2-(3,8-diazabicyclo[3.2.1]octan-8-yl)-7,8-dihydro-1,6-naphthyridin-6(5H)-yl)-2-(4-fluorophenoxy)ethan-1-one;
(2-(3,8-diazabicyclo[3.2.1]octan-8-yl)-7,8-dihydro-1,6-naphthyridin-6(5H)-yl)(cyclopentyl)methanone;
1-(2-(3,8-diazabicyclo[3.2.1]octan-8-yl)-7,8-dihydro-1,6-naphthyridin-6(5H)-yl)-2-(4-fluorophenyl)ethan-1-one;
1-(2-(3,8-diazabicyclo[3.2.1]octan-8-yl)-7,8-dihydro-1,6-naphthyridin-6(5H)-yl)-2,2-difluoro-2-phenylethan-1-one;
2-(3,8-diazabicyclo[3.2.1]octan-8-yl)-N-(4,4-difluorocyclohexyl)-7,8-dihydro-1,6-naphthyridine-6(5H)-carboxamide;
(2-(3,8-diazabicyclo[3.2.1]octan-8-yl)-7,8-dihydro-1,6-naphthyridin-6(5H)-yl)(pyrrolidin-1-yl)methanone;
2-(3,8-diazabicyclo[3.2.1]octan-8-yl)-N-cyclopentyl-N-methyl-7,8-dihydro-1,6-naphthyridine-6(5H)-carboxamide;
2-(3,8-diazabicyclo[3.2.1]octan-8-yl)-N-cyclopentyl-N-ethyl-7,8-dihydro-1,6-naphthyridine-6(5H)-carboxamide;
1-(2-(3,8-diazabicyclo[3.2.1]octan-8-yl)-5,7-dihydro-6H-pyrrolo[3,4-b]pyridin-6-yl)-2-(4,4-difluorocyclohexyl)ethan-1-one;
1-(2-(3,8-diazabicyclo[3.2.1]octan-8-yl)-5,7-dihydro-6H-pyrrolo[3,4-b]pyridin-6-yl)-2-cyclopentylethan-1-one;
2-(3,8-diazabicyclo[3.2.1]octan-8-yl)-N-(4,4-difluorocyclohexyl)-5,7-dihydro-6H-pyrrolo[3,4-b]pyridine-6-carboxamide;
2-(3,8-diazabicyclo[3.2.1]octan-8-yl)-N-cyclopentyl-5,7-dihydro-6H-pyrrolo[3,4-b]pyridine-6-carboxamide;
N-(4,4-difluorocyclohexyl)-2-(3-methyl-3,8-diazabicyclo[3.2.1]octan-8-yl)-5,7-dihydro-6H-pyrrolo[3,4-b]pyridine-6-carboxamide;
N-cyclopentyl-2-(3-methyl-3,8-diazabicyclo[3.2.1]octan-8-yl)-5,7-dihydro-6H-pyrrolo[3,4-b]pyridine-6-carboxamide;
2-(4,4-difluorocyclohexyl)-1-(2-(3-methyl-3,8-diazabicyclo[3.2.1]octan-8-yl)-5,7-dihydro-6H-pyrrolo[3,4-b]pyridin-6-yl)ethan-1-one;
2-cyclopentyl-1-(2-(3-methyl-3,8-diazabicyclo[3.2.1]octan-8-yl)-5,7-dihydro-6H-pyrrolo[3,4-b]pyridin-6-yl)ethan-1-one;
(2-(3,8-diazabicyclo[3.2.1]octan-8-yl)-5,7-dihydro-6H-pyrrolo[3,4-b]pyridin-6-yl)(pyrrolidin-1-yl)methanone;
2-(3,8-diazabicyclo[3.2.1]octan-8-yl)-N,N-diethyl-5,7-dihydro-6H-pyrrolo[3,4-b]pyridine-6-carboxamide;
N-(4,4-difluorocyclohexyl)-2-(3-methyl-3,8-diazabicyclo[3.2.1]octan-8-yl)-7,8-dihydro-1,6-naphthyridine-6(5H)-carboxamide;
2-(4,4-difluorocyclohexyl)-1-(2-(3-methyl-3,8-diazabicyclo[3.2.1]octan-8-yl)-7,8-dihydro-1,6-naphthyridin-6(5H)-yl)ethan-1-one;
2-(4,4-difluorocyclohexyl)-1-(2-(piperazin-1-yl)-7,8-dihydro-1,6-naphthyridin-6(5H)-yl)ethan-1-one;
(R)-2-cyclopentyl-1-(2-(hexahydropyrrolo[1,2-a]pyrazin-2 (1H)-yl)-7,8-dihydro-1,6-naphthyridin-6(5H)-yl)ethan-1-one;
(S)-2-cyclopentyl-1-(2-(hexahydropyrrolo[1,2-a]pyrazin-2 (1H)-yl)-7,8-dihydro-1,6-naphthyridin-6(5H)-yl)ethan-1-one;
2-cyclopentyl-1-(2-(piperidin-4-yl)-7,8-dihydro-1,6-naphthyridin-6(5H)-yl)ethan-1-one;
2-cyclopentyl-1-(2-(1-(2-hydroxyethyl)piperidin-4-yl)-7,8-dihydro-1,6-naphthyridin-6(5H)-yl)ethan-1-one;
1-(2-(3,8-diazabicyclo[3.2.1]octan-8-yl)-7,8-dihydro-1,6-naphthyridin-6(5H)-yl)-2-(1-methylcyclopentyl)ethan-1-one;
(2-(3,8-diazabicyclo[3.2.1]octan-8-yl)-7,8-dihydro-1,6-naphthyridin-6(5H)-yl)(phenyl)methanone;
(2-(3,8-diazabicyclo[3.2.1]octan-8-yl)-7,8-dihydro-1,6-naphthyridin-6(5H)-yl)(2-methoxyphenyl)methanone;
(2-(3,8-diazabicyclo[3.2.1]octan-8-yl)-7,8-dihydro-1,6-naphthyridin-6(5H)-yl)(o-tolyl)methanone;
(2-(3,8-diazabicyclo[3.2.1]octan-8-yl)-7,8-dihydro-1,6-naphthyridin-6(5H)-yl)(4-fluorophenyl)methanone;
(2R)-1-(2-(3,8-diazabicyclo[3.2.1]octan-8-yl)-7,8-dihydro-1,6-naphthyridin-6(5H)-yl)-2-cyclopentyl-2-hydroxyethan-1-one;
cyclopentyl 2-(3,8-diazabicyclo[3.2.1]octan-8-yl)-7,8-dihydro-1,6-naphthyridine-6(5H)-carboxylate;
isopropyl 2-(3,8-diazabicyclo[3.2.1]octan-8-yl)-7,8-dihydro-1,6-naphthyridine-6(5H)-carboxylate;
benzyl 2-(3,8-diazabicyclo[3.2.1]octan-8-yl)-7,8-dihydro-1,6-naphthyridine-6(5H)-carboxylate;
(2S)-1-(2-(3,8-diazabicyclo[3.2.1]octan-8-yl)-5,7-dihydro-6H-pyrrolo[3,4-b]pyridin-6-yl)-2,3-dimethylbutan-1-one;
1-(2-(3,8-diazabicyclo[3.2.1]octan-8-yl)-5,7-dihydro-6H-pyrrolo[3,4-b]pyridin-6-yl)-2-(1-methylcyclopentyl)ethan-1-one;
1-(2-(3,8-diazabicyclo[3.2.1]octan-8-yl)-5,7-dihydro-6H-pyrrolo[3,4-b]pyridin-6-yl)-2,2-difluoro-2-phenylethan-1-one;
(R)-2-cyclopentyl-1-(2-(3-(hydroxymethyl) piperazin-1-yl)-7,8-dihydro-1,6-naphthyridin-6(5H)-yl)ethan-1-one;
(S)-2-cyclopentyl-1-(2-(3-(hydroxymethyl) piperazin-1-yl)-7,8-dihydro-1,6-naphthyridin-6(5H)-yl)ethan-1-one;
1-(2-(3,8-diazabicyclo[3.2.1]octan-8-yl)-7,8-dihydropyrido[4,3-d]pyrimidin-6(5H)-yl)-2-phenylethan-1-one;
2-phenyl-1-(2-(piperazin-1-yl)-7,8-dihydropyrido[4,3-d]pyrimidin-6(5H)-yl)ethan-1-one;
1-(2-(3,8-diazabicyclo[3.2.1]octan-3-yl)-7,8-dihydropyrido[4,3-d]pyrimidin-6(5H)-yl)-2-phenylethan-1-one;
4-(2-(2-(3,8-diazabicyclo[3.2.1]octan-8-yl)-7,8-dihydropyrido[4,3-d]pyrimidin-6(5H)-yl)-2-oxoethyl)benzonitrile;
4-(2-oxo-2-(2-(piperazin-1-yl)-7,8-dihydropyrido[4,3-d]pyrimidin-6(5H)-yl)ethyl)benzonitrile;
4-(2-(2-(3,8-diazabicyclo[3.2.1]octan-3-yl)-7,8-dihydropyrido[4,3-d]pyrimidin-6(5H)-yl)-2-oxoethyl)benzonitrile;
1-(2-(3,8-diazabicyclo[3.2.1]octan-8-yl)-7,8-dihydropyrido[4,3-d]pyrimidin-6(5H)-yl)-2-(4-fluorophenyl)ethan-1-one;
2-(4-fluorophenyl)-1-(2-(piperazin-1-yl)-7,8-dihydropyrido[4,3-d]pyrimidin-6(5H)-yl)ethan-1-one;
1-(2-(3,8-diazabicyclo[3.2.1]octan-3-yl)-7,8-dihydropyrido[4,3-d]pyrimidin-6(5H)-yl)-2-(4-fluorophenyl)ethan-1-one;
1-(2-(3,8-diazabicyclo[3.2.1]octan-8-yl)-7,8-dihydropyrido[4,3-d]pyrimidin-6(5H)-yl)-2-cyclopentylethan-1-one;
2-cyclopentyl-1-(2-(piperazin-1-yl)-7,8-dihydropyrido[4,3-d]pyrimidin-6(5H)-yl)ethan-1-one;
1-(2-(3,8-diazabicyclo[3.2.1]octan-8-yl)-7,8-dihydropyrido[4,3-d]pyrimidin-6(5H)-yl)-2-cyclohexylethan-1-one;
2-cyclohexyl-1-(2-(piperazin-1-yl)-7,8-dihydropyrido[4,3-d]pyrimidin-6(5H)-yl)ethan-1-one;
1-(2-(3,8-diazabicyclo[3.2.1]octan-8-yl)-7,8-dihydropyrido[4,3-d]pyrimidin-6(5H)-yl)-3,3-dimethylbutan-1-one;
(4-fluorophenyl)(2-(piperazin-1-yl)-7,8-dihydropyrido[4,3-d]pyrimidin-6(5H)-yl)methanone;
1-(2-(3,8-diazabicyclo[3.2.1]octan-8-yl)-5,8-dihydropyrido[3,4-d]pyrimidin-7 (6H)-yl)-2-cyclopentylethan-1-one;
3,3-dimethyl-1-(2-(piperazin-1-yl)-7,8-dihydro-1,6-naphthyridin-6(5H)-yl) butan-1-one;
1-(2-(4-ethylpiperazin-1-yl)-7,8-dihydro-1,6-naphthyridin-6(5H)-yl)-3,3-dimethylbutan-1-one;
1-(2-(3,9-diazabicyclo[3.3.1]nonan-9-yl)-7,8-dihydro-1,6-naphthyridin-6(5H)-yl)-2-cyclopentylethan-1-one;
1-(2-(3,9-diazabicyclo[3.3.1]nonan-3-yl)-7,8-dihydro-1,6-naphthyridin-6(5H)-yl)-2-cyclopentylethan-1-one;
1-(2-(2,5-diazabicyclo[2.2.2]octan-2-yl)-7,8-dihydro-1,6-naphthyridin-6(5H)-yl)-2-cyclopentylethan-1-one;
1-(2-(3,8-diazabicyclo[3.2.1]octan-3-yl)-7,8-dihydro-1,6-naphthyridin-6(5H)-yl)-2-cyclopentylethan-1-one;
1-(2-((1S,4S)-2,5-diazabicyclo[2.2.1]heptan-2-yl)-7,8-dihydro-1,6-naphthyridin-6(5H)-yl)-2-cyclopentylethan-1-one;
1-(2-((1R,4R)-2,5-diazabicyclo[2.2.1]heptan-2-yl)-7,8-dihydro-1,6-naphthyridin-6(5H)-yl)-2-cyclopentylethan-1-one;
2-cyclopentyl-1-(2-(7-hydroxy-3,9-diazabicyclo[3.3.1]nonan-9-yl)-7,8-dihydro-1,6-naphthyridin-6(5H)-yl)ethan-1-one;
2-(3,8-diazabicyclo[3.2.1]octan-8-yl)-6-((4-fluorobenzyl) sulfonyl)-5,6,7,8-tetrahydro-1,6-naphthyridine;
2-(3-ethyl-3,8-diazabicyclo[3.2.1]octan-8-yl)-6-((4-fluorobenzyl) sulfonyl)-5,6,7,8-tetrahydro-1,6-naphthyridine;
6-((4-fluorobenzyl) sulfonyl)-2-(piperazin-1-yl)-5,6,7,8-tetrahydro-1,6-naphthyridine;
2-(4-ethylpiperazin-1-yl)-6-((4-fluorobenzyl) sulfonyl)-5,6,7,8-tetrahydro-1,6-naphthyridine;
6-(benzylsulfonyl)-2-(3,8-diazabicyclo[3.2.1]octan-8-yl)-5,6,7,8-tetrahydro-1,6-naphthyridine;
6-(benzylsulfonyl)-2-(3-ethyl-3,8-diazabicyclo[3.2.1]octan-8-yl)-5,6,7,8-tetrahydro-1,6-naphthyridine;
6-(benzylsulfonyl)-2-(piperazin-1-yl)-5,6,7,8-tetrahydro-1,6-naphthyridine;
6-(benzylsulfonyl)-2-(4-ethylpiperazin-1-yl)-5,6,7,8-tetrahydro-1,6-naphthyridine;
2-(3,8-diazabicyclo[3.2.1]octan-8-yl)-N—((S)-chroman-4-yl)-7,8-dihydro-1,6-naphthyridine-6(5H)-carboxamide;
N—((S)-chroman-4-yl)-2-(3-ethyl-3,8-diazabicyclo[3.2.1]octan-8-yl)-7,8-dihydro-1,6-naphthyridine-6(5H)-carboxamide;
(S)—N-(chroman-4-yl)-2-(piperazin-1-yl)-7,8-dihydro-1,6-naphthyridine-6(5H)-carboxamide;
(S)—N-(chroman-4-yl)-2-(4-ethylpiperazin-1-yl)-7,8-dihydro-1,6-naphthyridine-6(5H)-carboxamide;
N—((R)-chroman-4-yl)-2-(3-ethyl-3,8-diazabicyclo[3.2.1]octan-8-yl)-7,8-dihydro-1,6-naphthyridine-6(5H)-carboxamide;
1-(2-(3-ethyl-3,8-diazabicyclo[3.2.1]octan-8-yl)-7,8-dihydro-1,6-naphthyridin-6(5H)-yl)-2-(4-fluorophenoxy)ethan-1-one;
2-(4-fluorophenoxy)-1-(2-(3-propyl-3,8-diazabicyclo[3.2.1]octan-8-yl)-7,8-dihydro-1,6-naphthyridin-6(5H)-yl)ethan-1-one;
2-(4-fluorophenoxy)-1-(2-(3-(2-methoxyethyl)-3,8-diazabicyclo[3.2.1]octan-8-yl)-7,8-dihydro-1,6-naphthyridin-6(5H)-yl)ethan-1-one;
2-(4-fluorophenoxy)-1-(2-(3-(3-methoxypropyl)-3,8-diazabicyclo[3.2.1]octan-8-yl)-7,8-dihydro-1,6-naphthyridin-6(5H)-yl)ethan-1-one;
2-phenyl-1-(2-(piperazin-1-yl)-7,8-dihydro-1,6-naphthyridin-6(5H)-yl)ethan-1-one;
2-(4-fluorophenyl)-1-(2-(piperazin-1-yl)-7,8-dihydro-1,6-naphthyridin-6(5H)-yl)ethan-1-one;
1-(3-(3,8-diazabicyclo[3.2.1]octan-8-yl)-5,8-dihydro-1,7-naphthyridin-7 (6H)-yl)-2-cyclopentylethan-1-one;
1-(6-(3,8-diazabicyclo[3.2.1]octan-8-yl)-3,4-dihydro-2,7-naphthyridin-2 (1H)-yl)-2-cyclopentylethan-1-one;
1-(7-(3,8-diazabicyclo[3.2.1]octan-8-yl)-3,4-dihydro-2,6-naphthyridin-2 (1H)-yl)-2-cyclopentylethan-1-one;
2-(4-fluorophenoxy)-1-(2-(piperazin-1-yl)-7,8-dihydro-1,6-naphthyridin-6(5H)-yl)ethan-1-one;
2-cyclopentyl-1-(2-(2-methylpyridin-3-yl)-7,8-dihydro-1,6-naphthyridin-6(5H)-yl)ethan-1-one;
2-cyclopentyl-1-(2-(5-methylpyridin-3-yl)-7,8-dihydro-1,6-naphthyridin-6(5H)-yl)ethan-1-one;
and pharmaceutically acceptable salts thereof.

In a further embodiment of a compound of Formula I or Ib, R¹ may be as defined in any of the compounds of Formula I or Ib, above. In a further embodiment of a compound of Formula I or Ib, A may be as defined in any of the compounds of Formula I or Ib, above.

Further aspects and embodiments are as set out in the following numbered clauses.

Clause 1. A compound of Formula I

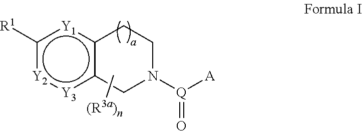

or a pharmaceutically acceptable salt or derivative thereof, wherein:
one or two of Y₁, Y₂ and Y₃ are N and the other(s) are CR^{3b};
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1;

for use in the treatment or prevention of a disease or disorder that can be ameliorated by activation of long isoforms of PDE4.

Clause 2. The compound for use of Clause 1, where in the compound is a compound of formula:

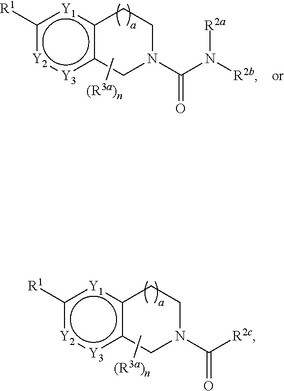

or a pharmaceutically acceptable salt or derivative thereof.

Clause 3. The compound, or a pharmaceutically acceptable salt or derivative thereof, for use of Clause 1 or 2, wherein R¹ is a 5- to 6-membered saturated, monocyclic ring containing at least 1 ring N heteroatom and optionally a ring O heteroatom; a 6-membered aromatic, monocyclic ring containing 1 or 2 ring N heteroatoms; a 7- to 9-membered saturated, bridged ring system containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R¹ is optionally substituted with 1, 2 or 3 R⁴.

Clause 4. The compound, or a pharmaceutically acceptable salt or derivative thereof, for use of any preceding Clause, wherein R¹ is a 4- to 10-membered non-aromatic ring that may be monocyclic, bridged or bicyclic containing at least 1 ring N heteroatom and optionally a ring O heteroatom; wherein R¹ is optionally substituted with 1 R⁴.

Clause 5. The compound, or a pharmaceutically acceptable salt or derivative thereof, for use of any one of Clauses 1 to 3, wherein R¹ is a 7- to 9-membered saturated, bridged ring system containing 2 ring N heteroatoms, optionally a 7- to 8-membered saturated, bridged ring system containing 2 ring N heteroatoms (for example a bridged piperazine, such as 3,8-diazabicyclo[3.2.1]octanyl), wherein R¹ is optionally substituted with 1 R⁴.

Clause 6. The compound, or a pharmaceutically acceptable salt or derivative thereof, for use of any preceding Clause, wherein:
a) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain, branched or cyclic, or a combination thereof; and wherein R^{2a} is optionally substituted with 1 or more R⁵; and
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵; or
b) R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵.

Clause 7. The compound, or a pharmaceutically acceptable salt or derivative thereof, for use of any preceding Clause, wherein:
a) R^{2a} is a (C5-10)alkyl group comprising a cyclic moiety; and wherein R^{2a} is optionally substituted with 1 or more R⁵; and R^{2b} is H or (C1-6)alkyl, and wherein (C1-6)alkyl is optionally substituted with 1 or more R⁵; or
b) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2a} is substituted with 1 or more R⁵ (optionally wherein R⁵ is halogen); and R^{2b} is H or (C1-6)alkyl, and wherein (C1-6)alkyl is optionally substituted with 1 or more R⁵.

Clause 8. The compound, or a pharmaceutically acceptable salt or derivative thereof, for use of any preceding Clause, wherein R^{2c} is:
a) a (C3-10)alkyl group comprising a cyclic moeity, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is substituted with 1 or more R⁵ (optionally wherein R⁵ is halogen).

Clause 9. The compound, or a pharmaceutically acceptable salt or derivative thereof, for use of any one of Clauses 1 to 7, wherein the compound is of formula:

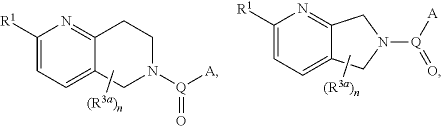

or a pharmaceutically acceptable salt or derivative thereof.

Clause 10. The compound, or a pharmaceutically acceptable salt or derivative thereof, for use of any preceding Clause, wherein each R^{3a} is-CH₃ or F, or two R^{3a} attached to the same carbon are joined together with the atom to which they are attached to form a cyclopropyl ring.

Clause 11. The compound, or a pharmaceutically acceptable salt or derivative thereof, for use of any preceding Clause, wherein n is 0, 1 or 2.

Clause 13. The compound, or a pharmaceutically acceptable salt or derivative thereof, for use of any preceding Clause, wherein Q is C and/or A is R^{2c}.

Clause 14. The compound, or a pharmaceutically acceptable salt or derivative thereof, for use of any preceding Clause, wherein
Q is C;
R¹ is a a 7- to 9-membered saturated, bridged ring system containing 2 ring N heteroatoms, wherein R¹ is optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2.

Clause 15. A compound of Formula IIb:

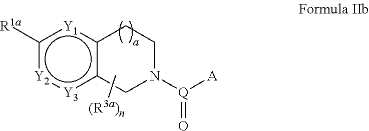

or a pharmaceutically acceptable salt or derivative thereof, wherein:
one or two of Y₁, Y₂ and Y₃ are N and the others are each CR^{3b};
Q is C or S(O);
R^{1a} is a 7- to 9-membered saturated, bridged ring system containing 2 ring N heteroatoms, and wherein R^{1a} is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered ring, optionally additionally containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

Clause 16. The compound, or a pharmaceutically acceptable salt or derivative thereof, of Clause 15, wherein R^{1a} is a 7- to 8-membered saturated, bridged ring system containing 2 ring N heteroatoms (for example a bridged piperazine, such as 3,8-diazabicyclo[3.2.1]octanyl), wherein R¹ is optionally substituted with 1 R⁴.

Clause 17. The compound, or a pharmaceutically acceptable salt or derivative thereof, of any Clause 15 or 16, wherein the compound is of formula:

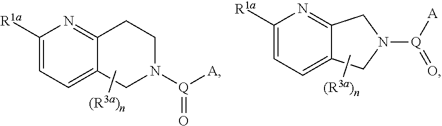

or a pharmaceutically acceptable salt or derivative thereof.

Clause 19. A compound of Formula IIIb:

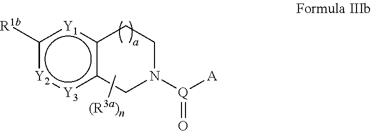

or a pharmaceutically acceptable salt or derivative thereof, wherein:
one or two of Y₁, Y₂ and Y₃ are N and the others are each CR^{3b};
Q is C or S(O);
R^{1b} is a 4- to 10-membered non-aromatic ring that may monocyclic, bridged or bicyclic containing at least 1 ring N heteroatom and optionally a ring O heteroatom, wherein at least 1 ring N heteroatom is not at the point of attachment of R^{1b}, and wherein R^{1b} is optionally substituted with 1 or more R⁴;
A is NR^{2a}R^{2b} or R^{2c}.
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered ring, optionally additionally containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

Clause 20. The compound or a pharmaceutically acceptable salt or derivative thereof of Clause 19, wherein R^{1b} is a 5- to 6-membered saturated, monocyclic ring containing at least 1 ring N heteroatom and optionally a ring O heteroatom; or a 7- to 9-membered saturated, bridged ring system containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R^{1b} is optionally substituted with 1, 2 or 3 R⁴.

Clause 21. The compound, or a pharmaceutically acceptable salt or derivative thereof, of Clause 19 or 20, wherein R^{1b} is a 6-membered saturated monocyclic ring containing 2 ring N heteroatoms or a 7- to 9-membered saturated, bridged ring system containing 2 ring N heteroatoms; wherein R^{1a} is optionally substituted with 1 R⁴, optionally wherein R^{1a} is a 7- to 8-membered saturated, bridged ring system containing 2 ring N heteroatoms (for example a bridged piperazine, such as 3,8-diazabicyclo[3.2.1]octanyl), wherein R^{1b} is optionally substituted with 1 R⁴.

Clause 22. The compound, or a pharmaceutically acceptable salt or derivative thereof, of any of Clause 15-21, wherein:
a) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain, branched or cyclic, or a combination thereof; and wherein R^{2a} is optionally substituted with 1 or more R⁵; and
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵; and/or
b) R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵.

Clause 23. The compound, or a pharmaceutically acceptable salt or derivative thereof, of any of Clause 15-22, wherein R^{2c} is:
a) a (C3-10)alkyl group comprising a cyclic moeity, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵;
b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is substituted with 1 or more R⁵ (optionally wherein R⁵ is halogen).

Clause 24. The compound, or a pharmaceutically acceptable salt or derivative thereof, of any of Clauses 15-23, wherein the compound is of formula:

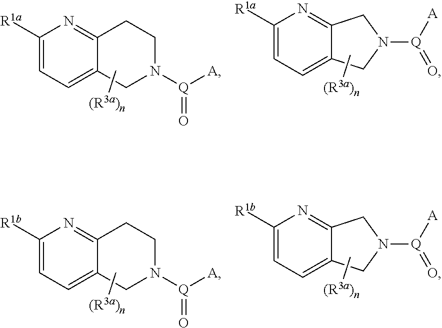

or a pharmaceutically acceptable salt or derivative thereof.

Clause 25. The compound, or a pharmaceutically acceptable salt or derivative thereof, of any of Clause 15-24, wherein Q is C and/or A is R^{2c}.

Clause 26. The compound, or a pharmaceutically acceptable salt or derivative thereof, of any of Clause 15-24, wherein
Q is C;
R^{1a} or R^{1b} is a 7- to 9-membered saturated, bridged ring system containing 2 ring N heteroatoms, wherein R^{1a} and R^{1b} are optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2.

Clause 27. A compound of Formula IVb:

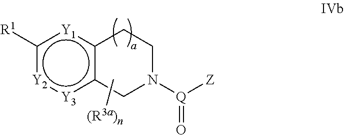

or a pharmaceutically acceptable salt or derivative thereof, wherein:
one or two of Y₁, Y₂ and Y₃ are N and the others are each CR^{3b};
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
Z is NR^{2d}R^{2b} or R^{2e};
R^{2b} is H or (C1-6)alkyl, and wherein (C1-6)alkyl is optionally substituted with 1 or more R⁵; and
R^{2d} is
   a) a (C5-10)alkyl group comprising a cyclic moeity; and wherein R^{2d} is optionally substituted with 1 or more R⁵; or
   b) a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2d} is substituted with 1 or more R⁵ (optionally wherein R⁵ is halogen);
R^{2e} is
   a) a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms; and wherein R^{2e} is optionally substituted with 1 or more R⁵; or
   b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; and wherein R^{2e} is substituted with 1 or more R⁵ (optionally wherein R⁵ is halogen);
each R^{3a} is independently (C1-6)alkyl, optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

Clause 28. The compound, or a pharmaceutically acceptable salt or derivative thereof, of Clause 27, wherein Z is R^{2e}.

Clause 29. The compound or a pharmaceutically acceptable salt or derivative thereof, of Clause 27 or 28, wherein R¹ is a 5- to 6-membered saturated, monocyclic ring containing at least 1 ring N heteroatom and optionally a ring O heteroatom; a 6-membered aromatic, monocyclic ring containing 1 or 2 ring N heteroatoms; a 7- to 9-membered saturated, bridged ring system containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R¹ is optionally substituted with 1, 2 or 3 R⁴.

Clause 30. The compound, or a pharmaceutically acceptable salt or derivative thereof, of any of Clauses 27-29, wherein R¹ is a 4- to 10-membered non-aromatic ring that may be monocyclic, bridged or bicyclic containing at least 1 ring N heteroatom and optionally a ring O heteroatom; wherein R¹ is optionally substituted with 1 R⁴.

Clause 31. The compound, or a pharmaceutically acceptable salt or derivative thereof, of any of Clauses 27-30, wherein R¹ is a a 7- to 9-membered saturated, bridged ring system containing 2 ring N heteroatoms (for example a bridged piperazine, such as 3,8-diazabicyclo[3.2.1]octanyl), wherein R¹ is optionally substituted with 1 R⁴.

Clause 32. The compound, or a pharmaceutically acceptable salt or derivative thereof, of any of Clauses 15-31, wherein each R^{3a} is-CH₃, or two R^{3a} attached to the same carbon are joined together with the atoms to which they are attached to form a cyclopropyl ring.

Clause 33. The compound, or a pharmaceutically acceptable salt or derivative thereof, of any of Clauses 15-32, wherein n is 0, 1 or 2.

Clause 34. A compound of Formula Vb:

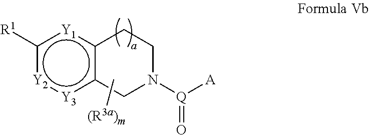

or a pharmaceutically acceptable salt or derivative thereof, wherein:
one or two of Y₁, Y₂ and Y₃ are N and the other(s) are CR^{3b};
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
m is 1, 2, 3 or 4; and
a is 0 or 1.

### Definitions

The term “aromatic ring” refers to an aromatic carbocyclic ring system. The term “heteroaromatic ring” refers to an aromatic ring system wherein one or more of the ring-forming atoms is a heteroatom such as O, S or N. An aromatic ring may be a 6-membered aromatic ring, i.e. a phenyl ring. A heteroaromatic ring may be a 6-membered heteroaromatic ring that contains one to three N atoms or a 5-membered heteroaromatic ring that contains one to three heteroatoms selected from O, S and N. Examples of such 6- or 5-membered heteroaromatic rings include pyridine, pyridazine, pyrazine, pyrimidine, thiophene, furan, thiazole, thiadiazole, oxazole, oxadiazole, imidazole, triazole and their isomers including isothiazole, isothiadiazole, isoxazole and isoxadiazole. In all instances described above, an aromatic ring may be optionally substituted as defined herein.

The term “carbocyclic ring” refers to a ring system with may be saturated, partially unsaturated or aromatic and wherein all ring forming atoms are carbon. The term “heterocyclic ring” refers to a ring system with may be saturated, partially unsaturated or aromatic and wherein one or more of the ring-forming atoms is a heteroatom such as O, S or N. A “non-aromatic carbocyclic or heterocyclic ring” may be saturated or partially unsaturated. Carbocyclic and heterocyclic rings may be bicyclic or multicyclic ring systems, for example bicyclic or multicyclic fused ring systems or bicyclic or multicyclic spiro ring systems or a combination thereof. Each ring within a fused ring system may independently be saturated, partially unsaturated or aromatic. Examples of such fused bicyclic ring systems include indane and chromane. A non-aromatic carbocyclic or heterocyclic ring may include fused ring systems, where for example two rings share two adjacent atoms, bridged ring systems, where for example two rings share three or more adjacent atoms, or spiro ring systems, where for example two rings share one adjacent atom. Examples of fused ring systems include octahydropyrrolo[1,2-a]pyrazine and octahydro-2H-pyrido[1,2-a]pyrazine. Bridged rings may comprise three or more rings. Examples of such bridged ring systems include 2,5-diazabicyclo[2.2.1]heptane, 2,5-diazabicyclo[2.2.2]octane and 3,8-diazabicyclo[3.2.1]octane. Examples of spiro ring systems include spiro[4.3]octane and 2,6-diazaspiro[3.4]octane. In all instances described above, a carbocyclic or heterocyclic ring may be optionally substituted as defined herein.

Where a ring is referred to herein as containing specified ring heteroatoms, it will be appreciated that no further ring heteroatoms are present beyond those specified.

A “monocyclic, bridged or bicyclic ring” includes monocyclic rings, bridged ring systems and bicyclic ring systems. A “monocyclic, bridged or bicyclic ring”, unless otherwise defined, may be saturated, partially unsaturated or aromatic. These may be aromatic, heteroaromatic, carbocyclic or heterocyclic rings or combinations thereof. Bicyclic ring systems may include fused and spiro rings.

Unless otherwise defined, the term “alkyl” as used herein refers to a saturated hydrocarbon which may be straight-chain, branched, cyclic or a combination thereof. Alkyl groups include linear, branched or cyclic alkyl groups and hybrids thereof, such as (cycloalkyl)alkyl. The term “(C1-6)alkyl” as used herein means an alkyl group having 1-6 carbon atoms, which may be branched or unbranched and optionally contains a ring. Examples of (C1-6)alkyl include hexyl, cyclohexyl, pentyl, cyclopentyl, butyl, isobutyl, cyclobutyl, tertiary butyl, propyl, isopropyl, cyclopropyl, cyclopropylmethyl, ethyl and methyl. The term “(C1-4)alkyl” as used herein means a branched or unbranched alkyl group having 1-4 carbon atoms, optionally containing a ring. Examples of (C1-4)alkyl include butyl, isobutyl, cyclobutyl, tertiary butyl, propyl, isopropyl, cyclopropyl, cyclopropylmethyl, ethyl and methyl. A (C1-4)alkyl as referenced herein may preferably be a (C1-2)alkyl. Where specified in the formulae above, (C1-4)alkyl may be substituted, for example with 1 to 3 fluoros. A particularly preferred example of a substituted (C1-4)alkyl is trifluoromethyl. Alternatively (C1-4)alkyl may be unsubstituted.

The term “alkylene” as used herein refers to a divalent alkyl group.

The term “cycloalkyl” refers to a cyclic alkyl group, for example cycloheptyl, cyclohexyl, cyclopentyl, cyclobutyl or cyclopropyl. Cycloalkyl may be substituted as defined herein.

The term “alkoxy” means-O-alkyl wherein alkyl has the meaning as defined above. Examples of (C1-4)alkoxy include methoxy, ethoxy, propoxy, isopropoxy, butoxy, isobutoxy and tertiary butoxy. A (C1-4)alkoxy as referenced herein may preferably be a (C1-2)alkoxy. Where specified in the formulae above, (C1-4)alkoxy may be substituted, for example with 1 to 3 fluoros. A particularly preferred example of a substituted (C1-4)alkoxy is trifluoromethoxy.

Alternatively, (C1-4)alkoxy may be unsubstituted. In the present invention, alkoxy is attached to the rest of the molecule by the “oxy” moiety.

A group that is referred to herein as being “substituted”, whether preceded by the term “optionally” or not, means that at least one hydrogen present on a group (e.g. a C or N atom) is replaced with a permissible substituent, for example a substituent which upon substitution results in a stable compound, e.g. a compound which does not spontaneously undergo transformation such as by rearrangement, cyclization, elimination or other reaction. Unless otherwise indicated, when more than one substituent is present, the substituent is either the same or different at each occurrence. Unless otherwise indicated, a “substituted” group has one or more substituents at one or more substitutable positions of the group, and when more than one position in any given structure is substituted, the substituent is either the same or different at each position.

The term “may” as used herein is interpreted as being optional. Where a feature is referred to as “may be” present, said feature is optionally present. For example, where a moiety is described as “may be substituted”, said moiety is optionally substituted, i.e. it is either unsubstituted or is substituted as described.

The term “halogen” means F, Cl, Br or I. F and Cl are particularly preferred, with F the most preferred.

### Activation of Long PDE4 Isoforms

PDE4 long isoforms have two regulatory regions, upstream conserved region 1 (UCR1) and upstream conserved region 2 (UCR2). These are between the isoform-specific N-terminal portion and the catalytic domain. The UCR1 domain is missing in the short forms, whereas the super-short forms not only lack UCR1, but also have a N-terminal truncated UCR2 domain (Houslay, M. D., Schafer, P. and Zhang, K. Drug Discovery Today 10:1503-1519, 2005).

There are four PDE4 families, PDE4A, PDE4B, PDE4C and PDE4D. The present invention concerns compounds that are capable of activating one or more of the long isoforms from one or more of these four families. The long isoform PDE4 may therefore be long isoform PDE4A, long isoform PDE4B, long isoform PDE4C or long isoform PDE4D. For the avoidance of doubt, a long isoform PDE4 contains a UCR1 region. In some embodiments, a long isoform PDE4 as referred to herein is human. UCR1 is conserved within mammalian species (Houslay, M D, Sullivan, M and Bolger G B Adv. Pharmacol. 44:225-342, 1998), so in other embodiments, the long isoform PDE4 can be from a non-human mammal.

Without wishing to be bound by theory, the compounds described herein may act as PDE4 long form activators. The compounds described herein are small molecules that are believed to bind directly to PDE4 long forms and induce structural changes that increase, stabilise, uncover and/or maintain the catalytic activity of these enzymes. Without wishing to be bound by theory, the activation of PDE4 long forms by PDE4 long form activators may be sensitive to the regulatory status of the enzyme, including post-translational modifications (such as phosphorylation) or the adoption of protein-protein complexes associated with a particular physiological localisation or with a cellular or biochemical assay context. PDE4 long form activators may manifest activation of the enzyme in one or more states but not necessarily all states.

In the field of pharmacology, and as used herein, a small molecule is defined as a low molecular weight organic compound that may serve as a regulator of biological processes. Preferred small molecule activators according to the present invention have a molecular weight of less than or equal to 700 Daltons. This allows for the possibility to rapidly diffuse across cell membranes and reach intracellular sites of action (Veber, D. F. et al., J. Med. Chem. 45:2615-2623, 2002). Especially preferred small molecule activators according to the present invention have molecular weights of greater than or equal to 250 Daltons and less than or equal to 500 Daltons (Lipinski, C. A. Drug Discovery Today: Technologies 1:337-341, 2004).

One suitable method of detecting whether or not a compound is capable of serving as an activator of a PDE4 long form is using a two-step radio-assay procedure described in Experiment 1. In summary, the method involves incubating a PDE4 long form with a test small molecule activator, together with [³H]-labelled CAMP to assess alterations in the breakdown of CAMP to the 5′-adenosine monophosphate (5′-AMP) product. A sample of the reaction mixture from such an incubation is subsequently treated with snake venom 5′-nucleotidase to allow conversion of the nucleotide [³H]-labelled 5′-AMP to the uncharged nucleoside [³H]-labelled adenosine, which can be separated and quantified to assess PDE4 activity and the effect of the test compound (Thompson, W. J. and Appleman, M. M. Biochemistry 10:311-316, 1971, with some modifications as described in: Marchmont, R. J. and Houslay, M. D. Biochem J. 187:381-92, 1980).

Using the above assay procedure, as described in detail in Experiment 1, preferred compounds described herein may produce an increase in the background activity of one or more PDE4 long forms of more than 20% or more than 30% at a test compound concentration of 100 micromolar or less. Especially preferred compounds described herein may produce an increase in the background activity of one or more PDE4 long forms of more than 20% or more than 30% at a test compound concentration of 10 micromolar, or less, for example 3 micromolar.

The compounds described herein may be selective for the long form of the PDE4 enzyme and, as such, do not act or act to a lesser extent as activators of the short or super-short isoforms of the PDE4 enzyme. The short or super-short isoform PDE4 may be short or super-short isoform PDE4A, short or super-short isoform PDE4B, short or super-short isoform PDE4C, or short or super-short isoform PDE4D. For the avoidance of doubt, short and super-short isoforms of PDE4 lack a UCR1 domain. Super-short isoforms are characterised by a truncated UCR2 domain and lack of a UCR1 domain. The short or super-short isoform PDE4 is, for example, human, but may also be from other mammalian species (where UCR2 is conserved, see Houslay, M D, Sullivan, M and Bolger G B Adv. Pharmacol. 44:225-342, 1998).

Under the same assay conditions, as described in Experiment 1, the compounds described herein may produce a less than 30% or less than 20% increase in the background activity of the short or super-short forms of the PDE4A, PDE4B, PDE4C or PDE4D enzymes at a test compound concentration of 100 micromolar, or less.

Compounds described herein may therefore provide a positive result in an assay for activation of a long form PDE4 and a negative result in an assay for activation of a short form (or super-short form) of PDE4.

PDE4 long isoforms include those now known as PDE4A4, PDE4A4/5, PDE4A5, PDE4A8, PDE4A10, PDE4A11, PDE4B1, PDE4B3, PDE4B4, PDE4C1, PDE4C2, PDE4C3, PDE4C4, PDE4D3, PDE4D4, PDE4D5, PDE4D7, PDE4D8, PDE4D9 and PDE4D11. Further long isoforms may be or have already been identified or called by different nomenclature from any of the four PDE4 sub-families.

PDE4 short and super-short isoforms include PDE4A1, PDE4B2, PDE4B5, PDE4D1, PDE4D2, PDE4D6 and PDE4D10. Further short and super-short isoforms may be or have already been identified or called by different nomenclature from any of the four PDE4 sub-families.

The Examples below exemplify activity of compounds described herein in an assay for activation of the human PDE4D5 long isoforms and a lack of activity in an assay for activation of the human PDE4B2 short isoform. Details of these isoforms and a number of the other known isoforms, including GenBank accession numbers, are provided in Tables A to D immediately below.

TABLE A
Examples of known PDE4A Isoforms

| | | | Calculated | |
|---|---|---|---|---|
| | | | molecular | |
| Isoform | Species | Accession | weight (kDa) | Type |
| PDE4A1 | Human | NM_006202 | 73 | Short |
| PDE4A1 | Rodent | L27062 | 68 | Short |
| PDE4A4* | Human | L20965 | 98 | Long |
| PDE4A5 | Rodent | L27057 | 93 | Long |
| PDE4A7** | Human | U18088 | 37 | Dead-Short |
| PDE4A8 | Human | AY593872 | 96 | Long |
| PDE4A8 | Rodent | L36467 | 85 | Long |
| PDE4A10 | Human | AF073745 | 91 | Long |
| PDE4A11 | Human | AY618547 | 95 | Long |

*Note that the PDE4A4B clone is correct while PDE4A4A has a cloning artefact and PDE4A4C is a truncation artefact.
**Note that this species is C- as well as N-terminally truncated

TABLE B
Examples of known PDE4B Isoforms

| | | | Calculated | |
|---|---|---|---|---|
| | | | molecular | |
| Isoform | Species | Accession | weight (kDa) | Type |
| PDE4B1 | Human | NM_001037341.1 | 83 | Long |
| PDE4B2 | Human | NM_001037339.1 | 64 | Short |
| PDE4B3 | Human | NM_001037340 | 83 | Long |
| PDE4B4 | Rodent | AF202733.1 | 75 | Long |
| PDE4B5 | Human | EF595686.1 | 57 | Super-short |

TABLE C
Examples of known PDE4C Isoforms

| | | | Calculated | |
|---|---|---|---|---|
| | | | molecular | |
| Isoform | Species | Accession | weight (kDa) | Type |
| PDE4C1 | Human | NM_000923 | 79 | Long |
| PDE4C2 | Human | NM_001098819 | 67 | Long |
| PDE4C3 | Human | NM_001098818 | 76 | Long |
| PDE4C4 | Human | U66346 | 88 | Long |
| PDE4C5 | Human | U66347 | 47 | Partial |
| PDE4C6 | Human | U66348 | 58 | Partial |
| PDE4C7 | Human | U66349 | 48 | Partial |

TABLE D
Examples of known PDE4D Isoforms

| | | | Calculated | |
|---|---|---|---|---|
| | | | molecular | |
| Isoform | Species | Accession | weight (kDa) | Type |
| PDE4D1 | Human | NM_001197222 | 66 | Short |
| PDE4D2 | Human | NM_001197221 | 58 | Super-short |
| PDE4D3 | Human | NM_006203 | 76 | Long |
| PDE4D4 | Human | NM_001104631 | 91 | Long |
| PDE4D5 | Human | NM_001197218 | 84 | Long |
| PDE4D6 | Human | NM_001197223 | 59 | Super-short |
| PDE4D7 | Human | NM_001165899 | 85 | Long |
| PDE4D8 | Human | NM_001197219 | 78 | Long |
| PDE4D9 | Human | NM_001197220 | 77 | Long |
| PDE4D10 | Rodent | DQ665896.1 | 58 | Super-short |
| PDE4D11 | Rodent | EU489880.1 | 79 | Long |

*PDE4D8 was originally called PDE4D6 in the literature

Reduction of cAMP Levels

Without wishing to be bound by theory, the compounds described herein may function by reducing cAMP levels in one or more intracellular compartments. The PDE4 long form activators described herein may thus provide a means to regulate certain cellular processes that are dependent upon cAMP. Excessive intracellular cAMP signalling mediates a number of diseases and disorders. Therefore, the compounds described herein are expected to be of utility for the treatment of diseases associated with abnormally elevated CAMP levels, increased CAMP-mediated signalling and/or reduced CAMP elimination, enzymatic or otherwise (e.g. via efflux). The treatment is typically of a human, but may also be of a non-human animal, such as a non-human mammal (e.g. veterinary treatment).

In one aspect, the present invention provides a compound described here (i.e. a small molecule activator of a PDE4 long form), for use in a method for the treatment or prevention of disorders where a reduction of second messenger responses mediated by cyclic 3′,5′-adenosine monophosphate (CAMP) is required.

For example, gain-of-function gene mutations in proteins involved in driving CAMP signalling upstream of adenylyl cyclase, such as GPCRs and Gsα, can lead to abnormal excessive CAMP activity with pathological consequences (Lania A, Mantovani G, Spada A. Ann Endocrinol (Paris). 73:73-75, 2012; Thompson, M. D. et al., Methods Mol. Biol. 448:109-137, 2008; Weinstein L S, Liu J, Sakamoto A, Xie T, Chen M. Endocrinology. 145:5459-5464, 2004; Lania A, Mantovani G, Spada A. Eur J Endocrinol. 145:543-559, 2001). PDE4 long form activators described herein, possessing the ability to accelerate the termination of CAMP action, would therefore be expected to be effective in the treatment, prevention or partial control of diseases characterised by undesirably high CAMP levels, or activity, as detailed below.

The treatment or prevention described herein may be treatment or prevention of a disease or disorder that can be ameliorated by activation of long isoforms of PDE4. The treatment or prevention described herein may be treatment or prevention of a disease or disorder mediated by excessive intracellular CAMP signalling. In these diseases, a reduction of second messenger responses mediated by cyclic 3′,5′-adenosine monophosphate (CAMP) should provide a therapeutic benefit.

### Diseases Ameliorated by Activation of Long Isoforms of PDE4 or Characterised by Elevated CAMP Levels

### Hyperthyroidism

Stimulation of the thyroid-stimulating hormone (TSH) receptor (TSHR) leads to increased generation and release of thyroid hormones, thyroxine and triiodothyronine, through a cAMP-dependent signalling mechanism involving Gsα-mediated activation of adenylyl cyclase. Gain-of-function mutations in the TSHR have been reported to be involved in the development of hyperthyroidism (Duprez, L. et al., Nat. Genet. 7:396-401, 1994; Biebermann, H. et al., J. Clin. Endocrinol. Metab. 86:4429-4433, 2001; Karges, B. et al., J. Endocrinol. 186:377-385, 2005). Activating mutations of both TSHR and Gsα have also been found in goitre and thyroid adenomas (Arturi, F. et al., Exp. Clin. Endocrinol. Diabetes 106:234-236, 1998). The increased CAMP activity in thyroid adenomas, as a result of the activating TSHR or Gsα mutations, has been reported to produce a protective adaptive increase in PDE4 activity to counteract abnormal rise in cAMP levels and signal transduction (Persani, L. et al., J. Clin. Endocrinol. Metab. 85:2872-2878, 2000).

The most common cause of hyperthyroidism is Graves' disease, an autoimmune disorder in which antibodies mimic TSH action at the TSHR, leading to excessive CAMP activity in thyroid follicle cells and consequently a state of hyperthyroidism.

PDE4 long form activators described herein are therefore expected to be effective in the treatment, prevention or partial control of hyperthyroidism. In one embodiment, the hyperthyroidism is associated with Graves' disease.

### Jansens's Metaphyseal Chondrodysplasia

Jansens's Metaphyseal Chondrodysplasia (JMC) is a very rare disease resulting from gain-of-function mutations of the parathyroid hormone (PTH) receptor 1 (PTHR1) (Thompson, M. D. et al., Methods Mol. Biol. 448:109-137, 2008). The constitutive activation of the PTHR1 which couples to adenylyl cyclase as effector is associated with excessive CAMP signalling primarily in bone and kidney, leading to dysregulation of ion homeostasis characterised by hypercalcemia and hypophosphatemia (Calvi, L. M. and Schipani, E. J. Endocrinol. Invest. 23:545-554, 2000) and developmental (e.g. short stature) and physical (e.g. protruding eyes) abnormalities. PDE4 long form activators described herein are therefore expected to be effective in the treatment, prevention or partial control of JMC.

### Hyperparathyroidism

Hyperparathyroidism (HPT) is characterized by excessive secretion from the parathyroid gland of PTH, which regulates plasma calcium and phosphate concentrations via PTHR1 receptors in the kidney, bone and GI tract. The resulting excessive stimulation of these receptors causes disruption of plasma ion homeostasis with patients showing hypercalcemia and hypophosphatemia. Primary HPT is driven by parathyroid gland hyperplasia or dysfunction, whereas secondary HPT is associated with underlying medical conditions, predominantly chronic renal disease. Left untreated, HPT causes a variety of debilitating symptoms and can become life-threatening.

By acting to down-regulate excessive cAMP generated by sustained PTH signalling, PDE4 long form activators described herein are expected to be effective in the treatment, prevention or partial control of hyperparathyroidism.

### Familial Male Precocious Puberty (Testotoxicosis)

Familial male-limited precocious puberty (FMPP), also known as familial sexual precocity or gonadotropin-independent testotoxicosis, is a disorder in which boys generally develop signs of precocious puberty in early childhood.

The spinal length in boys may be short due to a rapid advance in epiphyseal maturation. FMPP is an autosomal dominant condition with constitutively activating mutations in the luteinizing hormone (LH) receptor, which leads to increased cAMP production, associated with Leydig cell hyperplasia and low sperm cell count (Latronico, A. C. et al., J Clin. Endocrinol. Metab. 80:2490-2494, 1995; Kosugi, S. et al., Hum. Mol. Genet. 4:183-188, 1995). PDE4 long form activators described herein are therefore expected to be effective in the treatment, prevention or partial control of FMPP.

### Pituitary Adenomas and Cushing's Disease

Non-cancerous tumours of the pituitary gland are collectively referred to as pituitary adenomas and can lead to hypersecretion of adenohypophyseal hormones (e.g. growth hormone, thyroid stimulating hormone, luteinizing hormone, follicle stimulating hormone and adrenocorticotrophic hormone), which exert their action through GPCRs coupled to Gs and CAMP generation. Thus pituitary adenomas can lead to a state of enhanced CAMP mediated signalling in a variety of endocrine tissues which can precipitate a number of hormonal disorders such as acromegly (mainly due to excess growth hormone secretion), Cushing's disease (due to overproduction of adrenocorticotrophic hormone (ACTH) and the subsequent hypercortisolemia) and/or general hyperpituitarism (associated with excess release of multiple anterior pituitary hormones). Current treatment options for pituitary adenomas include treatment with dopamine receptor agonists, which reduce tumour size and lower pituitary hormonal output through a mechanism involving lowering of intracellular cAMP levels. PDE4 long form activators described herein may also be expected to attenuate the pathological effects of pituitary hormones in their target tissues, such as the adrenal glands.

In Cushing's disease, pituitary adenoma related overproduction of ACTH can lead to hypercortisolemia through an overactivation of melanocortin 2 receptor (MC2) and subsequent CAMP mediated stimulation of steroidogenesis and release of cortisol from the adrenal cortex (Tritos, N. A. and Biller, B. M. Discov. Med. 13:171-179, 2012). PDE4 long form activators described herein are therefore expected to be effective in the treatment, prevention or partial control of Cushing's disease.

### Polycystic Kidney Disease

Polycystic kidney disease (PKD) is a genetic disorder of the kidneys characterised by development of pathological cysts, which damage renal structure and compromise kidney function (Takiar, V. and Caplan, M. J. Biochim. Biophys. Acta. 1812:1337-1343, 2011; Masoumi, A. et al., Drugs 67:2495-2510, 2007). There are two types of PKD: autosomal dominant polycystic kidney disease (ADPKD) and autosomal recessive polycystic kidney disease (ARPKD). ADPKD affects between 0.1% and 0.2% of the population worldwide and is characterized by progressive cyst development and enlarged kidneys. Approximately 50% of people with this disease will develop end stage kidney disease, usually between 40 and 70 years of age and require dialysis or kidney transplantation. ARPKD affects around 1:20,000 live births and is typically identified in the first few weeks after birth. Pulmonary hypoplasia results in a 30-50% death rate in neonates with ARPKD.

Defects in two genes are thought to be responsible for ADPKD. In around 85% of patients, development of ADPKD can be linked to mutations in the gene PKD1, encoding polycystin-1 (PC-1); in around 15% of patients mutations in PKD2, encoding polycystin-2 (PC-2) are implicated. Cyclic AMP has been identified as an important stimulus for proliferation and cyst expansion in polycystic kidney cells but not in normal human kidney cells (Yamaguchi, T. et al., Kidney Int. 57:1460-1471, 2000). A considerable body of evidence has now developed to implicate cAMP as an important facilitator of renal cystogenesis (Masoumi, A. et al., Drugs 67:2495-2510, 2007; Wallace, D. P. Biochim. Biophys. Acta. 1812:1291-1300, 2011). Consistent with the role of CAMP in cyst formation, agents that lower cAMP levels (e.g. vasopressin V2 receptor antagonists and the somatostatin receptor agonist octreotide) showed efficacy in rodent models of PKD (Torres, V. E. et al., Nat. Med. 10:363-364, 2004; Gattone, V. H. 2^{nd} et al., Nat. Med. 9:1323-1326, 2003; Belibi, F. A. and Edelstein, C. L. Expert Opin. Investig. Drugs. 19:315-328, 2010). In zebrafish embryos, depletion of a CAMP-hydrolysing PDE enzyme subtype, PDE1A, resulted in development of a cystic phenotype, while PDE1A over-expression partially rescued cystic phenotypes resulting from PC2 depletion (Sussman, C. R., Ward, C. J., Leightner, A. C., Smith, J. L., Agarwal, R., Harris, P. C., Torres, V. E. J. Am. Soc. Nephrol. 25:2222-2230, 2014). Phosphodiesterase activation has been suggested as a therapeutic strategy for PKD treatment (Sun, Y., Zhou, H. and Yang, B-X. Acta Pharmacologica Sinica 32:805-816, 2011).

PDE4 long form activators described herein are therefore expected to be effective in the treatment, prevention or partial control of polycystic kidney disease.

### Polycystic Liver Disease

Polycystic liver disease (PLD) is a rare inherited condition associated with hepatic cystogenesis (usually defined when number of cysts exceeds 20), which often occurs in association with ADPKD (Strazzabosco, M. and Somlo, S. Gastroenterology 140:1855-1859, 2011; Gevers, T. J. and Drenth, J. P. Curr. Opin. Gastroenterol. 27:294-300, 2010). PLD may have a different genetic pathology when compared to ADPKD, driven by mutated proteins associated with the endoplasmic reticulum and the cilium. Increased cholangiocyte proliferation, neovascularisation and elevated fluid secretion act to drive liver cyst formation through dysregulation of multiple signal transduction pathways, including cAMP-mediated signalling. Elevation of hepatic CAMP levels stimulates cAMP-dependent chloride and fluid secretion in biliary epithelial cells and increases cholangiocyte proliferation (Janssen, M. J. et al., J. Hepatol. 52:432-440, 2010). Somatostatin, which acts through a Gi-coupled mechanism to lower cAMP levels, reduced cholangiocyte proliferation and fluid secretion (Gong, A. Y. et al., Am. J. Physiol. Cell. Physiol. 284: C1205-1214, 2003). Furthermore, the synthetic somatostatin analogue, octreotide, showed efficacy in an animal model of PLD through a mechanism involving reduction in cAMP signalling (Masyuk, T. V. et al., Gastroenterology 132:1104-1116, 2007). PDE4 long form activators described herein may therefore be effective in the treatment, prevention or partial control of polycystic liver disease due at least in part to CAMP.

### Maturity Onset Diabetes of Young Type 5 (MODY5)

MODY5 is a form of non-insulin-dependent diabetes mellitus associated with renal cysts. It is an autosomal dominant disorder caused by mutations in the gene encoding hepatocyte nuclear factor-1β (HNF-1β). The predominant clinical feature of patients affected by MODY5 is renal dysfunction, frequently diagnosed before the onset of diabetes. In some patients, HNF-1β mutations can result in additional phenotypic features, such as pancreatic atrophy, abnormal liver function and genital tract abnormalities. Studies in mice suggest that the mechanism responsible for renal cyst formation, associated with mutations of HNF-1β, involves a severe defect of the transcriptional activation of PKD2, in addition to effects on uromodulin (UMOD) and PKD1 genes. Down-regulation of PKD1 and PKD2 is associated with CAMP-driven formation of renal cysts (Mancusi, S. et al., J. Nephrol. 26:207-12, 2013). HNF-1β also binds to the PDE4C promoter and regulates the expression of PDE4C (Ma et al., PNAS 104:20386, 2007).

PDE4 long form activators described herein are therefore expected to be effective in the treatment, prevention or partial control of the symptoms of MODY5.

### Cardiac Hypertrophy, Heart Failure and Arrhythmia

Localized regulation and integration of cAMP signalling are important for proper cardiac function and perturbation of this signalling can lead to heart failure. Upon chronic β-adrenergic receptor stimulation, cardiomyocyte hypertrophy is induced via elevated cAMP and activation of its downstream effectors, including PKA and Epac (Wang, L. et al., Cell. Signal. 27:908-922, 2015 and references therein). Cardiomyocyte hypertrophy increases the risk of heart failure and arrhythmia.

PDE4 long form activators described herein may therefore be effective in the treatment, prevention or partial control of cardiac hypertrophy, heart failure and/or arrhythmia.

Diseases Associated with Increased cAMP-Mediated Signalling

Disorders associated with activating mutations of the alpha subunit of the G protein (GNAS1)

The G-protein Gs acts as a transducer for GPCRs that stimulate adenylyl cyclase activity and exert their biological effects by increasing intracellular cAMP levels. Gs is a heterotrimeric protein composed of α, β and γ subunits. Activating mutations in the gene, GNAS1, for the α-subunit have been identified which lead to exaggerated abnormal cAMP signalling in a variety of tissues and give rise to a range of disorders.

### McCune-Albright Syndrome

McCune-Albright syndrome (MAS) is a rare genetic disorder typically characterised by three dominating features of precocious puberty, fibrous dysplasia of bone and café au lait lesions. The underlying molecular pathology for MAS involves an activating mutation of the GNAS1 gene (Diaz, A. Danon, M. and Crawford, J. J. Pediatr. Endocrinol. Metab. 20:853-880, 2007). PDE4 long form activators described herein would therefore be expected to be effective in the treatment, prevention or partial control of disorders associated with activating mutations of GNAS1, including McCune-Albright syndrome.

### Amelioration of Toxin-Induced Increases in Adenylyl Cyclase Activity in Infectious Diseases.

Adenylyl cyclase, the enzyme responsible for production of CAMP, is a key biological target thought to be involved in mediating the effects of many bacterial toxins (Ahuja et al., Critical Reviews in Microbiology, 30:187-196, 2004). These toxins produce their effects by raising CAMP levels through enhancement of host immune cell and/or pathogen related adenylyl cyclase activity. PDE4 long form activators described herein, by reducing cAMP levels, would therefore be expected to be of utility in the treatment or partial control of symptoms of infectious diseases that are associated with elevated CAMP activity. The following are some examples of such infectious diseases:

### Cholera

Vibrio cholerae produces cholera toxin, which through adenosine disphosphate ribosylation of the a subunit of Gs leads to host cell adenylyl cyclase activation and cAMP production. Diarrhoea caused by cholera toxin is believed to be a result of excessive CAMP accumulation in the cells of the gastrointestinal tract.

### Whooping Cough

Bordetella pertussis is the pathogen responsible for the childhood disease whooping cough. Bordetella pertussis toxin stimulates adenosine disphosphate ribosylation of the a subunit of Gi and indirectly augments CAMP levels in target cells. The bacterium also secretes an invasive adenylyl cyclase, which produces toxic CAMP levels and impairs host immune defence.

### Anthrax

Anthrax is caused by Bacillus anthracis and whilst it is primarily an animal disease it can be transmitted to humans through contact. Anthrax infections are associated with widespread oedema, the development of which is thought to be driven by oedema toxin. The latter is an adenylyl cyclase and is activated by host calmodulin to produce abnormally high levels of CAMP that have a toxic effect on host immune cells.

### Tuberculosis

Mycobactrium tuberculosis expresses a large and diverse range of adenylyl cyclases, which may play a role in virulence and generation of disease pathology. One adenylyl cyclase subtype, RV0386, has been demonstrated to enter host macrophages and elevate intracellular CAMP to cause toxicity (Agarwal et al., Nature, 460:98-102, 2009).

PDE4 long form activators described herein may therefore be effective in the treatment, prevention or partial control of infectious diseases such as cholera, whooping cough, anthrax and tuberculosis.

Diseases Dependent Upon Activation of PKA by Elevated cAMP.

In eukaryotes, cAMP activates protein kinase A (PKA), which is also known as cAMP-dependent protein kinase. PKA is normally inactive as a tetrameric holoenzyme, consisting of two catalytic and two regulatory units, with the regulatory units blocking the catalytic centres of the catalytic units. CAMP binds to specific locations on the regulatory units of PKA and causes dissociation between the regulatory and catalytic units, thus activating the catalytic units. The active catalytic units catalyse the transfer of phosphate from ATP to specific residues of protein substrates, which may modulate the function of those protein substrates.

PDE4 long form activation reduces CAMP levels and CAMP mediated activation of PKA. PDE4 long form activators described herein would therefore be expected to be of utility in the treatment or partial control of disorders where inhibitors of PKA show evidence of therapeutic effects.

Disorders that are dependent upon activation of PKA by cAMP may be identified by their response to PKA inhibitors such as Rp-8-Br-CAMPS. Rp-8-Br-cAMPS is an analogue of CAMP that occupies the CAMP binding sites of PKA, preventing its dissociation and activation.

### HIV Infection and AIDS

T cells from HIV-infected patients have increased levels of CAMP and are more sensitive to inhibition by Rp-8-Br-CAMPS than are normal T cells. Excessive activation of PKA by cAMP has been associated with the progressive T cell dysfunction in HIV infection (Aandahl, E. M. et al., FASEB J. 12:855-862, 1998). Furthermore, in vivo administration of Rp-8-Br-CAMPS has been shown to restore T cell responses in retrovirus-infected mice (Nayjib, B. et al., The Open Immunology Journal, 1:20-24, 2008). PDE4 long form activators described herein are therefore expected to be of utility in the treatment, prevention or partial control of HIV infection and AIDS.

### Common Variable Immunodeficiency (CVID)

In vitro administration of Rp-8-Br-CAMPS has been shown to correct impaired secretion of the cytokine IL-10 by T cells from patients with Common Variable Immunodeficiency (CVID) (Holm, A. M. et al., J. Immunol. 170:5772-5777, 2003). PDE4 long form activators described herein are therefore expected to be of utility in the treatment, prevention or partial control of CVID.

Diseases Dependent Upon Activation of Either or Both of Epac1 and Epac2 by Elevated cAMP.

In addition to PKA, cAMP activates another intracellular receptor, known as exchange protein directly activated by cAMP (Epac). There are two isoforms of Epac, Epac1 and Epac2, both consisting of a regulatory region that binds CAMP and a catalytic region that promotes the exchange of GDP for GTP on the small G proteins, Rap1 and Rap2 of the Ras family. In addition, Epac proteins exert their functions through interactions with a number of other cellular partners at specific cellular loci. Pathophysiological changes in Epac signalling have been associated with a wide range of diseases (Breckler, M. et al., Cell. Signal. 23:1257-1266, 2011).

Relevant disorders that are dependent upon activation of Epac proteins by cAMP may be identified by their response to Epac inhibitors, such as ESI-09, a novel non-cyclic nucleotide Epac1 and Epac2 antagonist that is capable of specifically blocking intracellular Epac-mediated Rap1 activation and Akt phosphorylation, as well as Epac-mediated insulin secretion in pancreatic beta cells (Almahariq, M. et al., Mol. Pharmacol. 83:122-128, 2013).

### Melanoma

Epac1 has been implicated in promoting migration and metastasis in melanoma (Baljinnyam, E. et al., Pigment Cell Melanoma Res. 24:680-687, 2011 and references cited therein). PDE4 long form activators described herein are therefore expected to be of utility in the treatment, prevention or partial control of melanoma.

### Pancreatic Cancer

It has recently been shown that Epac1 is markedly elevated in human pancreatic cancer cells as compared with normal pancreas or surrounding tissue (Lorenz, R. et al., Pancreas 37:102-103, 2008).

Pancreatic cancer is often resistant to treatments that are usually effective for other types of cancer. Using the Epac inhibitor ESI-09, a functional role of Epac1 overexpression in pancreatic cancer cell migration and invasion was demonstrated (Almahariq, M. et al., Mol. Pharmacol. 83:122-128, 2013). These findings are consistent with results based on RNAi silencing techniques and suggest that inhibition of Epac1 signalling could be an effective therapeutic strategy for pancreatic cancer.

PDE4 long form activators described herein would therefore be expected to be of utility in the treatment, prevention or partial control of pancreatic cancer.

Diseases Dependent Upon Modulation of cAMP-Gated Ion Channels by Elevated cAMP.

In addition to activation of PKA and Epac, another effector pathway for elevated CAMP is the activation of cAMP-gated ion channels. PDE4 long form activators described herein would therefore be expected to be of utility in the treatment of disorders where inhibitors of cAMP-gated ion channels show evidence of therapeutic effects.

Diseases Associated with Increased Activity of cAMP Response Element Binding Protein.

The CAMP response element binding protein (CREB) is an important transcription factor involved in the regulation of a variety of cellular functions such as cell proliferation, differentiation, survival, and apoptosis (Cho et al., Crit Rev Oncog, 16:37-46, 2011). CREB activity is regulated by kinase dependant phosphorylation through a range of extracellular signals, such as stress, growth factors and neurotransmitters. Phosphorylation leads to dimerisation of CREB, and together with other co-activator partner proteins, enables it to bind to promoter regions of target genes containing the CAMP response element (CRE sites) and initiate transcriptional activity. The CAMP pathway (e.g. through cAMP-dependant protein kinase mediated phosphorylation) is an important positive modulator of CREB mediated biological activity. PDE4 long form activators described herein are therefore expected to be of utility in the treatment, prevention or partial control of disorders associated with elevated CREB activity.

### Leukaemia

Bone marrow cells from acute lymphoid and myeloid leukaemia patients have been reported to overexpress CREB protein and mRNA (Crans-Vargas et al., Blood, 99:2617-9, 2002; Cho et al., Crit Rev Oncog, 16:37-46, 2011). Furthermore, the increased CREB level correlates with poor clinical response in subjects with acute myeloid leukaemia (Crans-Vargas et al., Blood, 99:2617-9, 2002; Shankar et al., Cancer Cell, 7:351-62, 2005). Upregulation of CREB is associated with stimulation of human leukaemia cell growth whilst downregulation inhibits myeloid cell proliferation and survival. PDE4 long form activators described herein would be expected to reduce CREB activity and function through attenuation of CAMP mediated stimulation of CREB and therefore expected to have utility in the treatment, prevention or partial control of acute lymphoid and myeloid leukaemia.

### Prostate Cancer

Abnormal excessive androgen activity is an important driver in the development of prostate cancer as it stimulates the development of intraepithelial neoplasias (Merkle et al., Cellular Signalling, 23:507-515, 2011). This is strongly supported by the use of androgen ablation approaches, involving chemical or surgical castration, in the treatment of prostate cancer. Cyclic AMP elevating agents such as forskolin can enhance androgen receptor activity through multiple intracellular mechanisms including androgen receptor activation through phosphorylation and/or interaction with CREB. Epac1 activation has also been implicated in promoting cellular proliferation in prostate cancer (Misra, U. K. and Pizzo, S. V. J. Cell. Biochem. 108:998-1011, 2009; Misra, U. K. and Pizzo, S. V. J. Cell. Biochem. 113:1488-1500, 2012). PDE4 long form activators described herein are therefore expected to have utility in the treatment, prevention or partial control of prostate cancer.

Diseases Associated with Reduced Activity of cAMP-Hydrolysing PDE Enzymes

Loss-of-function mutations in gene(s) for cAMP-hydrolysing PDE isoforms other than PDE4, such as PDE8 and PDE11, have been detected in a number of diseases (Vezzosi, D. and Bertherat, J., Eur. J. Endocrinol. 165:177-188, 2011; Levy, I. et al., Curr. Opin. Pharmacol. 11:689-697, 2011; Azevedo, M. F. and Stratakis, C. A. Endocr. Pract. 17 Suppl 3:2-7, 2011). These mutations can lead to abnormally high CAMP levels and/or duration of CAMP action with pathological consequences as detailed below. PDE4 long form activators described herein are therefore expected to be of utility in the treatment, prevention or partial control of these diseases, such as adrenocortical tumours, testicular cancer, PPNAD and Carney Complex.

### Adrenocortical Tumours

Adrenocortical tumours associated with an inactivating point mutation in the gene encoding PDE11A4 have decreased expression of PDE11A4 and increased cAMP levels (Horvath, A. et al., Nat Genet. 38:794-800, 2006; Horvath, A. et al., Cancer Res. 66:11571-11575, 2006; Libé, R., et al., Clin. Cancer Res. 14:4016-4024, 2008).

### Testicular Cancer

Mutations that reduce PDE11A activity and increase CAMP levels have been observed in some forms of testicular cancer (Horvath. A. et al., Cancer Res. 69:5301-5306, 2009).

### Primary Pigmented Nodular Adrenocortical Diseases (PPNAD)

Mutations in the PDE8B gene have also been identified as a predisposing factor for PPNAD and the mutant protein shows reduced ability to degrade cAMP (Horvath, A., Mericq, V. and Stratakis, C. A. N. Engl. J. Med. 358:750-752, 2008; Horvath, A. et al., Eur. J. Hum. Genet. 16:1245-1253, 2008).

### Carney Complex

In Carney Complex (CNC) caused by PRKAR1A mutations, some patients also have defects in PDE11A that may exert a synergistic effect to enhance abnormal activation of the CAMP signal transduction pathway, leading to adrenal and testicular cancer (Libé, R. et al., J. Clin. Endocrinol. Metab. 96: E208-214, 2011).

### Treatment and Posology

By “treatment” herein is meant the treatment by therapy, whether of a human or a non-human animal (e.g., in veterinary applications) typically a non-human mammal, in which some desired therapeutic effect on the condition is achieved; for example, the inhibition of the progress of the disorder, including a reduction in the rate of progress, a halt in the rate of progress, amelioration of the disorder or cure of the condition. Treatment as a prophylactic measure is also included. References herein to prevention or prophylaxis do not indicate or require complete prevention of a condition; its manifestation may instead be reduced or delayed via prophylaxis or prevention according to the present invention.

Compounds or compositions as described herein, when used for preventing or treating a disorder, may be administered in an “effective amount”, which may also be referred to as a “therapeutically effective amount”. By a “therapeutically effective amount” herein is meant an amount of the one or more compounds described herein or a pharmaceutical formulation comprising such one or more compounds, which is effective for producing such a therapeutic effect, commensurate with a reasonable benefit/risk ratio.

It will be appreciated that appropriate dosages of the compounds described herein may vary from patient to patient. Determining the optimal dosage will generally involve the balancing of the level of therapeutic benefit against any risk or deleterious side effects of the treatments of the present invention. The selected dosage level will depend on a variety of factors including the activity of the particular compound, the route of administration, the time of administration, the rate of excretion of the compound, the duration of the treatment, other drugs, compounds or materials used in combination and the age, sex, weight, condition, general health and prior medical history of the patient. The amount of compound(s) and route of administration will ultimately be at the discretion of the physician, although generally the dosage will be to achieve local concentrations at the site of action so as to achieve the desired effect. Administration in vivo can be effected in one dose, continuously or intermittently throughout the course of treatment. Methods of determining the most effective means and dosage of administration are well known to a person skilled in the art and will vary with the formulation used for therapy, the purpose of the therapy, the target cell being treated, and the subject being treated. Single or multiple administrations can be carried out with the dose level and pattern being selected by the treating physician.

In general, a suitable dose of the one or more compounds described herein may be in the range of about 0.001 to 50 mg/kg body weight of the subject per day, preferably in a dosage of 0.01-25 mg per kg body weight per day, e.g., 0.01, 0.05, 0.10, 0.25, 0.50, 1.0, 2.5, 10 or 25 mg/kg per day. Where the compound(s) is a salt, solvate, prodrug or the like, the amount administered may be calculated on the basis of the parent compound and so the actual weight to be used may be increased proportionately.

### Combination Therapies

The compounds described herein may also find application in mimicking or enhancing the effects of drugs known to produce their therapeutic effect through lowering of intracellular CAMP levels.

A number of therapeutically beneficial drugs have a primary mode of action involving lowering intracellular cAMP levels and/or cAMP-mediated activity, as summarised below. Since PDE4 long form activators described herein will also act to lower cAMP levels it is expected that these agents will mimic and/or augment the pharmacological properties and therapeutic utility of drugs operating through a down-regulation of CAMP-mediated signalling. In certain embodiments, a compound described herein is therefore provided as part of a combination therapy with another agent that lowers intracellular CAMP levels and/or cAMP-mediated activity. The combination therapy may be administered simultaneously, contemporaneously, sequentially or separately. In one embodiment, the compound described herein and the separate cAMP lowering agent are provided in a single composition, as described in more detail below. The combination therapy may comprise a described herein and one or more of:
(i) a presynaptic α-2 adrenergic receptor agonist, optionally clonidine, dexmedetomidine, or guanfacine;
(ii) a β-1 Adrenergic receptor antagonist (“beta-blocker”), optionally Atenolol, Metoprolol, Bisoprolol, Acebutolol, or Betaxolol.
   Combination with α-2 Adrenergic Receptor Agonist

α-2 Adrenergic receptor stimulation is known to reduce cAMP levels through a Gi protein-mediated inhibition of adenylyl cyclase activity in a broad range of tissues. In noradrenergic neurones in the brain and peripheral sympathetic nervous system, presynaptic α-2 adrenergic receptor activation inhibits noradrenaline release and noradrenergic activity. Drugs (e.g. clonidine, dexmedetomidine, guanfacine) that act as agonists at these receptors are effective in the treatment of a variety of clinical conditions. Clonidine, the prototypic agent, has shown therapeutic utility in the treatment of hypertension, neuropathic pain, opioid detoxification, insomnia, ADHD, Tourette syndrome, sleep hyperhidrosis, addiction (narcotic, alcohol and nicotine withdrawal symptoms), migraine, hyperarousal, anxiety and also as a veterinary anaesthetic. Lowering of CAMP levels by PDE4 long form activation may be expected to yield similar effects to drugs acting through α-2 adrenergic receptor stimulation. Furthermore, PDE4 long form activators described herein may be expected to potentiate the pharmacodynamic effects of α-2 adrenergic receptor agonists when used in combination.

Combination with β-1 Adrenergic Receptor Antagonist

β-1 Adrenergic receptor antagonists are used in the treatment a range of cardiovascular indications including hypertension, cardiac arrhythmias and cardioprotection following myocardial infarction. Their primary mechanism of action involves reducing the effects of excessive circulating adrenaline and sympathetic activity, mediated by noradrenaline, particularly at cardiac β-1 adrenergic receptors. Endogenous and synthetic β-1 adrenergic receptor agonists stimulate adenylyl cyclase activity through Gₛ activation and raise intracellular cAMP levels in a variety of tissues such as heart and kidney. Consequently, drugs that block β-1 adrenergic receptor mediated activity exert their pharmacological effects by attenuating the increase in cAMP mediated signalling. Given that PDE4 long form activation will also lower CAMP concentration and transduction in cardiac tissue, PDE4 long form activators described herein may be expected to find utility in the treatment or partial control of hypertension, cardiac arrhythmias, congestive heart failure and cardioprotection. Additional non-cardiovascular therapeutic utility may be expected in disorders such as post-traumatic stress related disorder, anxiety, essential tremor and glaucoma, which also respond to β-1 adrenergic antagonist treatment. Furthermore, PDE4 long form activators described herein may be expected to potentiate the pharmacodynamic effects of β-1 adrenergic receptor antagonists when used in combination.

### Methods of Treatment

Compounds as described herein may be used for treating or preventing a disease or disorder that can be ameliorated by activation of long isoforms of PDE4. Compounds as described herein may be used for treating or preventing a disease or disorder mediated by excessive intracellular cyclic AMP signalling. Compounds as described herein may be used for treating or preventing a disease or disorder that can be ameliorated by activation of long isoforms of PDE4, wherein the disease or disorder that can be ameliorated by activation of long isoforms of PDE4 is a disease or disorder mediated by excessive intracellular cyclic AMP signalling. In a further aspect, the present invention provides a small molecule activator of a PDE4 long form described herein for use in a method for the treatment or prevention of a disease or disorder in a patient in need of such therapy. The invention also provides a method of treating or preventing a disease or disorder in a patient in need thereof, comprising administering to a patient in need thereof an effective amount of a compound described herein. The invention provides a method of treating or preventing a disease or disorder that can be ameliorated by activation of long isoforms of PDE4, comprising administering to a patient in need thereof a therapeutically effective amount of any compound or a pharmaceutically acceptable salt or derivative as described herein. The invention provides a method of treating or preventing a disease or disorder mediated by excessive intracellular cyclic AMP signalling, comprising administering to a patient in need thereof a therapeutically effective amount of any compound or a pharmaceutically acceptable salt or derivative as described herein. The disease or disorder may be any disease of disorder described herein, including: a disease associated with increased CAMP production and signalling (such as hyperthyroidism, Jansens's metaphyseal chondrodysplasia, hyperparathyroidism, familial male-limited precocious puberty, pituitary adenomas, Cushing's disease, polycystic kidney disease, polycystic liver disease, MODY5 and cardiac hypertrophy); diseases known to be associated with increased CAMP-mediated signalling, including disorders associated with activating mutations of the alpha subunit of the G protein (GNAS1) (such as McCune-Albright syndrome); amelioration of toxin-induced increases in adenylyl cyclase activity in infectious diseases (such as cholera, whooping cough, anthrax, and tuberculosis); treatment of diseases known to be dependent upon activation of PKA by elevated CAMP (such as HIV infection and AIDS, and Common Variable Immunodeficiency (CVID)); treatment of diseases known to be dependent upon activation of either or both of Epac1 and Epac2 by elevated CAMP (such as melanoma and pancreatic cancer); treatment of diseases dependent upon modulation of cAMP-gated ion channels by elevated cAMP; treatment of diseases known to be associated with increased activity of cAMP response element binding protein (such as leukaemia and prostate cancer); treatment of diseases known to be associated with reduced activity of cAMP-hydrolysing PDE enzymes (such as adrenocortical tumours, testicular cancer, primary pigmented nodular adrenocortical diseases (PPNAD) and Carney Complex); and mimicking or enhancing the effects of drugs known to produce their therapeutic effect through lowering of intracellular CAMP levels.

As used herein, the terms “compound of the invention”, “compound of the disclosure” “compound described herein” and “compound of Formula I”, etc, include pharmaceutically acceptable salts and derivatives thereof and polymorphs, isomers (e.g. stereoisomers and tautomers) and isotopically labelled variants thereof. For example, reference to compounds of Formula I includes pharmaceutically acceptable salts thereof. Reference to compounds of Formula Ib includes pharmaceutically acceptable salts thereof. Furthermore, these terms include all the sub-embodiments of those compounds disclosed herein, including compounds of Formula II to V and IIb to Vb, and all embodiments thereof.

A compound described herein may be provided as a solvate, for example a hydrate.

Pharmaceutically acceptable derivatives of a compound described herein include pharmaceutically acceptable esters, amides, prodrugs (e.g. a pyridine N-oxide) or isotopically labelled variants thereof. The present invention further provides pharmaceutical compositions comprising a compound described herein, including a pharmaceutically acceptable salt, solvate, ester, hydrate or amide thereof, in admixture with a pharmaceutically acceptable excipient(s), and optionally other therapeutic agents. The term “acceptable” means being compatible with the other ingredients of the composition and not deleterious to the recipient thereof. Compositions include e.g. those suitable for oral, sublingual, subcutaneous, intravenous, epidural, intrathecal, intramuscular, transdermal, intranasal, pulmonary, topical, local, or rectal administration, and the like, typically in unit dosage forms for administration.

The term “pharmaceutically acceptable salt” includes a salt prepared from pharmaceutically acceptable non-toxic acids or bases including inorganic or organic acids and bases. Compounds which contain basic, e.g. amino, groups are capable of forming pharmaceutically acceptable salts with acids. Examples of pharmaceutically acceptable acid addition salts of the compounds described herein include acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids.

Compounds which contain acidic, e.g. carboxyl, groups are capable of forming pharmaceutically acceptable salts with bases. Pharmaceutically acceptable basic salts of the compounds described herein include, but are not limited to, metal salts such as alkali metal or alkaline earth metal salts (e.g. sodium, potassium, magnesium or calcium salts) and zinc or aluminium salts and salts formed with ammonia or pharmaceutically acceptable organic amines or heterocyclic bases such as ethanolamines (e.g. diethanolamine), benzylamines, N-methyl-glucamine, amino acids (e.g. lysine) or pyridine.

Hemisalts of acids and bases may also be formed, e.g. hemisulphate salts.

Pharmaceutically acceptable salts of compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002).

### Prodrugs

Compounds described herein may be provided as a prodrug. Prodrugs are derivatives of compounds described herein (which may have little or no pharmacological activity themselves), which can, when administered in vivo, be converted into compounds described herein.

Prodrugs can, for example, be produced by replacing functionalities present in the compounds described herein with appropriate moieties which are metabolised in vivo to form a compound described herein. The design of prodrugs is well-known in the art, as discussed in Bundgaard, Design of Prodrugs 1985 (Elsevier), The Practice of Medicinal Chemistry 2003, 2^{nd} Ed, 561-585 and Leinweber, Drug Metab. Res. 1987, 18:379.

In vivo metabolism of prodrugs of compounds described herein may for example involve hydrolysis, oxidative metabolism or reductive metabolism of the prodrug. Examples of prodrugs of compounds described herein are amides and esters of those compounds that may be hydrolysed in vivo. For example, where the compound described herein contains a carboxylic acid group (—COOH), the hydrogen atom of the carboxylic acid group may be replaced in order to form an ester (e.g. the hydrogen atom may be replaced by C₁₋₆alkyl). Where a compound contains an alcohol group (—OH), the hydrogen atom of the alcohol group may be replaced in order to form an ester (e.g. the hydrogen atom may be replaced by —C(O)C₁₋₆alkyl). Further examples of prodrugs of compounds described herein include pyridine N-oxides that may be reductively metabolised in vivo to form compounds described herein containing a pyridine ring.

### Solvates

It may be convenient or desirable to prepare, purify, and/or handle a corresponding solvate of the compounds described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

### Isomers

It will be appreciated that the compounds described herein may exist in various isomeric forms and the compounds described herein include all stereoisomeric forms and mixtures thereof, including enantiomers and racemic mixtures. The present invention includes within its scope the use of any such stereoisomeric form or mixture of stereoisomers, including the individual enantiomers of the compounds described herein as well as wholly or partially racemic mixtures of such enantiomers. Where appropriate, isomers can be separated from their mixtures by the application or adaptation of known methods (e.g. chromatographic techniques and recrystallisation techniques). Where appropriate, isomers can be prepared by the application or adaptation of known methods (e.g. asymmetric synthesis). In addition, it will be appreciated that in some instances, compounds described herein may exist in tautomeric forms and the compounds described herein include all tautomers and mixtures thereof.

### Isotopes

The compounds described herein include pharmaceutically acceptable isotopically-labelled compounds wherein one or more atoms are replaced by atoms having the same atomic number, but an atomic mass or mass number different from the atomic mass or mass number usually found in nature.

Examples of isotopes suitable for inclusion in the compounds described herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, and sulphur, such as ³⁵S. Certain isotopically-labelled compounds, for example, those incorporating a radioactive isotope, are useful in drug and/or substrate tissue distribution studies. The radioactive isotopes ³H and ¹⁴C are particularly useful for this purpose in view of their ease of incorporation and ready means of detection. Substitution with positron emitting isotopes, such as ¹¹C, ¹⁸F, ¹⁵O and ¹³N, can be useful in Positron Emission Topography (PET) studies for examining substrate receptor occupancy. It is well known in the art that isotope substitution of a hydrogen atom that is bonded to carbon with deuterium [²H] may positively influence the ADME properties of drug candidates by slowing CYP-mediated metabolism [for a review see Nat. Rev. Drug Discov. 15(4): 219-21 (2016)].

Isotopically-labelled compounds can generally be prepared by conventional techniques known to those skilled in the art or by processes analogous to those described herein using an appropriate isotopically-labelled reagent in place of the non-labelled reagent previously employed.

### Pharmaceutical Compositions

A pharmaceutical composition may comprise any compound or a pharmaceutically acceptable salt or derivative as described herein, and a pharmaceutically acceptable excipient. A pharmaceutical composition as described herein may comprise one or more pharmaceutically acceptable excipients, for example pharmaceutically acceptable carriers, diluents, preserving agents, solubilising agents, stabilising agents, disintegrating agents, binding agents, lubricating agents, wetting agents, emulsifiers, sweeteners, colourants, odourants, salts, buffers, coating agents and antioxidants. Suitable excipients and techniques for formulating pharmaceutical compositions are well known in the art (see, e.g. Remington: The Science and Practice of Pharmacy, 20th Ed., ed. A. Gennaro, Lippincott Williams & Wilkins, 2000). Suitable excipients include, without limitation, pharmaceutical grade starch, mannitol, lactose, corn starch, magnesium stearate, stearic acid, alginic acid, sodium saccharin, talcum, cellulose, cellulose derivatives (e.g. hydroxypropylmethylcellulose, carboxymethylcellulose) glucose, sucrose (or other sugar), sodium carbonate, calcium carbonate, magnesium carbonate, sodium phosphate, calcium phosphate, gelatin, agar, pectin, liquid paraffin oil, olive oil, alcohol, detergents, emulsifiers or water (preferably sterile).

A pharmaceutical composition may further comprise an adjuvant and/or one or more additional therapeutically active agent(s).

A pharmaceutical composition may be provided in unit dosage form, will generally be provided in a sealed container and may be provided as part of a kit. Such a kit would normally (although not necessarily) include instructions for use. It may include a plurality of said unit dosage forms.

A pharmaceutical composition may be adapted for administration by any appropriate route, for example by oral, buccal or sublingual routes or parenteral routes, including subcutaneous, intramuscular, intravenous, intraperitoneal, and intradermal, rectal and topical administration, and inhalation. Such compositions may be prepared by any method known in the art of pharmacy, for example by admixing the active ingredient with a excipient(s) under sterile conditions.

For oral administration, the active ingredient may be presented as discrete units, such as tablets, capsules, powders, granulates, solutions, suspensions, and the like.

Formulations suitable for oral administration may also be designed to deliver the compounds described herein in an immediate release manner or in a rate-sustaining manner, wherein the release profile can be delayed, pulsed, controlled, sustained, or delayed and sustained or modified in such a manner which optimises the therapeutic efficacy of the said compounds. Means to deliver compounds in a rate-sustaining manner are known in the art and include slow release polymers that can be formulated with the said compounds to control their release.

Examples of rate-sustaining polymers include degradable and non-degradable polymers that can be used to release the said compounds by diffusion or a combination of diffusion and polymer erosion. Examples of rate-sustaining polymers include hydroxypropyl methylcellulose, hydroxypropyl cellulose, methyl cellulose, ethyl cellulose, sodium carboxymethyl cellulose, polyvinyl alcohol, polyvinyl pyrrolidone, xanthum gum, polymethacrylates, polyethylene oxide and polyethylene glycol.

Liquid (including multiple phases and dispersed systems) formulations include emulsions, suspensions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds described herein may also be used in fast-dissolving, fast-disintegrating dosage forms such as those described in Liang and Chen, Expert Opinion in Therapeutic Patents 2001, 11(6): 981-986.

The formulation of tablets is discussed in H. Lieberman and L. Lachman, Pharmaceutical Dosage Forms: Tablets 1980, vol. 1 (Marcel Dekker, New York).

For administration intranasally or by inhalation, the active ingredient may be presented in the form of a dry powder from a dry powder inhaler or in the form of an aerosol spray of a solution or suspension from a pressurised container, pump, spray, atomiser or nebuliser.

For parenteral administration, the pharmaceutical composition of the invention may be presented in unit-dose or multi-dose containers, e.g. injection liquids in predetermined amounts, for example in sealed vials and ampoules, and may also be stored in a freeze dried (lyophilized) condition requiring only the addition of sterile liquid carrier, e.g. water, prior to use.

For parenteral administration, the compounds described herein may be administered directly into the blood stream, into subcutaneous tissue, into muscle, or into an internal organ. Suitable means for administration include intravenous, intraarterial, intrathecal, intraventricular, intraurethral, intrasternal, intracranial, intramuscular, intrasynovial and subcutaneous. Suitable devices for administration include needle (including microneedle) injectors, needle-free injectors and infusion techniques.

Parenteral formulations are typically aqueous or oily solutions. Where the solution is aqueous, excipients such as sugars (including but not restricted to glucose, mannitol, sorbitol, etc.) salts, carbohydrates and buffering agents (preferably to a pH of from 3 to 9) may be used. For some applications, the compounds described herein may be more suitably formulated as a sterile non-aqueous solution or as a dried form to be used in conjunction with a suitable vehicle such as sterile, pyrogen-free water (WFI).

Parenteral formulations may include implants derived from degradable polymers such as polyesters (e.g. polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds described herein used in the preparation of parenteral solutions may be increased by the use of appropriate formulation techniques, such as the incorporation of co-solvents and/or solubility-enhancing agents such as surfactants, micelle structures and cyclodextrins.

Mixed with such pharmaceutically acceptable excipients, e.g. as described in the standard reference, Gennaro, A. R. et al, Remington: The Science and Practice of Pharmacy (21st Edition, Lippincott Williams & Wilkins, 2005, see especially Part 5: Pharmaceutical Manufacturing), the active agent may be compressed into solid dosage units, such as pills, tablets, or be processed into capsules, suppositories or patches. By means of pharmaceutically acceptable liquids the active agent can be applied as a fluid composition, e.g. as an injection preparation or as an aerosol spray, in the form of a solution, suspension, or emulsion.

For making solid dosage units, the use of conventional additives such as fillers, colorants, polymeric binders and the like is contemplated. In general any pharmaceutically acceptable additive that does not interfere with the function of the active compounds can be used. Suitable carriers with which the active agent described herein can be administered as solid compositions include lactose, starch, cellulose derivatives and the like, or mixtures thereof, used in suitable amounts. For parenteral administration, aqueous suspensions, isotonic saline solutions and sterile injectable solutions may be used, containing pharmaceutically acceptable dispersing agents and/or wetting agents, such as propylene glycol or butylene glycol.

The invention further includes a pharmaceutical composition, as hereinbefore described, in combination with packaging material suitable for said composition, said packaging material including instructions for the use of the composition for the use as hereinbefore described. In some embodiments, the one or more compounds described herein may be used in combination therapies for the treatment of the described conditions i.e., in conjunction with other therapeutic agents. For the case of active compounds combined with other therapies the two or more treatments may be given in individually varying dose schedules and via different routes.

The combination of the agents listed above with a compound described herein would be at the discretion of the physician who would select dosages using his common general knowledge and dosing regimens known to a skilled practitioner.

Where a compound described herein is administered in combination therapy with one, two, three, four or more, preferably one or two, preferably one other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer period apart where required), the precise dosage regimen being commensurate with the properties of the therapeutic agent(s).

In one embodiment, the invention provides a product comprising a compound described herein and another therapeutic agent as a combined preparation for simultaneous, separate or sequential use in therapy. In one embodiment, the therapy is the treatment or prevention of disorders where a reduction of second messenger responses mediated by cyclic 3′,5′-adenosine monophosphate (CAMP) is required. Products provided as a combined preparation include a composition comprising a compound described herein and the other therapeutic agent together in the same pharmaceutical composition, or the compound described herein and the other therapeutic agent in separate form, e.g. in the form of a kit.

In one embodiment, the invention provides a pharmaceutical composition comprising a compound of the invention and another therapeutic agent. Optionally, the pharmaceutical composition may comprise a pharmaceutically acceptable excipient, as described above.

In one embodiment, the invention provides a kit comprising two or more separate pharmaceutical compositions, at least one of which contains a compound described herein. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound described herein and the other therapeutic agent may be manufactured and/or formulated by the same or different manufacturers. Moreover, the compound described herein and the other therapeutic may be brought together into a combination therapy: (i) prior to release of the combination product to physicians (e.g. in the case of a kit comprising the compound described herein and the other therapeutic agent); (ii) by the physician themselves (or under the guidance of the physician) shortly before administration; (iii) in the patient themselves, e.g. during sequential administration of the compound described herein and the other therapeutic agent.

### Method of Manufacture & Method of Treatment

The invention also provides the use of a compound described herein in the manufacture of a medicament for the treatment or prevention of disorders where a reduction of second messenger responses mediated by cyclic 3′,5′-adenosine monophosphate (CAMP) is required, wherein the medicament is prepared for administration with another therapeutic agent. The invention also provides the use of another therapeutic agent in the manufacture of medicament for treating a disease or condition mediated by CAMP for the treatment or prevention of disorders where a reduction of second messenger responses mediated by cAMP is required, wherein the medicament is prepared for administration with a compound described herein.

The invention also provides a compound described herein for use in the treatment or prevention of disorders where a reduction of second messenger responses mediated by cAMP is required, wherein the compound described herein is prepared for administration with another therapeutic agent. The invention also provides another therapeutic agent for use in the treatment or prevention of disorders where a reduction of second messenger responses mediated by CAMP is required, wherein the other therapeutic agent is prepared for administration with a compound described herein. The invention also provides a compound described herein for use in for the treatment or prevention of disorders where a reduction of second messenger responses mediated by cAMP is required, wherein the compound described herein is administered with another therapeutic agent. The invention also provides another therapeutic agent for use in the treatment or prevention of disorders where a reduction of second messenger responses mediated by CAMP is required, wherein the other therapeutic agent is administered with a compound described herein.

The invention also provides the use of a compound described herein in the manufacture of a medicament for the treatment or prevention of disorders where a reduction of second messenger responses mediated by CAMP is required, wherein the patient has previously (e.g. within 24 hours) been treated with another therapeutic agent. The invention also provides the use of another therapeutic agent in the manufacture of a medicament for the treatment or prevention of disorders where a reduction of second messenger responses mediated by cAMP is required, wherein the patient has previously (e.g. within 24 hours) been treated with a compound described herein.

In one embodiment, the other therapeutic agent is:
(i) a presynaptic α-2 adrenergic receptor agonist, optionally clonidine, dexmedetomidine, or guanfacine;
(ii) a β-1 Adrenergic receptor antagonist (“beta-blocker”), optionally Atenolol, Metoprolol, Bisoprolol, Acebutolol, or Betaxolol.

### EXAMPLES

The present invention will now be further described by way of the following non-limiting examples and with reference to the Tables:

Table 1 shows the structures of small molecule PDE4 long form activators according to the present invention.

Table 2 shows enzyme assay data for PDE4D5, a long form of PDE4 and PDE4B2, a short form of PDE4.

Table 3 shows a reduction of cAMP levels in a 3D culture of m-IMCD3 kidney cells treated with compounds of the present invention.

Table 4 shows inhibition of PGE2-stimulated cyst formation in a 3D culture of m-IMCD3 kidney cells treated with compounds of the present invention.

### General Experimental Details

Reactions were monitored by thin layer chromatography (Merck Millipore TLC Silica Gel 60 F₂₅₄). Flash column chromatography was performed on Biotage Isolera® or Büchi Reveleris® X2 Flash Chromatography systems using pre-packed silica gel columns. NMR spectra were recorded using Bruker 300 or 400 MHz spectrometers, using residual signal of deuterated solvent as internal reference at 25° C. (unless otherwise specified). Exchangeable NH and OH residues were not identifiable in the ¹H-NMR spectra in some cases.

### UPLC Methods:

Method A: Instrument: Agilent 1290 Infinity II, 1290 G7120A Bin. Pump, 1290 G7167B Multisampler, 1290 MCT G7116B Column Comp., 1290 G7117B DAD (210-320 nm), PDA (210-320 nm), G6135B MSD (ESI pos/neg) mass range: 90-1500, Column: XSelect CSH XP C18 (50×2.1 mm, 2.5 μm) Flow: 0.8 mL/min Column temp: 40° C., Eluent A: 0.1% Formic acid in Water, Eluent B: 0.1% Formic acid in Acetonitrile, Gradient: t=0 min 5% B, t=0.5 min 5% B, t=4.5 min 98% B; t=5 min 98% B, Postrun: 0.5 min.

Method B: Instrument: Agilent 1290 Infinity II, 1290 G7120A Bin. Pump, 1290 G7167B Multisampler, 1290 MCT G7116B Column Comp., 1290 G7117B DAD (210-320 nm), PDA (210-320 nm), G6135B MSD (ESI pos/neg) mass range: 90-1500, Column: XSelect CSH XP C18 (50×2.1 mm, 2.5 μm) Flow: 0.8 mL/min Column temp: 25, Eluent A: 10 mM Ammonium Bicarbonate in Water (pH 9.5), Eluent B: Acetonitrile, Gradient: t=0 min 5% B, t=0.5 min 5% B, t=4.5 min 98% B; t=5 min 98% B, Postrun: 0.5 min.

Method C: Instrument: Agilent 1290 Infinity II, 1290 G7120A Bin. Pump, 1290 G7167B Multisampler, 1290 MCT G7116B Column Comp., 1290 G7117B DAD (210, 210-320 nm), PDA (210-320 nm), G6135B MSD (ESI pos/neg) mass range: 90-1500, Column: Atlantis T3 (100×3.0 mm, 3 μm) Flow: 0.8 mL/min Column temp: 40° C., Eluent A: 0.1% Formic acid in Water, Eluent B: 0.1% Formic acid in Acetonitrile, Gradient: t=0 min 5% B, t=1 min 5% B, t=10 min 98% B; t=12 min 98% B, Postrun: 2.5 min.

Method D: Instrument: Agilent 1290 Infinity II, 1290 G7120A Bin. Pump, 1290 G7167B Multisampler, 1290 MCT G7116B Column Comp., 1290 G7117B DAD (210, 210-320 nm), PDA (210-320 nm), G6135B MSD (ESI pos/neg) mass range: 90-1500, Column: Atlantis T3 (100×2.1 mm, 1.7 μm) Flow: 0.8 mL/min Column temp: 40° C., Eluent A: 0.1% Formic acid in Water, Eluent B: 0.1% Formic acid in Acetonitrile, Gradient: t=0 min 2% B, t=2 min 2% B, t=10 min 30% B; t=12 min 30% B, Postrun: 2.5 min.

### LCMS Methods

Method A: Instrument: Agilent 1260 Infinity II, 1260 G7112B Bin. Pump, 1260 G7167A Multisampler, 1290 MCT G7116B Column Comp. 1260 G7115A DAD (210, 220 and 210-320 nm), PDA (210-320 nm), G6135B MSD (ESI pos/neg) mass range 90-1500, 1290 G7102A ELSD (Evap: 50° C., Neb: 50° C., gasflow: 1.3 mL/min), Column: XSelect CSH C18 (30×2.1 mm 3.5 μm) Flow: 1 mL/min, Column temp.: 40° C., Eluent A: 0.1% Formic acid in Water, Eluent B: 0.1% Formic acid in Acetonitrile, Gradient: t=0 min 5% B, t=1.6 min 98% B, t=3 min 98% B, Postrun: 1.3 min.

Method B: Instrument: Agilent 1260 Infinity, 1260 G1312B Bin. Pump, 1260 G1367E WPS, 1260 TCC G1316A Column Comp. 1260 G1315C DAD (210-320 nm, 210 and 220 nm), PDA (210-320 nm), G6130B MSD (ESI pos/neg) mass range: 100-1000, Column: Waters XSelect CSH C18 (30×2.1 mm, 3.5 μm), Flow: 1 mL/min Column temp: 25, Eluent A: 10 mM ammonium bicarbonate in water (pH=9), Eluent B: Acetonitrile, Gradient: t=0 min 5% B, t=1.6 min 98% B, t=3 min 98% B, Postrun: 1.4 min.

### Abbreviations:

| | Name | CAS nr |
|---|---|---|
| RuPhos | 2-dicyclohexylphosphino-2′,6′-diisopropoxybiphenyl | 787618-22-8 |
| Pd(Ph₃P)₄ | tetrakis(triphenylphosphine)palladium(0) | 14221-01-3 |
| Xantphos | 9,9-dimethyl-4,5-bis(diphenylphosphino)xanthene | 161265-03-8 |
| Pd₂(dba)₃ | tris(dibenzylideneacetone)dipalladium(0) | 51364-51-3 |
| XPhos | 2-dicyclohexylphosphino-2′,4′,6′-triisopropylbiphenyl | 564483-18-7 |
| (^{t}Bu)PhCPhos | 2-[(tert-butyl)phenylphosphino]-2′,6′- | 1660153-91-2 |
| | bis(N,N)dimethylamino)biphenyl | |
| PdCl₂(dppf)- | [1,1′-bis(diphenylphosphino)ferrocene] | 95464-05-4 |
| CH₂Cl₂ | dichloropalladium(II), complex with dichloromethane | |

The following abbreviations may also be used in the experimental details: CDI (1,1′-carbonyldiimidazole), DCM (dichloromethane), DIPEA (N,N-diisopropylethylamine), DMF (N,N-dimethylformamide), EDC (N-ethyl-N′-(3-dimethylaminopropyl) carbodiimide), h (hours), HOBt (hydroxybenzotriazole), MW (microwave), r.t. (room temperature), SEM [2-(trimethylsilyl) ethoxymethyl], TBDPS (tert-butyldiphenylsilyl), THF (tetrahydrofuran). The following abbreviations are used in the assignment of NMR signals: s (singlet), d (doublet), t (triplet), q (quartet), m (multiplet), app. (approximate), br. (broad), dd (double doublet), dt (double triplet), td (triple doublet).

### Synthetic Procedures for Key Intermediates:

### Intermediate 1: Synthesis of benzyl 3,8-diazabicyclo[3.2.1]octane-3-carboxylate

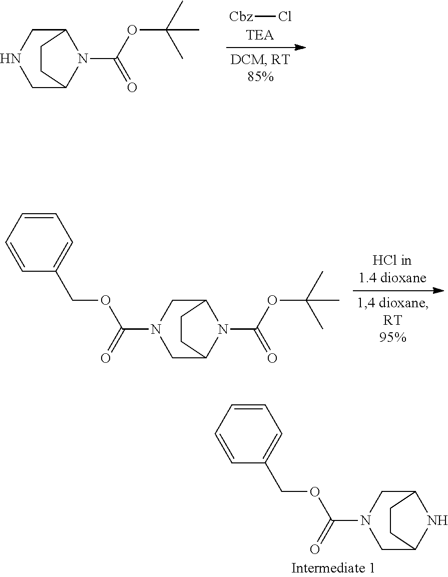 [SMILES: CC(C)(C)OC(=O)N1C2CCC1CNC2 ; O=C(Cl)OCc1ccccc1 ; CC(C)(C)OC(=O)N1C2CCC1CN(C(=O)OCc1ccccc1)C2 ; O=C(OCc1ccccc1)N1CC2CCC(C1)N2]

Under a nitrogen atmosphere, tert-butyl 3,8-diazabicyclo[3.2.1]octane-8-carboxylate (5.00 g, 23.6 mmol) and triethylamine (6.57 mL, 47.1 mmol) were dissolved in dichloromethane (100 mL) and benzyl chloroformate (3.7 mL, 26 mmol) was slowly added. The mixture was stirred at room temperature for 30 minutes, poured into saturated aqueous sodium bicarbonate solution and the layers were separated. The aqueous layer was extracted with dichloromethane twice. The combined organic layers were washed with brine, dried with sodium sulfate and concentrated in vacuo to afford a yellow oil. The crude oil was purified with silica column chromatography (0% to 50% ethyl acetate in n-heptane) and concentrated in vacuo to afford 3-benzyl 8-(tert-butyl) 3,8-diazabicyclo[3.2.1]octane-3,8-dicarboxylate (8.15 g, 85%) as a colorless oil.

To a solution of 3-benzyl 8-(tert-butyl) 3,8-diazabicyclo[3.2.1]octane-3,8-dicarboxylate (8.15 g, 20.0 mmol) in 1,4-dioxane (40 mL) was added 4 M hydrochloric acid in 1,4-dioxane (40 mL, 160 mmol) and the mixture was stirred at room temperature for 3 hours. The mixture was concentrated in vacuo and partitioned between ethyl acetate and saturated aqueous sodium bicarbonate solution. The layers were separated, and the aqueous layer was extracted with ethyl acetate twice. The combined organic layers were washed with brine, dried with sodium sulfate and concentrated in vacuo to afford benzyl 3,8-diazabicyclo[3.2.1]octane-3-carboxylate (4.95 g, 95%, Intermediate 1) as a light-yellow oil. ¹H-NMR: δ_{H} (400 MHZ, CDCl₃) 7.39-7.28 (5H, m), 5.13 (2H, s), 3.89-3.72 (2H, m), 3.57-3.41 (2H, m), 3.13-2.97 (2H, m), 1.86 (1H, s), 1.80-1.66 (4H, m).

### Intermediate 4: Synthesis of benzyl 8-(5,6,7,8-tetrahydro-1,6-naphthyridin-2-yl)-3,8-diazabicyclo[3.2.1]octane-3-carboxylate hydrochloride

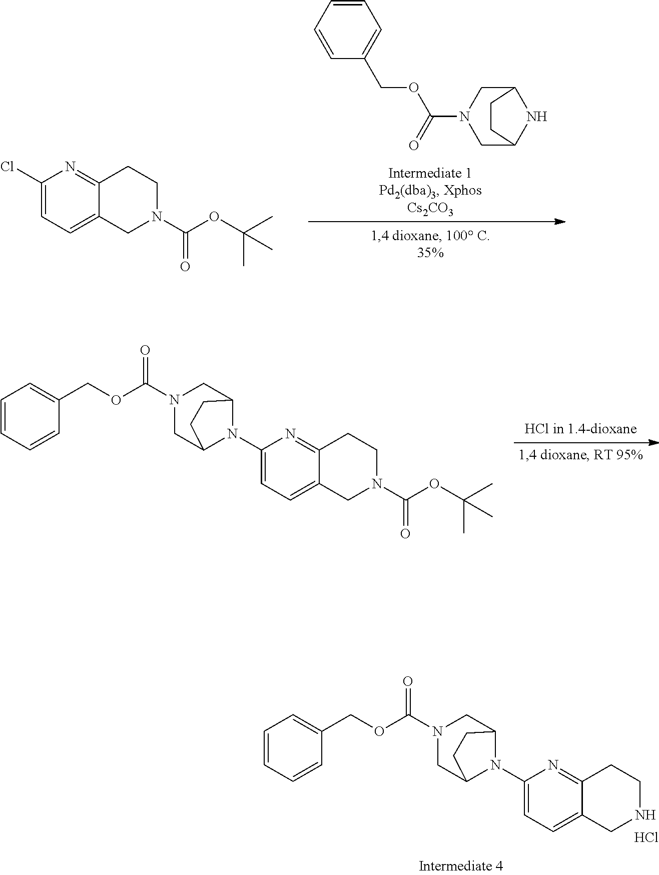 [SMILES: O=C(OCc1ccccc1)N1CC2CCC(C1)N2 ; CC(C)(C)OC(=O)N1CCc2nc(Cl)ccc2C1 ; CC(C)(C)OC(=O)N1CCc2nc(N3C4CCC3CN(C(=O)OCc3ccccc3)C4)ccc2C1 ; O=C(OCc1ccccc1)N1CC2CCC(C1)N2c1ccc2c(n1)CCNC2 ; Cl]

Under an argon atmosphere, Pd₂(dba)₃ (50 mg, 0.06 mmol) was added to a solution of tert-butyl 2-chloro-7,8-dihydro-1,6-naphthyridine-6(5H)-carboxylate (199 mg, 0.74 mmol), benzyl 3,8-diazabicyclo[3.2.1]octane-3-carboxylate (275 mg, 1.12 mmol, Intermediate 1), XPhos (27 mg, 0.06 mmol) and cesium carbonate (620 mg, 1.90 mmol) in 1,4-dioxane (5 mL). The mixture was heated to 100° C. in a sealed vial for 21 hours. The mixture was cooled to room temperature and partitioned between saturated aqueous sodium bicarbonate solution and ethyl acetate. The layers were separated, and the aqueous layer was extracted with ethyl acetate twice. The combined organic layers were dried with sodium sulfate and concentrated in vacuo to afford a brown oil. The oil was purified with silica column chromatography (2% to 75% ethyl acetate in n-heptane) and concentrated in vacuo to afford tert-butyl 2-(3-((benzyloxy) carbonyl)-3,8-diazabicyclo[3.2.1]octan-8-yl)-7,8-dihydro-1,6-naphthyridine-6(5H)-carboxylate (187 mg, 35%) as an off-white solid.

To a solution of tert-butyl 2-(3-((benzyloxy) carbonyl)-3,8-diazabicyclo[3.2.1]octan-8-yl)-7,8-dihydro-1,6-naphthyridine-6(5H)-carboxylate (187 mg, 0.39 mmol) in methanol (5 mL) was added 4 M hydrochloric acid in 1,4-dioxane (0.5 mL, 2.00 mmol) and the mixture was stirred at room temperature for 22 hours. The mixture was concentrated in vacuo to afford benzyl 8-(5,6,7,8-tetrahydro-1,6-naphthyridin-2-yl)-3,8-diazabicyclo[3.2.1]octane-3-carboxylate hydrochloride (188 mg, 96%, Intermediate 4) as a yellow solid. LCMS (Method A): t_{R} 1.28 min, 90%, MS (ESI) 379.2 (M+H)⁺.

### Example 39: Synthesis of 1-(2-(3,8-diazabicyclo[3.2.1]octan-8-yl)-7,8-dihydro-1,6-naphthyridin-6(5H)-yl)-2-cyclopentylethan-1-one

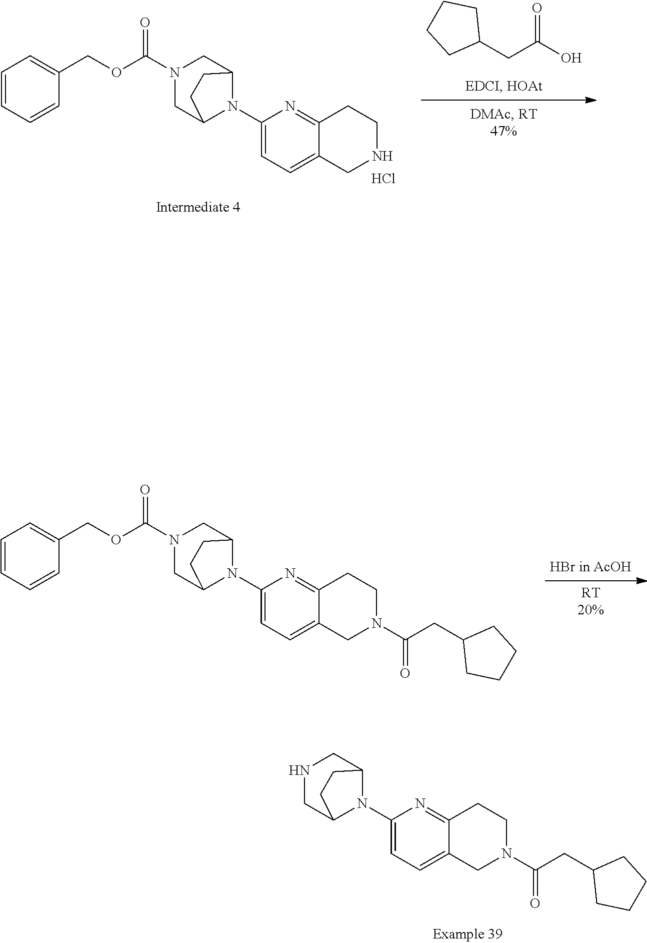 [SMILES: O=C(OCc1ccccc1)N1CC2CCC(C1)N2c1ccc2c(n1)CCNC2 ; O=C(O)CC1CCCC1 ; Cl ; O=C(CC1CCCC1)N1CCc2nc(N3C4CCC3CN(C(=O)OCc3ccccc3)C4)ccc2C1 ; O=C(CC1CCCC1)N1CCc2nc(N3C4CCC3CNC4)ccc2C1]

To a solution of benzyl 8-(5,6,7,8-tetrahydro-1,6-naphthyridin-2-yl)-3,8-diazabicyclo[3.2.1]octane-3-carboxylate hydrochloride (76 mg, 0.18 mmol, Intermediate 4) and cyclopentylacetic acid (26 μL, 0.21 mmol) in N,N-dimethylformamide (2 mL) were added triethylamine (80 μL, 0.58 mmol), N-(3-dimethylaminopropyl)-N′-ethylcarbodiimide hydrochloride (42 mg, 0.22 mmol) and 1-hydroxy-7-azabenzotriazole (3.5 mg, 0.026 mmol) and the reaction was stirred at room temperature for 16 hours. The mixture was concentrated in vacuo and the residue was purified by preparative reversed phase chromatography (Waters XSelect™ CSH C18 column, ammonium bicarbonate in water/acetonitrile) to afford benzyl 8-(6-(2-cyclopentylacetyl)-5,6,7,8-tetrahydro-1,6-naphthyridin-2-yl)-3,8-diazabicyclo[3.2.1]octane-3-carboxylate (58 mg, 65%).

To a solution of benzyl 8-(6-(2-cyclopentylacetyl)-5,6,7,8-tetrahydro-1,6-naphthyridin-2-yl)-3,8-diazabicyclo[3.2.1]octane-3-carboxylate (58 mg, 0.12 mmol) in acetic acid (2.5 mL) was added 33% hydrogen bromide in acetic acid (0.3 mL, 1.71 mmol) and the reaction was stirred at room temperature for 16 hours. The mixture was concentrated in vacuo, the residue was transferred to an SCX-cartridge, the product was eluted with 2N ammonia in methanol, and the resulting solution was concentrated. The residue was lyophilized to afford 1-(2-(3,8-diazabicyclo[3.2.1]octan-8-yl)-7,8-dihydro-1,6-naphthyridin-6(5H)-yl)-2-cyclopentylethan-1-one (36 mg, 86%, Example 39) as a white solid. 1H-NMR: δ_{H} (400 MHZ, CDCl₃, mixture of rotamers) 7.21 (0.6H, d, J 8.5), 7.17 (0.4H, d, J 8.4), 6.53-6.40 (1H, m), 4.82-4.38 (4H, m), 3.88 (0.8H, t, J 5.9), 3.74 (1.2H, t, J 6.0), 3.14 (2H, m), 2.95-2.77 (2H, m), 2.64 (2H, m), 2.43 (2H, m), 2.38-2.23 (1H, m), 2.13-1.83 (6H, m), 1.70-1.49 (4H, m), 1.33-1.07 (2H, m); UPLC (Method C): t_{R} 3.58 min, 100%, MS (ESI) 355.2 (M+H)⁺.

The following examples (40-49) were prepared using procedures analogous to Example 39, using the appropriate starting materials.

### Example 40: Synthesis of 1-(2-(3,8-diazabicyclo[3.2.1]octan-8-yl)-7,8-dihydro-1,6-naphthyridin-6(5H)-yl)-2-(4,4-difluorocyclohexyl)ethan-1-one

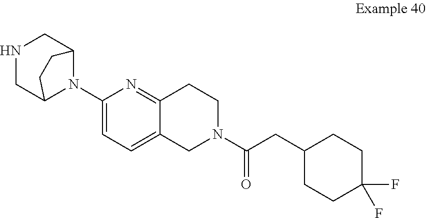 [SMILES: O=C(CC1CCC(F)(F)CC1)N1CCc2nc(N3C4CCC3CNC4)ccc2C1]

Prepared using 2-(4,4-difluorocyclohexyl) acetic acid as the acid component.

¹H-NMR: δ_{H} (400 MHZ, DMSO-d₆) 7.29 (1H, d, J 8.4), 6.56 (1H, t, J 8.0), 4.53-4.42 (2H, m), 4.34 (2H, br s), 3.72 (2H, m), 2.85 (2H, m), 2.72 (1H, t, J 5.9), 2.62 (1H, t, J 6.0), 2.45 (2H, dd, J 12.2, 2.2), 2.36 (2H, t, J 7.1), 2.06-1.94 (2H, m), 1.92-1.65 (10H, m), 1.29-1.14 (2H, m); UPLC (Method A): t_{R} 1.33 min, 97%, MS (ESI) 405.4 (M+H)⁺.

### Example 41: Synthesis of 1-(2-(3,8-diazabicyclo[3.2.1]octan-8-yl)-7,8-dihydro-1,6-naphthyridin-6(5H)-yl)-2-phenoxyethan-1-one

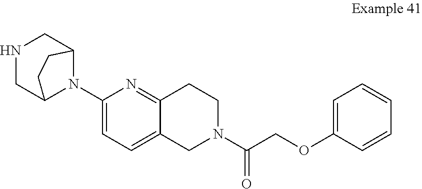 [SMILES: O=C(COc1ccccc1)N1CCc2nc(N3C4CCC3CNC4)ccc2C1]

Prepared using phenoxyacetic acid as the acid component.

¹H-NMR: δ_{H} (400 MHZ, CDCl₃, mixture of rotamers) 7.33-7.27 (2H, m), 7.20 (0.6H, d, J 8.5), 7.16 (0.4H, d, J 8.5), 7.12-6.93 (3H, m), 6.43 (1H, d, J 8.6), 4.90-4.74 (2H, m), 4.70-4.58 (2H, m), 4.43 (2H, br s), 3.88 (1H, t, J 5.9), 3.84 (1H, t, J 5.8), 3.13 (2H, m), 2.97-2.79 (2H, m), 2.64 (2H, m), 2.09-1.91 (4H, m); UPLC (Method C): t_{R} 3.39 min, 100%, MS (ESI) 379.2 (M+H)⁺.

### Example 42: Synthesis of 1-(2-(3,8-diazabicyclo[3.2.1]octan-8-yl)-7,8-dihydro-1,6-naphthyridin-6(5H)-yl)-3,3-dimethylbutan-1-one

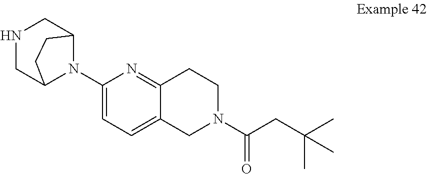 [SMILES: CC(C)(C)CC(=O)N1CCc2nc(N3C4CCC3CNC4)ccc2C1]

Prepared using 3,3-dimethylbutanoic acid as the acid component.

¹H-NMR: δ_{H} (400 MHZ, CDCl₃, mixture of rotamers) 7.21 (0.6H, d, J 8.5), 7.15 (0.4H, d, J 8.5), 6.46-6.39 (1H, m), 4.61 (1.2H, s), 4.52 (0.8H, s), 4.43 (2H, br s), 3.89 (0.8H, t, J 6.0), 3.77 (1.2H, t, J 6.0), 3.18-3.10 (2H, m), 2.89-2.77 (2H, m), 2.68-2.60 (2H, m), 2.38-2.32 (2H, m), 2.06-1.90 (4H, m), 1.09 (5.4H, s), 1.05 (3.6H, s); UPLC (Method C): t_{R} 3.37 min, 100%, MS (ESI) 343.2 (M+H)⁺.

### Example 43: Synthesis of 1-(2-(3,8-diazabicyclo[3.2.1]octan-8-yl)-7,8-dihydro-1,6-naphthyridin-6(5H)-yl)-3-methylbutan-1-one

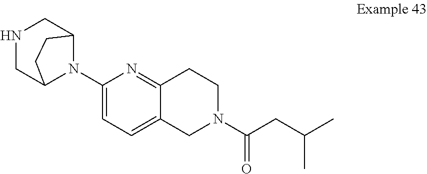 [SMILES: CC(C)CC(=O)N1CCc2nc(N3C4CCC3CNC4)ccc2C1]

Prepared using isovaleric acid as the acid component.

¹H-NMR: δ_{H} (400 MHZ, CDCl₃, mixture of rotamers) 7.21 (0.6H, d, J 8.5), 7.17 (0.4H, d, J 8.5), 6.43 (1H, m), 4.61 (1H, s), 4.49 (1H, s), 4.44 (2H, br s), 3.88 (0.8H, t, J 6.0), 3.74 (1.2H, t, J 5.9), 3.15 (2H, d, J 12.2), 2.88-2.77 (2H, m), 2.67 (2H, d, J 11.5), 2.30 (2H, m), 2.18 (1H, m), 2.05-1.94 (4H, m), 0.98 (6H, m); UPLC (Method C): t_{R} 3.09 min, 100%, MS (ESI) 329.2 (M+H)⁺.

### Example 44: Synthesis of 1-(2-(3,8-diazabicyclo[3.2.1]octan-8-yl)-7,8-dihydro-1,6-naphthyridin-6(5H)-yl)-2,3-dimethylbutan-1-one

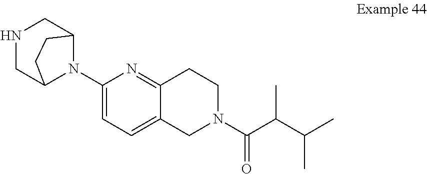 [SMILES: CC(C)C(C)C(=O)N1CCc2nc(N3C4CCC3CNC4)ccc2C1]

Prepared using 2,3-dimethylbutanoic acid as the acid component.

¹H-NMR: δ_{H} (400 MHZ, CDCl₃, mixture of rotamers) 7.21 (0.6H, d, J 8.5), 7.17 (0.4H, d, J 8.5), 6.43 (1H, d, J 8.5), 4.62 (1.2H, d, J 8.7), 4.55 (0.8H, d, J 8.7), 4.43 (2H, br s), 3.93-3.86 (0.8H, m), 3.84-3.75 (1.2H, m), 3.14 (2H, dd, J 12.3, 1.8), 2.90-2.77 (2H, m), 2.63 (2H, d, J 12.4), 2.57-2.46 (1H, m), 2.06-1.89 (5H, m), 1.16-1.06 (3H, m), 0.99-0.84 (6H, m); UPLC (Method C): t_{R} 3.31 min, 98%, MS (ESI) 343.2 (M+H)⁺.

### Example 45: Synthesis of 1-(2-(3,8-diazabicyclo[3.2.1]octan-8-yl)-7,8-dihydro-1,6-naphthyridin-6(5H)-yl)-2-phenylethan-1-one

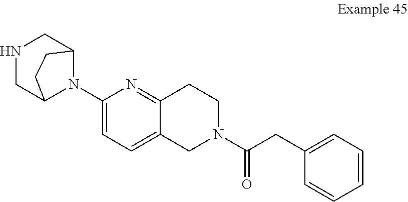 [SMILES: O=C(Cc1ccccc1)N1CCc2nc(N3C4CCC3CNC4)ccc2C1]

Prepared using 2-phenylacetic acid as the acid component.

¹H-NMR: δ_{H} (400 MHZ, CDCl₃, mixture of rotamers) 7.35-7.22 (5H, m), 7.20 (0.6H, d, J 8.5), 7.05 (0.4H, d, J 8.5), 6.42 (0.6H, d, J 8.6), 6.37 (0.4H, d, J 8.6), 4.63 (1.2H, s), 4.46 (0.8H, s), 4.41 (2H, s), 3.90 (0.8H, t, J 6.0), 3.82 (2H, m), 3.70 (1.2H, t, J 6.0), 3.11 (2H, d, J 12.3), 2.82 (0.8H, m), 2.68-2.58 (3.2H, m), 2.05-1.90 (4H, m); UPLC (Method A): t_{R} 1.17 min, 100%, MS (ESI) 363.2 (M+H)⁺.

### Example 46: Synthesis of 1-(2-(3,8-diazabicyclo[3.2.1]octan-8-yl)-7,8-dihydro-1,6-naphthyridin-6(5H)-yl)-2-(4-fluorophenoxy)ethan-1-one

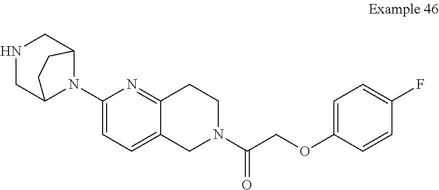 [SMILES: O=C(COc1ccc(F)cc1)N1CCc2nc(N3C4CCC3CNC4)ccc2C1]

Prepared using 2-(4-fluorophenoxy) acetic acid as the acid component.

¹H-NMR: δ_{H} (400 MHZ, CDCl₃, mixture of rotamers) 7.20 (0.6H, d, J 8.6), 7.17 (0.4H, d, J 8.6), 7.02-6.87 (4H, m), 6.43 (1H, d, J 8.6), 4.76-4.70 (2H, m), 4.64-4.56 (2H, m), 4.43 (2H, br s), 3.88 (0.8H, t, J 6.0), 3.82 (1.2H, t, J 6.0), 3.13 (2H, d, J 12.0), 2.87 (1.2H, t, J 6.0), 2.82 (0.8H, t, J 6.0), 2.65 (2H, d, J 12.2), 2.00-1.90 (4H, m); UPLC (Method A): t_{R} 1.28 min, 100%, MS (ESI) 397.4 (M+H)⁺.

### Example 47: Synthesis of (2-(3,8-diazabicyclo[3.2.1]octan-8-yl)-7,8-dihydro-1,6-naphthyridin-6(5H)-yl)(cyclopentyl)methanone

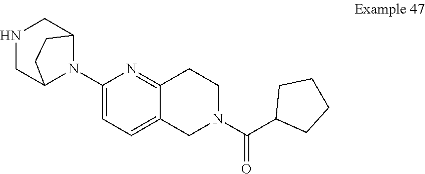 [SMILES: O=C(C1CCCC1)N1CCc2nc(N3C4CCC3CNC4)ccc2C1]

Prepared using cyclopentane carboxylic acid as the acid component.

¹H-NMR: δ_{H} (400 MHZ, CDCl₃, mixture of rotamers) 7.25-7.07 (1H, m), 6.50-6.34 (1H, m), 4.61 (1.2H, s), 4.54 (0.8H, s), 4.43 (2H, br s), 3.94-3.75 (2H, s), 3.20-3.11 (2H, m), 3.06-2.92 (1H, m), 2.92-2.72 (2H, m), 2.69-2.60 (2H, m), 2.10-1.73 (10H, m), 1.64-1.53 (2H, m); UPLC (Method A): t_{R} 1.12 min, 99%, MS (ESI) 341.2 (M+H)⁺.

### Example 48: Synthesis of 1-(2-(3,8-diazabicyclo[3.2.1]octan-8-yl)-7,8-dihydro-1,6-naphthyridin-6(5H)-yl)-2-(4-fluorophenyl)ethan-1-one

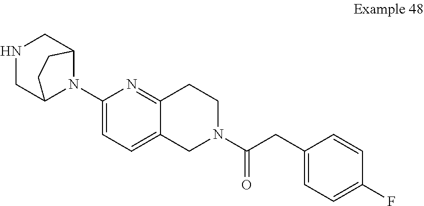 [SMILES: O=C(Cc1ccc(F)cc1)N1CCc2nc(N3C4CCC3CNC4)ccc2C1]

Prepared using 2-(4-fluorophenyl) acetic acid as the acid component.

¹H-NMR: δ_{H} (400 MHZ, CDCl₃, mixture of rotamers) 7.26-7.17 (3H, m), 7.08-6.95 (2H, m), 6.42 (0.6H, d, J 8.4), 6.38 (0.4H, d, J 8.5), 4.62 (1.2H, s), 4.47 (0.8H, s), 4.41 (2H, br s), 3.89 (0.8H, t, J 6.0), 3.81-3.75 (2H, m), 3.71 (1.2H, t, J 6.0), 3.16-3.08 (2H, m), 2.85-2.78 (0.8H, m), 2.71-2.58 (3.2H, m), 2.04-1.90 (4H, m); UPLC (Method A): t_{R} 0.61 min, 99%, MS (ESI) 381.7 (M+H)⁺.

### Example 49: Synthesis of 1-(2-(3,8-diazabicyclo[3.2.1]octan-8-yl)-7,8-dihydro-1,6-naphthyridin-6(5H)-yl)-2,2-difluoro-2-phenylethan-1-one

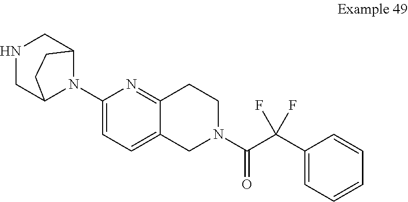 [SMILES: O=C(N1CCc2nc(N3C4CCC3CNC4)ccc2C1)C(F)(F)c1ccccc1]

Prepared using 2,2-difluoro-2-phenylacetic acid as the acid component.

¹H-NMR: δ_{H} (400 MHZ, CDCl₃, mixture of rotamers) 7.65-7.36 (5H, m), 7.20 (0.7H, d, J 8.6), 6.90 (0.3H, d, J 8.6), 6.43 (0.7H, d, J 8.5), 6.31 (0.3H, d, J 8.4), 4.68 (1.4H, s), 4.46 (0.6H, s), 4.40 (2H, br s), 3.95 (0.6H, t, J 6.0), 3.71 (1.4H, t, J 5.9), 3.14-3.07 (2H, m), 2.90-2.84 (0.6H, m), 2.64-2.56 (3.4H, m), 2.03-1.88 (4H, m); UPLC (Method A): t_{R} 0.74 min, 100%, MS (ESI) 399.7 (M+H)⁺.

### Example 59: Synthesis of 2-(3,8-diazabicyclo[3.2.1]octan-8-yl)-N-(4,4-difluorocyclohexyl)-7,8-dihydro-1,6-naphthyridine-6(5H)-carboxamide

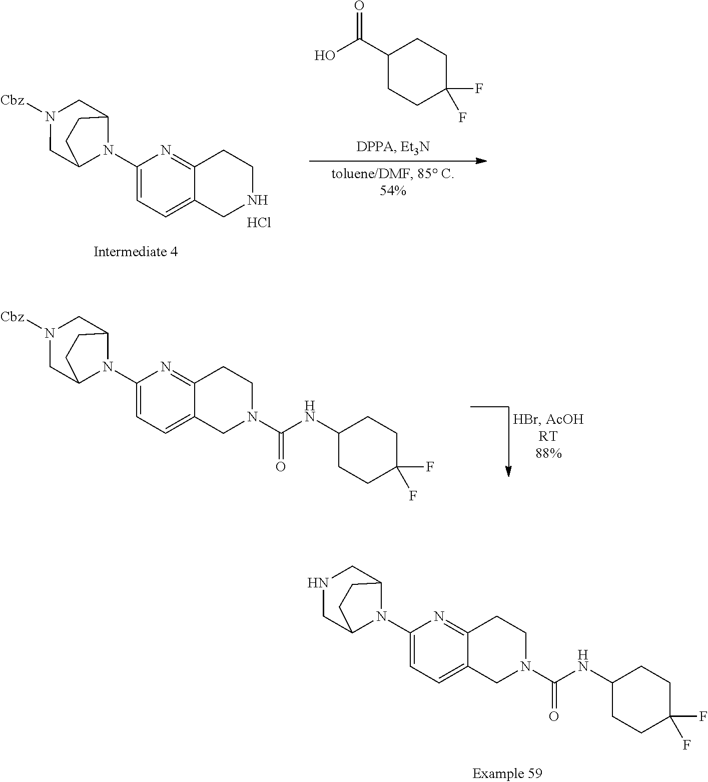 [SMILES: O=C(OCc1ccccc1)N1CC2CCC(C1)N2c1ccc2c(n1)CCNC2 ; O=C(O)C1CCC(F)(F)CC1 ; Cl ; O=C(NC1CCC(F)(F)CC1)N1CCc2nc(N3C4CCC3CN(C(=O)OCc3ccccc3)C4)ccc2C1 ; O=C(NC1CCC(F)(F)CC1)N1CCc2nc(N3C4CCC3CNC4)ccc2C1]

Under argon atmosphere, to a solution of 4,4-difluorocyclohexanecarboxylic acid (57 mg, 0.35 mmol) and triethylamine (49 μL, 0.35 mmol) in toluene (2.5 mL) was added diphenylphosphoryl azide (75 μL, 0.35 mmol) and the mixture was heated to 85° C. for 2 hours. The mixture was cooled to room temperature and added to a solution of benzyl 8-(5,6,7,8-tetrahydro-1,6-naphthyridin-2-yl)-3,8-diazabicyclo[3.2.1]octane-3-carboxylate hydrochloride (84 mg, 0.20 mmol, Intermediate 4) and triethylamine (57 μL, 0.41 mmol) in toluene (1 mL) and N,N-dimethylformamide (0.5 mL). The reaction was stirred at room temperature under argon atmosphere for 16 hours. The mixture was partitioned between saturated aqueous sodium bicarbonate solution and ethyl acetate. The layers were separated and the aqueous layer was extracted with ethyl acetate twice. The combined organic layers were washed with brine, dried with sodium sulfate and concentrated in vacuo. The residue was purified with silica column chromatography (30% to 100% ethyl acetate in n-heptane) and concentrated in vacuo to afford benzyl 8-(6-((4,4-difluorocyclohexyl) carbamoyl)-5,6,7,8-tetrahydro-1,6-naphthyridin-2-yl)-3,8-diazabicyclo[3.2.1]octane-3-carboxylate (59 mg, 50%) as a colourless gum. LCMS (Method A): t_{R} 1.70 min, 98%, MS (ESI) 540.4 (M+H)⁺.

To a solution of benzyl 8-(6-((4,4-difluorocyclohexyl) carbamoyl)-5,6,7,8-tetrahydro-1,6-naphthyridin-2-yl)-3,8-diazabicyclo[3.2.1]octane-3-carboxylate (59 mg, 0.11 mmol) in acetic acid (2.5 mL) was added 33% hydrogen bromide in acetic acid (290 μL, 1.66 mmol) and the reaction was stirred at room temperature for 16 hours. The mixture was concentrated in vacuo, purified with SCX (ion exchange) chromatography (washed with methanol and eluted with 3.5 M ammonia in methanol) to afford 2-(3,8-diazabicyclo[3.2.1]octan-8-yl)-N-(4,4-difluorocyclohexyl)-7,8-dihydro-1,6-naphthyridine-6(5H)-carboxamide (39 mg, 88%, Example 59) as a white solid. ¹H-NMR: δ_{H} (400 MHZ, CDCl₃): 7.18 (1H, d, J 8.5), 6.42 (1H, d, J 8.6), 4.43 (2H, br s), 4.39 (2H, s), 4.30 (1H, d, J 7.5), 3.84 (1H, d, J 9.8), 3.64 (2H, t, J 5.9), 3.13 (2H, dd, J 12.4, 1.8), 2.84 (2H, t, J 5.9), 2.63 (2H, dd, J 12.2, 2.3), 2.12-1.80 (10H, m), 1.57-1.46 (2H, m); UPLC (Method A): t_{R} 1.11 min, 99%, MS (ESI) 406.2 (M+H)⁺.

### Example 61: Synthesis of (2-(3,8-diazabicyclo[3.2.1]octan-8-yl)-7,8-dihydro-1,6-naphthyridin-6(5H)-yl)(pyrrolidin-1-yl)methanone

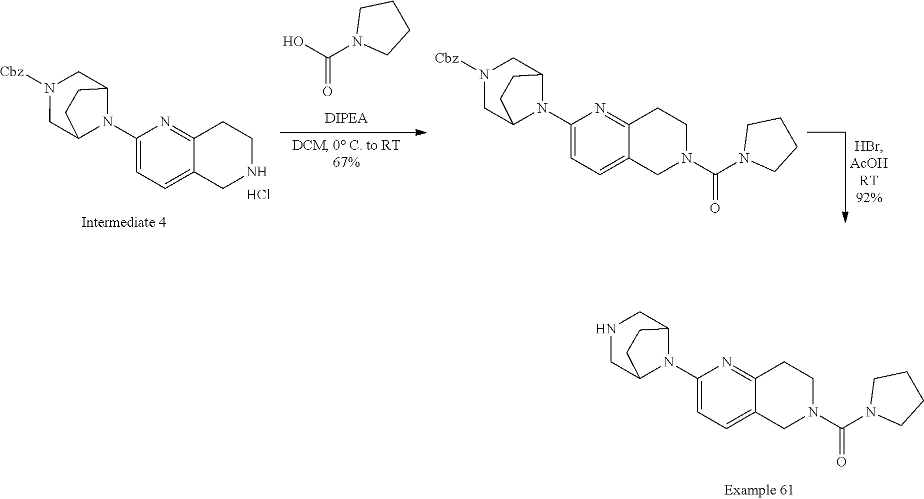 [SMILES: O=C(OCc1ccccc1)N1CC2CCC(C1)N2c1ccc2c(n1)CCNC2 ; Cl ; O=C(O)N1CCCC1 ; O=C(OCc1ccccc1)N1CC2CCC(C1)N2c1ccc2c(n1)CCN(C(=O)N1CCCC1)C2 ; O=C(N1CCCC1)N1CCc2nc(N3C4CCC3CNC4)ccc2C1]

To a solution of benzyl 8-(5,6,7,8-tetrahydro-1,6-naphthyridin-2-yl)-3,8-diazabicyclo[3.2.1]octane-3-carboxylate hydrochloride (76 mg, 0.18 mmol, Intermediate 4) in dichloromethane (3 mL) and diisopropylethylamine (96 μL, 0.55 mmol) at 0° C. was added 1-pyrrolidinecarbonyl chloride (40 μL, 0.36 mmol) and the mixture was stirred at 0° C. for 10 minutes. The mixture was concentrated in vacuo, the residue was purified with preparative reversed phase chromatography (Waters XSelect™ CSH C18 column, ammonium bicarbonate in water/acetonitrile) and lyophilized to afford benzyl 8-(6-(pyrrolidine-1-carbonyl)-5,6,7,8-tetrahydro-1,6-naphthyridin-2-yl)-3,8-diazabicyclo[3.2.1]octane-3-carboxylate (59 mg, 67%) as a white solid. LCMS (Method A): t_{R} 1.62 min, 98%, MS (ESI) 476.4 (M+H)⁺. To a solution of benzyl 8-(6-(pyrrolidine-1-carbonyl)-5,6,7,8-tetrahydro-1,6-naphthyridin-2-yl)-3,8-diazabicyclo[3.2.1]octane-3-carboxylate (58 mg, 0.12 mmol) in acetic acid (2 mL) was added 33% hydrogen bromide in acetic acid (290 μL, 1.66 mmol) and the reaction was stirred at room temperature for 16 hours. The mixture was concentrated in vacuo, purified with SCX (ion exchange) chromatography (washed with methanol and eluted with 3.5 M ammonia in methanol) to afford (2-(3,8-diazabicyclo[3.2.1]octan-8-yl)-7,8-dihydro-1,6-naphthyridin-6(5H)-yl)(pyrrolidin-1-yl) methanon (38 mg, 92%, Example 61) as an off white solid. ¹H-NMR: δ_{H} (400 MHz, CDCl₃) 7.17 (1H, d, J 8.5), 6.41 (1H, d, J 8.6), 4.46-4.39 (2H, m), 4.30 (2H, s), 3.55 (2H, t, J 5.9), 3.46-3.38 (4H, m), 3.14 (2H, dd, J 12.2, 1.8), 2.86 (2H, t, J 5.8), 2.63 (2H, dd, J 12.4, 2.4), 2.06-1.96 (2H, m), 1.96-1.88 (2H, m), 1.87-1.80 (4H, m). UPLC (Method D): t_{R} 4.01 min, 99%, MS (ESI) 342.2 (M+H)⁺.

### Example 63: Synthesis of 2-(3,8-diazabicyclo[3.2.1]octan-8-yl)-N-cyclopentyl-N-methyl-7,8-dihydro-1,6-naphthyridine-6(5H)-carboxamide

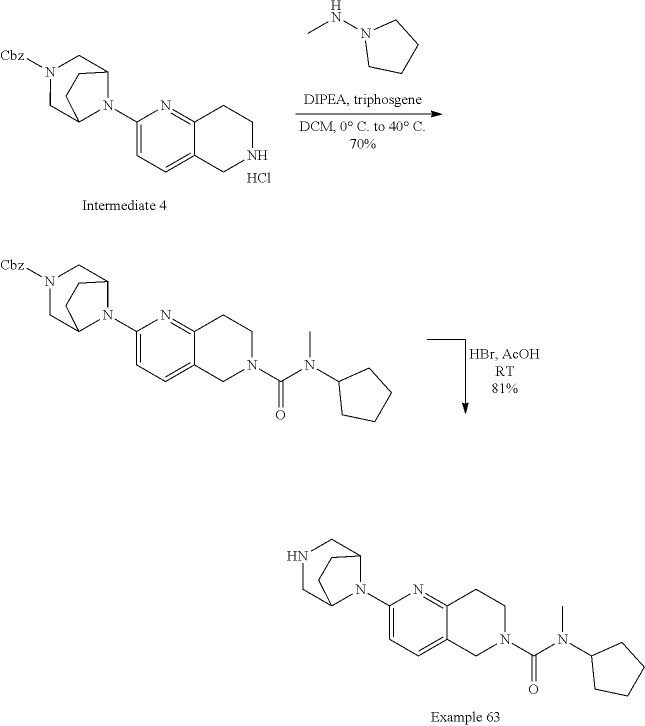

To a solution of benzyl 8-(5,6,7,8-tetrahydro-1,6-naphthyridin-2-yl)-3,8-diazabicyclo[3.2.1]octane-3-carboxylate hydrochloride (76 mg, 0.18 mmol, Intermediate 4) and diisopropylethylamine (80 μL, 0.46 mmol) in dichloromethane (3 mL) at 0° C. was added triphosgene (38 mg, 0.13 mmol) and the reaction was stirred at 0° C. for 30 minutes. Next, N-methylcyclopentanamine (158 μL, 0.13 mmol) was added and the reaction was heated to 40° C. for 2 days. The mixture was cooled to room temperature and concentrated in vacuo. The residue was purified with silica column chromatography (20% to 100% ethyl acetate in n-heptane) and concentrated in vacuo to afford benzyl 8-(6-(cyclopentyl(methyl) carbamoyl)-5,6,7,8-tetrahydro-1,6-naphthyridin-2-yl)-3,8-diazabicyclo[3.2.1]octane-3-carboxylate (65 mg, 70%) as a colorless gum. LCMS (Method A): t_{R} 1.76 min, 99%, MS (ESI) 504.4 (M+H)⁺. To a solution of benzyl 8-(6-(cyclopentyl(methyl) carbamoyl)-5,6,7,8-tetrahydro-1,6-naphthyridin-2-yl)-3,8-diazabicyclo[3.2.1]octane-3-carboxylate (65 mg, 0.13 mmol) in acetic acid (2 mL) was added 33% hydrogen bromide in acetic acid (340 μL, 1.94 mmol) and the reaction was stirred at room temperature for 16 hours. The mixture was concentrated in vacuo, purified with SCX (ion exchange) chromatography (washed with methanol and eluted with 3.5 M ammonia in methanol) to afford 2-(3,8-diazabicyclo[3.2.1]octan-8-yl)-N-cyclopentyl-N-methyl-7,8-dihydro-1,6-naphthyridine-6(5H)-carboxamide (38 mg, 81%, Example 63) as an off-white solid. ¹H-NMR: δ_{H} (400 MHZ, CDCl₃) 7.17 (1H, d, J 8.5), 6.41 (1H, d, J 8.5), 4.43 (2H, m), 4.24 (2H, s), 4.23-4.13 (1H, m), 3.50 (2H, t, J 5.8), 3.15 (2H, m), 2.87 (2H, m), 2.76 (3H, s), 2.64 (2H, m), 2.06-1.89 (4H, m), 1.88-1.80 (2H, m), 1.71-1.64 (2H, m), 1.62-1.52 (4H, m); UPLC (Method A): t_{R} 1.27 min, 99%, MS (ESI) 370.2 (M+H)⁺.

### Example 64: Synthesis of 2-(3,8-diazabicyclo[3.2.1]octan-8-yl)-N-cyclopentyl-N-ethyl-7,8-dihydro-1,6-naphthyridine-6(5H)-carboxamide

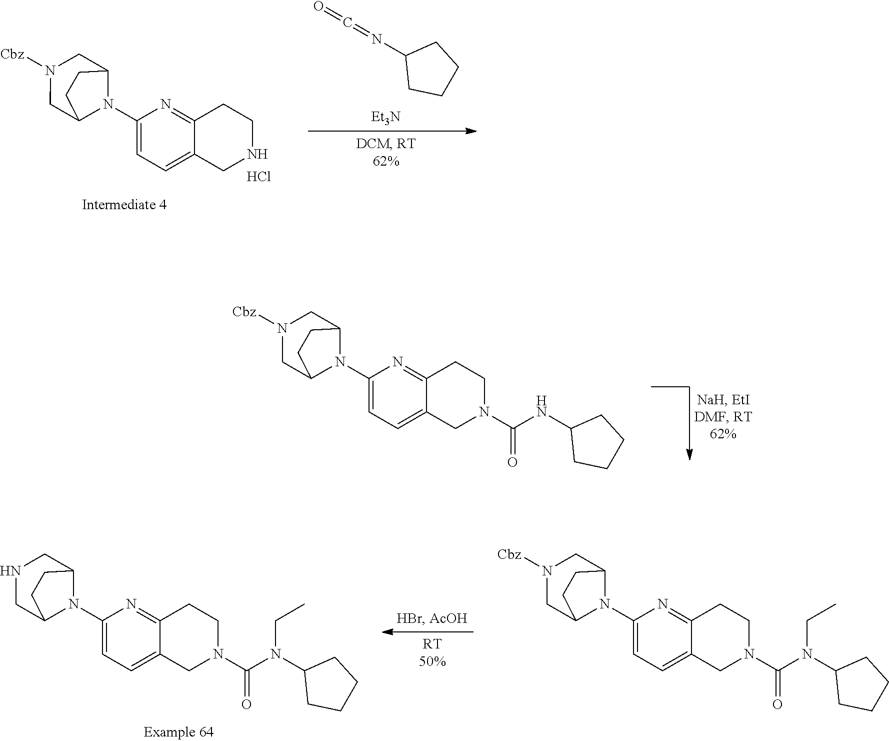

To a solution of benzyl 8-(5,6,7,8-tetrahydro-1,6-naphthyridin-2-yl)-3,8-diazabicyclo[3.2.1]octane-3-carboxylate hydrochloride (66 mg, 0.16 mmol, Intermediate 4) and triethylamine (66 μL, 0.48 mmol) in dichloromethane (2 mL) was added cyclopentyl isocyanate (22 μL, 0.20 mmol) and the reaction was stirred at room temperature for 16 hours. The mixture was cooled to room temperature and concentrated in vacuo. The residue was purified with silica column chromatography (0% to 5% methanol in dichloromethane) and concentrated in vacuo to afford benzyl 8-(6-(cyclopentyl(ethyl) carbamoyl)-5,6,7,8-tetrahydro-1,6-naphthyridin-2-yl)-3,8-diazabicyclo[3.2.1]octane-3-carboxylate (49 mg, 62%) as a colorless gum. LCMS (Method A): t_{R} 1.68 min, 99%, MS (ESI) 490.4 (M+H)⁺.

To a solution of benzyl 8-(6-(cyclopentylcarbamoyl)-5,6,7,8-tetrahydro-1,6-naphthyridin-2-yl)-3,8-diazabicyclo[3.2.1]octane-3-carboxylate (44 mg, 0.09 mmol) in N,N-dimethylformamide (1.5 mL) was added 60% sodium hydride in mineral oil (26 mg, 1.08 mmol) and the mixture was stirred for 5 minutes. Next, iodoethane (172 μL, 2.15 mmol) was added and the mixture was stirred at room temperature for 16 hours. The mixture was quenched with methanol (5 mL) and concentrated in vacuo. The residue was purified with silica column chromatography (20% to 100% ethyl acetate in n-heptane) and concentrated in vacuo to afford benzyl 8-(6-(cyclopentyl(ethyl) carbamoyl)-5,6,7,8-tetrahydro-1,6-naphthyridin-2-yl)-3,8-diazabicyclo[3.2.1]octane-3-carboxylate (33 mg, 62%) as a yellow solid. LCMS (Method A): t_{R} 1.83 min, 93%, MS (ESI) 518.4 (M+H)⁺.

To a solution of benzyl 8-(6-(cyclopentyl(ethyl) carbamoyl)-5,6,7,8-tetrahydro-1,6-naphthyridin-2-yl)-3,8-diazabicyclo[3.2.1]octane-3-carboxylate (33 mg, 0.06 mmol) in acetic acid (2 mL) was added 33% hydrogen bromide in acetic acid (167 μL, 0.95 mmol) and the reaction was stirred at room temperature for 16 hours. The mixture was concentrated in vacuo, purified with SCX (ion exchange) chromatography (washed with methanol and eluted with 3.5 M ammonia in methanol) to afford 2-(3,8-diazabicyclo[3.2.1]octan-8-yl)-N-cyclopentyl-N-ethyl-7,8-dihydro-1,6-naphthyridine-6(5H)-carboxamide (12 mg, 50%, Example 64) as a white solid. ¹H-NMR: δ_{H} (400 MHZ, CDCl₃) 7.18 (1H, d, J 8.5), 6.41 (1H, d, J 8.6), 4.44 (2H, br s), 4.26 (2H, br s), 4.01-3.94 (1H, m), 3.52 (2H, t, J 5.9), 3.21-3.11 (4H, m), 2.86 (2H, t, J 6.0), 2.68-2.61 (2H, m), 2.04-1.83 (6H, m), 1.65-1.49 (6H, m), 1.10 (3H, t, J 7.0); UPLC (Method A): t_{R} 1.41 min, 93%, MS (ESI) 384.4 (M+H)⁺.

### Example 87: Synthesis of 1-(2-(3,8-diazabicyclo[3.2.1]octan-8-yl)-5,7-dihydro-6H-pyrrolo[3,4-b]pyridin-6-yl)-2-(4,4-difluorocyclohexyl)ethan-1-one

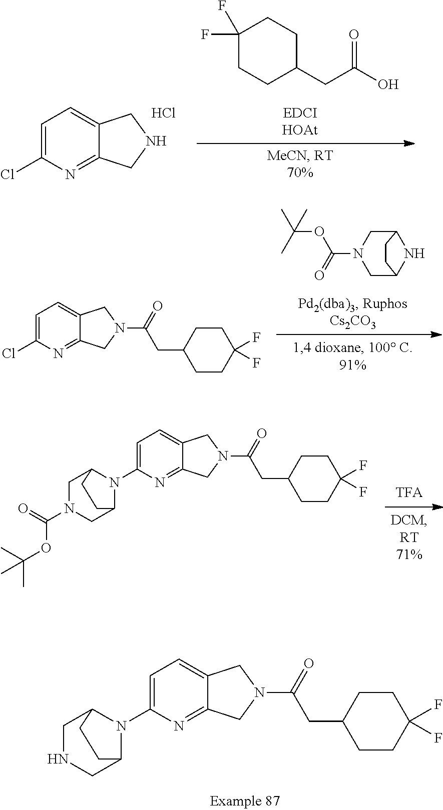

To a solution of 2-chloro-6,7-dihydro-5H-pyrrolo[3,4-b]pyridine hydrochloride (120 mg, 0.63 mmol), 2-(4,4-difluorocyclohexyl) acetic acid (134 mg, 0.75 mmol) and triethylamine (0.19 mL, 1.38 mmol) in acetonitrile (4 mL) were added N-(3-dimethylaminopropyl)-N′-ethylcarbodiimide hydrochloride (144 mg, 0.75 mmol) and 1-hydroxy-7-azabenzotriazole (8.55 mg, 0.06 mmol). The mixture was stirred at room temperature for 16 hours. The mixture was filtered over Celite®, washed with dichloromethane and concentrated in vacuo. The residue was dissolved in dichloromethane (3 mL), purified with silica column chromatography (0% to 100% ethyl acetate in n-heptane) and concentrated in vacuo to afford 1-(2-chloro-5,7-dihydro-6H-pyrrolo[3,4-b]pyridin-6-yl)-2-(4,4-difluorocyclohexyl)ethan-1-one (140 mg, 71%) as a white solid.

Under argon atmosphere, RuPhos (44 mg, 0.10 mmol) and Pd₂(dba)₃ (39 mg, 0.04 mmol) were suspended in 1,4-dioxane (0.3 mL) and heated to 80° C. for 5 minutes. The mixture was cooled to room temperature and transferred under argon atmosphere to a stirring solution of 1-(2-chloro-5,7-dihydro-6H-pyrrolo[3,4-b]pyridin-6-yl)-2-(4,4-difluorocyclohexyl)ethan-1-one (134 mg, 0.43 mmol), tert-butyl 3,8-diazabicyclo[3.2.1]octane-3-carboxylate (117 mg, 0.55 mmol) and cesium carbonate (347 mg, 1.06 mmol) in 1,4-dioxane (1.2 mL) and the mixture was heated to 100° C. for 16 hours. The mixture was filtered over Celite®, washed with acetonitrile and concentrated in vacuo. The residue was purified with silica column chromatography (0% to 100% ethyl acetate in n-heptane) and concentrated in vacuo to afford tert-butyl 8-(6-(2-(4,4-difluorocyclohexyl) acetyl)-6,7-dihydro-5H-pyrrolo[3,4-b]pyridin-2-yl)-3,8-diazabicyclo[3.2.1]octane-3-carboxylate (193 mg, 91%) as an orange foam.

To a solution of tert-butyl 8-(6-(2-(4,4-difluorocyclohexyl) acetyl)-6,7-dihydro-5H-pyrrolo[3,4-b]pyridin-2-yl)-3,8-diazabicyclo[3.2.1]octane-3-carboxylate (190 mg, 0.39 mmol) in dichloromethane (2 mL) was added trifluoroacetic acid (0.30 mL, 3.87 mmol) and the mixture was stirred at room temperature for 1 hour The mixture was concentrated, dissolved in methanol (2.5 mL) and 7N ammonia in methanol (0.5 mL) and purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-5,7-dihydro-6H-pyrrolo[3,4-b]pyridin-6-yl)-2-(4,4-difluorocyclohexyl)ethan-1-one (109 mg, 72%, Example 87) as a white solid. ¹H-NMR: δ_{H} (400 MHZ, CDCl₃, mixture of rotamers) 7.42-7.28 (1H, m), 6.52-6.41 (1H, m), 4.74-4.58 (4H, m), 4.43-4.39 (2H, m), 3.20-3.05 (2H, m), 2.71-2.56 (2H, m), 2.32-2.28 (2H, m), 2.15-1.67 (11H, m), 1.40-1.26 (2H, m); UPLC (Method C): t_{R} 4.17 min, 99%, MS (ESI) 391.2 (M+H)+

The following example (88) was prepared using procedures analogous to Example 87, using the appropriate starting materials.

### Example 88: Synthesis of 1-(2-(3,8-diazabicyclo[3.2.1]octan-8-yl)-5,7-dihydro-6H-pyrrolo[3,4-b]pyridin-6-yl)-2-cyclopentylethan-1-one

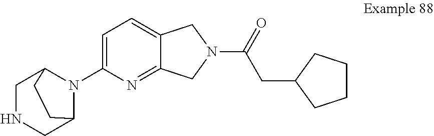 [SMILES: O=C(CC1CCCC1)N1Cc2ccc(N3C4CCC3CNC4)nc2C1]

Prepared using cyclopentylacetic acid as the acid component.

¹H-NMR: δ_{H} (400 MHZ, CDCl₃, mixture of rotamers) 7.40-7.28 (1H, m), 6.51-6.41 (1H, m), 4.74-4.61 (4H, m), 4.50-4.34 (2H, m), 3.17-3.06 (2H, m), 2.69-2.57 (2H, m), 2.41-2.35 (3H, m), 2.10-1.99 (2H, m), 1.99-1.84 (4H, m), 1.70-1.49 (4H, m), 1.27-1.13 (2H, m); UPLC (Method C): t_{R} 4.11 min, 100%, MS (ESI) 341.2 (M+H)⁺.

### Example 89: Synthesis of 2-(3,8-diazabicyclo[3.2.1]octan-8-yl)-N-(4,4-difluorocyclohexyl)-5,7-dihydro-6H-pyrrolo[3,4-b]pyridine-6-carboxamide

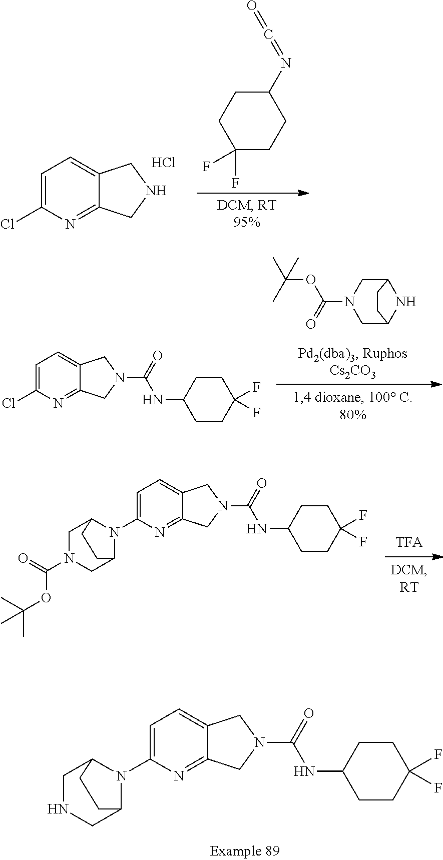

To a solution of 2-chloro-6,7-dihydro-5H-pyrrolo[3,4-b]pyridine hydrochloride (128 mg, 0.67 mmol) and triethylamine (0.11 mL, 0.80 mmol) in dichloromethane (2 mL) was added 1,1-difluoro-4-isocyanatocyclohexane (130 mg, 0.80 mmol) and the mixture was stirred at room temperature for 1 hour. The mixture was coated onto Celite®, purified with silica column chromatography (0% to 100% ethyl acetate in n-heptane) and concentrated in vacuo to afford 2-chloro-N-(4,4-difluorocyclohexyl)-5,7-dihydro-6H-pyrrolo[3,4-b]pyridine-6-carboxamide (218 mg, 95%) as a white solid.

Under argon atmosphere, RuPhos (50 mg, 0.11 mmol) and Pd₂(dba)₃ (44 mg, 0.05 mmol) were suspended in 1,4-dioxane (0.3 mL) and heated to 80° C. for 5 minutes. The mixture was cooled to room temperature and transferred under argon atmosphere to a stirring solution of 2-chloro-N-(4,4-difluorocyclohexyl)-5,7-dihydro-6H-pyrrolo[3,4-b]pyridine-6-carboxamide (153 mg, 0.49 mmol), tert-butyl-3,8-diazabicyclo[3.2.1]octane-3-carboxylate (134 mg, 0.63 mmol) and cesium carbonate (395 mg, 1.21 mmol) in 1,4-dioxane (1.5 mL) and the mixture was heated to 100° C. for 16 hours. The mixture was filtered over Celite®, washed with acetonitrile and concentrated in vacuo. The residue was purified with silica column chromatography (0% to 100% ethyl acetate in n-heptane) and concentrated in vacuo to afford tert-butyl 8-(6-((4,4-difluorocyclohexyl) carbamoyl)-6,7-dihydro-5H-pyrrolo[3,4-b]pyridin-2-yl)-3,8-diazabicyclo[3.2.1]octane-3-carboxylate (279 mg, 80%) as an orange solid.

To a solution of tert-butyl 8-(6-((4,4-difluorocyclohexyl) carbamoyl)-6,7-dihydro-5H-pyrrolo[3,4-b]pyridin-2-yl)-3,8-diazabicyclo[3.2.1]octane-3-carboxylate (270 mg, 0.55 mmol) in dichloromethane (2 mL) was added trifluoroacetic acid (0.42 mL, 5.49 mmol) and the mixture was stirred at room temperature for 1 hour The mixture was concentrated, dissolved in methanol (3 mL) and 7N ammonia in methanol (0.5 mL) and purified with preparative reversed phase chromatography (Waters XSelect™ CSH C18 column, ammonium bicarbonate in water/acetonitrile) and lyophilized to afford 2-(3,8-diazabicyclo[3.2.1]octan-8-yl)-N-(4,4-difluorocyclohexyl)-5,7-dihydro-6H-pyrrolo[3,4-b]pyridine-6-carboxamide (162 mg, 75%, Example 89) as a white solid. ¹H-NMR: δ_{H} (400 MHZ, CDCl₃) 7.34 (1H, d, J 8.5), 6.46 (1H, d, J 8.6), 4.58 (2H, s), 4.51 (2H, s), 4.42 (2H, t, J 3.5), 4.13 (1H, d, J 7.6), 3.87 (1H, d, J 8.4), 3.20-3.08 (2H, m), 2.68-2.57 (2H, m), 2.18-1.99 (6H, m), 1.99-1.78 (4H, m), 1.54 (2H, m); UPLC (Method A): t_{R} 2.07 min, 99%, MS (ESI) 392.2 (M+H)⁺.

The following example (90) was prepared using procedures analogous to Example 89, using the appropriate starting materials.

### Example 90: Synthesis of 2-(3,8-diazabicyclo[3.2.1]octan-8-yl)-N-cyclopentyl-5,7-dihydro-6H-pyrrolo[3,4-b]pyridine-6-carboxamide

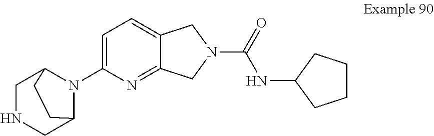 [SMILES: O=C(NC1CCCC1)N1Cc2ccc(N3C4CCC3CNC4)nc2C1]

Prepared using cyclopentyl isocyanate as the isocyanate component.

¹H-NMR: δ_{H} (400 MHZ, CDCl₃) 7.34 (1H, d, J 8.5), 6.45 (1H, d, J 8.6), 4.64-4.55 (2H, m), 4.55-4.46 (2H, m), 4.46-4.37 (2H, m), 4.26-4.13 (2H, m), 3.18-3.07 (2H, m), 2.68-2.56 (2H, m), 2.11-1.98 (4H, m), 1.98-1.89 (2H, m), 1.70-1.65 (2H, m), 1.65-1.55 (2H, m), 1.47-1.33 (2H, m); UPLC (Method A): t_{R} 1.88 min, 99%, MS (ESI) 342.2 (M+H)⁺.

### Example 91: Synthesis of N-(4,4-difluorocyclohexyl)-2-(3-methyl-3,8-diazabicyclo[3.2.1]octan-8-yl)-5,7-dihydro-6H-pyrrolo[3,4-b]pyridine-6-carboxamide

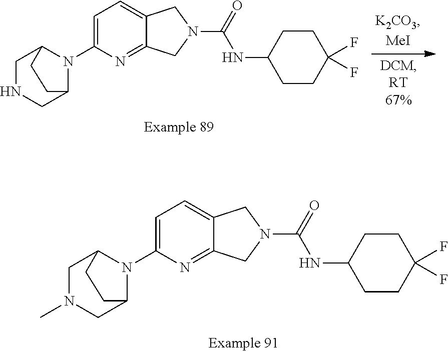 [SMILES: O=C(NC1CCC(F)(F)CC1)N1Cc2ccc(N3C4CCC3CNC4)nc2C1 ; CN1CC2CCC(C1)N2c1ccc2c(n1)CN(C(=O)NC1CCC(F)(F)CC1)C2]

To a solution of 2-(3,8-diazabicyclo[3.2.1]octan-8-yl)-N-(4,4-difluorocyclohexyl)-5,7-dihydro-6H-pyrrolo[3,4-b]pyridine-6-carboxamide (132 mg, 0.34 mmol, Example 89) and potassium carbonate (117 mg, 0.84 mmol) in dichloromethane (2 mL) was added methyl iodide (63 μL, 1.01 mmol), The mixture was stirred at room temperature for 1 hour and concentrated in vacuo. The residue was purified with preparative reversed phase chromatography (Waters XSelect™ CSH C18 column, ammonium bicarbonate in water/acetonitrile) and lyophilized to afford N-(4,4-difluorocyclohexyl)-2-(3-methyl-3,8-diazabicyclo[3.2.1]octan-8-yl)-5,7-dihydro-6H-pyrrolo[3,4-b]pyridine-6-carboxamide (92 mg, 67%, Example 91) as a white solid. ¹H-NMR: δ_{H} (400 MHZ, CDCl₃) 7.33 (1H, d, J 8.6), 6.46 (1H, d, J 8.6), 4.58 (2H, s), 4.49 (4H, d, J 14.2), 4.12 (1H, d, J 7.7), 3.87 (1H, d, J 7.5), 2.68-2.56 (2H, m), 2.40-2.28 (2H, m), 2.19 (3H, s), 2.15-2.03 (4H, m), 2.03-1.79 (6H, m), 1.59-1.47 (2H, m); UPLC (Method B): t_{R} 1.26 min, 100%, MS (ESI) 406.7 (M+H)⁺.

The following examples (92-94) were prepared using procedures analogous to Example 91, using the appropriate starting materials.

### Example 92: Synthesis of N-cyclopentyl-2-(3-methyl-3,8-diazabicyclo[3.2.1]octan-8-yl)-5,7-dihydro-6H-pyrrolo[3,4-b]pyridine-6-carboxamide

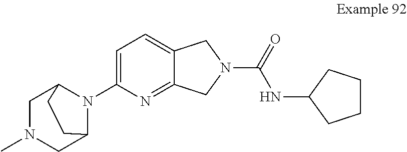 [SMILES: CN1CC2CCC(C1)N2c1ccc2c(n1)CN(C(=O)NC1CCCC1)C2]

Prepared using Example 90 as a starting reagent.

¹H-NMR: δ_{H} (400 MHZ, CDCl₃) 7.33 (1H, d, J 8.5), 6.45 (1H, d, J 8.5), 4.65-4.54 (2H, m), 4.54-4.42 (4H, m), 4.18-4.05 (2H, m), 2.61 (2H, dd, J 11.0, 2.5), 2.35 (2H, dd, J 11.0, 1.8), 2.18 (3H, s), 2.10-1.97 (4H, m), 1.97-1.90 (2H, m), 1.75-1.60 (4H, m), 1.45-1.35 (2H, m); UPLC (Method A): t_{R} 1.90 min, 100%, MS (ESI) 356.2 (M+H)⁺.

### Example 93: Synthesis of 2-(4,4-difluorocyclohexyl)-1-(2-(3-methyl-3,8-diazabicyclo[3.2.1]octan-8-yl)-5,7-dihydro-6H-pyrrolo[3,4-b]pyridin-6-yl)ethan-1-one

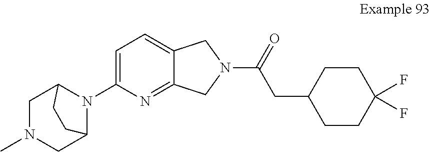 [SMILES: CN1CC2CCC(C1)N2c1ccc2c(n1)CN(C(=O)CC1CCC(F)(F)CC1)C2]

Prepared using Example 87 as a starting reagent.

¹H-NMR: δ_{H} (400 MHZ, CDCl₃, mixture of rotamers) 7.40-7.28 (1H, m), 6.54-6.38 (1H, m), 4.75-4.56 (4H, m), 4.47 (2H, s), 2.68-2.56 (2H, m), 2.36-2.32 (2H, m), 2.29-2.24 (2H, m), 2.19 (3H, s), 2.08 (3H, m), 2.03-1.67 (8H, m), 1.42-1.27 (2H, m); UPLC (Method B): t_{R} 1.41 min, 99%, MS (ESI) 405.7 (M+H)⁺.

### Example 94: Synthesis of 2-cyclopentyl-1-(2-(3-methyl-3,8-diazabicyclo[3.2.1]octan-8-yl)-5,7-dihydro-6H-pyrrolo[3,4-b]pyridin-6-yl)ethan-1-one

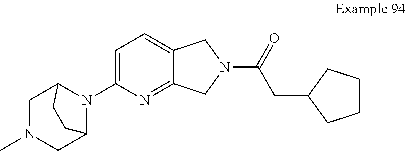 [SMILES: CN1CC2CCC(C1)N2c1ccc2c(n1)CN(C(=O)CC1CCCC1)C2]

Prepared using Example 88 as a starting reagent.

¹H-NMR: δ_{H} (400 MHZ, CDCl₃, mixture of rotamers) 7.39-7.27 (1H, m), 6.51-6.41 (1H, m), 4.73-4.62 (4H, m), 4.46 (2H, m), 2.68-2.54 (2H, m), 2.44-2.29 (5H, m), 2.19 (3H, s), 2.06-1.83 (6H, m), 1.76-1.55 (2H, m), 1.58-1.54 (2H, m), 1.28-1.13 (2H, m); UPLC (Method B): t_{R} 2.67 min, 99%, MS (ESI) 355.2 (M+H)⁺.

### Example 95: Synthesis of (2-(3,8-diazabicyclo[3.2.1]octan-8-yl)-5,7-dihydro-6H-pyrrolo[3,4-b]pyridin-6-yl)(pyrrolidin-1-yl)methanone

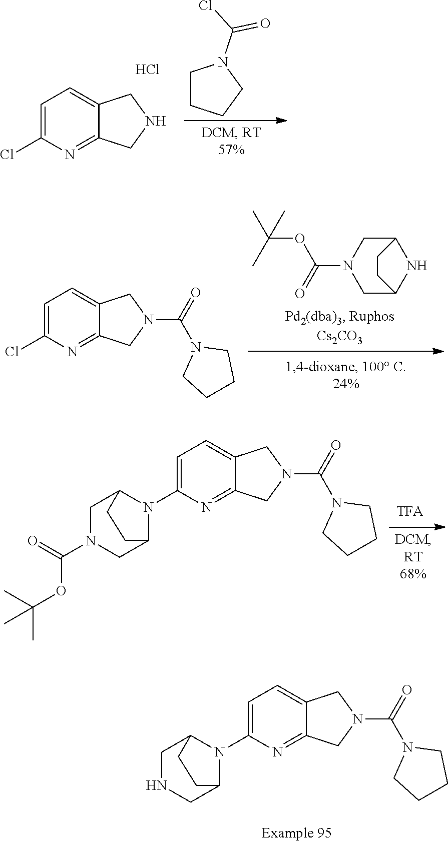

To a solution of 2-chloro-6,7-dihydro-5H-pyrrolo[3,4-b]pyridine hydrochloride (125 mg, 0.65 mmol) and triethylamine (0.24 mL, 1.70 mmol) in dichloromethane (3 mL) was added 1-pyrrolidinecarbonyl chloride (0.09 mL, 0.79 mmol) and the mixture was stirred at room temperature for 1 hour. The mixture was concentrated in vacuo, suspended in methanol (1 mL) and diluted with water. The precipitate was collected by filtration, thoroughly washed with water and dried to afford (2-chloro-5,7-dihydro-6H-pyrrolo[3,4-b]pyridin-6-yl)(pyrrolidin-1-yl)methanone (93 mg, 57%) as a pink solid.

Under an argon atmosphere, RuPhos (31 mg, 0.07 mmol) and Pd₂(dba)₃ (27 mg, 0.03 mmol) were suspended in 1,4-dioxane (1 mL) and heated to 80° C. for 5 minutes. The mixture was cooled to room temperature and transferred under an argon atmosphere to a stirring solution of (2-chloro-5,7-dihydro-6H-pyrrolo[3,4-b]pyridin-6-yl)(pyrrolidin-1-yl)methanone (93 mg, 0.37 mmol), tert-butyl 3,8-diazabicyclo[3.2.1]octane-3-carboxylate (102 mg, 0.48 mmol) and cesium carbonate (301 mg, 0.92 mmol) in 1,4-dioxane (3 mL) and the mixture was heated to 100° C. for 16 hours. The mixture was filtered over Celite®, washed with acetonitrile and concentrated in vacuo. The residue was purified with silica column chromatography (0% to 100% ethyl acetate in n-heptane) to afford tert-butyl 8-(6-(pyrrolidine-1-carbonyl)-6,7-dihydro-5H-pyrrolo[3,4-b]pyridin-2-yl)-3,8-diazabicyclo[3.2.1]octane-3-carboxylate (40 mg, 24%) as a light yellow solid.

To a solution of tert-butyl 8-(6-(pyrrolidine-1-carbonyl)-6,7-dihydro-5H-pyrrolo[3,4-b]pyridin-2-yl)-3,8-diazabicyclo[3.2.1]octane-3-carboxylate (40 mg, 0.09 mmol) in dichloromethane (1 mL) was added trifluoroacetic acid (0.180 mL, 2.34 mmol) and the mixture was stirred at room temperature for 1 hour. The mixture was concentrated in vacuo, redissolved in methanol (2.5 mL) and 7N ammonia in methanol (0.5 mL) and was purified with preparative reversed phase chromatography (Waters XSelect™ CSH C18 column, ammonium bicarbonate in water/acetonitrile) and lyophilized to afford (2-(3,8-diazabicyclo[3.2.1]octan-8-yl)-5,7-dihydro-6H-pyrrolo[3,4-b]pyridin-6-yl)(pyrrolidin-1-yl)methanone (21 mg, 68%, Example 95) as a white solid. ¹H-NMR: δ_{H} (400 MHZ, CDCl₃) 7.33 (1H, d, J 8.5), 6.44 (1H, d, J 8.5), 4.74-4.60 (4H, m), 4.43 (2H, d, J 4.6), 3.53-3.41 (4H, m), 3.20-3.09 (2H, m), 2.69-2.59 (2H, m), 2.08-1.98 (2H, m), 1.98-1.91 (2H, m), 1.91-1.84 (4H, m); UPLC (Method A): t_{R} 1.70 min, 99%, MS (ESI) 328.2 (M+H)⁺.

The following example (96) was prepared using procedures analogous to Example 95, using the appropriate starting materials.

### Example 96: Synthesis of 2-(3,8-diazabicyclo[3.2.1]octan-8-yl)-N,N-diethyl-5,7-dihydro-6H-pyrrolo[3,4-b]pyridine-6-carboxamide

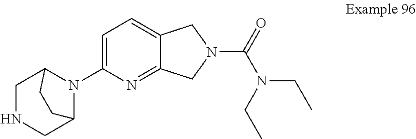 [SMILES: CCN(CC)C(=O)N1Cc2ccc(N3C4CCC3CNC4)nc2C1]

Prepared using diethylcarbamoyl chloride.

¹H-NMR: δ_{H} (400 MHZ, CDCl₃) 7.32 (1H, d, J 8.5), 6.44 (1H, d, J 8.5), 4.70-4.66 (2H, m), 4.62-4.58 (2H, m), 4.48-4.36 (2H, m), 3.28 (4H, q, J 7.1), 3.20-3.08 (2H, m), 2.69-2.56 (2H, m), 2.08-1.98 (2H, m), 1.98-1.89 (2H, m), 1.19 (6H, t, J 7.1H); UPLC (Method A): t_{R} 1.91 min, 99%, MS (ESI) 330.2 (M+H)⁺.

### Example 105: Synthesis of N-(4,4-difluorocyclohexyl)-2-(3-methyl-3,8-diazabicyclo[3.2.1]octan-8-yl)-7,8-dihydro-1,6-naphthyridine-6(5H)-carboxamide

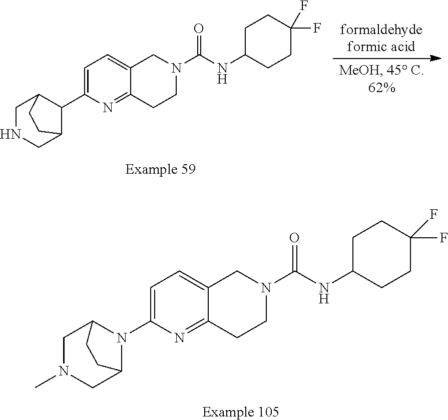 [SMILES: O=C(NC1CCC(F)(F)CC1)N1CCc2nc(C3C4CCC3CNC4)ccc2C1 ; "Example 59" ; CN1CC2CCC(C1)N2c1ccc2c(n1)CCN(C(=O)NC1CCC(F)(F)CC1)C2 ; "Example 105"]

To a solution of 2-(3,8-diazabicyclo[3.2.1]octan-8-yl)-N-(4,4-difluorocyclohexyl)-7,8-dihydro-1,6-naphthyridine-6(5H)-carboxamide (18.7 mg, 0.05 mmol, Example 59) in methanol (1 mL) were added 37% wt formaldehyde in water (17 μL, 0.23 mmol) and formic acid (9 μL, 0.24 mmol). The mixture was heated to 45° C. for 16 hours and the mixture was concentrated in vacuo. The residue was purified with reversed phase chromatography (Waters XSelect™ CSH C18 column, ammonium bicarbonate in water/acetonitrile followed by Phenomenex LUNA C18 column, formic acid in water/acetonitrile) and lyophilized to afford N-(4,4-difluorocyclohexyl)-2-(3-methyl-3,8-diazabicyclo[3.2.1]octan-8-yl)-7,8-dihydro-1,6-naphthyridine-6(5H)-carboxamide (12 mg, 62%, Example 105) as a white solid. ¹H-NMR: δ_{H} (400 MHZ, CDCl₃) 7.17 (1H, d, J 8.5), 6.42 (1H, d, J 8.5), 4.47 (2H, s), 4.39 (2H, s), 4.30 (1H, d, J 7.5), 3.90-3.78 (1H, m), 3.64 (2H, t, J 5.9), 2.84 (2H, t, J 5.9), 2.67-2.57 (2H, m), 2.39-2.31 (2H, m), 2.19 (3H, s), 2.15-1.78 (11H, m), 1.54-1.45 (2H, m); UPLC (Method B): t_{R} 2.46 min, 99%, MS (ESI) 420.2 (M+H)⁺.

The following example (106) was prepared using procedures analogous to Example 105, using the appropriate starting materials.

### Example 106: Synthesis of 2-(4,4-difluorocyclohexyl)-1-(2-(3-methyl-3,8-diazabicyclo[3.2.1]octan-8-yl)-7,8-dihydro-1,6-naphthyridin-6(5H)-yl)ethan-1-one

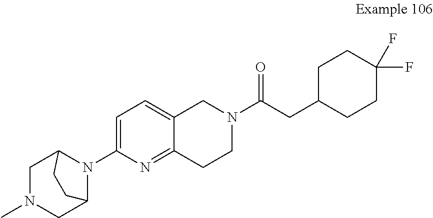 [SMILES: CN1CC2CCC(C1)N2c1ccc2c(n1)CCN(C(=O)CC1CCC(F)(F)CC1)C2]

Prepared using Example 40 as starting reagent.

¹H-NMR: δ_{H} (400 MHZ, CDCl₃, mixture of rotamers) 7.20 (0.6H, d, J 8.6), 7.15 (0.4H, d, J 8.6), 6.47-6.40 (1H, m), 4.63-4.44 (4H, m), 3.88 (0.8H, t, J 6.0), 3.72 (1.2H, t, J 6.0), 2.88-2.77 (2H, m), 2.66-2.57 (2H, m), 2.39-2.29 (4H, m), 2.19 (3H, s), 2.13-1.66 (11H, m), 1.40-1.24 (2H, m); UPLC (Method B): 2.73 min, 99%, MS (ESI) 419.4 (M+H)⁺.

### Example 111: Synthesis of 2-(4,4-difluorocyclohexyl)-1-(2-(piperazin-1-yl)-7,8-dihydro-1,6-naphthyridin-6(5H)-yl)ethan-1-one

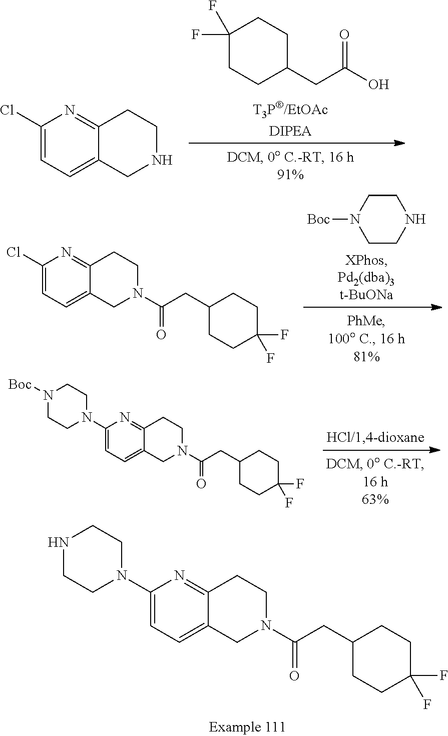 [SMILES: O=C(O)CC1CCC(F)(F)CC1 ; Clc1ccc2c(n1)CCNC2 ; CC(C)(C)OC(=O)N1CCNCC1 ; O=C(CC1CCC(F)(F)CC1)N1CCc2nc(Cl)ccc2C1 ; CC(C)(C)OC(=O)N1CCN(c2ccc3c(n2)CCN(C(=O)CC2CCC(F)(F)CC2)C3)CC1 ; O=C(CC1CCC(F)(F)CC1)N1CCc2nc(N3CCNCC3)ccc2C1]

To a stirred solution of 2-(4,4-difluorocyclohexyl) acetic acid (1.00 g, 5.61 mmol) in DCM (15 mL) at 0° C. was added DIPEA (4.90 mL, 28.1 mmol) and n-propylphosphonic acid anhydride, cyclic trimer (50% in ethyl acetate; 3.57 g, 5.61 mmol). After 15 minutes, 2-chloro-5,6,7,8-tetrahydro-1,6-naphthyridine hydrochloride (1.15 g, 5.61 mmol) was added. The mixture was stirred at r.t. for 16 h, then diluted with water (10 mL) and extracted with 5% MeOH in DCM (3×20 mL). The combined organic extract was washed with brine (20 mL), dried over anhydrous sodium sulfate, filtered, and concentrated under reduced pressure. The residue was purified by flash chromatography, eluting with 0% to 100% ethyl acetate in petroleum ether, to obtain 1-(2-chloro-7,8-dihydro-1,6-naphthyridin-6(5H)-yl)-2-(4,4-difluorocyclohexyl)ethan-1-one (1.68 g, 91%) as an off-white solid.

To a de-gassed mixture of 1-(2-chloro-7,8-dihydro-1,6-naphthyridin-6(5H)-yl)-2-(4,4-difluorocyclohexyl)ethan-1-one (1.10 g, 3.35 mmol) and tert-butyl piperazine-1-carboxylate (1.25 g, 6.69 mmol) in toluene (10 mL) under continuous bubbling of nitrogen was added sodium tert-butoxide (0.965 g, 10.0 mmol), XPhos (0.319 g, 0.669 mmol) and Pd₂(dba)₃ (0.306 g, 0.335 mmol). The mixture was stirred in a sealed vial at 100° C. for 16 h, then concentrated under reduced pressure. The residue was diluted with 10% MeOH in DCM (40 mL) and filtered through Celite®, washing with 10% MeOH in DCM (20 mL). The filtrate was concentrated under reduced pressure. The residue was purified by flash chromatography, eluting with 40% to 80% ethyl acetate in petroleum ether. The product was washed with 20% ethyl acetate in petroleum ether and dried to obtain tert-butyl 4-(6-(2-(4,4-difluorocyclohexyl) acetyl)-5,6,7,8-tetrahydro-1,6-naphthyridin-2-yl) piperazine-1-carboxylate (1.3 g, 81%) as a pale yellow solid. To a stirred solution of tert-butyl 4-(6-(2-(4,4-difluorocyclohexyl) acetyl)-5,6,7,8-tetrahydro-1,6-naphthyridin-2-yl) piperazine-1-carboxylate (5.00 g, 10.45 mmol) in DCM (10 mL) at 0° C. was added HCl (4 M in 1,4 dioxane; 13.1 mL, 52.2 mmol). The reaction mixture was stirred at r.t. for 16 h, then concentrated under reduced pressure. The residue was dissolved in water (50 mL) and washed with EtOAc (2×15 mL). The separated aqueous layer was basified with 10% sodium bicarbonate solution (10 mL) and extracted with 15% MeOH in DCM (3×50 mL). The combined organic extract was washed with brine (25 mL), dried over anhydrous sodium sulfate, filtered, and concentrated under reduced pressure. The residue was washed with 20% EtOAc in petroleum ether (20 mL) then dissolved in MeCN: water (7:3 ratio, 10 mL) and lyophilised to obtain 2-(4,4-difluorocyclohexyl)-1-(2-(piperazin-1-yl)-7,8-dihydro-1,6-naphthyridin-6(5H)-yl)ethan-1-one (2.5 g, 63%, Example 111) as an off-white solid. ¹H-NMR: δ_{H} (400 MHZ, DMSO-d₆, 80° C.) 7.36 (1H, d, J 8.8), 6.67 (1H, d, J 8.8), 4.51 (2H, br s), 3.74 (2H, t, J 6.0), 3.49-3.46 (4H, m), 2.91-2.89 (4H, m), 2.74-2.68 (2H, m), 2.38-2.36 (2H, m), 2.00-1.92 (3H, m), 1.78-1.75 (4H, m), 1.32-1.23 (2H, m).

### Example 112: Synthesis of (R)-2-cyclopentyl-1-(2-(hexahydropyrrolo[1,2-a]pyrazin-2 (1H)-yl)-7,8-dihydro-1,6-naphthyridin-6(5H)-yl)ethan-1-one

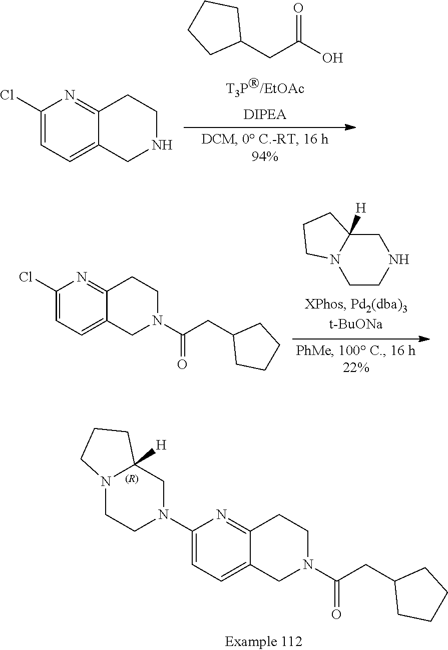

To a solution of 2-cyclopentylacetic acid (5.00 g, 39.0 mmol) in DCM (20 mL) at 0° C. were added DIPEA (34.0 mL, 195 mmol), n-propylphosphonic acid anhydride, cyclic trimer (50% in ethyl acetate; 45.6 mL, 78.0 mmol) and 2-chloro-5,6,7,8-tetrahydro-1,6-naphthyridine hydrochloride (9.60 g, 46.8 mmol). The mixture was stirred at r.t. for 16 h, then concentrated under reduced pressure, diluted with water (50 mL), and extracted with 10% methanol in DCM (3×50 mL). The combined organic extract was washed with brine (100 mL), dried over anhydrous sodium sulfate, filtered, and concentrated under reduced pressure. The residue was purified by flash chromatography, eluting with 0% to 50% ethyl acetate in petroleum ether, to obtain 1-(2-chloro-7,8-dihydro-1,6-naphthyridin-6(5H)-yl)-2-cyclopentylethan-1-one (10.2 g, 94%) as a pale yellow solid.

To a stirred solution of 1-(2-chloro-7,8-dihydro-1,6-naphthyridin-6(5H)-yl)-2-cyclopentylethan-1-one (200 mg, 0.717 mmol) in toluene (10 mL) under continuous bubbling of nitrogen were added (R)-octahydropyrrolo[1,2-a]pyrazine (181 mg, 1.44 mmol), sodium tert-butoxide (207 mg, 2.15 mmol) and X-Phos (68.4 mg, 0.143 mmol). After stirring for 5 minutes at r.t., Pd₂ (dba)₃ (65.7 mg, 0.072 mmol) was added and the mixture was stirred in a sealed tube at 100° C. for 16 h. The reaction mixture was concentrated under reduced pressure and the residue was diluted with water (10 mL) and extracted with 10% methanol in DCM (3×50 mL). The combined organic extract was washed with brine (20 mL), dried over anhydrous sodium sulfate, filtered, and concentrated under reduced pressure. The residue was purified by preparative HPLC (acidic method). The product-containing fractions were combined, concentrated under reduced pressure, basified with saturated NaHCO₃ solution (20 mL) and extracted with 10% MeOH in DCM (2×20 mL). The combined organic extract was dried over anhydrous sodium sulfate, filtered and concentrated under reduced pressure to obtain (R)-2-cyclopentyl-1-(2-(hexahydropyrrolo[1,2-a]pyrazin-2 (1H)-yl)-7,8-dihydro-1,6-naphthyridin-6(5H)-yl)ethan-1-one (58 mg, 22%, Example 112) as a gummy solid. ¹H-NMR: δ_{H} (400 MHZ, DMSO-d₆) 7.35 (1H, d, J 8.4), 6.73-6.69 (1H, m), 4.52-4.50 (2H, m), 4.46-4.42 (1H, m), 4.31-4.33 (1H, m), 3.72-3.70 (2H, m), 3.03-3.01 (2H, m), 2.77-2.74 (2H, m), 2.68-2.64 (1H, m), 2.45-2.41 (3H, m), 2.34-2.02 (3H, m), 1.78-1.69 (6H, m), 1.59-1.48 (4H, m), 1.16-1.14 (1H, m), 1.13-1.11 (2H, m).

The following example (113) was prepared using procedures analogous to Example 112, using the appropriate starting materials.

### Example 113: Synthesis of (S)-2-cyclopentyl-1-(2-(hexahydropyrrolo[1,2-a]pyrazin-2 (1H)-yl)-7,8-dihydro-1,6-naphthyridin-6(5H)-yl)ethan-1-one

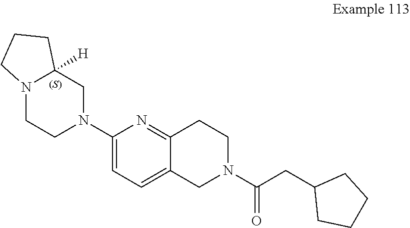

Prepared using(S)-octahydropyrrolo[1,2-a]pyrazine instead of (R)-octahydropyrrolo[1,2-a]pyrazine. ¹H-NMR: δ_{H} (400 MHZ, DMSO-d₆) 7.34 (1H, d, J 8.8), 6.72-6.69 (1H, m), 4.52-4.50 (2H, m), 4.42-4.44 (1H, m), 4.31-4.33 (1H, m), 3.72-3.70 (2H, m), 3.03-3.01 (2H, m), 2.77-2.74 (2H, m), 2.68-2.64 (1H, m), 2.45-2.41 (3H, m), 2.34-2.02 (3H, m), 1.78-1.69 (6H, m), 1.59-1.48 (4H, m), 1.16-1.14 (1H, m), 1.13-1.11 (2H, m).

### Example 114: Synthesis of 2-cyclopentyl-1-(2-(piperidin-4-yl)-7,8-dihydro-1,6-naphthyridin-6(5H)-yl)ethan-1-one

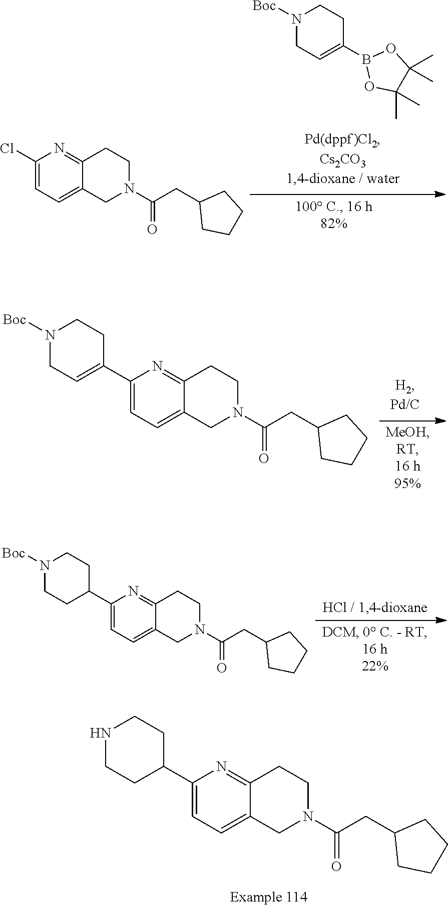 [SMILES: CC(C)(C)OC(=O)N1CC=C(B2OC(C)(C)C(C)(C)O2)CC1 ; O=C(CC1CCCC1)N1CCc2nc(Cl)ccc2C1 ; CC(C)(C)OC(=O)N1CC=C(c2ccc3c(n2)CCN(C(=O)CC2CCCC2)C3)CC1 ; CC(C)(C)OC(=O)N1CCC(c2ccc3c(n2)CCN(C(=O)CC2CCCC2)C3)CC1 ; O=C(CC1CCCC1)N1CCc2nc(C3CCNCC3)ccc2C1]

To a stirred solution of 1-(2-chloro-7,8-dihydro-1,6-naphthyridin-6(5H)-yl)-2-cyclopentylethan-1-one (2 g, 7.17 mmol) in 1,4 dioxane (18 mL) and water (2 mL) under continuous bubbling of nitrogen were added tert-butyl 4-(4,4,5,5-tetramethyl-1,3,2-dioxaborolan-2-yl)-3,6-dihydropyridine-1 (2H)-carboxylate (3.33 g, 10.8 mmol), Cs₂CO₃ (7.01 g, 21.5 mmol) and PdCl₂(dppf)-CH₂Cl₂ complex (0.586 g, 0.717 mmol). The reaction mixture was heated to 100° C. and stirred for 16 h, then diluted with water (50 mL) and extracted with 5% MeOH in DCM (3×50 mL). The combined organic extract was washed with brine (20 mL), dried over anhydrous sodium sulfate, filtered and concentrated under reduced pressure. The residue was purified by flash chromatography, eluting with 0% to 20% methanol in DCM, to afford tert-butyl 4-(6-(2-cyclopentylacetyl)-5,6,7,8-tetrahydro-1,6-naphthyridin-2-yl)-3,6-dihydropyridine-1 (2H)-carboxylate (2.5 g, 82%) as an off-white solid.

To a stirred solution of tert-butyl 4-(6-(2-cyclopentylacetyl)-5,6,7,8-tetrahydro-1,6-naphthyridin-2-yl)-3,6-dihydropyridine-1(2H)-carboxylate (3.00 g, 7.05 mmol) in MeOH (50 mL) under nitrogen was added Pd/C (10%, dry; 300 mg). The mixture was stirred at r.t. under H₂ bladder pressure for 16 h, then filtered through Celite®, washing with methanol (50 mL) and concentrated under reduced pressure to obtain crude tert-butyl 4-(6-(2-cyclopentylacetyl)-5,6,7,8-tetrahydro-1,6-naphthyridin-2-yl) piperidine-1-carboxylate (2.9 g, 95%), which was taken forward to the next step without further purification.

To a stirred solution of tert-butyl 4-(6-(2-cyclopentylacetyl)-5,6,7,8-tetrahydro-1,6-naphthyridin-2-yl) piperidine-1-carboxylate (300 mg, 0.702 mmol) in DCM (5 mL) at 0° C. was added HCl in dioxane (4 M; 1.0 mL, 4.0 mmol). The mixture was stirred at r.t. for 16 h, then concentrated under reduced pressure. The residue was purified by preparative HPLC to afford 2-cyclopentyl-1-(2-(piperidin-4-yl)-7,8-dihydro-1,6-naphthyridin-6(5H)-yl)ethan-1-one (50 mg, 22%, Example 114) as a gum. ¹H-NMR: δ_{H} (400 MHZ, DMSO-d₆) 7.52 (1H, d, J 8.0), 7.10-7.07 (1H, m), 4.60-4.70 (2H, m), 3.78-3.75 (2H, m), 2.99-2.91 (2H, m), 2.90-2.80 (1H, m), 2.60-2.78 (1H, m), 2.56-2.51 (2H, m), 2.50-2.41 (3H, m), 2.19-2.16 (1H, m), 1.76-1.68 (4H, m), 1.61-1.57 (4H, m), 1.48-1.49 (2H, m), 1.14-1.10 (2H, m).

### Example 115: Synthesis of 2-cyclopentyl-1-(2-(1-(2-hydroxyethyl)piperidin-4-yl)-7,8-dihydro-1,6-naphthyridin-6(5H)-yl)ethan-1-one

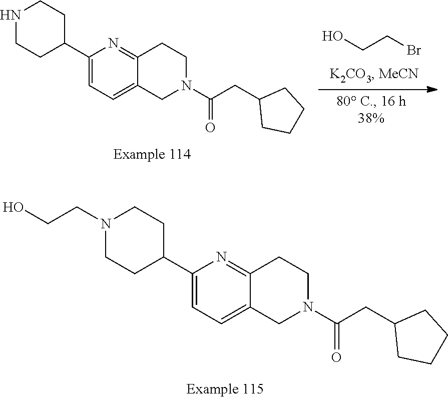

To a solution of 2-cyclopentyl-1-(2-(piperidin-4-yl)-7,8-dihydro-1,6-naphthyridin-6(5H)-yl)ethan-1-one (Example 114; 700 mg, 2.14 mmol) in acetonitrile (30 mL) were added 2-bromoethan-1-ol (321 mg, 2.57 mmol) and potassium carbonate (886 mg, 6.41 mmol). The mixture was stirred at 80° C. for 16 h, then concentrated under reduced pressure. The residue was diluted with water (20 mL) and extracted with 10% MeOH in DCM (3×20 mL). The combined organic extract was washed with brine (10 mL), dried over anhydrous sodium sulfate, filtered and concentrated under reduced pressure. The residue was purified by preparative HPLC to afford 2-cyclopentyl-1-(2-(1-(2-hydroxyethyl)piperidin-4-yl)-7,8-dihydro-1,6-naphthyridin-6(5H)-yl)ethan-1-one (120 mg, 38%, Example 115) as a pale yellow gel. ¹H-NMR: δ_{H} (400 MHZ, DMSO-d₆) 7.52 (1H, d, J 8.0), 7.13-7.09 (1H, m), 4.60-4.70 (2H, m), 4.36 (1H, m), 3.78-3.75 (2H, m), 3.53-3.48 (2H, m), 2.97-2.91 (2H, m), 2.89-2.79 (1H, m), 2.78 (1H, m), 2.57-2.52 (1H, m), 2.43-2.39 (4H, m), 2.17 (1H, m), 2.07-2.01 (2H, m), 1.77-1.71 (6H, m), 1.69-1.59 (2H, m), 1.49-1.48 (2H, m), 1.15-1.12 (2H, m).

The following examples (119-124) were prepared using procedures analogous to Example 39, using the appropriate starting materials.

### Example 119: Synthesis of 1-(2-(3,8-diazabicyclo[3.2.1]octan-8-yl)-7,8-dihydro-1,6-naphthyridin-6(5H)-yl)-2-(1-methylcyclopentyl)ethan-1-one

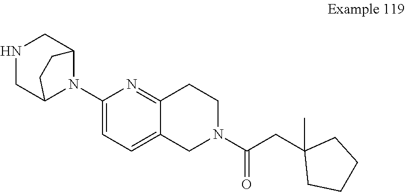 [SMILES: CC1(CC(=O)N2CCc3nc(N4C5CCC4CNC5)ccc3C2)CCCC1]

Prepared using 2-(1-methylcyclopentyl) acetic acid as the acid component.

¹H-NMR: δ_{H} (400 MHZ, CDCl₃, mixture of rotamers); 7.21 (0.6H, d, J 8.5), 7.16 (0.4H, d, J 8.5), 6.46-6.40 (1H, m), 4.61 (1.2H, s), 4.52 (0.8H, s), 4.44 (2H, s), 3.88 (0.8H, t, J 6.0), 3.76 (1.2H, t, J 5.9), 3.14 (2H, m), 2.89-2.77 (2H, m), 2.68-2.61 (2H, m), 2.48-2.42 (2H, m), 2.07-1.91 (4H, m), 1.64-1.54 (5H, m), 1.54-1.46 (2H, m), 1.09 (1.8H, s), 1.06 (1.2H, s). UPLC (Method A): t_{R} 1.45 min, 100%, MS (ESI) 369.2 (M+H)⁺.

### Example 120: Synthesis of (2-(3,8-diazabicyclo[3.2.1]octan-8-yl)-7,8-dihydro-1,6-naphthyridin-6(5H)-yl)(phenyl)methanone

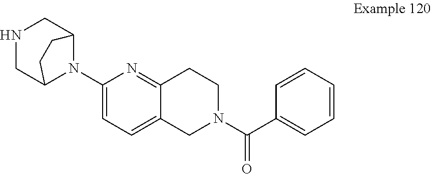 [SMILES: O=C(c1ccccc1)N1CCc2nc(N3C4CCC3CNC4)ccc2C1]

Prepared using benzoic acid as the acid component.

¹H-NMR: δ_{H} (400 MHZ, CDCl₃, mixture of rotamers) 7.50-7.37 (5H, m), 7.26-6.31 (2H, m), 4.84-4.39 (4H, m), 4.11-3.59 (2H, m), 3.14 (2H, d, J 12.2), 3.01-2.75 (2H, m), 2.63 (2H, d, J 2.2), 2.07-1.87 (4H, m); UPLC (Method A): t_{R} 1.05 min, 100%, MS (ESI) 349.2 (M+H)⁺.

### Example 121: Synthesis of (2-(3,8-diazabicyclo[3.2.1]octan-8-yl)-7,8-dihydro-1,6-naphthyridin-6(5H)-yl)(2-methoxyphenyl)methanone

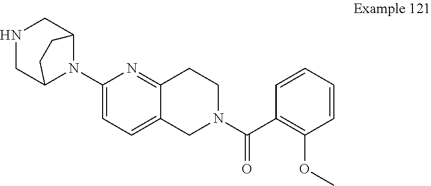 [SMILES: COc1ccccc1C(=O)N1CCc2nc(N3C4CCC3CNC4)ccc2C1]

Prepared using 2-methoxybenzoic acid as the acid component.

¹H-NMR: δ_{H} (400 MHZ, CDCl₃, mixture of rotamers) 7.40-7.26 (2H, m), 7.26-7.23 (1H, m), 7.03-6.91 (2H, m), 6.49-6.34 (1H, m), 4.87-4.72 (1H, m), 4.52-4.39 (2H, m), 4.35-4.19 (1H, m), 3.94-3.73 (3H, m), 3.61-3.44 (1H, m), 3.20-3.07 (2H, m), 2.97-2.70 (2H, m), 2.69-2.61 (2H, m), 2.07-1.90 (4H, m); UPLC (Method A): t_{R} 1.09 min, 100%, MS (ESI) 379.2 (M+H)⁺.

### Example 122: Synthesis of (2-(3,8-diazabicyclo[3.2.1]octan-8-yl)-7,8-dihydro-1,6-naphthyridin-6(5H)-yl)(o-tolyl)methanone

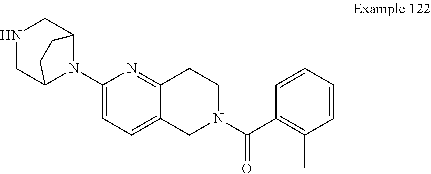 [SMILES: Cc1ccccc1C(=O)N1CCc2nc(N3C4CCC3CNC4)ccc2C1]

Prepared using 2-methylbenzoic acid as the acid component.

¹H-NMR: δ_{H} (400 MHZ, CDCl₃, mixture of rotamers) 7.33-7.26 (2H, m), 7.24-6.92 (3H, m), 6.52-6.32 (1H, m), 4.99-3.45 (6H, m), 3.18-3.09 (2H, m), 2.99-2.68 (2H, m), 2.66-2.59 (2H, m), 2.35-2.21 (3H, m), 2.06-1.89 (4H, m); UPLC (Method A): t_{R} 1.19 min, 100%, MS (ESI) 363.2 (M+H)⁺.

### Example 123: Synthesis of (2-(3,8-diazabicyclo[3.2.1]octan-8-yl)-7,8-dihydro-1,6-naphthyridin-6(5H)-yl)(4-fluorophenyl)methanone

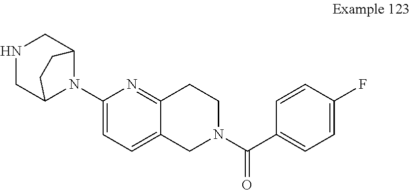 [SMILES: O=C(c1ccc(F)cc1)N1CCc2nc(N3C4CCC3CNC4)ccc2C1]

Prepared using 4-fluorobenzoic acid as the acid component.

¹H-NMR: δ_{H} (400 MHZ, CDCl₃, mixture of rotamers) 7.53-7.41 (2H, m), 7.26-6.87 (3H, m), 6.54-6.30 (1H, m), 4.85-4.36 (4H, m), 4.15-3.58 (2H, m), 3.14 (2H, d, J 12.2), 2.99-2.76 (2H, m), 2.65 (2H, d, J 2.3), 2.06-1.91 (4H, m); UPLC (Method A): t_{R} 1.12 min, 99%, MS (ESI) 367.2 (M+H)⁺.

### Example 124: Synthesis of (2R)-1-(2-(3,8-diazabicyclo[3.2.1]octan-8-yl)-7,8-dihydro-1,6-naphthyridin-6(5H)-yl)-2-cyclopentyl-2-hydroxyethan-1-one

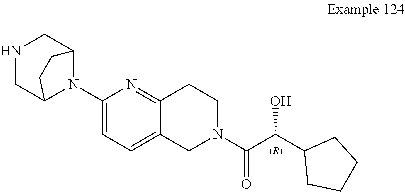

Prepared using (2R)-2-cyclopentyl-2-hydroxyacetic acid as the acid component.

¹H-NMR: δ_{H} (400 MHZ, CDCl₃, mixture of rotamers) 7.19 (1H, dd, J 16.5, 8.5), 6.44 (1H, t, J 7.6), 4.70-4.56 (1H, m), 4.56-4.39 (4H, m), 4.03-3.63 (3H, m), 3.13 (2H, d, J 12.1), 2.91-2.74 (2H, m), 2.63 (2H, dd, J 12.4, 2.2), 2.17-1.86 (5H, m), 1.82-1.61 (4H, m), 1.54-1.31 (4H, m); UPLC (Method A): t_{R} 1.15 min, 99%, MS (ESI) 371.2 (M+H)⁺.

### Example 125: Synthesis of cyclopentyl 2-(3,8-diazabicyclo[3.2.1]octan-8-yl)-7,8-dihydro-1,6-naphthyridine-6(5H)-carboxylate

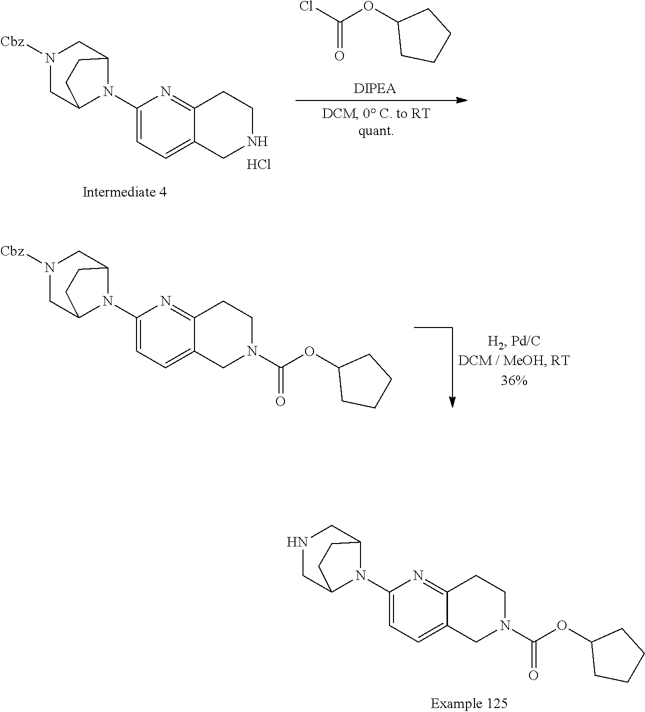 [SMILES: O=C(OCc1ccccc1)N1CC2CCC(C1)N2c1ccc2c(n1)CCNC2 ; O=C(Cl)OC1CCCC1 ; Cl ; O=C(OCc1ccccc1)N1CC2CCC(C1)N2c1ccc2c(n1)CCN(C(=O)OC1CCCC1)C2 ; O=C(OC1CCCC1)N1CCc2nc(N3C4CCC3CNC4)ccc2C1]

To a solution of benzyl 8-(5,6,7,8-tetrahydro-1,6-naphthyridin-2-yl)-3,8-diazabicyclo[3.2.1]octane-3-carboxylate (50 mg, 0.13 mmol) and diispropylethylamine (35 μL, 0.20 mmol) in dichloromethane (1.0 mL) at 0° C. was added cyclopentyl chloroformate (36 μL, 0.30 mmol) and the mixture was stirred at room temperature for 16 hours. The mixture was diluted with dichloromethane and washed with saturated aqueous sodium bicarbonate solution. The organic layers were dried with sodium sulfate, filtered and dried in vacuo. The residue was purified with silica column chromatography (0% to 50% ethyl acetate in n-heptane) and concentrated in vacuo to afford cyclopentyl 2-(3-((benzyloxy) carbonyl)-3,8-diazabicyclo[3.2.1]octan-8-yl)-7,8-dihydro-1,6-naphthyridine-6(5H)-carboxylate (65 mg, quant.) as a clear solid. UPLC (Method B): t_{R} 2.31 min, 94%, MS (ESI) 491.2 (M+H)⁺. Under nitrogen atmosphere, a solution of cyclopentyl 2-(3-((benzyloxy) carbonyl)-3,8-diazabicyclo[3.2.1]octan-8-yl)-7,8-dihydro-1,6-naphthyridine-6(5H)-carboxylate (33 mg, 67 μmol) in dichloromethane (1 mL) and methanol (1 mL) was added 10% palladium on carbon (7 mg, 6.7 μmol). Hydrogen atmosphere was introduced and the mixture was stirred at room temperature for 3 hours. The mixture was filtered through Celite®, concentrated in vacuo, purified with preparative reversed phase chromatography (Waters XSelect™ CSH C18 column, ammonium bicarbonate in water/acetonitrile) and lyophilized to afford cyclopentyl 2-(3,8-diazabicyclo[3.2.1]octan-8-yl)-7,8-dihydro-1,6-naphthyridine-6(5H)-carboxylate (8 mg, 36%, Example 125) as a beige solid. ¹H-NMR: δ_{H} (400 MHZ, CDCl₃) 7.17 (1H, d, J 8.6), 6.42 (1H, d, J 8.6), 5.19-5.12 (1H, m), 4.50-4.39 (4H, m), 3.71 (2H, m), 3.17-3.09 (2H, m), 2.84-2.75 (2H, m), 2.66-2.58 (2H, m), 2.04-1.82 (6H, m), 1.80-1.69 (4H, m), 1.65-1.59 (2H, m): UPLC (Method D): t_{R} 2.21 min, 99%, MS (ESI) 357.2 (M+H)⁺.

The following Example 126 was prepared using procedures analogous to Example 125, using the appropriate starting materials.

### Example 126: Synthesis of isopropyl 2-(3,8-diazabicyclo[3.2.1]octan-8-yl)-7,8-dihydro-1,6-naphthyridine-6(5H)-carboxylate

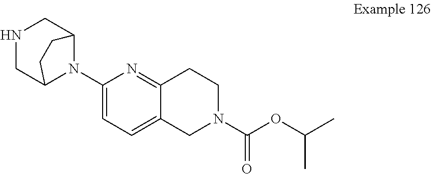 [SMILES: CC(C)OC(=O)N1CCc2nc(N3C4CCC3CNC4)ccc2C1]

Prepared using isopropyl chloroformate.

¹H-NMR: δ_{H} (400 MHZ, CDCl₃, mixture of rotamers) 7.17 (1H, d, J 8.6), 6.42 (1H, d, J 8.5), 4.97 (1H, hept, J 6.4), 4.50-4.38 (4H, m), 3.77-3.67 (2H, m), 3.18-3.08 (2H, m), 2.85-2.76 (2H, m), 2.67-2.58 (2H, m), 2.06-1.88 (4H, m), 1.27 (6H, d, J 6.3); UPLC (Method A): t_{R} 1.92 min, 95%, MS (ESI) 331.2 (M+H)⁺.

### Example 127: Synthesis of benzyl 2-(3,8-diazabicyclo[3.2.1]octan-8-yl)-7,8-dihydro-1,6-naphthyridine-6(5H)-carboxylate

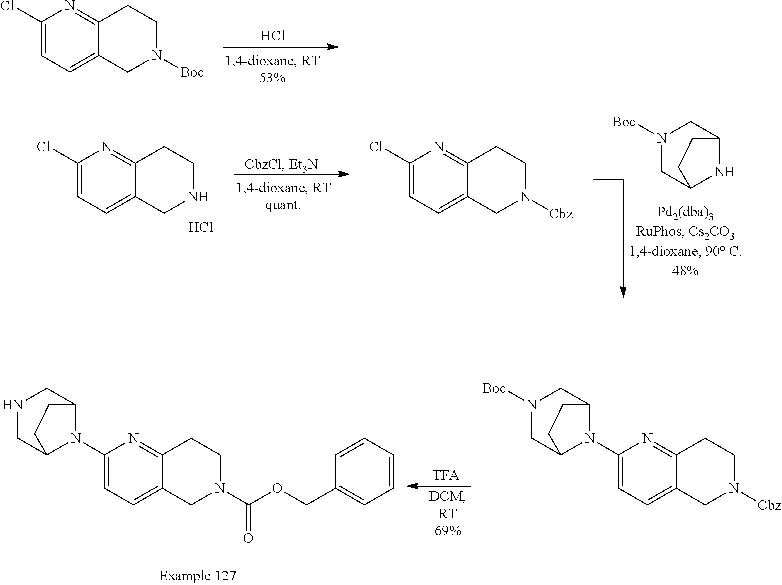

To a solution of tert-butyl 2-chloro-7,8-dihydro-1,6-naphthyridine-6(5H)-carboxylate (500 mg, 1.86 mmol) in 1,4-dioxane (5 mL) was added 4M hydrochloric acid in 1,4-dioxane (5.0 mL, 20 mmol) and the mixture was stirred at room temperature for 16 hours. The mixture was concentrated to afford 2-chloro-5,6,7,8-tetrahydro-1,6-naphthyridine hydrochloride (225 mg, 53%) as a yellow solid. LCMS (Method V): t_{R} 1.34 min, 89%, MS (ESI) 169.1 (M+H)⁺.

To a solution of benzyl 2-chloro-7,8-dihydro-1,6-naphthyridine-6(5H)-carboxylate (417 mg, 1.17 mmol) and triethylamine (382 μL, 2.74 mmol) in 1,4-dioxane (5 mL) was added benzyl chloroformate (172 μL, 1.21 mmol) and the mixture was stirred at room temperature for 3 days. The mixture was concentrated, partitioned between dichloromethane and saturated aqueous sodium bicarbonate solution and the layers were separated. The aqueous layer was extracted with dichloromethane, the combined organic layers were dried with sodium sulfate and concentrated to afford benzyl 2-chloro-7,8-dihydro-1,6-naphthyridine-6(5H)-carboxylate (417 mg, quant.) as a light yellow gum. LCMS (Method B): t_{R} 2.02 min, 84%, MS (ESI) 303.1 (M+H)⁺.

Under nitrogen atmosphere, a mixture of benzyl 2-chloro-7,8-dihydro-1,6-naphthyridine-6(5H)-carboxylate (208 mg, 583 μmol), tert-butyl 3,8-diazabicyclo[3.2.1]octane-3-carboxylate (186 mg, 874 μmol), cesium carbonate (342 mg, 1.05 mmol), 2-dicyclohexylphosphino-2′,6′-diisopropoxybiphenyl (15 mg, 32.0 μmol) and Pd₂(dba)₃ (26 mg, 29 μmol) in 1,4-dioxane (5 mL) was heated at 90° C. for 16 hours. The mixture was filtered through Celite® and concentrated in vacuo. The residue was purified with silica column chromatography (0% to 50% ethyl acetate in n-heptane) and concentrated in vacuo to afford benzyl 2-(3-(tert-butoxycarbonyl)-3,8-diazabicyclo[3.2.1]octan-8-yl)-7,8-dihydro-1,6-naphthyridine-6(5H)-carboxylate (138 mg, 48%) as a sticky clear solid. LCMS (Method B): t_{R} 2.29 min, 97%, MS (ESI) 479.2 (M+H)⁺.

To a solution of benzyl 2-(3-(tert-butoxycarbonyl)-3,8-diazabicyclo[3.2.1]octan-8-yl)-7,8-dihydro-1,6-naphthyridine-6(5H)-carboxylate (69 mg, 0.14 mmol) in dichloromethane (1.5 mL) was added trifluoroacetic acid (1.5 mL, 19 mmol). The mixture was stirred at room temperature for 16 hours. The mixture was concentrated in vacuo, purified with preparative reversed phase chromatography (Waters XSelect™ CSH C18 column, ammonium bicarbonate in water/acetonitrile) and lyophilized to afford benzyl 2-(3,8-diazabicyclo[3.2.1]octan-8-yl)-7,8-dihydro-1,6-naphthyridine-6(5H)-carboxylate (38 mg, 69%, Example 127) as a yellow solid. ¹H-NMR: δ_{H} (400 MHZ, CDCl₃, 7.42-7.29 (5H, m), 7.18 (1H, s), 6.42 (1H, d, J 8.5), 5.18 (2H, s), 4.52 (2H, s), 4.48-4.41 (2H, m), 3.77 (2H, t, J 6.0), 3.20-3.12 (2H, m), 2.87-2.76 (2H, m), 2.73-2.65 (2H, m), 2.13-1.92 (4H, m); UPLC (Method A): t_{R} 1.97 min, 97%, MS (ESI) 379.2 (M+H)⁺

The following examples (128-130) were prepared using procedures analogous to Example 87, using the appropriate starting materials.

### Example 128: Synthesis of (2S)-1-(2-(3,8-diazabicyclo[3.2.1]octan-8-yl)-5,7-dihydro-6H-pyrrolo[3,4-b]pyridin-6-yl)-2,3-dimethylbutan-1-one

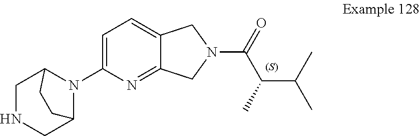

Prepared using(S)-2,3-dimethylbutanoic acid as the acid component.

¹H-NMR: δ_{H} (400 MHZ, CDCl₃, mixture of rotamers) 7.41-7.29 (1H, m), 6.49-6.44 (1H, m), 4.88-4.36 (7H, m), 3.14 (2H, d, J 12.2), 2.64 (2H, dd, J 12.3, 2.2), 2.43-2.28 (1H, m), 2.10-1.90 (5H, m), 1.15 (3H, d, J 6.7), 1.00-0.91 (6H, m); UPLC (Method C): t_{R} 1.79 min, 100%, MS (ESI) 329.2 (M+H)⁺.

### Example 129: Synthesis of 1-(2-(3,8-diazabicyclo[3.2.1]octan-8-yl)-5,7-dihydro-6H-pyrrolo[3,4-b]pyridin-6-yl)-2-(1-methylcyclopentyl)ethan-1-one

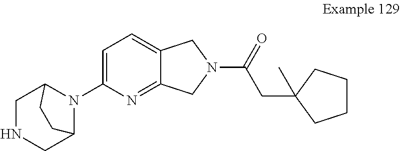 [SMILES: CC1(CC(=O)N2Cc3ccc(N4C5CCC4CNC5)nc3C2)CCCC1]

Prepared using 2-(1-methylcyclopentyl) acetic acid as the acid component.

¹H-NMR: δ_{H} (400 MHZ, CDCl₃, mixture of rotamers) 7.54-7.38 (1H, m), 6.61-6.42 (1H, m), 4.82-4.52 (6H, m), 3.30 (2H, d, J 12.3), 3.04 (2H, d, J 12.4), 2.40 (2H, d, J 4.5), 2.31-2.14 (4H, m), 1.80-1.46 (9H, m), 1.13 (3H, s); UPLC (Method C): t_{R} 2.09 min, 100%, MS (ESI) 355.2 (M+H)⁺.

### Example 130: Synthesis of 1-(2-(3,8-diazabicyclo[3.2.1]octan-8-yl)-5,7-dihydro-6H-pyrrolo[3,4-b]pyridin-6-yl)-2,2-difluoro-2-phenylethan-1-one

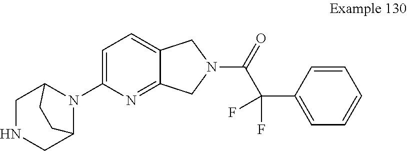 [SMILES: O=C(N1Cc2ccc(N3C4CCC3CNC4)nc2C1)C(F)(F)c1ccccc1]

Prepared using 2,2-difluoro-2-phenylacetic acid as the acid component.

¹H-NMR: δ_{H} (400 MHZ, CDCl₃, mixture of rotamers 7.73-7.62 (2H, m), 7.58-7.30 (5H, m), 6.51-6.39 (1H, m), 4.85-4.66 (4H, m), 4.41 (2H, d, J 14.1), 3.10 (2H, dd, J 12.4, 5.6), 2.63 (2H, d, J 12.3), 2.13-1.89 (4H, m); UPLC (Method A): t_{R} 1.69 min, 98%, MS (ESI) 385.2 (M+H)⁺.

### Examples 131 and 132: Synthesis of both enantiomers of 2-cyclopentyl-1-(2-(3-(hydroxymethyl) piperazin-1-yl)-7,8-dihydro-1,6-naphthyridin-6(5H)-yl)ethan-1-one (TFA salts)

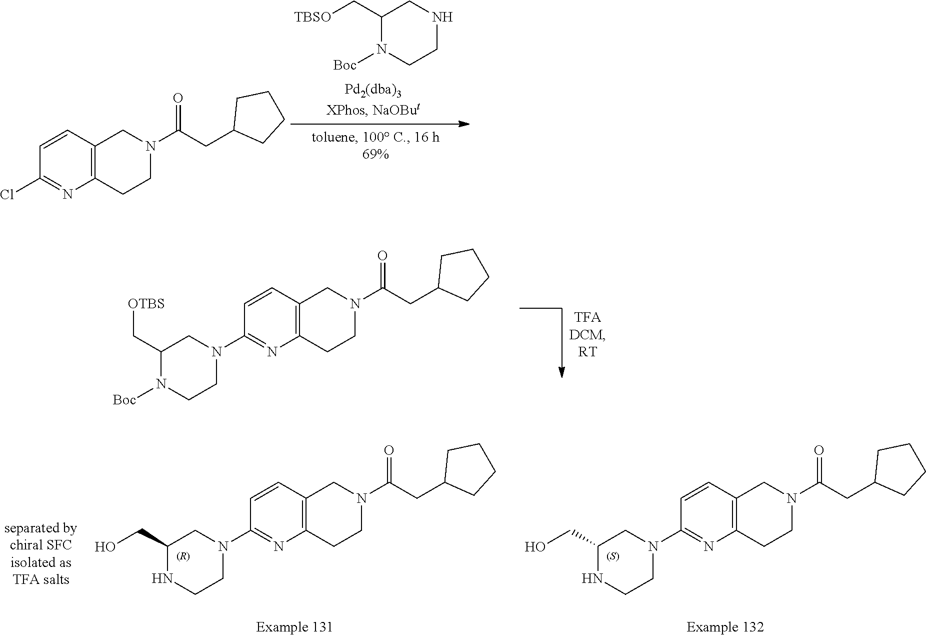

To a stirred solution of 1-(2-chloro-7,8-dihydro-1,6-naphthyridin-6(5H)-yl)-2-cyclopentylethan-1-one (200 mg, 0.717 mmol) and tert-butyl 2-(((tert-butyldimethylsilyl)oxy)methyl) piperazine-1-carboxylate (474 mg, 1.44 mmol) in toluene (10 mL) under continuous nitrogen bubbling were added sodium tert-butoxide (207 mg, 2.15 mmol) and XPhos (68 mg, 0.14 mmol). After 5 minutes Pd₂(dba)₃ (65 mg, 0.072 mmol) was added and the resulting mixture was heated at 100° C. for 16 h. After completion of the reaction as monitored by TLC, the reaction mixture was concentrated to get a residue that was diluted with water (15 mL) and extracted with 20% MeOH in DCM (20 mL×2). The combined organic extracts were washed with brine (20 mL), dried over anhydrous sodium sulphate, filtered and concentrated under reduced pressure to afford the crude material. The crude product was purified by flash column chromatography using EtOAc in petroleum ether (0 to 100%) as an eluent to obtain desired product tert-butyl 2-(((tert-butyldimethylsilyl)oxy)methyl)-4-(6-(2-cyclopentylacetyl)-5,6,7,8-tetrahydro-1,6-naphthyridin-2-yl) piperazine-1-carboxylate (570 mg, 69%) as a yellow gum.

To a stirred solution of tert-butyl 2-(((tert-butyldimethylsilyl)oxy)methyl)-4-(6-(2-cyclopentylacetyl)-5,6,7,8-tetrahydro-1,6-naphthyridin-2-yl) piperazine-1-carboxylate (470 mg, 0.820 mmol) in DCM (10 mL) at 0° C. was added TFA (3.14 mL, 41.0 mmol) and the resultant mixture was stirred at room temperature for 16 h. After completion, the reaction mixture was concentrated under reduced pressure to afford the crude material that was purified by reversed-phase preparative HPLC. The fractions were concentrated, and the enantiomers were separated by chiral preparative SFC. Both the fractions were collected and concentrated under lyophilisation to afford fraction-1 (30 mg, Example 131) and fraction-2 (36 mg, Example 132) respectively.

First eluting isomer (Example 131): ¹H-NMR: δ_{H} (400 MHZ, DMSO-d₆, mixture of rotamers) 9.12-9.01 (1H, m), 8.69-8.62 (1H, m), 7.47-7.44 (1H, m), 6.82-6.80 (1H, m), 5.45 (1H, br s), 4.55-4.41 (2H, m), 4.28-4.21 (2H, m), 3.74-3.72 (2H, m), 3.69-3.61 (1H, m), 3.59-3.58 (2H, m), 3.07-3.04 (2H, m), 2.91-2.77 (1H, m), 2.68-2.60 (2H, m), 2.55-2.41 (2H, m), 2.39-2.33 (2H, m), 1.81-1.75 (2H, m), 1.59-1.48 (4H, m), 1.26-1.12 (2H, m); MS (ESI) 359.2 (M+H)⁺.

Second eluting isomer (Example 132): ¹H-NMR: δ_{H} (400 MHZ, DMSO-d₆, mixture of rotamers) 9.12-9.01 (1H, m), 8.69-8.62 (1H, m), 7.47-7.44 (1H, m), 6.82-6.80 (1H, m), 5.45 (1H, br s), 4.55-4.41 (2H, m), 4.28-4.21 (2H, m), 3.74-3.61 (4H, m), 3.59-3.58 (2H, m), 3.07-3.04 (2H, m), 2.91-2.77 (1H, m), 2.68-2.55 (2H, m), 2.39-2.33 (2H, m), 2.33-2.11 (1H, m), 1.81-1.75 (2H, m), 1.59-1.48 (4H, m), 1.26-1.12 (m, 2H); MS (ESI) 359.2 (M+H)⁺.

### Example 133: Synthesis of 1-(2-(3,8-diazabicyclo[3.2.1]octan-8-yl)-7,8-dihydropyrido[4,3-d]pyrimidin-6(5H)-yl)-2-phenylethan-1-one

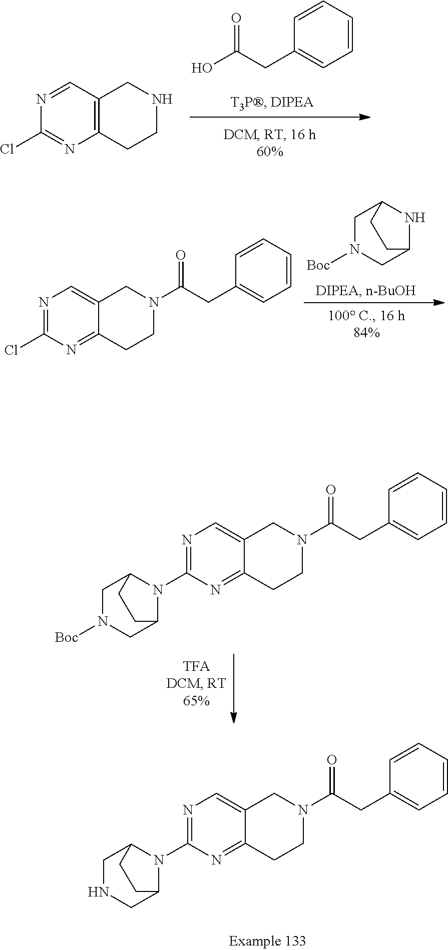

To a stirred solution of 2-chloro-5,6,7,8-tetrahydropyrido[4,3-d]pyrimidine (400 mg, 2.36 mmol) in DCM (7 mL) at 0° C. were added n-propyl phosphonic acid anhydride, cyclic trimer (50% in EtOAc) (2.25 mL, 7.07 mmol) and DIPEA (1.92 mL, 11.8 mmol). After 15 min 2-phenylacetic acid (642 mg, 4.72 mmol) was added and stirred at room temperature for 16 h. After completion, the reaction mixture was diluted with water (15 mL) and extracted with 5% MeOH in DCM (15 mL×3). The combined organic extracts were washed with brine (10 mL), dried over anhydrous sodium sulphate, filtered, and concentrated under reduced pressure to afford 1-(2-chloro-7,8-dihydropyrido[4,3-d]pyrimidin-6(5H)-yl)-2-phenylethan-1-one (1.8 g, 60%) as an off-white solid.

To a stirred solution of 1-(2-chloro-7,8-dihydropyrido[4,3-d]pyrimidin-6(5H)-yl)-2-phenylethan-1-one (1.8 g, 6.26 mmol) in n-butanol (10 mL) were added tert-butyl 3,8-diazabicyclo[3.2.1]octane-3-carboxylate (2.66 g, 12.5 mmol) and DIPEA (3.07 mL, 18.8 mmol). The resulting mixture was heated 100° C. for 16 h. Afterwards, the reaction mixture was diluted with water (15 mL) and extracted with 5% MeOH in DCM (15 mL×3). The combined organic extracts were washed with brine (10 mL), dried over anhydrous sodium sulphate, filtered, and concentrated under reduced pressure to afford tert-butyl 8-(6-(2-phenylacetyl)-5,6,7,8-tetrahydropyrido[4,3-d]pyrimidin-2-yl)-3,8-diazabicyclo[3.2.1]octane-3-carboxylate (2.5 g, 84%) as an off white solid.

To a stirred solution of tert-butyl 8-(6-(2-phenylacetyl)-5,6,7,8-tetrahydropyrido[4,3-d]pyrimidin-2-yl)-3,8-diazabicyclo[3.2.1]octane-3-carboxylate (150 mg, 0.32 mmol) in DCM (3 mL) at 0° C. was added TFA (0.075 mL, 0.97 mmol) and the resulting mixture was stirred at room temperature for 16 h. After completion, the reaction mixture was concentrated, diluted with water (15 mL), basified with sat. NH₄HCO₃ solution and extracted with 5% MeOH in DCM (15 mL×3). The combined organic extracts were washed with brine (10 mL), dried over anhydrous sodium sulphate, filtered, and concentrated under reduced pressure to afford crude material that was purified by reversed-phase preparative HPLC using ammonium bicarbonate as a buffer. Pure fractions were lyophilized to obtain 1-(2-(3,8-diazabicyclo[3.2.1]octan-8-yl)-7,8-dihydropyrido[4,3-d]pyrimidin-6(5H)-yl)-2-phenylethan-1-one (65.1 mg, 65%, Example 133) as an off-white solid. ¹H-NMR: δ_{H} (400 MHZ, DMSO-d₆, 80° C.) 8.13 (1H, s), 7.35-7.20 (5H, m), 4.60-4.50 (4H, m), 3.81-3.75 (4H, m), 2.86-2.83 (2H, m), 2.68-2.53 (4H, m), 1.95-1.75 (4H, m); MS (ESI) 364.2 (M+H)⁺.

The following examples (134-148) were prepared using procedures analogous to Example 133, using the appropriate starting materials.

### Example 134: Synthesis of 2-phenyl-1-(2-(piperazin-1-yl)-7,8-dihydropyrido[4,3-d]pyrimidin-6(5H)-yl)ethan-1-one

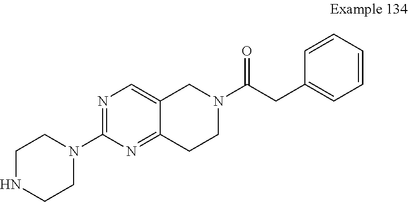 [SMILES: O=C(Cc1ccccc1)N1CCc2nc(N3CCNCC3)ncc2C1]

Prepared by analogy to Example 133 using 2-chloro-5,6,7,8-tetrahydropyrido[4,3-d]pyrimidine and 2-phenylacetic acid in the first step and tert-butyl piperazine-1-carboxylate in the second step. ¹H-NMR: δ_{H} (DMSO-d₆, 400 MHZ, mixture of rotamers) 8.19 (0.6H, s), 8.12 (0.4H, s), 7.33-7.20 (5H, m), 4.57-4.48 (2H, m), 3.81-3.73 (4H, m), 3.62-3.58 (4H, m), 2.70-2.68 (4H, m), 2.65-2.62 (2H, m); MS (ESI) 338.2 (M+H)⁺.

### Example 135: Synthesis of 1-(2-(3,8-diazabicyclo[3.2.1]octan-3-yl)-7,8-dihydropyrido[4,3-d]pyrimidin-6(5H)-yl)-2-phenylethan-1-one

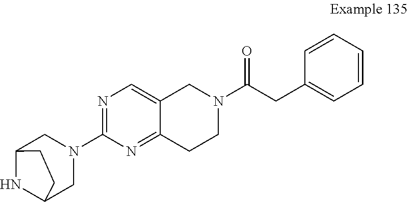 [SMILES: O=C(Cc1ccccc1)N1CCc2nc(N3CC4CCC(C3)N4)ncc2C1]

Prepared by analogy to Example 133 using 2-chloro-5,6,7,8-tetrahydropyrido[4,3-d]pyrimidine and 2-phenylacetic acid in the first step and tert-butyl 3,8-diazabicyclo[3.2.1]octane-8-carboxylate in the second step. ¹H-NMR: δ_{H} (DMSO-d₆, 400 MHZ, mixture of rotamers) 8.17-8.10 (1H, m), 7.35-7.15 (5H, m), 4.57-4.48 (2H, m), 4.16-4.11 (2H, m), 3.81-3.78 (2H, m), 3.77-3.72 (2H, m), 3.45-3.35 (2H, m), 2.91-2.88 (2H, m), 2.63-2.55 (2H, m), 2.55-2.30 (1H, br m), 1.70-1.45 (4H, m); MS (ESI) 364.1 (M+H)⁺.

### Example 136: Synthesis of 4-(2-(2-(3,8-diazabicyclo[3.2.1]octan-8-yl)-7,8-dihydropyrido[4,3-d]pyrimidin-6(5H)-yl)-2-oxoethyl)benzonitrile

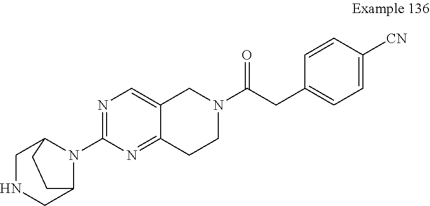 [SMILES: N#Cc1ccc(CC(=O)N2CCc3nc(N4C5CCC4CNC5)ncc3C2)cc1]

Prepared by analogy to Example 133 using 2-chloro-5,6,7,8-tetrahydropyrido[4,3-d]pyrimidine and 2-(4-cyanophenyl) acetic acid in the first step and tert-butyl 3,8-diazabicyclo[3.2.1]octane-3-carboxylate in the second step. ¹H-NMR: δ_{H} (DMSO-d₆, 400 MHZ, mixture of rotamers) 8.19 & 8.13 (1H, 2×s, rotamers), 7.79-7.75 (2H, m), 7.47-7.42 (2H, m), 4.59-4.49 (4H, m), 3.95-3.93 (2H, m), 3.81-3.74 (2H, m), 2.81-2.78 (2H, m), 2.71-2.63 (2H, m), 2.61-2.53 (2H, m), 1.95-1.75 (4H, m); MS (ESI) 389.2 (M+H)⁺.

### Example 137: Synthesis of 4-(2-oxo-2-(2-(piperazin-1-yl)-7,8-dihydropyrido[4,3-d]pyrimidin-6(5H)-yl)ethyl)benzonitrile

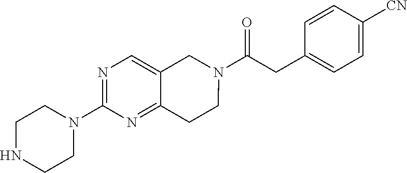 [SMILES: N#Cc1ccc(CC(=O)N2CCc3nc(N4CCNCC4)ncc3C2)cc1]

Prepared by analogy to Example 133 using 2-chloro-5,6,7,8-tetrahydropyrido[4,3-d]pyrimidine and 2-(4-cyanophenyl) acetic acid in the first step and tert-butyl piperazine-1-carboxylate in the second step. ¹H NMR: δ_{H} (400 MHZ, DMSO-d₆, mixture of rotamers) 8.20-8.14 (1H, m), 7.79-7.74 (2H, m), 7.46-7.42 (2H, m), 4.59-4.49 (2H, m), 3.95-3.93 (2H, m), 3.80-3.72 (2H, m), 3.66-3.55 (4H, m), 2.76-2.63 (6H, m); MS (ESI) 363.1 (M+H)⁺.

### Example 138: Synthesis of 4-(2-(2-(3,8-diazabicyclo[3.2.1]octan-3-yl)-7,8-dihydropyrido[4,3-d]pyrimidin-6(5H)-yl)-2-oxoethyl)benzonitrile

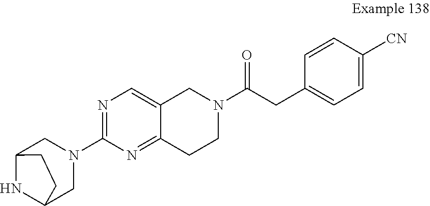 [SMILES: N#Cc1ccc(CC(=O)N2CCc3nc(N4CC5CCC(C4)N5)ncc3C2)cc1]

Prepared by analogy to Example 133 using 2-chloro-5,6,7,8-tetrahydropyrido[4,3-d]pyrimidine and 2-(4-cyanophenyl) acetic acid in the first step and tert-butyl 3,8-diazabicyclo[3.2.1]octane-8-carboxylate in the second step. ¹H NMR: δ_{H} (400 MHZ, DMSO-d₆, mixture of rotamers) 8.18-8.12 (1H, m), 7.79-7.74 (2H, m), 7.46-7.42 (2H, m), 4.59-4.48 (2H, m), 4.16-4.13 (2H, m), 3.95-3.92 (2H, m), 3.80-3.72 (2H, m), 3.50-3.35 (2H, m), 2.91-2.88 (2H, m), 2.70-2.62 (2H, m), 2.45-2.30 (1H, m), 1.70-1.45 (4H, m); MS (ESI) 389.2 (M+H)⁺.

### Example 139: Synthesis of 1-(2-(3,8-diazabicyclo[3.2.1]octan-8-yl)-7,8-dihydropyrido[4,3-d]pyrimidin-6(5H)-yl)-2-(4-fluorophenyl)ethan-1-one

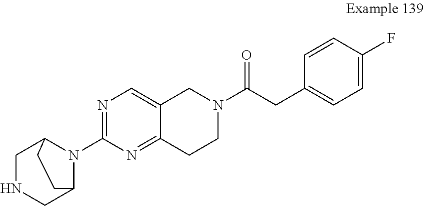 [SMILES: O=C(Cc1ccc(F)cc1)N1CCc2nc(N3C4CCC3CNC4)ncc2C1]

Prepared by analogy to Example 133 using 2-chloro-5,6,7,8-tetrahydropyrido[4,3-d]pyrimidine and 2-(4-fluorophenyl) acetic acid in the first step and tert-butyl 3,8-diazabicyclo[3.2.1]octane-3-carboxylate in the second step. ¹H-NMR: δ_{H} (400 MHZ, DMSO-d₆, 80° C.) 8.14 (1H, s), 7.29 (2H, J 8.3, 5.7, dd), 7.13-7.05 (2H, m), 4.61-4.47 (4H, m), 3.83-3.74 (4H, m), 2.87-2.82 (2H, m), 2.67-2.64 (2H, m), 2.59-2.55 (2H, m), 2.24 (1H, br s), 1.98-1.87 (2H, m), 1.86-1.76 (2H, m); MS (ESI) 382.2 (M+H)⁺.

### Example 140: Synthesis of 2-(4-fluorophenyl)-1-(2-(piperazin-1-yl)-7,8-dihydropyrido[4,3-d]pyrimidin-6(5H)-yl)ethan-1-one

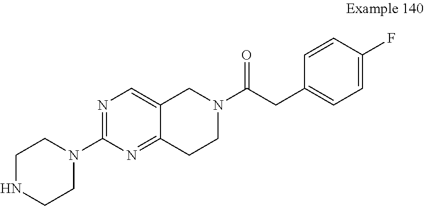 [SMILES: O=C(Cc1ccc(F)cc1)N1CCc2nc(N3CCNCC3)ncc2C1]

Prepared by analogy to Example 133 using 2-chloro-5,6,7,8-tetrahydropyrido[4,3-d]pyrimidine and 2-(4-fluorophenyl) acetic acid in the first step and tert-butyl piperazine-1-carboxylate in the second step. ¹H NMR: δ_{H} (400 MHZ, DMSO-d₆, mixture of rotamers) 8.22-8.10 (1H, m), 7.33-7.22 (2H, m), 7.17-7.07 (2H, m), 4.60-4.45 (2H, m), 3.83-3.70 (4H, m), 3.67-3.55 (4H, m), 2.73-2.66 (4H, m), 2.65-2.60 (2H, m); MS (ESI) 356.2 (M+H)⁺.

### Example 141: Synthesis of 1-(2-(3,8-diazabicyclo[3.2.1]octan-3-yl)-7,8-dihydropyrido[4,3-d]pyrimidin-6(5H)-yl)-2-(4-fluorophenyl)ethan-1-one

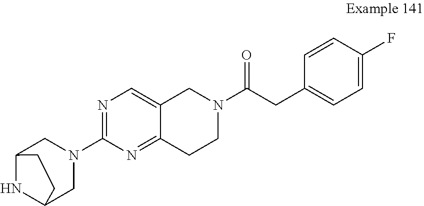 [SMILES: O=C(Cc1ccc(F)cc1)N1CCc2nc(N3CC4CCC(C3)N4)ncc2C1]

Prepared by analogy to Example 133 using 2-chloro-5,6,7,8-tetrahydropyrido[4,3-d]pyrimidine and 2-(4-fluorophenyl) acetic acid in the first step and tert-butyl 3,8-diazabicyclo[3.2.1]octane-8-carboxylate in the second step. ¹H-NMR: δ_{H} (400 MHZ, DMSO-d₆, mixture of rotamers) 8.17-8.11 (1H, m), 7.30-7.24 (2H, m), 7.15-7.08 (2H, m), 4.61-4.44 (2H, m), 4.18-4.10 (2H, m), 3.83-3.67 (4H, m), 3.49-3.36 (2H, m), 2.94-2.83 (2H, m), 2.66-2.59 (2H, m), 2.43-2.33 (1H, br m), 1.66-1.55 (2H, m), 1.53-1.45 (2H, m); MS (ESI) 382.2 (M+H)⁺.

### Example 142: Synthesis of 1-(2-(3,8-diazabicyclo[3.2.1]octan-8-yl)-7,8-dihydropyrido[4,3-d]pyrimidin-6(5H)-yl)-2-cyclopentylethan-1-one

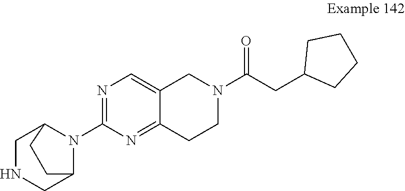 [SMILES: O=C(CC1CCCC1)N1CCc2nc(N3C4CCC3CNC4)ncc2C1]

Prepared by analogy to Example 133 using 2-chloro-5,6,7,8-tetrahydropyrido[4,3-d]pyrimidine and 2-cyclopentylacetic acid in the first step and tert-butyl 3,8-diazabicyclo[3.2.1]octane-3-carboxylate in the second step. ¹H-NMR: δ_{H} (DMSO-d₆, 400 MHZ, mixture of rotamers) 8.17 (1H, s), 4.51-4.45 (2H, m), 4.16-4.12 (2H, m), 3.72-3.69 (2H, m), 3.44-3.32 (2H, m), 2.90-2.87 (2H, m), 2.75-2.55 (2H, m), 2.49-2.32 (2H, m), 2.21-2.16 (1H, m), 1.80-1.70 (2H, m), 1.67-1.40 (8H, m), 1.20-1.05 (2H, m); MS (ESI) 356.2 (M+H)⁺.

### Example 143: Synthesis of 2-cyclopentyl-1-(2-(piperazin-1-yl)-7,8-dihydropyrido[4,3-d]pyrimidin-6(5H)-yl)ethan-1-one

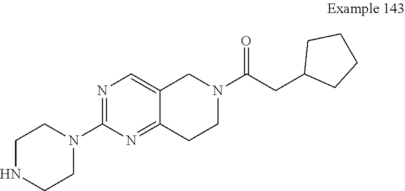 [SMILES: O=C(CC1CCCC1)N1CCc2nc(N3CCNCC3)ncc2C1]

Prepared by analogy to Example 133 using 2-chloro-5,6,7,8-tetrahydropyrido[4,3-d]pyrimidine and 2-cyclopentylacetic acid in the first step and tert-butyl piperazine-1-carboxylate in the second step. ¹H-NMR: δ_{H} (DMSO-d₆, 400 MHZ, mixture of rotamers) 8.19 (1H, s), 4.52-4.45 (2H, m), 3.71 (2H, J 6.0, t), 3.65-3.58 (4H, m), 2.76-2.55 (6H, m), 2.49-2.33 (2H, m), 2.15 (1H, m), 1.80-1.70 (2H, m), 1.58 (4H, m), 1.17-1.07 (2H, m); MS (ESI) 330.3 (M+H)⁺.

### Example 144: Synthesis of 1-(2-(3,8-diazabicyclo[3.2.1]octan-8-yl)-7,8-dihydropyrido[4,3-d]pyrimidin-6(5H)-yl)-2-cyclohexylethan-1-one

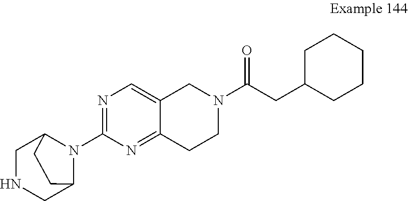 [SMILES: O=C(CC1CCCCC1)N1CCc2nc(N3C4CCC3CNC4)ncc2C1]

Prepared by analogy to Example 133 using 2-chloro-5,6,7,8-tetrahydropyrido[4,3-d]pyrimidine and 2-cyclohexylacetic acid in the first step and tert-butyl 3,8-diazabicyclo[3.2.1]octane-3-carboxylate in the second step. ¹H-NMR: δ_{H} (DMSO-d₆, 400 MHZ, mixture of rotamers) 8.18 (1H, s), 4.53-4.45 (4H, m), 3.72 (2H, t, J 6.0), 2.81-2.78 (2H, m), 2.75-2.65 (2H, m), 2.65-2.45 (2H, m), 2.30-2.20 (2H, m), 1.95-1.86 (2H, m), 1.86-1.76 (2H, m), 1.75-1.55 (6H, m), 1.20-1.00 (3H, m), 1.00-0.85 (2H, m); MS (ESI) 370.3 (M+H)⁺.

### Example 145: Synthesis of 2-cyclohexyl-1-(2-(piperazin-1-yl)-7,8-dihydropyrido[4,3-d]pyrimidin-6(5H)-yl)ethan-1-one

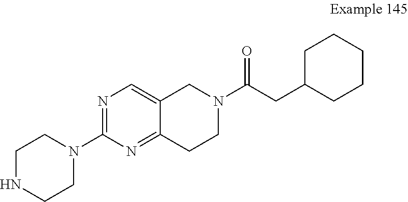 [SMILES: O=C(CC1CCCCC1)N1CCc2nc(N3CCNCC3)ncc2C1]

Prepared by analogy to Example 133 using 2-chloro-5,6,7,8-tetrahydropyrido[4,3-d]pyrimidine and 2-cyclohexylacetic acid in the first step and tert-butyl piperazine-1-carboxylate in the second step. ¹H-NMR: δ_{H} (400 MHZ, DMSO-d₆, mixture of rotamers) 8.19 (1H, s), 4.54-4.39 (2H, m), 3.73-3.70 (2H, m), 3.65-3.59 (4H, m), 2.75-2.69 (4H, m), 2.65-2.45 (2H, m), 2.28-2.21 (2H, m), 1.75-1.55 (6H, m), 1.60-1.05 (3H, m), 1.00-0.85 (2H, m); MS (ESI) 344.2 (M+H)⁺.

### Example 146: Synthesis of 1-(2-(3,8-diazabicyclo[3.2.1]octan-8-yl)-7,8-dihydropyrido[4,3-d]pyrimidin-6(5H)-yl)-3,3-dimethylbutan-1-one

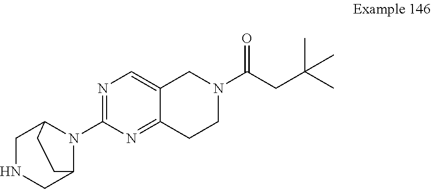 [SMILES: CC(C)(C)CC(=O)N1CCc2nc(N3C4CCC3CNC4)ncc2C1]

Prepared by analogy to Example 133 using 2-chloro-5,6,7,8-tetrahydropyrido[4,3-d]pyrimidine and 3,3-dimethylbutanoic acid in the first step and tert-butyl 3,8-diazabicyclo[3.2.1]octane-3-carboxylate in the second step. ¹H NMR: δ_{H} (400 MHZ, DMSO-d₆, mixture of rotamers) 8.18 (1H, s), 4.56-4.46 (2H, m), 4.18-4.14 (2H, m), 3.78-3.70 (2H, m), 3.58-3.42 (2H, m), 2.95-2.87 (2H, m), 2.73-2.55 (2H, m), 2.32-2.29 (2H, m), 1.68-1.60 (2H, m), 1.55-1.45 (2H, m), 1.01 (9H, 2×s, rotamers); MS (ESI) 344.2 (M+H)⁺.

### Example 147: Synthesis of (4-fluorophenyl)(2-(piperazin-1-yl)-7,8-dihydropyrido[4,3-d]pyrimidin-6(5H)-yl)methanone

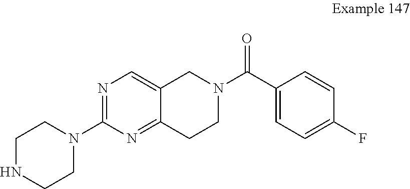 [SMILES: O=C(c1ccc(F)cc1)N1CCc2nc(N3CCNCC3)ncc2C1]

Prepared by analogy to Example 133 using 2-chloro-5,6,7,8-tetrahydropyrido[4,3-d]pyrimidine and 4-fluorobenzoic acid in the first step and tert-butyl piperazine-1-carboxylate in the second step. ¹H-NMR: δ_{H} (400 MHZ, DMSO-d₆, 80° C.) 8.16 (1H, s), 7.54-7.50 (2H, m), 7.30-7.25 (2H, m), 4.54 (2H, s), 3.77-3.68 (2H, m), 3.67-3.63 (4H, m), 2.78-2.73 (6H, m); MS (ESI) 342.2 (M+H)⁺.

### Example 148: Synthesis of 1-(2-(3,8-diazabicyclo[3.2.1]octan-8-yl)-5,8-dihydropyrido[3,4-d]pyrimidin-7 (6H)-yl)-2-cyclopentylethan-1-one

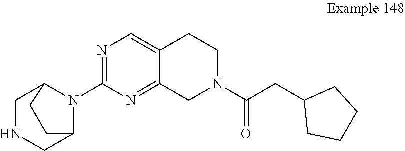 [SMILES: O=C(CC1CCCC1)N1CCc2cnc(N3C4CCC3CNC4)nc2C1]

Prepared by analogy to Example 133 using 2-chloro-5,6,7,8-tetrahydropyrido[4,3-d]pyrimidine 2-chloro-5,6,7,8-tetrahydropyrido[3,4-d]pyrimidine and 2-cyclopentylacetic acid in the first step and tert-butyl 3,8-diazabicyclo[3.2.1]octane-3-carboxylate in the second step. ¹H-NMR: OH (DMSO-d₆, 400 MHZ, 80° C.) 8.14 (1H, s), 4.55-4.50 (2H, m), 4.43 (2H, s), 3.69 (2H, t, J 6.1), 2.85-2.81 (2H, m), 2.68-2.57 (2H, m), 2.57-2.54 (2H, m), 2.42-2.40 (2H, m), 2.18 (1H, septet, J 7.5), 1.95-1.70 (6H, m), 1.65-1.40 (4H, m), 1.20-1.08 (2H, m); MS (ESI) 356.2 (M+H)⁺.

### Examples 149 and 150: Synthesis of 3,3-dimethyl-1-(2-(piperazin-1-yl)-7,8-dihydro-1,6-naphthyridin-6(5H)-yl) butan-1-one and 1-(2-(4-ethylpiperazin-1-yl)-7,8-dihydro-1,6-naphthyridin-6(5H)-yl)-3,3-dimethylbutan-1-one

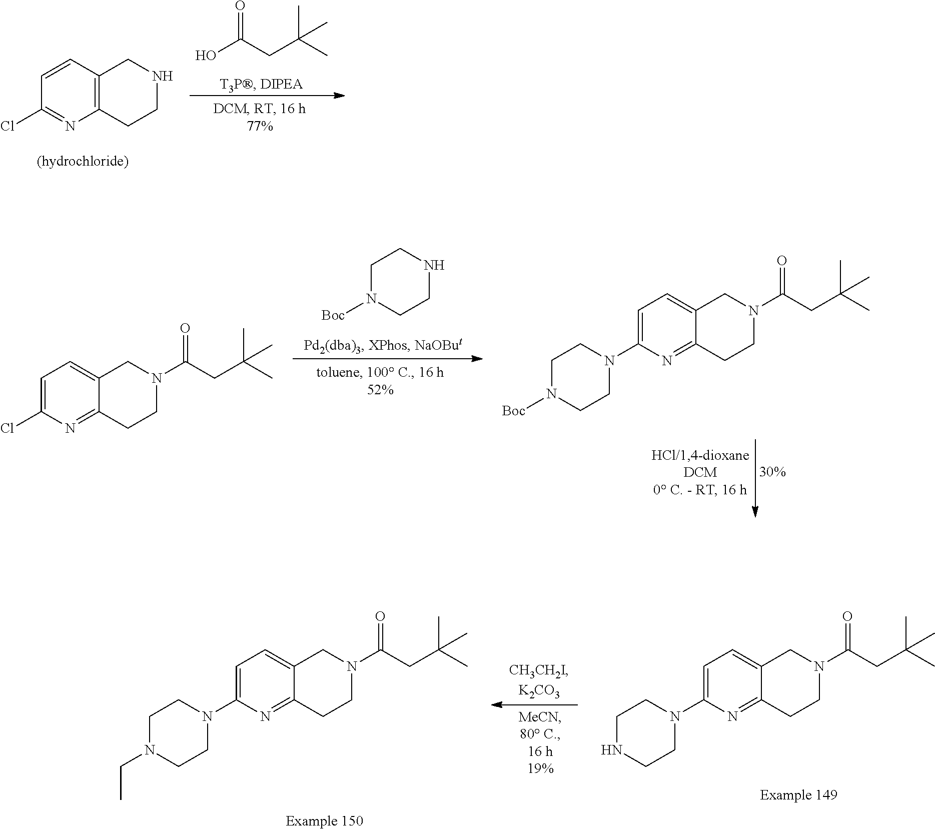

To a stirred solution of 3,3-dimethylbutanoic acid (5.66 g, 48.8 mmol) in DCM (150 mL) at 0° C. were added n-propylphosphonic acid anhydride, cyclic trimer (50% in EtOAc; 46.5 g, 73.1 mmol) and DIPEA (15.8 g, 122 mmol). After 15 min, 2-chloro-5,6,7,8-tetrahydro-1,6-naphthyridine hydrochloride (5.00 g, 24.4 mmol) was added and the resulting mixture was stirred at room temperature for 16 h. After completion, the reaction mixture was diluted with water (200 mL) and extracted with 10% MeOH in DCM (500 mL×2). The combined organic extracts were washed with brine (200 mL), dried (Na₂SO₄), filtered and concentrated under reduced pressure to afford the crude material, which was then processed by flash column chromatography (0 to 30% EtOAc/petroleum ether eluent) to obtain 1-(2-chloro-7,8-dihydro-1,6-naphthyridin-6(5H)-yl)-3,3-dimethylbutan-1-one (5.0 g, 77%) as an off-white solid.

To a stirred solution of 1-(2-chloro-7,8-dihydro-1,6-naphthyridin-6(5H)-yl)-3,3-dimethylbutan-1-one (700 mg, 2.62 mmol) and tert-butyl piperazine-1-carboxylate (586 mg, 3.15 mmol), in toluene (30 mL) were added sodium tert-butoxide (757 mg, 7.87 mmol), XPhos (250 mg, 0.525 mmol) and Pd₂(dba)₃ (240 mg, 0.262 mmol) under continuous nitrogen bubbling. The resulting mixture was heated at 100° C. for 16 h. After completion, the reaction mixture was concentrated, diluted with DCM and filtered through a pad of Celite®. The filtrate was extracted with 10% MeOH in DCM (100 mL×2). The combined organic extracts were washed with brine (50 mL), dried (Na₂SO₄), filtered, and concentrated under reduced pressure to afford the crude material, which was then processed by flash column chromatography (0 to 50% EtOAc/petroleum ether eluent) to obtain tert-butyl 4-(6-(3,3-dimethylbutanoyl)-5,6,7,8-tetrahydro-1,6-naphthyridin-2-yl) piperazine-1-carboxylate (570 mg, 52%) as an off-white solid.

To a stirred solution of tert-butyl 4-(6-(3,3-dimethylbutanoyl)-5,6,7,8-tetrahydro-1,6-naphthyridin-2-yl) piperazine-1-carboxylate (1.00 g, 2.40 mmol) in DCM (10 mL) at 0° C. was added 4M HCl in dioxane (10 mL, 40.0 mmol) and the resulting mixture was stirred at room temperature for 16 h. After completion, the reaction mixture was concentrated, basified (10% NaHCO₃ aq) and extracted with 10% MeOH in DCM (5 mL×2). The combined organic extracts were washed with brine (5 mL), dried (Na₂SO₄), filtered, and concentrated under reduced pressure to afford crude solid, which was then processed by a reversed-phase preparative HPLC using ammonium bicarbonate as a buffer. Pure fractions were lyophilized to obtain 3,3-dimethyl-1-(2-(piperazin-1-yl)-7,8-dihydro-1,6-naphthyridin-6(5H)-yl) butan-1-one (230 mg, 30%, Example 149) as an off-white solid. ¹H-NMR: δ_{H} (400 MHZ, DMSO-d₆, 80° C.) 7.32 (1H, J 8.5, d), 6.62 (1H, J 8.6, d), 4.52 (2H, s), 3.76 (2H, J 6.1, t), 3.40-3.36 (4H, m), 2.89-2.71 (4H, m), 2.53-2.53 (2H, m), 2.32 (2H, s), 1.02 (9H, s); MS (ESI) 317.3 (M+H)⁺.

A stirred mixture of 3,3-dimethyl-1-(2-(piperazin-1-yl)-7,8-dihydro-1,6-naphthyridin-6(5H)-yl) butan-1-one (80.0 mg, 0.252 mmol), K₂CO₃ (86.0 mg, 0.622 mmol) and ethyl iodide (35.5 mg, 0.228 mmol) in MeCN (5 mL) was heated at 80° C. for 16 hours. After completion, the reaction mixture was concentrated under reduced pressure to obtain a residue that was diluted with water (10 mL) and extracted with EtOAc (10 mL×2). The combined organic extracts were washed with brine (10 mL), dried (Na₂SO₄), filtered, and concentrated under reduced pressure. The crude residue was processed by reversed-phase preparative HPLC using ammonium bicarbonate as a buffer. Pure fractions were lyophilized to obtain 1-(2-(4-ethylpiperazin-1-yl)-7,8-dihydro-1,6-naphthyridin-6(5H)-yl)-3,3-dimethylbutan-1-one (15 mg, 19%, Example 150) as an off-white solid. ¹H-NMR: δ_{H} (400 MHZ, DMSO-d₆, mixture of rotamers) 7.37-7.33 (1H, m), 6.71-6.67 (1H, m), 4.57-4.47 (2H, m), 3.75 (2H, q, J 6.2), 3.45-3.39 (4H, m), 2.76-2.62 (2H, m), 2.45-2.40 (4H, m), 2.36-2.29 (4H, m), 1.05 (3H, t, J 6.2), 1.00 & 0.97 (9H, 2×s, rotamers); MS (ESI) 345.3 (M+H)⁺.

The following examples (151-156) were prepared using procedures analogous to Example 149, using the appropriate starting materials.

### Example 151: Synthesis of 1-(2-(3,9-diazabicyclo[3.3.1]nonan-9-yl)-7,8-dihydro-1,6-naphthyridin-6(5H)-yl)-2-cyclopentylethan-1-one

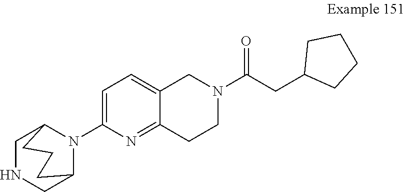 [SMILES: O=C(CC1CCCC1)N1CCc2nc(N3C4CCCC3CNC4)ccc2C1]

Prepared by analogy to Example 149 using 2-chloro-5,6,7,8-tetrahydro-1,6-naphthyridine and 2-cyclopentylacetic acid in the first step and tert-butyl 3,9-diazabicyclo[3.3.1]nonane-3-carboxylate in the second step. ¹H-NMR: δ_{H} (400 MHZ, DMSO-d₆, mixture of rotamers) 7.31-7.28 (1H, m), 6.63-6.59 (1H, m), 4.49-4.44 (2H, m), 4.34-4.28 (2H, m), 3.72-3.69 (2H, m), 3.05-2.95 (4H, m), 2.80-2.60 (3H, m), 2.49-2.39 (2H, m), 2.20-2.10 (1H, m), 1.77-1.71 (4H, m), 1.70-1.47 (7H, m), 1.20-1.05 (2H, m); MS (ESI) 369.3 (M+H)⁺.

### Example 152: Synthesis of 1-(2-(3,9-diazabicyclo[3.3.1]nonan-3-yl)-7,8-dihydro-1,6-naphthyridin-6(5H)-yl)-2-cyclopentylethan-1-one

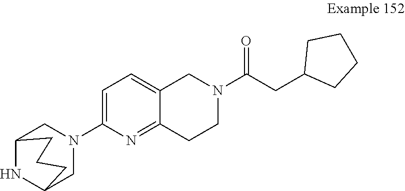 [SMILES: O=C(CC1CCCC1)N1CCc2nc(N3CC4CCCC(C3)N4)ccc2C1]

Prepared by analogy to Example 149 using 2-chloro-5,6,7,8-tetrahydro-1,6-naphthyridine and 2-cyclopentylacetic acid in the first step and tert-butyl 3,9-diazabicyclo[3.3.1]nonane-9-carboxylate in the second step. ¹H-NMR: δ_{H} (400 MHZ, DMSO-d₆, mixture of rotamers) 7.33 (1H, d, J 8.6), 6.62-6.60 (1H, m), 4.52-4.46 (2H, m), 4.10-4.05 (2H, m), 3.73-3.69 (2H, m), 3.08-2.94 (4H, m), 2.75-2.60 (2H, m), 2.50-2.39 (2H, m), 2.24-1.94 (2H, m), 1.82-1.41 (11H, m), 1.21-1.05 (2H, m); MS (ESI) 369.3 (M+H)⁺.

### Example 153: Synthesis of 1-(2-(2,5-diazabicyclo[2.2.2]octan-2-yl)-7,8-dihydro-1,6-naphthyridin-6(5H)-yl)-2-cyclopentylethan-1-one

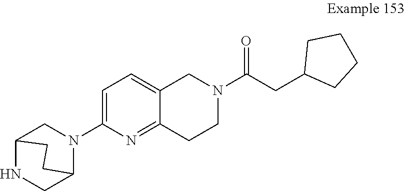 [SMILES: O=C(CC1CCCC1)N1CCc2nc(N3CC4CCC3CN4)ccc2C1]

Prepared by analogy to Example 149 using 2-chloro-5,6,7,8-tetrahydro-1,6-naphthyridine and 2-cyclopentylacetic acid in the first step and tert-butyl 2,5-diazabicyclo[2.2.2]octane-2-carboxylate in the second step. ¹H NMR: δ_{H} (400 MHZ, DMSO-d₆, 80° C.) 7.29 (1H, d, J 8.5), 6.34 (1H, d, J 8.5), 4.55-4.40 (3H, s), 3.72 (2H, t, J 6.1), 3.53-3.51 (1H, m), 3.46-3.39 (1H, m), 3.20-2.90 (3H, m), 2.77-2.60 (2H, m), 2.45-2.35 (2H, m), 2.20 (1H, septet, J 7.5), 1.95-1.45 (10H, m), 1.25-1.15 (2H, m); MS (ESI) 355.3 (M+H)⁺.

### Example 154: Synthesis of 1-(2-(3,8-diazabicyclo[3.2.1]octan-3-yl)-7,8-dihydro-1,6-naphthyridin-6(5H)-yl)-2-cyclopentylethan-1-one

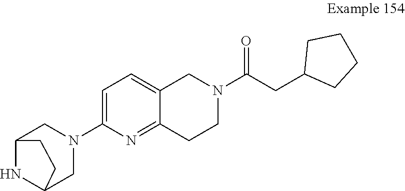 [SMILES: O=C(CC1CCCC1)N1CCc2nc(N3CC4CCC(C3)N4)ccc2C1]

Prepared by analogy to Example 149 using 2-chloro-5,6,7,8-tetrahydro-1,6-naphthyridine and 2-cyclopentylacetic acid in the first step and tert-butyl 3,8-diazabicyclo[3.2.1]octane-8-carboxylate in the second step. ¹H NMR: δ_{H} (400 MHZ, DMSO-d₆, mixture of rotamers) 7.30 (1H, d, J 8.6), 6.52 & 6.51 (1H, 2× overlapping d, J 8.6, rotamers), 4.51-4.45 (2H, m), 3.79-3.66 (4H, m), 3.50-3.42 (2H, m), 2.81-2.58 (4H, m), 2.42-2.38 (2H, m), 2.22-2.09 (1H, m), 1.80-1.68 (2H, m), 1.68-1.39 (8H, m), 1.19-1.05 (2H, m); MS (ESI) 355.2 (M+H)⁺.

### Example 155: Synthesis of 1-(2-((1S,4S)-2,5-diazabicyclo[2.2.1]heptan-2-yl)-7,8-dihydro-1,6-naphthyridin-6(5H)-yl)-2-cyclopentylethan-1-one

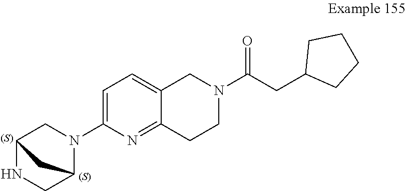

Prepared by analogy to Example 149 using 2-chloro-5,6,7,8-tetrahydro-1,6-naphthyridine and 2-cyclopentylacetic acid in the first step and tert-butyl (1S,4S)-2,5-diazabicyclo[2.2.1]heptane-2-carboxylate in the second step. ¹H NMR: δ_{H} (400 MHZ, DMSO-d₆, mixture of rotamers) 7.27 (1H, d, J 8.3), 6.33 & 6.32 (1H, 2× overlapping d, J 8.3, rotamers), 4.61-4.55 (1H, m), 4.51-4.42 (2H, m), 3.80-3.56 (3H, m), 3.42-3.36 (1H, m), 3.17-3.09 (1H, m), 2.89-2.59 (4H, m), 2.44-2.37 (2H, m), 2.17 (1H, septet, J 7.6), 1.81-1.41 (8H, m), 1.19-1.06 (2H, m); MS (ESI) 341.2 (M+H)⁺.

### Example 156: Synthesis of 1-(2-((1R,4R)-2,5-diazabicyclo[2.2.1]heptan-2-yl)-7,8-dihydro-1,6-naphthyridin-6(5H)-yl)-2-cyclopentylethan-1-one

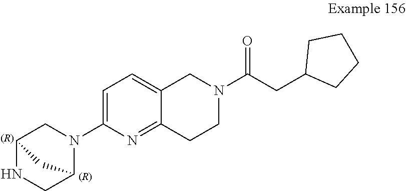

Prepared by analogy to Example 149 using 2-chloro-5,6,7,8-tetrahydro-1,6-naphthyridine and 2-cyclopentylacetic acid in the first step and tert-butyl (1R,4R)-2,5-diazabicyclo[2.2.1]heptane-2-carboxylate in the second step. ¹H NMR: δ_{H} (400 MHZ, DMSO-d₆, 80° C.) 7.26 (1H, d, J 8.5), 6.31 (1H, d, J 8.5), 4.62 (1H, app. br s), 4.47 (2H, s), 3.77-3.66 (2H, m), 3.64 (1H, app. br s), 3.41 (1H, dd, J 9.2, 2.2), 3.15 (1H, app. br d, J 9.2), 2.91 (1H, dd, J 9.3, 1.6, dd), 2.79 (1H, app. br d, J 9.4), 2.76-2.64 (2H, m), 2.41 (2H, d, J 7.1), 2.11 (1H, septet, J 7.5), 1.83-1.68 (3H, m), 1.68-1.44-(5H, m), 1.24-1.11 (2H, m); MS (ESI) 341.2 (M+H)⁺.

### Example 157: Synthesis of 2-cyclopentyl-1-(2-(7-hydroxy-3,9-diazabicyclo[3.3.1]nonan-9-yl)-7,8-dihydro-1,6-naphthyridin-6(5H)-yl)ethan-1-one

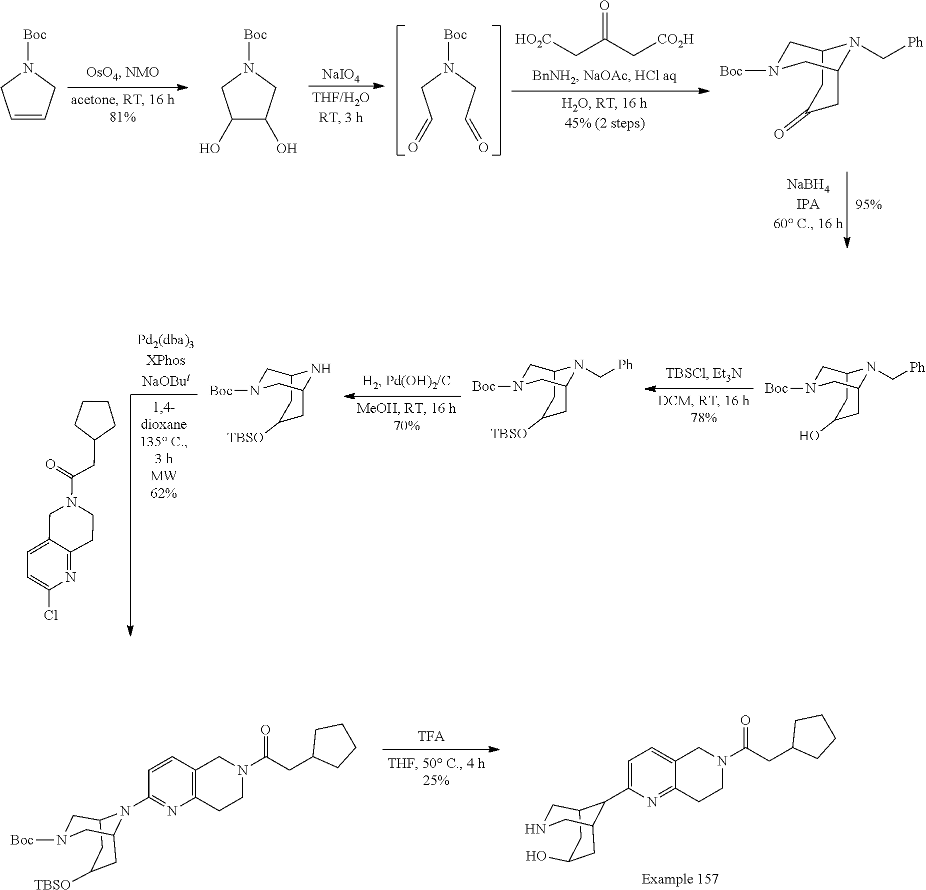

To a stirred solution of NMO (6.92 g, 59.1 mmol) in acetone (50 mL) and water (50 mL) was added osmium tetroxide (4 wt % in water, 10.8 mL, 1.70 mmol) at 0° C. and stirred for 30 min. To this tert-butyl 2,5-dihydro-1H-pyrrole-1-carboxylate (10.0 g, 59.1 mmol) in acetone (10 mL) was added and the resulting mixture was stirred at room temperature for 16 h. After completion, the reaction mixture was quenched with sodium bisulphite and concentrated to remove acetone. The mixture was acidified with dilute sulphuric acid (25 mL), diluted with EtOAc (25 mL), and filtered through a pad of Celite®. The filtrate was extracted with EtOAc (100 mL×3). The combined organic extracts were washed with brine (100 mL), dried (Na₂SO₄), filtered and concentrated under reduced pressure to afford crude tert-butyl 3,4-dihydroxypyrrolidine-1-carboxylate (9.8 g, 81%) as a purple liquid.

To a stirred solution of sodium periodate (1.05 g, 4.92 mmol) in water (10 mL) and THF (25 mL) was added tert-butyl 3,4-dihydroxypyrrolidine-1-carboxylate (1.00 g, 4.92 mmol) in THF (10 mL) at 0° C. The resulting mixture was stirred at room temperature for 3 h. After completion of the reaction, the mixture was filtered through a pad of Celite®, washing with THF (10 mL). The filtrate was concentrated to remove THF and the aqueous solution phase containing the crude tert-butyl bis(2-oxoethyl) carbamate was taken directly to the next step.

To a stirred solution of tert-butyl bis(2-oxoethyl) carbamate (ca. 86 mmol) in water (100 mL) at 0° C. was added sodium acetate (7.14 g, 87.0 mmol) and 3-oxopentanedioic acid (12.6 g, 86.1 mmol). The resulting mixture was stirred at 0° C. for 30 min. To this, a solution of benzylamine (9.23 g, 86.1 mmol) in 3M HCl (57.4 mL, 172 mmol) was added dropwise and stirred at room temperature for 16 hours. After completion, the reaction mixture was basified with K₂CO₃ solution (100 mL), diluted with 10% MeOH in DCM (25 mL) and filtered through a pad of Celite®. The filtrate was extracted with 10% MeOH in DCM (400 mL×3). The combined organic extracts were washed with brine (200 mL), dried (Na₂SO₄), filtered and concentrated under reduced pressure to give crude material that was processed by a flash column chromatography (0-60% EtOAc/petroleum ether eluent), affording tert-butyl 9-benzyl-7-oxo-3,9-diazabicyclo[3.3.1]nonane-3-carboxylate (13 g, 45%) as an off-white solid.

To a stirred solution of tert-butyl 9-benzyl-7-oxo-3,9-diazabicyclo[3.3.1]nonane-3-carboxylate (500 mg, 1.51 mmol) in 2-propanol (10 mL) at 0° C. was added NaBH₄ (343 mg, 9.08 mmol) and the resulting mixture was heated at 60° C. for 16 h. Afterwards, the reaction mixture was concentrated, diluted with ice-cold water (10 mL) and extracted with EtOAc (15 mL×3). The combined organic extracts were washed with brine (15 mL), dried (Na₂SO₄), filtered and concentrated under reduced pressure to afford tert-butyl 9-benzyl-7-hydroxy-3,9-diazabicyclo[3.3.1]nonane-3-carboxylate (490 mg, 95%) as a yellow oil.

To a stirred solution of tert-butyl 9-benzyl-7-hydroxy-3,9-diazabicyclo[3.3.1]nonane-3-carboxylate (1.60 g, 4.81 mmol) in DCM (10 mL) at 0° C. were added Et₃N (2.01 mL, 14.4 mmol), TBDMSCl (2.90 g, 19.2 mmol) and DMAP (59 mg, 0.48 mmol) and the resulting mixture was stirred at room temperature for 16 h. Afterwards, the mixture was quenched with 10% aqueous NaHCO₃ solution (20 mL), diluted with water (10 mL) and extracted with DCM (25 mL×3). The combined organic extracts were washed with brine (25 mL), dried (Na₂SO₄), filtered, and concentrated under reduced pressure to give crude material that was processed by flash column chromatography (0-30% EtOAc/petroleum ether eluent), affording tert-butyl 9-benzyl-7-((tert-butyldimethylsilyl)oxy)-3,9-diazabicyclo[3.3.1]nonane-3-carboxylate (1.68 g, 78%) as a yellow oil.

To a stirred solution of tert-butyl 9-benzyl-7-((tert-butyldimethylsilyl)oxy)-3,9-diazabicyclo[3.3.1]nonane-3-carboxylate (1.60 g, 3.58 mmol) in MeOH (15 mL) was added palladium hydroxide on carbon (20% dry basis) (0.45 g) under nitrogen atmosphere. The reaction mixture was stirred under a hydrogen gas bladder at room temperature for 16 h. After completion, the reaction mixture was filtered through a pad of Celite®, washing with MeOH (200 mL). The filtrate was concentrated under reduced pressure to give crude material that was processed by flash column chromatography (0-10% MeOH/DCM eluent), affording tert-butyl 7-((tert-butyldimethylsilyl)oxy)-3,9-diazabicyclo[3.3.1]nonane-3-carboxylate (900 mg, 70%) as an pale yellow semi solid.

In a 20 mL microwave vial a mixture of 1-(2-chloro-7,8-dihydro-1,6-naphthyridin-6(5H)-yl)-2-cyclopentylethan-1-one (300 mg, 1.08 mmol; prepared as schematised in the first step for Example 149 above but using 2-cyclopentylacetic acid in place of 3,3-dimethylbutanoic acid), tert-butyl 7-((tert-butyldimethylsilyl)oxy)-3,9-diazabicyclo[3.3.1]nonane-3-carboxylate (460 mg, 1.29 mmol), sodium tert-butoxide (310 mg, 3.23 mmol) in xylene (7 mL) and 1,4-dioxane (3.0 mL) were added XPhos (103 mg, 0.22 mmol) and Pd₂(dba)₃ (99 mg, 0.11 mmol) under continuous nitrogen bubbling. The vial was sealed and irradiated using a Biotage microwave initiator at 135° C. temperature for 3 h. After completion, the reaction mixture was concentrated and the residue processed by flash column chromatography (0-60% EtOAc/petroleum ether eluent), affording tert-butyl 7-((tert-butyldimethylsilyl)oxy)-9-(6-(2-cyclopentylacetyl)-5,6,7,8-tetrahydro-1,6-naphthyridin-2-yl)-3,9-diazabicyclo[3.3.1]nonane-3-carboxylate (400 mg, 62%) as a pale brown solid.

To a stirred solution of tert-butyl 7-((tert-butyldimethylsilyl)oxy)-9-(6-(2-cyclopentylacetyl)-5,6,7,8-tetrahydro-1,6-naphthyridin-2-yl)-3,9-diazabicyclo[3.3.1]nonane-3-carboxylate (400 mg, 0.67 mmol) in THF (8 mL) at 0° C. was added TFA (2.0 mL, 26 mmol) and the resulting mixture was heated at 50° C. for 4 h. Afterwards, the reaction mixture was concentrated and the residue was processed by reversed-phase preparative HPLC using ammonium bicarbonate as a buffer. Pure fractions were lyophilized to furnish 2-cyclopentyl-1-(2-(7-hydroxy-3,9-diazabicyclo[3.3.1]nonan-9-yl)-7,8-dihydro-1,6-naphthyridin-6(5H)-yl)ethan-1-one (65 mg, 25%, Example 157) as an off-white solid. ¹H-NMR: δ_{H} (400 MHZ, DMSO-d₆, 80° C.) 8.40 (1H, br s), 7.31 (1H, d, J 8.6), 6.61 (1H, d, J 8.6), 4.54-4.44 (4H, m), 3.73 (2H, t, J 6.1), 3.64 (1H, app. br t, J 4.8), 2.88-2.80 (4H, m), 2.76-2.65 (2H, m), 2.41 (2H, d, J 7.2), 2.21 (1H, septet, J 7.4), 2.03 (2H, dt, J 14.5 and 5.4), 1.82-1.72 (2H, m), 1.66-1.45 (6H, m), 1.23-1.11 (2H, m); MS (ESI) 385.3 (M+H)⁺.

### Examples 158 and 159: Synthesis of 2-(3,8-diazabicyclo[3.2.1]octan-8-yl)-6-((4-fluorobenzyl) sulfonyl)-5,6,7,8-tetrahydro-1,6-naphthyridine and 2-(3-ethyl-3,8-diazabicyclo[3.2.1]octan-8-yl)-6-((4-fluorobenzyl) sulfonyl)-5,6,7,8-tetrahydro-1,6-naphthyridine

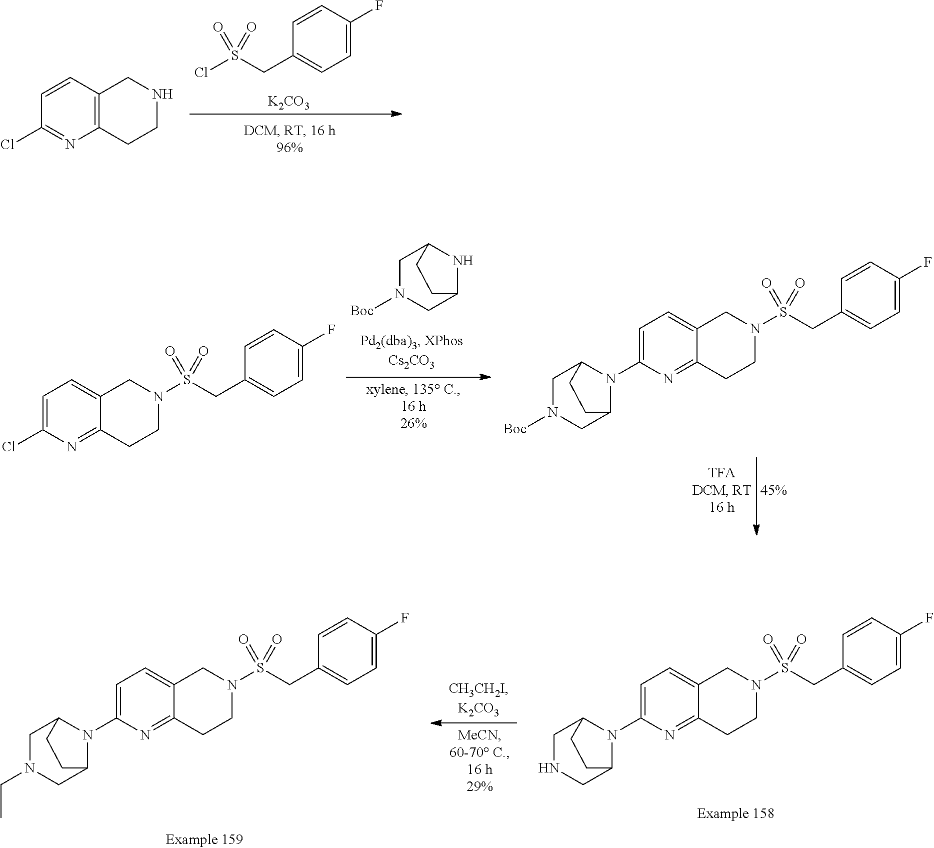

To a stirred solution of 2-chloro-5,6,7,8-tetrahydro-1,6-naphthyridine (2.00 g, 11.9 mmol) and (4-fluorophenyl) methane sulfonyl chloride (4.95 g, 23.7 mmol) in DCM (15 mL) was added K₂CO₃ (4.92 g, 35.6 mmol) and the resulting mixture was stirred at RT for 16 h. After completion, the reaction mixture was concentrated, diluted with water (10 mL) and extracted with 10% MeOH in DCM (10 mL×5). The combined organic extracts were washed with brine (10 mL), dried (Na₂SO₄), filtered and concentrated under reduced pressure. The crude residue was processed by flash column chromatography (0-100% EtOAc/petroleum ether eluent), affording 2-chloro-6-((4-fluorobenzyl) sulfonyl)-5,6,7,8-tetrahydro-1,6-naphthyridine (3.9 g, 96%) as an off-white solid.

To a stirred solution of 2-chloro-6-((4-fluorobenzyl) sulfonyl)-5,6,7,8-tetrahydro-1,6-naphthyridine (1.00 g, 2.93 mmol) and tert-butyl 3,8-diazabicyclo[3.2.1]octane-3-carboxylate (1.25 g, 5.87 mmol) in xylene (10 mL) were added XPhos (0.280 g, 0.587 mmol), Cs₂CO₃ (2.87 g, 8.81 mmol) and Pd₂(dba)₃ (0.269 g, 0.294 mmol) under continuous nitrogen bubbling. The resulting mixture was heated at 135° C. for 16 h. After completion, the reaction mixture was filtered through a pad of Celite®, washing with 10% MeOH in DCM. The filtrate was concentrated under reduced pressure and the residue processed by flash column chromatography (0-100% EtOAc/petroleum ether eluent), affording tert-butyl 8-(6-((4-fluorobenzyl) sulfonyl)-5,6,7,8-tetrahydro-1,6-naphthyridin-2-yl)-3,8-diazabicyclo[3.2.1]octane-3-carboxylate (389 mg, 26%) as an off-white solid.

To a stirred solution of tert-butyl 8-(6-((4-fluorobenzyl) sulfonyl)-5,6,7,8-tetrahydro-1,6-naphthyridin-2-yl)-3,8-diazabicyclo[3.2.1]octane-3-carboxylate (389 mg, 0.753 mmol) in DCM (5 mL) at 0° C. was added TFA (0.288 mL, 3.30 mmol) and the resulting mixture was stirred at room temperature for 16 h. Afterwards, the reaction mixture was concentrated under reduced pressure, basified with saturated aqueous NaHCO₃ solution and extracted with 10% MeOH in DCM (10 mL×5). The combined organic extracts were washed with brine (3 mL), dried (Na₂SO₄), filtered and concentrated under reduced pressure to afford crude material that was processed by reversed-phase preparative HPLC using ammonium bicarbonate as a buffer. Pure fractions were lyophilized to obtain 2-(3,8-diazabicyclo[3.2.1]octan-8-yl)-6-((4-fluorobenzyl) sulfonyl)-5,6,7,8-tetrahydro-1,6-naphthyridine (140 mg, 45%, Example 158) as an off-white solid. ¹H NMR: δ_{H} (400 MHZ, DMSO-d₆, 80° C.) 7.47-7.42 (2H, m), 7.22-7.12 (3H, m), 6.56-6.52 (1H, m), 4.46 (2H, s), 4.40-4.36 (2H, m), 4.22 (2H, s), 3.47 (2H, t, J 6.1), 2.91 (2H, d, J 10.8), 2.70 (2H, t, J 5.9), 2.54-2.52 (2H, m), 1.95-1.78 (4H, m); MS (ESI) 417.1 (M+H)⁺.

To a stirred solution of 2-(3,8-diazabicyclo[3.2.1]octan-8-yl)-6-((4-fluorobenzyl) sulfonyl)-5,6,7,8-tetrahydro-1,6-naphthyridine (40 mg, 0.096 mmol) in acetonitrile (5 mL) were added iodoethane (12 mg, 0.077 mmol) and K₂CO₃ (33.2 mg, 0.213 mmol) and the resulting mixture was heated at 60° C. for 16 h. LCMS indicated the presence of product along with reactants. Subsequently, another lot of iodoethane (12 mg, 0.077 mmol) and K₂CO₃ (33.2 mg, 0.213 mmol) were added and the mixture was heated at 70° C. for 16 h. After completion, the reaction mixture was diluted with 10% MeOH in DCM and filtered. The filtrate was concentrated under reduced pressure to obtain crude material that was processed by reversed-phase preparative HPLC using ammonium bicarbonate as a buffer. Pure fractions were lyophilized to obtain 2-(3-ethyl-3,8-diazabicyclo[3.2.1]octan-8-yl)-6-((4-fluorobenzyl) sulfonyl)-5,6,7,8-tetrahydro-1,6-naphthyridine (12.4 mg, 29%, Example 159) as an off-white solid. ¹H-NMR: δ_{H} (400 MHZ, DMSO-d₆) 7.47-7.41 (2H, m), 7.26-7.15 (3H, m), 6.63-6.58 (1H, m), 4.60 (2H, s), 4.50-4.42 (2H, m), 4.21 (2H, s), 3.42 (2H, t, J 6.1), 2.69 (2H, t, J 5.9), 2.61 (2H, dd, J 10.5 and 2.0), 2.26 (2H, q, J 7.2), 2.18 (2H, d, J 10.5), 1.90-1.70 (4H, m), 0.95 (3H, t, J 7.2); MS (ESI) 445.1 (M+H)⁺.

The following examples (160-165) were prepared using procedures analogous to Examples 158 and 159, using the appropriate starting materials.

### Examples 160 and 161: Synthesis of 6-((4-fluorobenzyl) sulfonyl)-2-(piperazin-1-yl)-5,6,7,8-tetrahydro-1,6-naphthyridine and 2-(4-ethylpiperazin-1-yl)-6-((4-fluorobenzyl) sulfonyl)-5,6,7,8-tetrahydro-1,6-naphthyridine

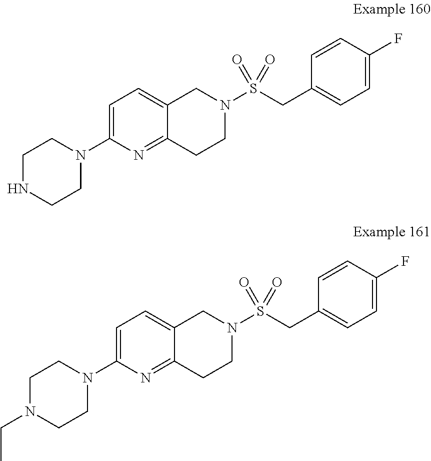 [SMILES: O=S(=O)(Cc1ccc(F)cc1)N1CCc2nc(N3CCNCC3)ccc2C1 ; CCN1CCN(c2ccc3c(n2)CCN(S(=O)(=O)Cc2ccc(F)cc2)C3)CC1]

Prepared by analogy to Examples 158/159 using tert-butyl piperazine-1-carboxylate in the second step. Example 160: ¹H-NMR: δ_{H} (400 MHZ, DMSO-d₆, 80° C.) 7.46-7.41 (2H, m), 7.27-7.23 (1H, m), 7.18-7.12 (2H, m), 6.63-6.59 (1H, m), 4.45 (2H, s), 4.23 (2H, s), 3.46 (2H, t, J 6.1), 3.41-3.37 (4H, m), 2.82-2.77 (4H, m), 2.68 (2H, t, J 5.9); MS (ESI) 391.1 (M+H)⁺. Example 161: ¹H-NMR: δ_{H} (400 MHZ, DMSO-d₆) 7.48-7.39 (2H, m), 7.31-7.25 (1H, m), 7.22-7.15 (2H, m), 6.71-6.65 (1H, m), 4.50 (2H, s), 4.24 (2H, s), 3.47-3.40 (6H, m) 2.71 (2H, t, J 5.9), 2.45-2.39 (4H, m), 2.35 (2H, q, J 7.2), 1.03 (3H, t, J 7.2); MS (ESI) 419.2 (M+H)⁺.

### Examples 162 and 163: Synthesis of 6-(benzylsulfonyl)-2-(3,8-diazabicyclo[3.2.1]octan-8-yl)-5,6,7,8-tetrahydro-1,6-naphthyridine and 6-(benzylsulfonyl)-2-(3-ethyl-3,8-diazabicyclo[3.2.1]octan-8-yl)-5,6,7,8-tetrahydro-1,6-naphthyridine

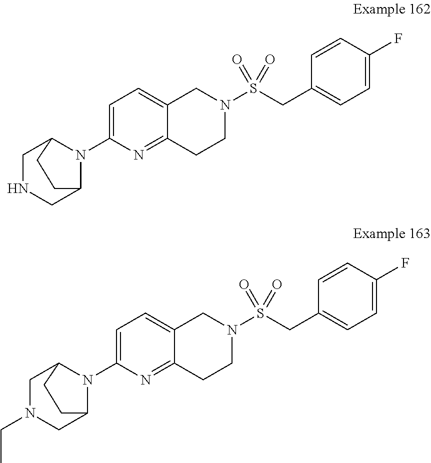 [SMILES: O=S(=O)(Cc1ccc(F)cc1)N1CCc2nc(N3C4CCC3CNC4)ccc2C1 ; CCN1CC2CCC(C1)N2c1ccc2c(n1)CCN(S(=O)(=O)Cc1ccc(F)cc1)C2]

Prepared by analogy to Examples 158/159 using phenylmethanesulfonyl chloride in the first step. Example 162: ¹H NMR: δ_{H} (400 MHZ, DMSO-d₆, 80° C.) 7.42-7.35 (5H, m), 7.22-7.19 (1H, m), 6.56-6.52 (1H, m), 4.44 (2H, s), 4.39-4.36 (2H, m), 4.21 (2H, s), 3.45 (2H, t, J 6.0), 2.90 (2H, d, J 11.2), 2.71-2.68 (2H, m), 2.55-2.50 (2H, m), 1.95-1.75 (4H, m); MS (ESI) 399.2 (M+H)⁺. Example 163: ¹H-NMR: δ_{H} (400 MHZ, DMSO-d₆) 7.42-7.34 (5H, m), 7.27-7.20 (1H, m), 6.65-6.56 (1H, m), 4.52-4.44 (4H, m), 4.21 (2H, s), 3.41 (2H, t, J 6.0), 2.69-2.66 (2H, m), 2.63-2.57 (2H, m), 2.23 (2H, q, J 7.2), 2.18 (2H, d, J 10.5), 1.90-1.70 (4H, m), 0.95 (3H, t, J 7.2); MS (ESI) 427.2 (M+H)⁺.

### Examples 164 and 165: Synthesis of 6-(benzylsulfonyl)-2-(piperazin-1-yl)-5,6,7,8-tetrahydro-1,6-naphthyridine and 6-(benzylsulfonyl)-2-(4-ethylpiperazin-1-yl)-5,6,7,8-tetrahydro-1,6-naphthyridine

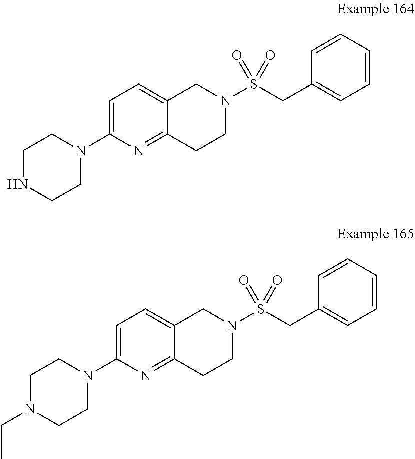 [SMILES: O=S(=O)(Cc1ccccc1)N1CCc2nc(N3CCNCC3)ccc2C1 ; CCN1CCN(c2ccc3c(n2)CCN(S(=O)(=O)Cc2ccccc2)C3)CC1]

Prepared by analogy to Examples 158/159 using phenylmethanesulfonyl chloride in the first step and tert-butyl piperazine-1-carboxylate in the second step. Example 164: ¹H-NMR: δ_{H} (400 MHZ, DMSO-d₆) 7.42-7.34 (5H, m), 7.31-7.24 (1H, m), 6.67-6.62 (1H, m), 4.49 (2H, s), 4.23 (2H, s), 3.42 (2H, t, J 6.0), 3.40-3.35 (4H, m), 2.77-2.72 (4H, m), 2.69-2.67 (2H, m); MS (ESI) 373.2 (M+H)⁺. Example 165: ¹H-NMR: δ_{H} (400 MHZ, DMSO-d₆): 7.42-7.35 (5H, m), 7.30-7.28 (1H, m), 6.70-6.67 (1H, m), 4.48 (2H, s), 4.24 (2H, s), 3.45-3.41 (6H, m), 2.70 (2H, t, J 6.0), 2.48-2.38 (4H, m), 2.34 (2H, q, J 7.2), 1.03 (3H, t, J 7.2); MS (ESI) 401.2 (M+H)⁺.

### Examples 166 and 167: Synthesis of 2-(3,8-diazabicyclo[3.2.1]octan-8-yl)-N—((S)-chroman-4-yl)-7,8-dihydro-1,6-naphthyridine-6(5H)-carboxamide and N—((S)-chroman-4-yl)-2-(3-ethyl-3,8-diazabicyclo[3.2.1]octan-8-yl)-7,8-dihydro-1,6-naphthyridine-6(5H)-carboxamide

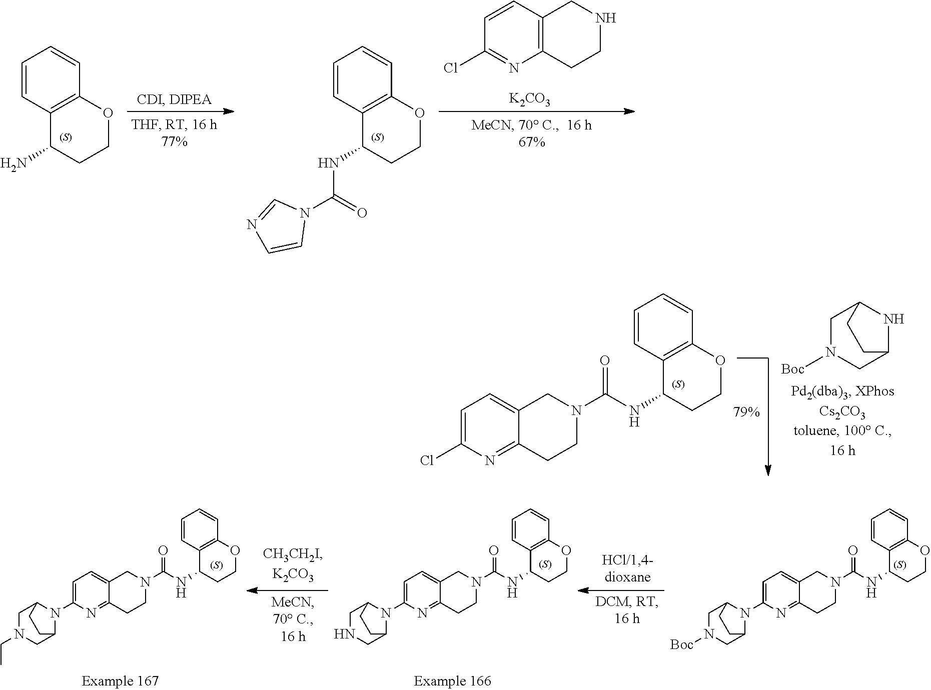

To a stirred solution of (S)-chroman-4-amine (2.00 g, 13.4 mmol) in THF (20 mL) at room temperature were added CDI (4.35 g, 26.8 mmol) and DIPEA (11.6 mL, 67.0 mmol). The reaction mixture was stirred at room temperature for 16 h. Afterwards, the reaction mixture was diluted with water (100 mL) and extracted with EtOAc (50 mL×2). The combined organic extracts were washed with brine (100 mL), dried (Na₂SO₄), filtered, and concentrated under reduced pressure to afford(S)—N-(chroman-4-yl)-1H-imidazole-1-carboxamide (2.5 g, 77%) as an off-white solid.

To a stirred solution of (S)—N-(chroman-4-yl)-1H-imidazole-1-carboxamide (1.00 g, 4.11 mmol) and 2-chloro-5,6,7,8-tetrahydro-1,6-naphthyridine (1.40 g, 8.32 mmol) in acetonitrile (10 mL) at room temperature was added K₂CO₃ (142 mg, 1.03 mmol) and the resulting mixture was heated at 70° C. for 16 h. After completion, the reaction mixture was diluted with water (100 mL) and extracted with EtOAc (50 mL×3). The combined organic extracts were washed with brine (100 mL), dried (Na₂SO₄), filtered, and concentrated under reduced pressure to a residue that was processed by flash column chromatography (0-100% EtOAc/petroleum ether eluent), affording(S)-2-chloro-N-(chroman-4-yl)-7,8-dihydro-1,6-naphthyridine-6(5H)-carboxamide (950 mg, 67%) as an off-white solid.

To a stirred solution of (S)-2-chloro-N-(chroman-4-yl)-7,8-dihydro-1,6-naphthyridine-6(5H)-carboxamide (250 mg, 0.727 mmol) and tert-butyl 3,8-diazabicyclo[3.2.1]octane-3-carboxylate (154 mg, 0.725 mmol) in toluene (5 mL) were added XPhos (34.7 mg, 0.0728 mmol), Cs₂CO₃ (711 mg, 2.18 mmol) and Pd₂(dba)₃ (133 mg, 0.145 mmol) under continuous nitrogen bubbling. The resulting mixture was heated at 100° C. for 16 h. After completion, the reaction mixture was diluted with water (20 mL) and extracted with EtOAc (20 mL×3). The combined organic extracts were washed with brine (50 mL), dried (Na₂SO₄), filtered, and concentrated under reduced pressure to a residue that was processed by flash column chromatography (0-100% EtOAc/petroleum ether eluent), affording tert-butyl 8-(6-(((S)-chroman-4-yl) carbamoyl)-5,6,7,8-tetrahydro-1,6-naphthyridin-2-yl)-3,8-diazabicyclo[3.2.1]octane-3-carboxylate (300 mg, 79%) as an off-white solid.

To a stirred solution of tert-butyl 8-(6-(((S)-chroman-4-yl) carbamoyl)-5,6,7,8-tetrahydro-1,6-naphthyridin-2-yl)-3,8-diazabicyclo[3.2.1]octane-3-carboxylate (450 mg, 0.866 mmol) in DCM (1 mL) at 0° C. was added 4M HCl in 1,4-dioxane (0.65 mL, 2.60 mmol) and the resulting mixture was stirred at room temperature for 16 h. After completion, the reaction mixture was evaporated and the residue processed by reversed-phase preparative HPLC using ammonium bicarbonate as a buffer. Pure fractions were lyophilized to obtain 2-(3,8-diazabicyclo[3.2.1]octan-8-yl)-N—((S)-chroman-4-yl)-7,8-dihydro-1,6-naphthyridine-6(5H)-carboxamide (14 mg, 4%, Example 166) as an off-white solid. ¹H-NMR: δ_{H} (400 MHZ, DMSO-d₆) 7.23 (1H, d, J 8.6), 7.18 (1H, app. d, J 7.8), 7.13 (1H, app. td, J 7.5 and 1.1), 6.95 (1H, d, J 8.2), 6.86 (1H, td, J 7.5 and 1.1), 6.76 (1H, dd, J 8.0 and 1.1), 6.56 (1H, d, J 8.6), 4.98-4.90 (1H, m), 4.45-4.31 (4H, m), 4.29-4.22 (1H, m), 4.21-4.13 (1H, m), 3.71-3.58 (2H, m), 2.88-2.83 (2H, m), 2.67-2.63 (2H, m), 2.50-2.40 (2H, m), 2.05-1.55 (6H, m); MS (ESI) 420.2 (M+H)⁺.

To a stirred solution of 2-(3,8-diazabicyclo[3.2.1]octan-8-yl)-N—((S)-chroman-4-yl)-7,8-dihydro-1,6-naphthyridine-6(5H)-carboxamide (300 mg, 0.762 mmol) and ethyl iodide (123 mg, 0.787 mmol) in MeCN (5 mL) at room temperature was added K₂CO₃ (494 mg, 3.58 mmol) and the resulting mixture was heated at 70° C. for 16 h. After completion, the reaction mixture was filtered through a pad of Celite®, washing with EtOAc. The filtrate was concentrated under reduced pressure to a residue that was processed by reversed-phase preparative HPLC using ammonium bicarbonate as a buffer. Pure fractions were lyophilized to obtain N—((S)-chroman-4-yl)-2-(3-ethyl-3,8-diazabicyclo[3.2.1]octan-8-yl)-7,8-dihydro-1,6-naphthyridine-6(5H)-carboxamide (6 mg, 2%, Example 167) as an off-white solid. ¹H-NMR: δ_{H} (400 MHZ, DMSO-d₆) 7.24 (1H, d, J 8.6), 7.18 (1H, app. d, J 7.5), 7.12 (1H, app. td, J 7.5 and 1.4), 6.93 (1H, d, J 8.3), 6.85 (1H, td, J 7.5 and 1.1), 6.76 (1H, J 8.1 and 1.1), 6.59 (1H, d, J 8.6), 4.99-4.89 (1H, m), 4.51-4.32 (4H, m), 4.29-4.16 (2H, m), 3.71-3.57 (2H, m), 2.66 (2H, t, J 5.7), 2.63-2.55 (2H, m), 2.24 (2H, q, J 7.2), 2.21-2.16 (2H, m), 2.05-1.70 (6H, m), 0.95 (3H, t, J 7.2); MS (ESI) 448.3 (M+H)⁺.

The following examples (168-170) were prepared using procedures analogous to Examples 166 and 167, using the appropriate starting materials.

### Examples 168 and 169: Synthesis of (S)—N-(chroman-4-yl)-2-(piperazin-1-yl)-7,8-dihydro-1,6-naphthyridine-6(5H)-carboxamide and(S)—N-(chroman-4-yl)-2-(4-ethylpiperazin-1-yl)-7,8-dihydro-1,6-naphthyridine-6(5H)-carboxamide

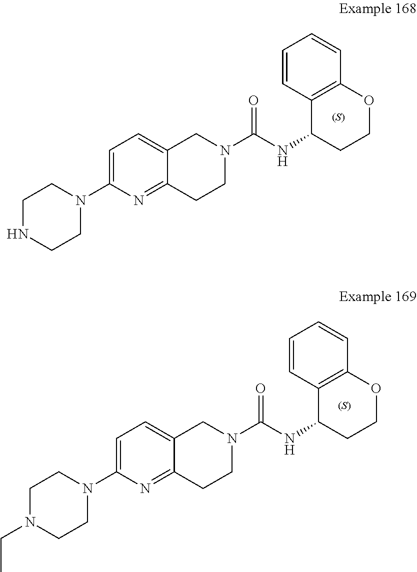

Prepared by analogy to Examples 166/167 using tert-butyl piperazine-1-carboxylate in the third step. Example 168: ¹H-NMR: δ_{H} (400 MHZ, DMSO-d₆) 7.27 (1H, d, J 8.6), 7.18 (1H, app. d, J 7.5), 7.12 (1H, app. td, J 7.5 and 1.4), 6.95 (1H, d, J 8.3), 6.85 (1H, td, J 7.5 and 1.1), 6.76 (1H, J 8.1 and 1.1), 6.64 (1H, d, J 8.6), 5.00-4.90 (1H, m), 4.48-4.34 (2H, m), 4.29-4.12 (2H, m), 3.71-3.59 (2H, m), 3.38-3.28 (4H, m), 2.80-2.73 (4H, m), 2.67 (2H, t, J 5.7), 2.05-1.90 (2H, m); MS (ESI) 394.2 (M+H)⁺. Example 169: ¹H-NMR: δ_{H} (400 MHZ, DMSO-d₆) 7.28 (1H, d, J 8.6), 7.18 (1H, app. d, J 7.5), 7.12 (1H, app. td, J 7.5 and 1.4), 6.94 (1H, d, J 8.3), 6.85 (1H, td, J 7.5 and 1.1), 6.76 (1H, J 8.1 and 1.1), 6.67 (1H, d, J 8.6), 4.98-4.91 (1H, m), 4.46-4.34 (2H, m), 4.29-4.14 (2H, m), 3.71-3.58 (2H, m), 3.48-3.38 (4H, m), 2.68 (2H, t, J 5.7), 2.46-2.39 (4H, m), 2.35 (2H, q, J 7.2), 2.08-1.87 (2H, m) 1.03 (3H, t, J 7.2); MS (ESI) 422.2 (M+H)⁺.

### Example 170: Synthesis of N—((R)-chroman-4-yl)-2-(3-ethyl-3,8-diazabicyclo[3.2.1]octan-8-yl)-7,8-dihydro-1,6-naphthyridine-6(5H)-carboxamide

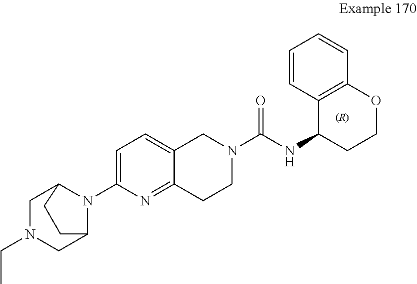

Prepared by analogy to Example 167 using (R)-chroman-4-amine in the first step. Example 170: ¹H-NMR: δ_{H} (400 MHZ, DMSO-d₆): 7.24 (1H, d, J 8.6), 7.18 (1H, app. d, J 7.5), 7.12 (1H, app. td, J 7.5 and 1.4), 6.93 (1H, d, J 8.3), 6.85 (1H, td, J 7.5 and 1.1), 6.76 (1H, J 8.1 and 1.1), 6.59 (1H, d, J 8.6), 4.99-4.89 (1H, m), 4.51-4.32 (4H, m), 4.29-4.16 (2H, m), 3.71-3.57 (2H, m), 2.66 (2H, t, J 5.7), 2.63-2.55 (2H, m), 2.24 (2H, q, J 7.2), 2.21-2.16 (2H, m), 2.05-1.70 (6H, m), 0.95 (3H, t, J 7.1); MS (ESI) 448.2 (M+H)⁺.

The following examples (171-174) were prepared by N-alkylation of Example 46 using a procedure analogous to that described for Example 115, but with the appropriate alkyl halide in place of 2-bromoethanol.

### Example 171: Synthesis of 1-(2-(3-ethyl-3,8-diazabicyclo[3.2.1]octan-8-yl)-7,8-dihydro-1,6-naphthyridin-6(5H)-yl)-2-(4-fluorophenoxy)ethan-1-one

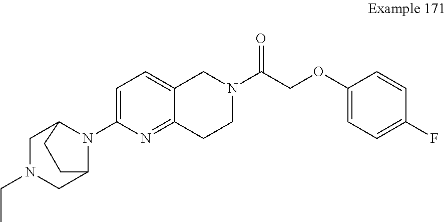 [SMILES: CCN1CC2CCC(C1)N2c1ccc2c(n1)CCN(C(=O)COc1ccc(F)cc1)C2]

Prepared by alkylation of Example 46 with iodoethane. ¹H-NMR: δ_{H} (300 MHZ, DMSO-d₆, mixture of rotamers) 7.36-7.25 (1H, m), 7.16-7.04 (2H, m), 7.00-6.91 (2H, m), 6.66-6.57 (1H, m), 4.93-4.88 (2H, m), 4.56-4.45 (4H, m) 3.73 (2H, t, J 5.9), 2.83-2.56 (4H, m), 2.29-2.15 (4H, m), 1.90-1.70 (4H, m), 0.95 (3H, t, J 7.2); MS (ESI) 425.2 (M+H)⁺.

### Example 172: Synthesis of 2-(4-fluorophenoxy)-1-(2-(3-propyl-3,8-diazabicyclo[3.2.1]octan-8-yl)-7,8-dihydro-1,6-naphthyridin-6(5H)-yl)ethan-1-one

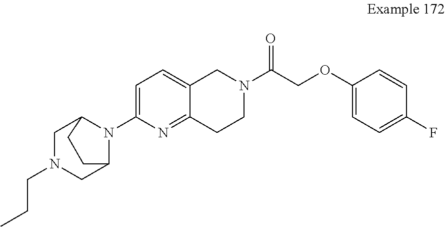 [SMILES: CCCN1CC2CCC(C1)N2c1ccc2c(n1)CCN(C(=O)COc1ccc(F)cc1)C2]

Prepared by alkylation of Example 46 with 1-iodopropane. ¹H-NMR: δ_{H} (400 MHZ, DMSO-d₆, mixture of rotamers) 7.35-7.24 (1H, m), 7.20-7.03 (2H, m), 7.01-6.88 (2H, m), 6.69-6.56 (1H, m), 4.94-4.86 (2H, m), 4.57-4.44 (4H, m), 3.73 (2H, t, J 5.9), 2.84-2.53 (4H, m), 2.23-2.11 (4H, m), 1.91-1.71 (4H, m), 1.39 (2H, sextet, J 7.2), 0.85 (3H, t, J 7.3); MS (ESI) 439.2 (M+H)⁺.

### Example 173: Synthesis of 2-(4-fluorophenoxy)-1-(2-(3-(2-methoxyethyl)-3,8-diazabicyclo[3.2.1]octan-8-yl)-7,8-dihydro-1,6-naphthyridin-6(5H)-yl)ethan-1-one

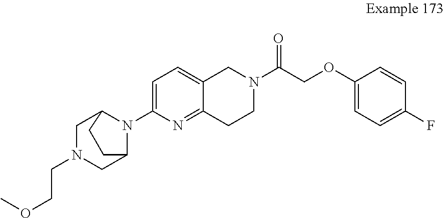 [SMILES: COCCN1CC2CCC(C1)N2c1ccc2c(n1)CCN(C(=O)COc1ccc(F)cc1)C2]

Prepared by alkylation of Example 46 with 1-bromo-2-methoxyethane. ¹H-NMR: δ_{H} (400 MHZ, DMSO-d₆, 80° C.) 7.30 (1H, d, J 8.4), 7.11-7.06 (2H, m), 6.99-6.96 (2H, m), 6.59 (1H, d, J 8.4), 4.86 (2H, s), 4.55-4.45 (4H, m), 3.76 (2H, t, J 6.0), 3.42 (2H, t, J 5.8), 3.25 (3H, s), 2.85-2.70 (2H, m), 2.65-2.62 (2H, m), 2.43 (2H, t, J 6.0), 2.40-2.30 (2H, m), 1.95-1.73 (4H, m); MS (ESI) 455.2 (M+H)⁺.

### Example 174: Synthesis of 2-(4-fluorophenoxy)-1-(2-(3-(3-methoxypropyl)-3,8-diazabicyclo[3.2.1]octan-8-yl)-7,8-dihydro-1,6-naphthyridin-6(5H)-yl)ethan-1-one

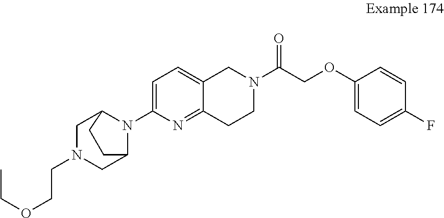 [SMILES: CCOCCN1CC2CCC(C1)N2c1ccc2c(n1)CCN(C(=O)COc1ccc(F)cc1)C2]

Prepared by alkylation of Example 46 with 1-bromo-3-methoxypropane. ¹H-NMR: δ_{H} (400 MHz, DMSO-d₆, mixture of rotamers) 7.34-7.28 (1H, m), 7.14-7.08 (2H, m), 7.00-6.90 (2H, m), 6.65-6.60 (1H, m), 4.95-4.85 (2H, m), 4.55-4.42 (4H, m), 3.73 (2H, t, J 5.9), 3.37-4.30 (2H, m), 3.32 (3H, s), 2.84-2.53 (4H, m), 2.25-2.15 (4H, m), 1.90-1.70 (4H, m), 1.60 (2H, sextet, J 7.2); MS (ESI) 469.2 (M+H)⁺.

The following examples (175-180) were prepared using procedures analogous to Example 149, using the appropriate starting materials.

### Example 175: Synthesis of 2-phenyl-1-(2-(piperazin-1-yl)-7,8-dihydro-1,6-naphthyridin-6(5H)-yl)ethan-1-one

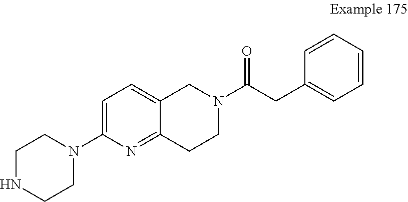 [SMILES: O=C(Cc1ccccc1)N1CCc2nc(N3CCNCC3)ccc2C1]

Prepared by analogy to Example 149 using 2-chloro-5,6,7,8-tetrahydro-1,6-naphthyridine and 2-phenylacetic acid in the first step and tert-butyl piperazine-1-carboxylate in the second step; the Pd₂(dba)₃/XPhos/Cs₂CO₃/toluene conditions were used for the amine coupling (as described for synthesis of Example 166). ¹H NMR: δ_{H} (400 MHZ, DMSO-d₆, mixture of rotamers) 7.35-7.18 (6H, m), 6.67-6.62 (1H, m), 4.58-4.49 (2H, m), 3.83-3.73 (4H, m), 3.38-3.27 (4H, m), 2.77-2.72 (4H, m), 2.69-2.56 (2H, m); MS (ESI) 337.2 (M+H)⁺.

### Example 176:2-(4-fluorophenyl)-1-(2-(piperazin-1-yl)-7,8-dihydro-1,6-naphthyridin-6(5H)-yl)ethan-1-one

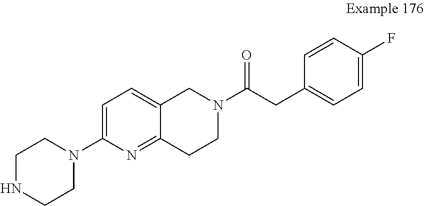 [SMILES: O=C(Cc1ccc(F)cc1)N1CCc2nc(N3CCNCC3)ccc2C1]

Prepared by analogy to Example 149 using 2-chloro-5,6,7,8-tetrahydro-1,6-naphthyridine and 2-(4-fluorophenyl) acetic acid in the first step and tert-butyl piperazine-1-carboxylate in the second step; the Pd₂(dba)₃/XPhos/Cs₂CO₃/toluene conditions were used for the amine coupling (as described for synthesis of Example 166). ¹H NMR: δ_{H} (400 MHZ, DMSO-d₆, 80° C.) 7.35-7.25 (3H, m), 7.13-7.04 (2H, m), 6.61 (1H, d, J 8.6), 4.53 (2H, s), 3.79 (2H, s), 3.77 (2H, t, J 5.9), 3.40-3.35 (4H, m), 2.81-2.76 (4H, m), 2.68 (2H, t, J 5.9); MS (ESI) 355.2 (M+H)⁺.

### Example 177: Synthesis of 1-(3-(3,8-diazabicyclo[3.2.1]octan-8-yl)-5,8-dihydro-1,7-naphthyridin-7 (6H)-yl)-2-cyclopentylethan-1-one

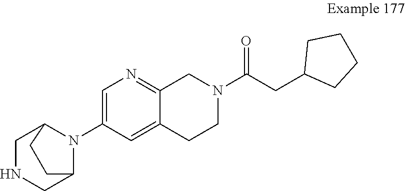 [SMILES: O=C(CC1CCCC1)N1CCc2cc(N3C4CCC3CNC4)cnc2C1]

Prepared by analogy to Example 149 using 3-bromo-5,6,7,8-tetrahydro-1,7-naphthyridine and 2-cyclopentylacetic acid in the first step and tert-butyl 3,8-diazabicyclo[3.2.1]octane-3-carboxylate in the second step; the Pd₂(dba)₃/XPhos/Cs₂CO₃/toluene conditions were used for the amine coupling (as described for synthesis of Example 166). ¹H-NMR: δ_{H} (400 MHZ, DMSO-d₆, 80° C.) 7.99 (1H, d, J 2.6), 6.95 (1H, d, J 2.6), 4.52 (2H, s), 4.16-4.09 (2H, m), 3.69 (2H, t, J 5.9), 3.01-2.95 (2H, m), 2.85-2.70 (2H, m), 2.48-2.44 (2H, m), 2.42 (2H, d, J 7.2), 2.21 (1H, septet, J 7.5), 1.97-1.91 (2H, m), 1.90-1.83 (2H, m), 1.81-1.70 (2H, m), 1.64-1.55 (2H, m), 1.55-1.44 (2H, m), 1.22-1.12 (2H, m); MS (ESI) 355.2 (M+H)⁺.

### Example 178: Synthesis of 1-(6-(3,8-diazabicyclo[3.2.1]octan-8-yl)-3,4-dihydro-2,7-naphthyridin-2 (1H)-yl)-2-cyclopentylethan-1-one

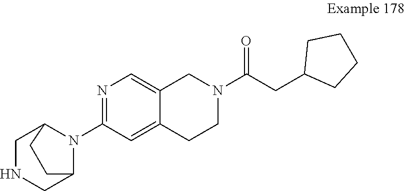 [SMILES: O=C(CC1CCCC1)N1CCc2cc(N3C4CCC3CNC4)ncc2C1]

Prepared by analogy to Example 149 using 6-chloro-1,2,3,4-tetrahydro-2,7-naphthyridine and 2-cyclopentylacetic acid in the first step and tert-butyl 3,8-diazabicyclo[3.2.1]octane-3-carboxylate in the second step; the Pd₂(dba)₃/XPhos/Cs₂CO₃/toluene conditions were used for the amine coupling (as described for synthesis of Example 166). ¹H NMR: δ_{H} (400 MHZ, DMSO-d₆, 80° C.) 7.93 (1H, s), 6.50 (1H, s), 4.51 (2H, s), 4.39-4.34 (2H, m), 3.64 (2H, t, J 5.9), 2.94-2.90 (2H, m), 2.80-2.65 (2H, m), 2.57-2.52 (2H, m), 2.41 (2H, d, J 7.2), 2.22 (1H, septet, J 7.5), 1.95-1.74 (6H, m), 1.64-1.47 (4H, m), 1.22-1.12 (2H, m); MS (ESI) 355.2 (M+H)⁺.

### Example 179: Synthesis of 1-(7-(3,8-diazabicyclo[3.2.1]octan-8-yl)-3,4-dihydro-2,6-naphthyridin-2 (1H)-yl)-2-cyclopentylethan-1-one

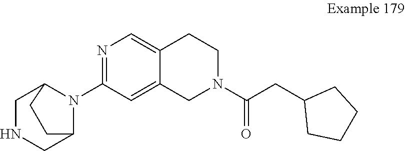 [SMILES: O=C(CC1CCCC1)N1CCc2cnc(N3C4CCC3CNC4)cc2C1]

Prepared by analogy to Example 149 using 7-chloro-1,2,3,4-tetrahydro-2,6-naphthyridine and 2-cyclopentylacetic acid in the first step and tert-butyl 3,8-diazabicyclo[3.2.1]octane-3-carboxylate in the second step; the Pd₂(dba)₃/XPhos/Cs₂CO₃/toluene conditions were used for the amine coupling (as described for synthesis of Example 166). ¹H NMR: δ_{H} (400 MHZ, DMSO-d₆, 80° C.) 7.90 (1H, s), 6.52 (1H, s), 4.55 (2H, s), 4.38-4.34 (2H, m), 3.67 (2H, t, J 5.9), 2.95-2.87 (2H, m), 2.75-2.60 (2H, m), 2.57-2.52 (2H, m), 2.41 (2H, d, J 7.2), 2.21 (1H, septet, J 7.5), 1.95-1.75 (6H, m), 1.65-1.45 (4H, m), 1.23-1.13 (2H, m); MS (ESI) 355.2 (M+H)⁺.

### Example 180: Synthesis of 2-(4-fluorophenoxy)-1-(2-(piperazin-1-yl)-7,8-dihydro-1,6-naphthyridin-6(5H)-yl)ethan-1-one

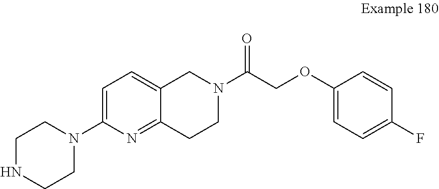 [SMILES: O=C(COc1ccc(F)cc1)N1CCc2nc(N3CCNCC3)ccc2C1]

Prepared by analogy to Example 149 using 2-chloro-5,6,7,8-tetrahydro-1,6-naphthyridine and 2-(4-fluorophenoxy) acetic acid in the first step and tert-butyl piperazine-1-carboxylate in the second step; the Pd₂(dba)₃/XPhos/Cs₂CO₃/toluene conditions were used for the amine coupling (as described for synthesis of Example 166). ¹H NMR: δ_{H} (400 MHZ, DMSO-d₆, mixture of rotamers) 7.38-7.28 (1H, m), 7.15-7.05 (2H, m), 7.00-6.90 (2H, m), 6.90-6.63 (1H, m), 4.92-4.87 (2H, m), 4.57-4.47 (2H, m), 3.73 (2H, t, J 5.9), 3.40-3.35 (4H, m), 2.84-2.63 (6H, m); MS (ESI) 371.1 (M+H)⁺.

### Example 181: Synthesis of 2-cyclopentyl-1-(2-(2-methylpyridin-3-yl)-7,8-dihydro-1,6-naphthyridin-6(5H)-yl)ethan-1-one

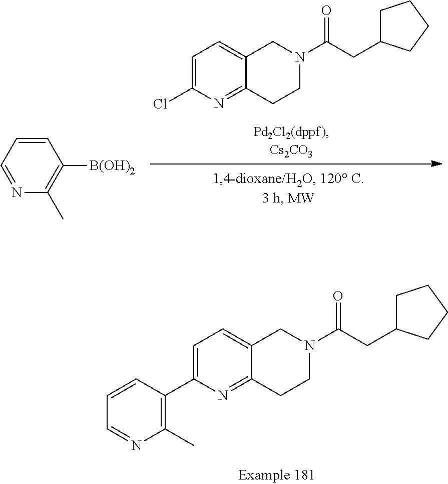

To a stirred suspension of 1-(2-chloro-7,8-dihydro-1,6-naphthyridin-6(5H)-yl)-2-cyclopentylethan-1-one (1 equiv.; prepared as described in the synthesis of Example 112), (2-methylpyridin-3-yl) boronic acid (1 equiv.) and Cs₂CO₃ (3 equiv.) in a mixture of 1,4-dioxane (4 vol.) and water (1 vol.) was added PdCl₂ (dppf).CH₂Cl₂ (0.1 equiv.) under continuous bubbling with nitrogen. The resulting mixture was then heated at 100° C. After 16 h the reaction mixture was cooled and filtered through a pad of Celite®, washing with EtOAc. The filtrate was concentrated to give a residue that was processed by reversed-phase preparative HPLC using ammonium bicarbonate as a buffer, affording 2-cyclopentyl-1-(2-(2-methylpyridin-3-yl)-7,8-dihydro-1,6-naphthyridin-6(5H)-yl)ethan-1-one as an off-white solid. ¹H NMR: δ_{H} (400 MHZ, DMSO-d₆, mixture of rotamers) 8.50 (1H, dd, J 4.8 and 1.8), 7.79-7.71 (2H, m), 7.48-7.43 (1H, m), 7.33 (1H, dd, J 7.7 and 4.8), 4.87-4.71 (2H, m), 3.84 (2H, t, J 6.0), 3.02-2.88 (2H, m), 2.53-2.44 (5H, m), 2.25-2.14 (1H, m), 1.83-1.72 (2H, m), 1.65-1.46 (4H, m), 1.21-1.10 (2H, m); MS (ESI) 336.2 (M+H)⁺.

### Example 182: Synthesis of 2-cyclopentyl-1-(2-(5-methylpyridin-3-yl)-7,8-dihydro-1,6-naphthyridin-6(5H)-yl)ethan-1-one

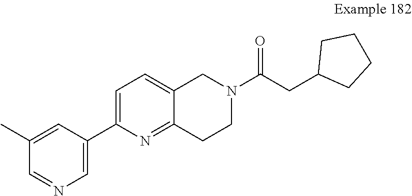 [SMILES: Cc1cncc(-c2ccc3c(n2)CCN(C(=O)CC2CCCC2)C3)c1]

Prepared as Example 181 but using 3-methyl-5-(4,4,5,5-tetramethyl-1,3,2-dioxaborolan-2-yl)pyridine in place of (2-methylpyridin-3-yl) boronic acid in the reaction with 1-(2-chloro-7,8-dihydro-1,6-naphthyridin-6(5H)-yl)-2-cyclopentylethan-1-one. ¹H NMR: δ_{H} (400 MHZ, DMSO-de, mixture of rotamers) 9.04 (1H, d, J 2.0), 8.47 (1H, d, J 1.6), 8.25-8.23 (1H, m), 7.93-7.86 (1H, m), 7.80-7.71 (1H, m), 4.76-4.69 (2H, m), 3.83 (2H, t, J 6.0), 3.05-2.91 (2H, m), 2.48-2.43 (2H, m), 2.39 (3H, s), 2.25-2.13 (1H, m), 1.83-1.72 (2H, m), 1.64-1.44 (4H, m), 1.22-1.10 (2H, m); MS (ESI) 336.2 (M+H)⁺.

### Biological Assays

### Cell Culture

HEK 293 and MDCK cells (Public Health England, Cell Culture Collections) were maintained in Dulbecco's Modified Eagle's Medium (DMEM) supplemented with 10% (v/v) fetal bovine serum (FBS) (Seralab), 2 mM L-glutamine and 100 U/mL penicillin streptomycin cocktail (ThermoFisher); termed complete media. Clonal HEK 293 cell lines were maintained in complete media supplemented with 0.6 mg/ml G418 (Enzo Life Sciences). Mouse inner medullary collecting duct (m-IMCD3) cells [American Type Culture Collection (ATCC)] were maintained in media consisting of DMEM/Hams F-12 50/50 Mix (DMEM F12; Corning) supplemented with penicillin streptomycin cocktail (100 U/mL) and FBS (10% v/v) (DMEM F12-10% FBS).

### Experiment 1: Measurement of Modulation of Enzyme Activity by PDE4 Long Form Activators of the Present Invention Using Full-Length Human PDE4 Isoforms; Long Forms PDE4D5, PDE4C3, PDE4B1 and Short Form PDE4B2

(Marchmont, R. J. and Houslay, M. D. Biochem. J. 187:381-92, 1980)

Exogenous expression of long form PDE4 enzymes and stable cell line generation For transient transfection of exogenous PDE4 long isoforms, HEK 293 cells were transfected with pcDNA3.1 or pDEST™ PDE4 expression vectors using Lipofectamine LTX/Plus reagent (Invitrogen) as outlined by the manufacturer.

Where stable cell lines were generated the clonal isolates were expanded to obtain cell lines that stably expressed the full-length human PDE4 long isoforms and the full length human PDE4B2 short isoform. These were called the HEK-PDE4D5, HEK-PDE4B1 and HEK-PDE4B2 cell lines, respectively.

### Lysate Preparation (Using PDE4D5 as a Typical Example)

HEK-PDE4D5 cells were seeded out in 100 mm plates and incubated at 37° C. in an atmosphere of 5% CO₂, 95% air. Cell lysates were prepared using KHEM buffer [50 mM KCl, 10 mM EGTA, 50 mM HEPES (pH 7.2), 1.92 mM MgCl₂].

To prepare the cell lysates, the 100 mm plates containing the cells were placed on ice and washed with ice-cold PBS (phosphate buffered saline, pH 7.4). KHEM buffer (500 μL) was added to the cells. Cells were then scraped off the plate and triturated using a needle (BD Microlance™ 0.8, 40 mm). The lysed cells were then centrifuged at 2000 rpm for 10 minutes to remove cell debris and the supernatant (cell lysate containing recombinant PDE4D5) was transferred to a fresh tube and kept on ice.

### Cytosol Fraction Preparation (Using PDE4D5 as a Typical Example)

The cell lysate containing recombinant PDE4D5 was transferred into a centrifuge tube and placed into an ultracentrifuge (BECKMAN COULTER) and spun at high speed (100,000g) for 30 minutes at 4° C. The cytosol fraction was then collected and its protein amount determined using a BCA protein assay.

### PDE Assay—(Using PDE4D5 as a Typical Example)

PDE assays were performed in thin-walled V-bottomed 96-well plates. The assays were performed at a final concentration of 10 mM Tris/5 mM MgCl₂ plus PDE4D5 cell lysate cytosol fraction, containing over-expressed PDE4D5, with and without test compound. The lysate/compound mix were incubated together for 15 min at room temperature on an orbital shaker prior to addition of [³H] cAMP (final concentration 1 μM [³H] cAMP; Perkin Elmer) to a final volume of 50 μL per reaction. The reactions were then incubated for 10 minutes at 30° C., terminated by heating for 2 min at 95 and allowed to cool. Snake venom (12.5 μL of 1 mg/mL; Crotalus atrox, Sigma) was then added and the plates were agitated and incubated for a further 15 minutes at 30° C. Dowex ion exchange resin (Sigma, chloride form, 200-400 mesh; 200 μL; prepared as a 1:1 Dowex: water stock, thoroughly re-suspended and diluted 2:1 with ethanol) was then added to each well and the plates incubated for 15 min at room temperature on an orbital shaker ensuring sufficient agitation for resin suspension (550 RPM). The reaction mixture was then transferred to a 96 well filter plate (Millipore; 0.45 μm pore size) and filtered into a receiving 96-well plate to remove the dowex suspension. 30 μL of the filtered solution was then transferred to the wells of an Opti-plate (Perkin Elmer) 96 well assay plate and 120 μL of Microscint 40 scintillation fluid was added. The plate was then placed on an orbital shaker for 10 min at high speed (900 RPM) to mix the sample with scintillation fluid prior to quantitation using a plate based scintillation counter (Top-Count).

The % increase in counts in the presence of test compound at a particular concentration indicates the % increase in enzyme activity at that concentration.

Data are shown in Table 2.

Experiment 2: Reduction of CAMP Levels in m-IMCD3 Cell Culture Treated with PDE4 Long Form Activators

Mouse Inner Medullary Collecting Duct (m-IMCD3) cells have been shown to express PDE4A, PDE4B, PDE4C and PDE4D long isoforms (Hansen et al., EMBO reports e54315|2022, see FIG. 4). Activation of intracellular PDE4 long forms leads to a reduction of both intracellular CAMP and externalised cAMP (Omar et al., PNAS 116:13320-13329, 2019). This reduction may be measured in the cell culture media supernatant of 3D cystic cell cultures, such as in the m-IMCD3 cell cyst suppression assay described in Experiment 3.

Following the completion of the m-IMCD3 cell cyst suppression assay (Experiment 3), the CAMP levels in the assay media from individual or pooled assay wells was measured using a CAMP ELISA kit (Enzo Life Sciences) or a LANCE Ultra CAMP kit (PerkinElmer) according to the manufacturer's instructions.

Treatment with PDE4 long form activators reduced CAMP levels in PGE2-stimulated m-IMCD3 cell culture. Data are shown in Table 3.

Experiment 3: Inhibition of In Vitro Cyst Formation in m-IMCD3 Cells Treated with PDE4 Long Form Activators

The mouse Inner Medullary Collecting Duct cell line (m-IMCD3) spontaneously forms cystic spheroids in 3D culture with a Type 1 collagen/Matrigel extracellular matrix. This process can be stimulated with agents which raise intracellular cAMP and is used as an in vitro model for the formation of cystic structures in the kidneys of patients with ADPKD.

Rat Collagen I (Fisher Scientific) is prepared on ice by neutralising with 1M NaOH and diluting with a 2× volume of DMEM/F12+10% FBS. This is mixed 1:1.1 with ice cold Matrigel (Corning) for coating plates (coating mix), and 1:0.93 with Matrigel for cell plating (plating mix).

The assay is conducted in the wells of a 96-well plate culture dish using a total volume of 130 μL of collagen/Matrigel/DMEM F12-10% FBS suspension in growth media for each matrix plug per individual well. Initially, 30 μL of collagen/Matrigel/DMEM F12-10% FBS (coating mix) is added into the well of a 96 well plate and the collagen is set to a gel by incubating at 37° C. for at least 15 minutes. A second layer of 100 μL collagen/Matrigel suspension (plating mix) containing m-IMCD3 cells (2.75×106 m-IMCD3 cells per 96-well plate) is layered over the coating mix and the collagen/Matrigel/cell mix again set to a gel by incubating at 37° C. Cell cultures are maintained at 37° C. in an atmosphere of 5% CO₂, 95% air.

Between 18 and 24 h after plating, test compound(s) as DMSO stock solutions [0.1% (v/v) final DMSO concentration] and PGE2 (100 nM final concentration) in DMEM F12-10% FBS are added in quadruplicate wells per condition. Media, together with the test compound and PGE2 are replenished after 2 or 3 days. After 6 days of culture z stack images of the wells are captured using the Nikon Eclipse Ti2-E microscope. Nikon General Analysis software is used to measure the following parameters in each well: the mean cyst area, the number of cysts, and the total cyst area.

PDE4 long form activators inhibited in vitro cyst formation in m-IMCD3 cells. Data are shown in Table 4 as mean cyst area (%), compared to 100% for (DMSO+PGE2) and 0% for DMSO control.

### Experiment 4: Inhibition of Proliferation of LNCaP Human Prostate Cancer Cells

In this study, the potential utility of PDE4 long form activators in the treatment of prostate cancer is studied using the LNCaP human prostate cancer cell line. The experiments are carried out according to the method described by Henderson et al. (Henderson, D. J. P., Byrne, A., Dulla, K., Jenster, G., Hoffmann, R., Baillie, G. S., Houslay, M. D. Br. J. Cancer 110:1278-1287, 2014).

### LNCaP Cell Culture

Androgen-sensitive (AS) LNCaP cells are maintained in RPMI1640 supplemented with 10% FBS (Seralabs), 2 mM L-glutamine and 1,000U penicillin-streptomycin. LNCaP androgen-insensitive (AI) cells are generated by culturing the LNCaP-AS cells in RPMI1640 supplemented with 10% charcoal stripped FBS, 2 mM L-glutamine and 1,000U penicillin-streptomycin for a minimum of four weeks. All tissue culture reagents are from Life Technologies.

### Xcelligence (Roche) Proliferation Assay

Cell proliferation is measured as a function of changing electrical impedance. Values are represented by cell index number, a dimensionless unit of measurement representing the cell status, which increases as cells adhere to 96-well electrode plates and divide.

LNCaP AI/AS cells are plated at a density of 25,000 cells per well in a 96-well electrode plate (in triplicate), in the presence/absence of various concentrations of test compound.

Cell indices are measured every 10 minutes for up to 100 hours, analysed using RTCA software and normalised to the cell index of vehicle-treated cells (n=3).

PDE4 long form activators inhibited the proliferation of AS and AI LNCaP human prostate cancer cells.

### Experiment 5: In Vivo Preclinical Model of Hyperparathyroidism: Inhibition of PTH-Induced CAMP Elevation in Urine in the Anaesthetised Rat

Within the kidney, the binding of parathyroid hormone (PTH) to PTH receptors results in the Gₐₛ-mediated elevation of intracellular CAMP. This increase of intracellular CAMP results in extrusion of CAMP to the urine (Yates et al., J Clin Invest 81:932-938, 1988). This experiment is based upon a modified Ellsworth-Howard assay (Kruse, K. and Kracht, U., European Journal of Pediatrics 146:373-377, 1987) and conducted in anaesthetized rats. In this experiment, rats were anaesthetized using isoflurane and catheterised to allow the collection of urine from the bladder. After an initial stabilization period, test compounds were administered by i.v. infusion (modelled to steady state) from time 0-120 min. PTH challenge infusion (33 ug/kg/hour) was started after 60 min of test compound infusion and sustained for one hour (60-120 min). Urine collection was conducted in 30-minute periods. Urine CAMP levels were assessed by ELISA (R&D systems). Urine samples were prepared for analysis as per the manufacturer's instructions. A standard curve for cAMP was assayed for each experiment and samples were assessed using a standard dilution range of 1:2, 1:4, 1:8 and 1:16, ensuring that the resulting data remained on the linear portion of the standard curve.

Control animals treated with vehicle alone (no PTH) showed no increase from baseline in urinary CAMP concentrations over the course of the experiment. Urine CAMP concentration remained below 50,000 pmol/mL. Control animals treated with PTH infusion plus vehicle (PTH challenge) showed an increase from baseline in cAMP concentration in urine collected from 90 to 120 min.

Treatment with PDE4 long form activators suppressed the elevation in cAMP concentration in urine in response to PTH challenge.

TABLE 1
Small molecule PDE4 long form activators according to the present invention

| | | Molecular | |
|---|---|---|---|
| Com- | | weight | |
| pound | Chemical name | (Daltons) | Chemical structure |
| Example 39 | 1-(2-(3,8-diazabicyclo[3.2.1]octan- 8-yl)-7,8-dihydro-1,6-naphthyridin- 6(5H)-yl)-2-cyclopentylethan-1-one | 354 | 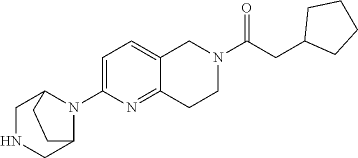 [SMILES: O=C(CC1CCCC1)N1CCc2nc(N3C4CCC3CNC4)ccc2C1] |
| Example 40 | 1-(2-(3,8-diazabicyclo[3.2.1]octan- 8-yl)-7,8-dihydro-1,6-naphthyridin- 6(5H)-yl)-2-(4,4- difluorocyclohexyl)ethan-1-one | 404 | 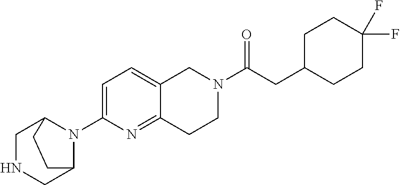 [SMILES: O=C(CC1CCC(F)(F)CC1)N1CCc2nc(N3C4CCC3CNC4)ccc2C1] |
| Example 41 | 1-(2-(3,8-diazabicyclo[3.2.1]octan- 8-yl)-7,8-dihydro-1,6-naphthyridin- 6(5H)-yl)-2-phenoxyethan-1-one | 378 | 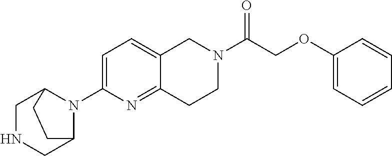 [SMILES: O=C(COc1ccccc1)N1CCc2nc(N3C4CCC3CNC4)ccc2C1] |
| Example 42 | 1-(2-(3,8-diazabicyclo[3.2.1]octan- 8-yl)-7,8-dihydro-1,6-naphthyridin- 6(5H)-yl)-3,3-dimethylbutan-1-one | 342 | 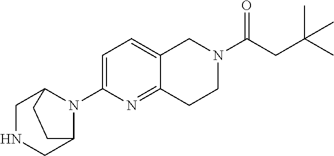 [SMILES: CC(C)(C)CC(=O)N1CCc2nc(N3C4CCC3CNC4)ccc2C1] |
| Example 43 | 1-(2-(3,8-diazabicyclo[3.2.1]octan- 8-yl)-7,8-dihydro-1,6-naphthyridin- 6(5H)-yl)-3-methylbutan-1-one | 328 | 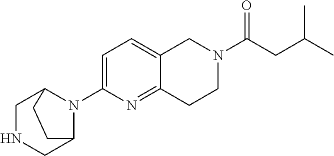 [SMILES: CC(C)CC(=O)N1CCc2nc(N3C4CCC3CNC4)ccc2C1] |
| Example 44 | 1-(2-(3,8-diazabicyclo[3.2.1]octan- 8-yl)-7,8-dihydro-1,6-naphthyridin- 6(5H)-yl)-2,3-dimethylbutan-1-one | 342 | 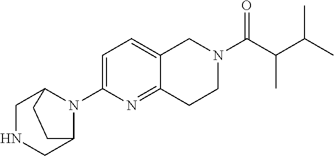 [SMILES: CC(C)C(C)C(=O)N1CCc2nc(N3C4CCC3CNC4)ccc2C1] |
| Example 45 | 1-(2-(3,8-diazabicyclo[3.2.1]octan- 8-yl)-7,8-dihydro-1,6-naphthyridin- 6(5H)-yl)-2-phenylethan-1-one | 362 | 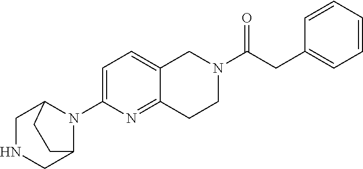 [SMILES: O=C(Cc1ccccc1)N1CCc2nc(N3C4CCC3CNC4)ccc2C1] |
| Example 46 | 1-(2-(3,8-diazabicyclo[3.2.1 ]octan- 8-yl)-7,8-dihydro-1,6-naphthyridin- 6(5H)-yl)-2-(4-fluorophenoxy)ethan- 1-one | 396 | 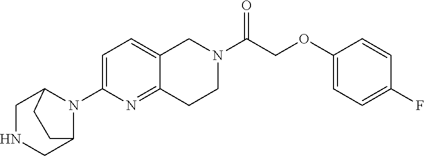 [SMILES: O=C(COc1ccc(F)cc1)N1CCc2nc(N3C4CCC3CNC4)ccc2C1] |
| Example 47 | (2-(3,8-diazabicyclo[3.2.1]octan-8- yl)-7,8-dihydro-1,6-naphthyridin- 6(5H)-yl)(cyclopentyl)methanone | 340 | 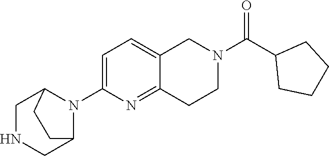 [SMILES: O=C(C1CCCC1)N1CCc2nc(N3C4CCC3CNC4)ccc2C1] |
| Example 48 | 1-(2-(3,8-diazabicyclo[3.2.1]octan- 8-yl)-7,8-dihydro-1,6-naphthyridin- 6(5H)-yl)-2-(4-fluorophenyl)ethan-1- one | 380 | 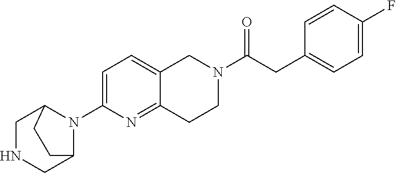 [SMILES: O=C(Cc1ccc(F)cc1)N1CCc2nc(N3C4CCC3CNC4)ccc2C1] |
| Example 49 | 1-(2-(3,8-diazabicyclo[3.2.1]octan- 8-yl)-7,8-dihydro-1,6-naphthyridin- 6(5H)-yl)-2,2-difluoro-2- phenylethan-1-one | 398 | 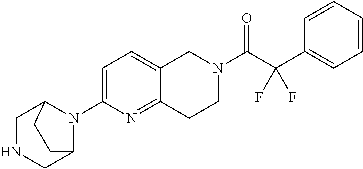 [SMILES: O=C(N1CCc2nc(N3C4CCC3CNC4)ccc2C1)C(F)(F)c1ccccc1] |
| Example 59 | 2-(3,8-diazabicyclo[3.2.1]octan-8- yl)-N-(4,4-difluorocyclohexyl)-7,8- dihydro-1,6-naphthyridine-6(5H)- carboxamide | 405 | 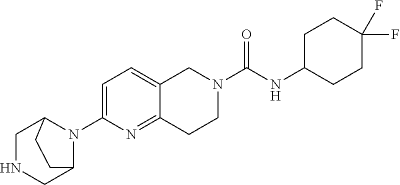 [SMILES: O=C(NC1CCC(F)(F)CC1)N1CCc2nc(N3C4CCC3CNC4)ccc2C1] |
| Example 61 | (2-(3,8-diazabicyclo[3.2.1]octan-8- yl)-7,8-dihydro-1,6-naphthyridin- 6(5H)-yl)(pyrrolidin-1-yl)methanone | 341 | 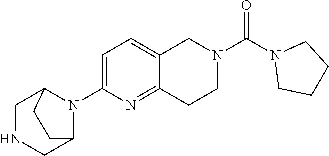 [SMILES: O=C(N1CCCC1)N1CCc2nc(N3C4CCC3CNC4)ccc2C1] |
| Example 63 | 2-(3,8-diazabicyclo[3.2.1]octan-8- yl)-N-cyclopentyl-N-methyl-7,8- dihydro-1,6-naphthyridine-6(5H)- carboxamide | 369 | 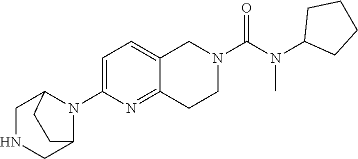 [SMILES: CN(C(=O)N1CCc2nc(N3C4CCC3CNC4)ccc2C1)C1CCCC1] |
| Example 64 | 2-(3,8-diazabicyclo[3.2.1]octan-8- yl)-N-cyclopentyl-N-ethyl-7,8- dihydro-1,6-naphthyridine-6(5H)- carboxamide | 383 | 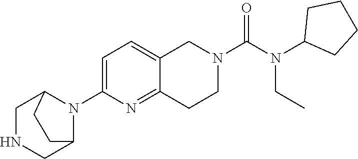 [SMILES: CCN(C(=O)N1CCc2nc(N3C4CCC3CNC4)ccc2C1)C1CCCC1] |
| Example 87 | 1-(2-(3,8-diazabicyclo[3.2.1]octan-8- yl)-5,7-dihydro-6H-pyrrolo[3,4- b]pyridin-6-yl)-2-(4,4- difluorocyclohexyl)ethan-1-one | 390 | 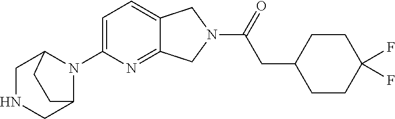 [SMILES: O=C(CC1CCC(F)(F)CC1)N1Cc2ccc(N3C4CCC3CNC4)nc2C1] |
| Example 88 | 1-(2-(3,8-diazabicyclo[3.2.1]octan-8- yl)-5,7-dihydro-6H-pyrrolo[3,4- b]pyridin-6-yl)-2-cyclopentylethan-1- one | 340 | 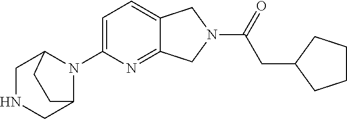 [SMILES: O=C(CC1CCCC1)N1Cc2ccc(N3C4CCC3CNC4)nc2C1] |
| Example 89 | 2-(3,8-diazabicyclo[3.2.1]octan-8-yl)- N-(4,4-difluorocyclohexyl)-5,7- dihydro-6H-pyrrolo[3,4-b]pyridine-6- carboxamide | 391 | 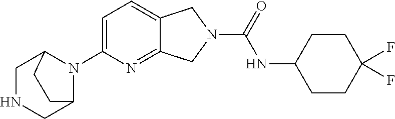 [SMILES: O=C(NC1CCC(F)(F)CC1)N1Cc2ccc(N3C4CCC3CNC4)nc2C1] |
| Example 90 | 2-(3,8-diazabicyclo[3.2.1]octan-8-yl)- N-cyclopentyl-5,7-dihydro-6H- pyrrolo[3,4-b]pyridine-6-carboxamide | 341 | 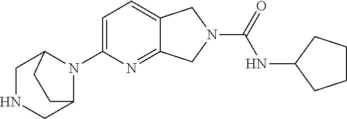 [SMILES: O=C(NC1CCCC1)N1Cc2ccc(N3C4CCC3CNC4)nc2C1] |
| Example 91 | N-(4,4-difluorocyclohexyl)-2-(3- methyl-3,8-diazabicyclo[3.2.1]octan- 8-yl)-5,7-dihydro-6H-pyrrolo[3,4- b]pyridine-6-carboxamide | 405 | 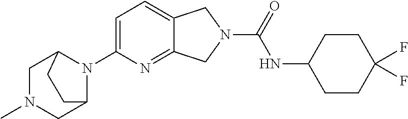 [SMILES: CN1CC2CCC(C1)N2c1ccc2c(n1)CN(C(=O)NC1CCC(F)(F)CC1)C2] |
| Example 92 | N-cyclopentyl-2-(3-methyl-3,8- diazabicyclo[3.2.1]octan-8-yl)-5,7- dihydro-6H-pyrrolo[3,4-b]pyridine-6- carboxamide | 355 | 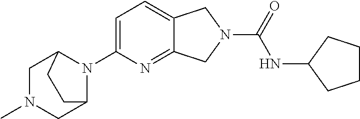 [SMILES: CN1CC2CCC(C1)N2c1ccc2c(n1)CN(C(=O)NC1CCCC1)C2] |
| Example 93 | 2-(4,4-difluorocyclohexyl)-1-(2-(3- methyl-3,8-diazabicyclo[3.2.1]octan- 8-yl)-5,7-dihydro-6H-pyrrolo[3,4- b]pyridin-6-yl)ethan-1-one | 404 | 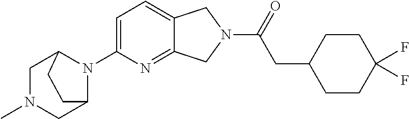 [SMILES: CN1CC2CCC(C1)N2c1ccc2c(n1)CN(C(=O)CC1CCC(F)(F)CC1)C2] |
| Example 94 | 2-cyclopentyl-1-(2-(3-methyl-3,8- diazabicyclo[3.2.1]octan-8-yl)-5,7- dihydro-6H-pyrrolo[3,4-b]pyridin-6- yl)ethan-1-one | 354 | 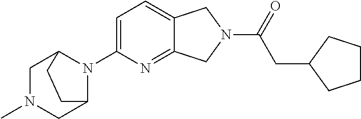 [SMILES: CN1CC2CCC(C1)N2c1ccc2c(n1)CN(C(=O)CC1CCCC1)C2] |
| Example 95 | (2-(3,8-diazabicyclo[3.2.1]octan-8- yl)-5,7-dihydro-6H-pyrrolo[3,4- b]pyridin-6-yl)(pyrrolidin-1- yl)methanone | 327 | 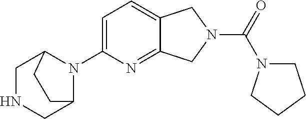 [SMILES: O=C(N1CCCC1)N1Cc2ccc(N3C4CCC3CNC4)nc2C1] |
| Example 96 | 2-(3,8-diazabicyclo[3.2.1]octan-8-yl)- N,N-diethyl-5,7-dihydro-6H- pyrrolo[3,4-b]pyridine-6-carboxamide | 329 | 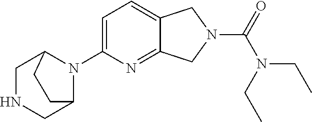 [SMILES: CCN(CC)C(=O)N1Cc2ccc(N3C4CCC3CNC4)nc2C1] |
| Example 105 | N-(4,4-difluorocyclohexyl)-2-(3- methyl-3,8-diazabicyclo[3.2.1]octan- 8-yl)-7,8-dihydro-1,6-naphthyridine- 6(5H)-carboxamide | 419 | 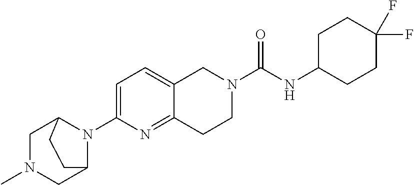 [SMILES: CN1CC2CCC(C1)N2c1ccc2c(n1)CCN(C(=O)NC1CCC(F)(F)CC1)C2] |
| Example 106 | 2-(4,4-difluorocyclohexyl)-1-(2-(3- methyl-3,8-diazabicyclo[3.2.1]octan- 8-yl)-7,8-dihydro-1,6-naphthyridin- 6(5H)-yl)ethan-1-one | 418 | 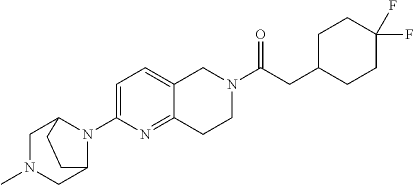 [SMILES: CN1CC2CCC(C1)N2c1ccc2c(n1)CCN(C(=O)CC1CCC(F)(F)CC1)C2] |
| Example 111 | 2-(4,4-difluorocyclohexyl)-1-(2- (piperazin-1-yl)-7,8-dihydro-1,6- naphthyridin-6(5H)-yl)ethan-1-one | 378 | 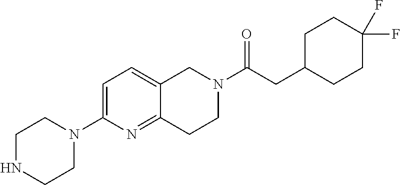 [SMILES: O=C(CC1CCC(F)(F)CC1)N1CCc2nc(N3CCNCC3)ccc2C1] |
| Example 112 | (R)-2-cyclopentyl-1-(2- (hexahydropyrrolo[1,2-a]pyrazin- 2(1H)-yl)-7,8-dihydro-1,6- naphthyridin-6(5H)-yl)ethan-1-one | 368 | 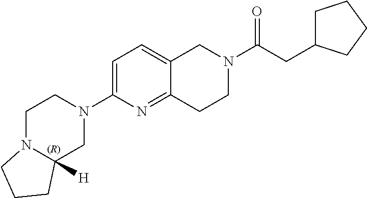 |
| Example 113 | (S)-2-cyclopentyl-1-(2- (hexahydropyrrolo[1,2-a]pyrazin- 2(1H)-yl)-7,8-dihydro-1,6- naphthyridin-6(5H)-yl)ethan-1-one | 368 | 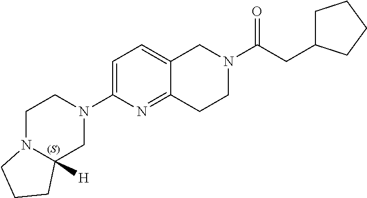 |
| Example 114 | 2-cyclopentyl-1-(2-(piperidin-4-yl)- 7,8-dihydro-1,6-naphthyridin-6(5H)- yl)ethan-1-one | 327 | 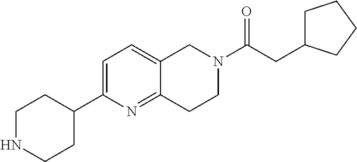 [SMILES: O=C(CC1CCCC1)N1CCc2nc(C3CCNCC3)ccc2C1] |
| Example 115 | 2-cyclopentyl-1-(2-(1-(2- hydroxyethyl)piperidin-4-yl)-7,8- dihydro-1,6-naphthyridin-6(5H)- yl)ethan-1-one | 37 | 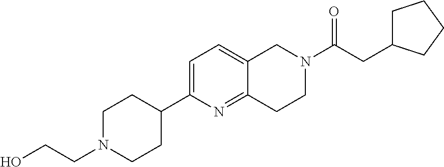 [SMILES: O=C(CC1CCCC1)N1CCc2nc(C3CCN(CCO)CC3)ccc2C1] |
| Example 119 | 1-(2-(3,8-diazabicyclo[3.2.1]octan-8- yl)-7,8-dihydro-1,6-naphthyridin- 6(5H)-yl)-2-(1- methylcyclopentyl)ethan-1-one | 368 | 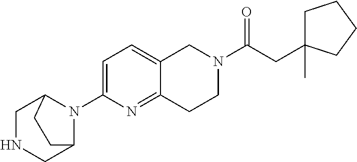 [SMILES: CC1(CC(=O)N2CCc3nc(N4C5CCC4CNC5)ccc3C2)CCCC1] |
| Example 120 | (2-(3,8-diazabicyclo[3.2.1]octan-8- yl)-7,8-dihydro-1,6-naphthyridin- 6(5H)-yl)(phenyl) methanone | 348 | 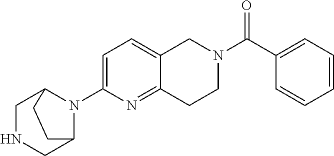 [SMILES: O=C(c1ccccc1)N1CCc2nc(N3C4CCC3CNC4)ccc2C1] |
| Example 121 | (2-(3,8-diazabicyclo[3.2.1]octan-8- yl)-7,8-dihydro-1,6-naphthyridin- 6(5H)-yl)(2- methoxyphenyl)methanone | 378 | 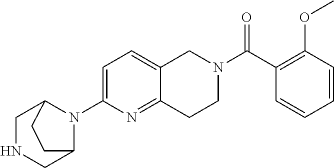 [SMILES: COc1ccccc1C(=O)N1CCc2nc(N3C4CCC3CNC4)ccc2C1] |
| Example 122 | (2-(3,8-diazabicyclo[3.2.1]octan-8- yl)-7,8-dihydro-1,6-naphthyridin- 6(5H)-yl)(o-tolyl)methanone | 362 | 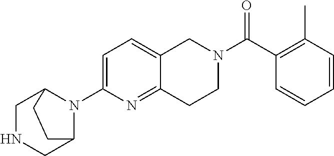 [SMILES: Cc1ccccc1C(=O)N1CCc2nc(N3C4CCC3CNC4)ccc2C1] |
| Example 123 | (2-(3,8-diazabicyclo[3.2.1]octan-8- yl)-7,8-dihydro-1,6-naphthyridin- 6(5H)-yl)(4-fluorophenyl)methanone | 366 | 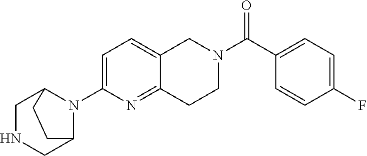 [SMILES: O=C(c1ccc(F)cc1)N1CCc2nc(N3C4CCC3CNC4)ccc2C1] |
| Example 124 | (2R)-1-(2-(3,8- diazabicyclo[3.2.1]octan-8-yl)-7,8- dihydro-1,6-naphthyridin-6(5H)-yl)-2- cyclopentyl-2-hydroxyethan-1-one | 370 | 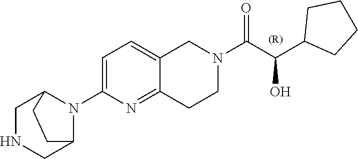 |
| Example 125 | cyclopentyl 2-(3,8- diazabicyclo[3.2.1]octan-8-yl)-7,8- dihydro-1,6-naphthyridine-6(5H)- carboxylate | 356 | 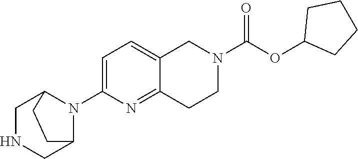 [SMILES: O=C(OC1CCCC1)N1CCc2nc(N3C4CCC3CNC4)ccc2C1] |
| Example 126 | isopropyl 2-(3,8- diazabicyclo[3.2.1]octan-8-yl)-7,8- dihydro-1,6-naphthyridine-6(5H)- carboxylate | 330 | 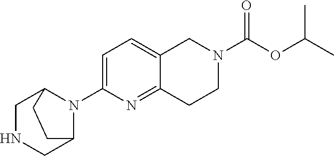 [SMILES: CC(C)OC(=O)N1CCc2nc(N3C4CCC3CNC4)ccc2C1] |
| Example 127 | benzyl 2-(3,8- diazabicyclo[3.2.1]octan-8-yl)-7,8- dihydro-1,6-naphthyridine-6(5H)- carboxylate | 378 | 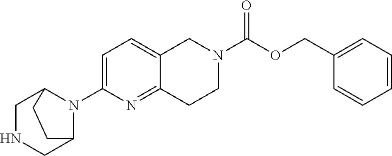 [SMILES: O=C(OCc1ccccc1)N1CCc2nc(N3C4CCC3CNC4)ccc2C1] |
| Example 128 | (2S)-1-(2-(3,8- diazabicyclo[3.2.1]octan-8-yl)-5,7- dihydro-6H-pyrrolo[3,4-b]pyridin-6- yl)-2,3-dimethylbutan-1-one | 328 | 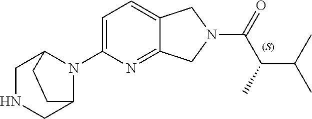 |
| Example 129 | 1-(2-(3,8-diazabicyclo[3.2.1]octan-8- yl)-5,7-dihydro-6H-pyrrolo[3,4- b]pyridin-6-yl)-2-(1- methylcyclopentyl)ethan-1-one | 354 | 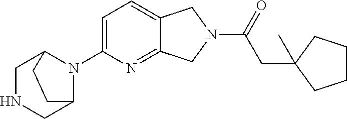 [SMILES: CC1(CC(=O)N2Cc3ccc(N4C5CCC4CNC5)nc3C2)CCCC1] |
| Example 130 | 1-(2-(3,8-diazabicyclo[3.2.1]octan-8- yl)-5,7-dihydro-6H-pyrrolo[3,4- b]pyridin-6-yl)-2,2-difluoro-2- phenylethan-1-one | 384 | 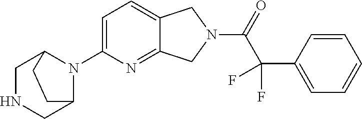 [SMILES: O=C(N1Cc2ccc(N3C4CCC3CNC4)nc2C1)C(F)(F)c1ccccc1] |
| Example 131 | (R)-2-cyclopentyl-1-(2-(3- (hydroxymethyl)piperazin-1-yl)-7,8- dihydro-1,6-naphthyridin-6(5H)- yl)ethan-1-one | 358 | 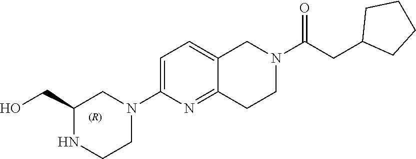 |
| Example 132 | (S)-2-cyclopentyl-1-(2-(3- (hydroxymethyl)piperazin-1-yl)-7,8- dihydro-1,6-naphthyridin-6(5H)- yl)ethan-1-one | 358 | 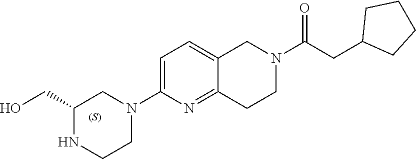 |
| Example 133 | 1-(2-(3,8-diazabicyclo[3.2.1]octan-8- yl)-7,8-dihydropyrido[4,3-d]pyrimidin- 6(5H)-yl)-2-phenylethan-1-one | 363 | 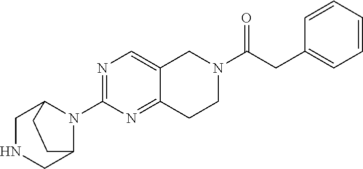 [SMILES: O=C(Cc1ccccc1)N1CCc2nc(N3C4CCC3CNC4)ncc2C1] |
| Example 134 | 2-phenyl-1-(2-(piperazin-1-yl)-7,8- dihydropyrido[4,3-d]pyrimidin-6(5H)- yl)ethan-1-one | 337 | 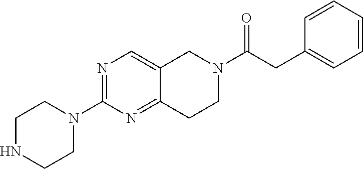 [SMILES: O=C(Cc1ccccc1)N1CCc2nc(N3CCNCC3)ncc2C1] |
| Example 135 | 1-(2-(3,8-diazabicyclo[3.2.1]octan-3- yl)-7,8-dihydropyrido[4,3-d]pyrimidin- 6(5H)-yl)-2-phenylethan-1-one | 363 | 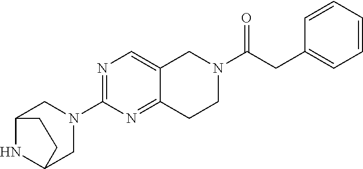 [SMILES: O=C(Cc1ccccc1)N1CCc2nc(N3CC4CCC(C3)N4)ncc2C1] |
| Example 136 | 4-(2-(2-(3,8-diazabicyclo[3.2.1]octan- 8-yl)-7,8-dihydropyrido[4,3- d]pyrimidin-6(5H)-yl)-2- oxoethyl)benzonitrile | 388 | 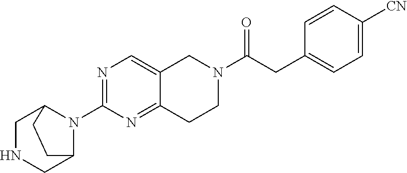 [SMILES: N#Cc1ccc(CC(=O)N2CCc3nc(N4C5CCC4CNC5)ncc3C2)cc1] |
| Example 137 | 4-(2-oxo-2-(2-(piperazin-1-yl)-7,8- dihydropyrido[4,3-d]pyrimidin-6(5H)- yl)ethyl)benzonitrile | 362 | 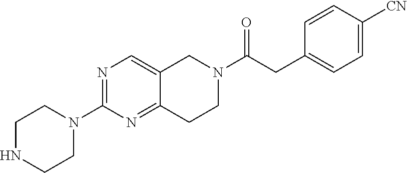 [SMILES: N#Cc1ccc(CC(=O)N2CCc3nc(N4CCNCC4)ncc3C2)cc1] |
| Example 138 | 4-(2-(2-(3,8-diazabicyclo[3.2.1]octan- 3-yl)-7,8-dihydropyrido[4,3- d]pyrimidin-6(5H)-yl)-2- oxoethyl)benzonitrile | 388 | 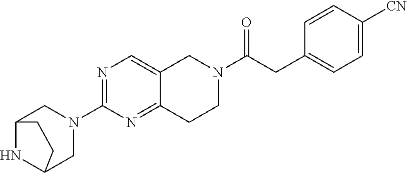 [SMILES: N#Cc1ccc(CC(=O)N2CCc3nc(N4CC5CCC(C4)N5)ncc3C2)cc1] |
| Example 139 | 1-(2-(3,8-diazabicyclo[3.2.1]octan-8- yl)-7,8-dihydropyrido[4,3-d]pyrimidin- 6(5H)-yl)-2-(4-fluorophenyl)ethan-1- one | 381 | 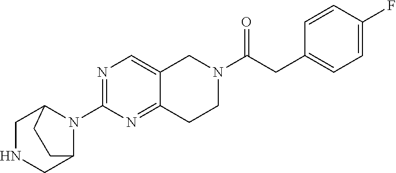 [SMILES: O=C(Cc1ccc(F)cc1)N1CCc2nc(N3C4CCC3CNC4)ncc2C1] |
| Example 140 | 2-(4-fluorophenyl)-1-(2-(piperazin-1- yl)-7,8-dihydropyrido[4,3-d]pyrimidin- 6(5H)-yl)ethan-1-one | 355 | 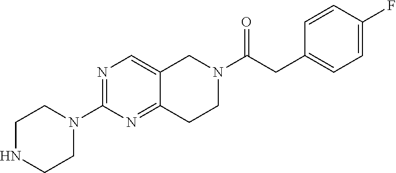 [SMILES: O=C(Cc1ccc(F)cc1)N1CCc2nc(N3CCNCC3)ncc2C1] |
| Example 141 | 1-(2-(3,8-diazabicyclo[3.2.1]octan-3- yl)-7,8-dihydropyrido[4,3-d]pyrimidin- 6(5H)-yl)-2-(4-fluorophenyl)ethan-1- one | 381 | 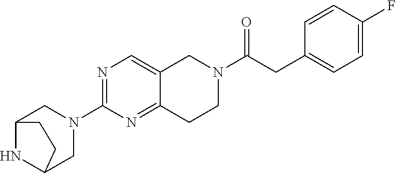 [SMILES: O=C(Cc1ccc(F)cc1)N1CCc2nc(N3CC4CCC(C3)N4)ncc2C1] |
| Example 142 | 1-(2-(3,8-diazabicyclo[3.2.1]octan-8- yl)-7,8-dihydropyrido[4,3-d]pyrimidin- 6(5H)-yl)-2-cyclopentylethan-1-one | 355 | 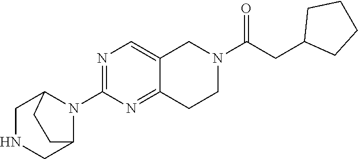 [SMILES: O=C(CC1CCCC1)N1CCc2nc(N3C4CCC3CNC4)ncc2C1] |
| Example 143 | 2-cyclopentyl-1-(2-(piperazin-1-yl)- 7,8-dihydropyrido[4,3-d]pyrimidin- 6(5H)-yl)ethan-1-one | 329 | 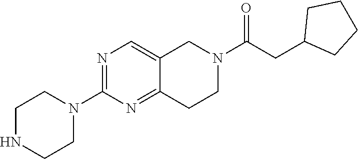 [SMILES: O=C(CC1CCCC1)N1CCc2nc(N3CCNCC3)ncc2C1] |
| Example 144 | 1-(2-(3,8-diazabicyclo[3.2.1]octan-8- yl)-7,8-dihydropyrido[4,3-d]pyrimidin- 6(5H)-yl)-2-cyclohexylethan-1-one | 369 | 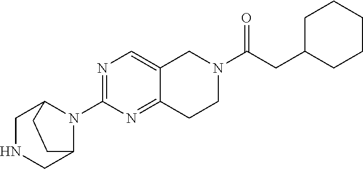 [SMILES: O=C(CC1CCCCC1)N1CCc2nc(N3C4CCC3CNC4)ncc2C1] |
| Example 145 | 2-cyclohexyl-1-(2-(piperazin-1-yl)- 7,8-dihydropyrido[4,3-d]pyrimidin- 6(5H)-yl)ethan-1-one | 343 | 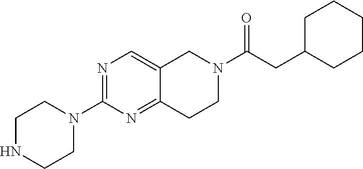 [SMILES: O=C(CC1CCCCC1)N1CCc2nc(N3CCNCC3)ncc2C1] |
| Example 146 | 1-(2-(3,8-diazabicyclo[3.2.1]octan-8- yl)-7,8-dihydropyrido[4,3-d]pyrimidin- 6(5H)-yl)-3,3-dimethylbutan-1-one | 343 | 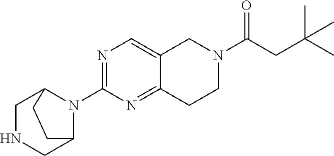 [SMILES: CC(C)(C)CC(=O)N1CCc2nc(N3C4CCC3CNC4)ncc2C1] |
| Example 147 | (4-fluorophenyl)(2-(piperazin-1-yl)- 7,8-dihydropyrido[4,3-d]pyrimidin- 6(5H)-yl)methanone | 341 | 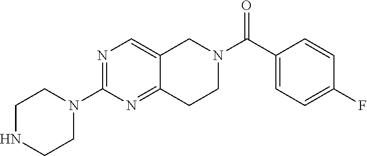 [SMILES: O=C(c1ccc(F)cc1)N1CCc2nc(N3CCNCC3)ncc2C1] |
| Example 148 | 1-(2-(3,8-diazabicyclo[3.2.1]octan-8- yl)-5,8-dihydropyrido[3,4-d]pyrimidin- 7(6H)-yl)-2-cyclopentylethan-1-one | 355 | 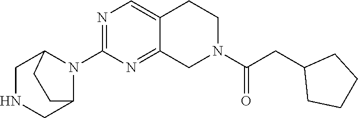 [SMILES: O=C(CC1CCCC1)N1CCc2cnc(N3C4CCC3CNC4)nc2C1] |
| Example 149 | 3,3-dimethyl-1-(2-(piperazin-1-yl)- 7,8-dihydro-1,6-naphthyridin-6(5H)- yl)butan-1-one | 316 | 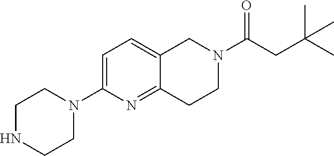 [SMILES: CC(C)(C)CC(=O)N1CCc2nc(N3CCNCC3)ccc2C1] |
| Example 150 | 1-(2-(4-ethylpiperazin-1-yl)-7,8- dihydro-1,6-naphthyridin-6(5H)-yl)- 3,3-dimethylbutan-1-one | 344 | 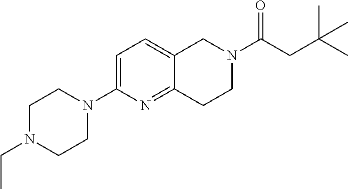 [SMILES: CCN1CCN(c2ccc3c(n2)CCN(C(=O)CC(C)(C)C)C3)CC1] |
| Example 151 | 1-(2-(3,9-diazabicyclo[3.3.1]nonan-9- yl)-7,8-dihydro-1,6-naphthyridin- 6(5H)-yl)-2-cyclopentylethan-1-one | 368 | 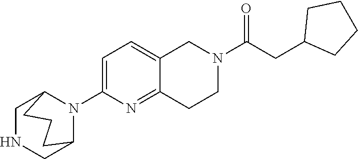 [SMILES: O=C(CC1CCCC1)N1CCc2nc(N3C4CCCC3CNC4)ccc2C1] |
| Example 152 | 1-(2-(3,9-diazabicyclo[3.3.1]nonan-3- yl)-7,8-dihydro-1,6-naphthyridin- 6(5H)-yl)-2-cyclopentylethan-1-one | 368 | 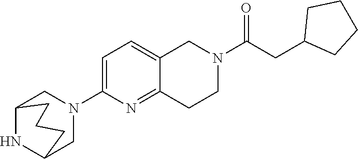 [SMILES: O=C(CC1CCCC1)N1CCc2nc(N3CC4CCCC(C3)N4)ccc2C1] |
| Example 153 | 1-(2-(2,5-diazabicyclo[2.2.2]octan-2- yl)-7,8-dihydro-1,6-naphthyridin- 6(5H)-yl)-2-cyclopentylethan-1-one | 354 | 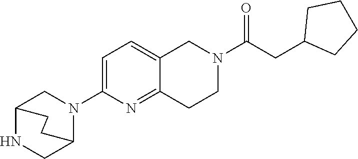 [SMILES: O=C(CC1CCCC1)N1CCc2nc(N3CC4CCC3CN4)ccc2C1] |
| Example 154 | 1-(2-(3,8-diazabicyclo[3.2.1]octan-3- yl)-7,8-dihydro-1,6-naphthyridin- 6(5H)-yl)-2-cyclopentylethan-1-one | 354 | 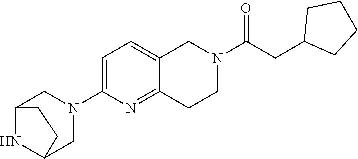 [SMILES: O=C(CC1CCCC1)N1CCc2nc(N3CC4CCC(C3)N4)ccc2C1] |
| Example 155 | 1-(2-((1S,4S)-2,5- diazabicyclo[2.2.1]heptan-2-yl)-7,8- dihydro-1,6-naphthyridin-6(5H)-yl)-2- cyclopentylethan-1-one | 340 | 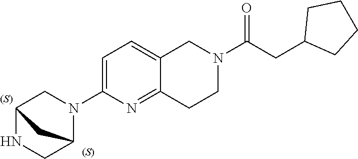 |
| Example 156 | 1-(2-((1R,4R)-2,5- diazabicyclo[2.2.1]heptan-2-yl)-7,8- dihydro-1,6-naphthyridin-6(5H)-yl)-2- cyclopentylethan-1-one | 340 | 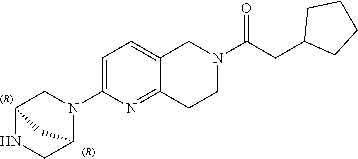 |
| Example 157 | 2-cyclopentyl-1-(2-(7-hydroxy-3,9- diazabicyclo[3.3.1]nonan-9-yl)-7,8- dihydro-1,6-naphthyridin-6(5H)- yl)ethan-1-one | 384 | 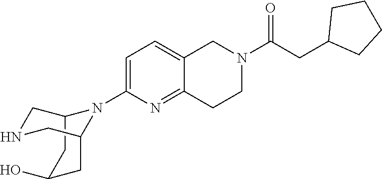 [SMILES: O=C(CC1CCCC1)N1CCc2nc(N3C4CNCC3CC(O)C4)ccc2C1] |
| Example 158 | 2-(3,8-diazabicyclo[3.2.1]octan-8-yl)- 6-((4-fluorobenzyl)sulfonyl)-5,6,7,8- tetrahydro-1,6-naphthyridine | 416 | 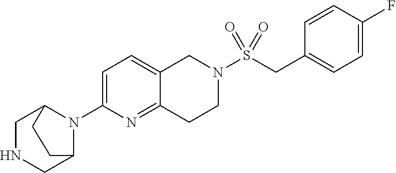 [SMILES: O=S(=O)(Cc1ccc(F)cc1)N1CCc2nc(N3C4CCC3CNC4)ccc2C1] |
| Example 159 | 2-(3-ethyl-3,8- diazabicyclo[3.2.1]octan-8-yl)-6-((4- fluorobenzyl)sulfonyl)-5,6,7,8- tetrahydro-1,6-naphthyridine | 444 | 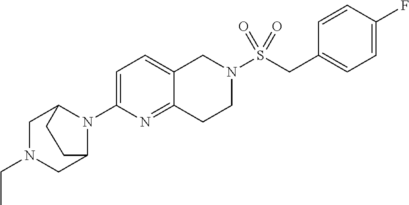 [SMILES: CCN1CC2CCC(C1)N2c1ccc2c(n1)CCN(S(=O)(=O)Cc1ccc(F)cc1)C2] |
| Example 160 | 6-((4-fluorobenzyl)sulfonyl)-2- (piperazin-1-yl)-5,6,7,8-tetrahydro- 1,6-naphthyridine | 390 | 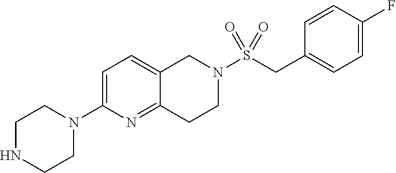 [SMILES: O=S(=O)(Cc1ccc(F)cc1)N1CCc2nc(N3CCNCC3)ccc2C1] |
| Example 161 | 2-(4-ethylpiperazin-1-yl)-6-((4- fluorobenzyl)sulfonyl)-5,6,7,8- tetrahydro-1,6-naphthyridine | 418 | 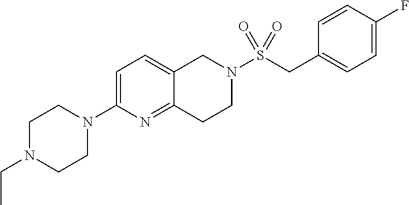 [SMILES: CCN1CCN(c2ccc3c(n2)CCN(S(=O)(=O)Cc2ccc(F)cc2)C3)CC1] |
| Example 162 | 6-(benzylsulfonyl)-2-(3,8- diazabicyclo[3.2.1]octan-8-yl)- 5,6,7,8-tetrahydro-1,6-naphthyridine | 398 | 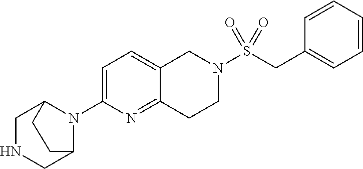 [SMILES: O=S(=O)(Cc1ccccc1)N1CCc2nc(N3C4CCC3CNC4)ccc2C1] |
| Example 163 | 6-(benzylsulfonyl)-2-(3-ethyl-3,8- diazabicyclo[3.2.1]octan-8-yl)- 5,6,7,8-tetrahydro-1,6-naphthyridine | 426 | 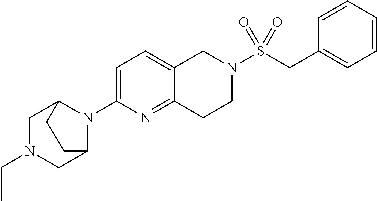 [SMILES: CCN1CC2CCC(C1)N2c1ccc2c(n1)CCN(S(=O)(=O)Cc1ccccc1)C2] |
| Example 164 | 6-(benzylsulfonyl)-2-(piperazin-1-yl)- 5,6,7,8-tetrahydro-1,6-naphthyridine | 372 | 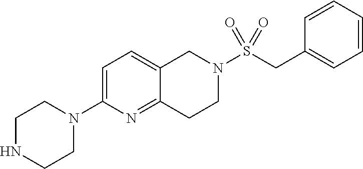 [SMILES: O=S(=O)(Cc1ccccc1)N1CCc2nc(N3CCNCC3)ccc2C1] |
| Example 165 | 6-(benzylsulfonyl)-2-(4- ethylpiperazin-1-yl)-5,6,7,8- tetrahydro-1,6-naphthyridine | 400 | 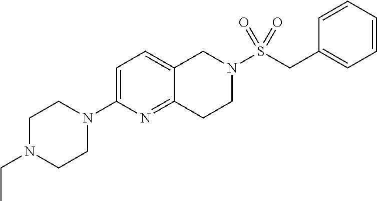 [SMILES: CCN1CCN(c2ccc3c(n2)CCN(S(=O)(=O)Cc2ccccc2)C3)CC1] |
| Example 166 | 2-(3,8-diazabicyclo[3.2.1]octan-8-yl)- N-((S)-chroman-4-yl)-7,8-dihydro- 1,6-naphthyridine-6(5H)- carboxamide | 419 | 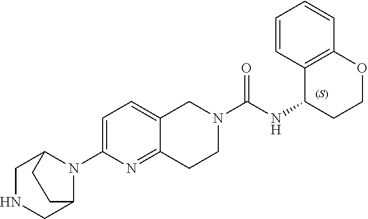 |
| Example 167 | N-((S)-chroman-4-yl)-2-(3-ethyl-3,8- diazabicyclo[3.2.1]octan-8-yl)-7,8- dihydro-1,6-naphthyridine-6(5H)- carboxamide | 447 | 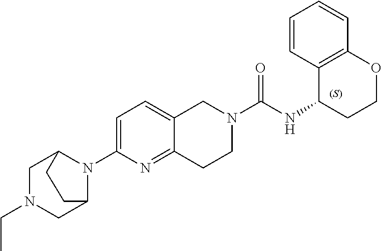 |
| Example 168 | (S)-N-(chroman-4-yl)-2-(piperazin-1- yl)-7,8-dihydro-1,6-naphthyridine- 6(5H)-carboxamide | 393 | 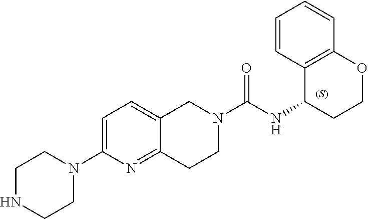 |
| Example 169 | (S)-N-(chroman-4-yl)-2-(4- ethylpiperazin-1-yl)-7,8-dihydro-1,6- naphthyridine-6(5H)-carboxamide | 421 | 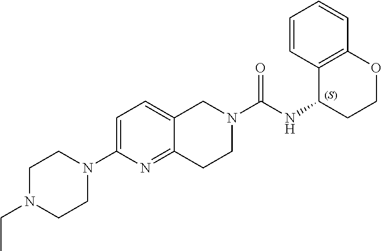 |
| Example 170 | N-((R)-chroman-4-yl)-2-(3-ethyl-3,8- diazabicyclo[3.2.1]octan-8-yl)-7,8- dihydro-1,6-naphthyridine-6(5H)- carboxamide | 447 | 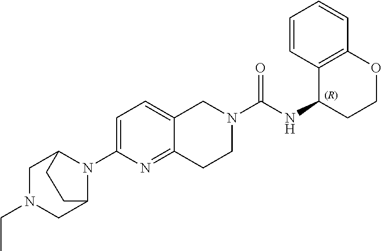 |
| Example 171 | 1-(2-(3-ethyl-3,8- diazabicyclo[3.2.1]octan-8-yl)-7,8- dihydro-1,6-naphthyridin-6(5H)-yl)-2- (4-fluorophenoxy)ethan-1-one | 424 | 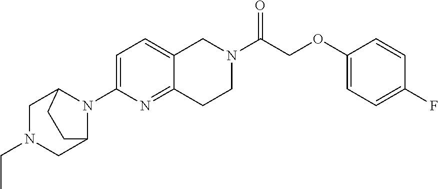 [SMILES: CCN1CC2CCC(C1)N2c1ccc2c(n1)CCN(C(=O)COc1ccc(F)cc1)C2] |
| Example 172 | 2-(4-fluorophenoxy)-1-(2-(3-propyl- 3,8-diazabicyclo[3.2.1]octan-8-yl)- 7,8-dihydro-1,6-naphthyridin-6(5H)- yl)ethan-1-one | 438 | 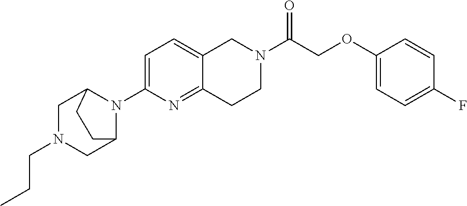 [SMILES: CCCN1CC2CCC(C1)N2c1ccc2c(n1)CCN(C(=O)COc1ccc(F)cc1)C2] |
| Example 173 | 2-(4-fluorophenoxy)-1-(2-(3-(2- methoxyethyl)-3,8- diazabicyclo[3.2.1]octan-8-yl)-7,8- dihydro-1,6-naphthyridin-6(5H)- yl)ethan-1-one | 454 | 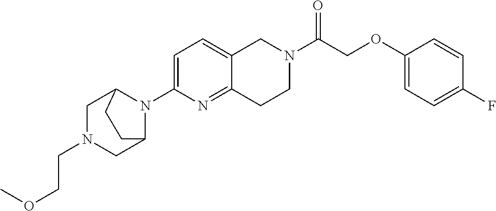 [SMILES: COCCN1CC2CCC(C1)N2c1ccc2c(n1)CCN(C(=O)COc1ccc(F)cc1)C2] |
| Example 174 | 2-(4-fluorophenoxy)-1-(2-(3-(3- methoxypropyl)-3,8- diazabicyclo[3.2.1]octan-8-yl)-7,8- dihydro-1,6-naphthyridin-6(5H)- yl)ethan-1-one | 468 | 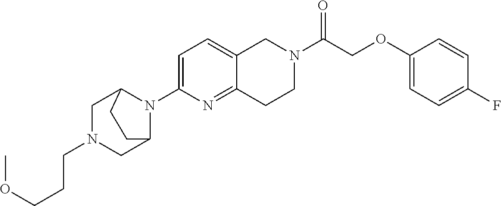 [SMILES: COCCCN1CC2CCC(C1)N2c1ccc2c(n1)CCN(C(=O)COc1ccc(F)cc1)C2] |
| Example 175 | 2-phenyl-1-(2-(piperazin-1-yl)-7,8- dihydro-1,6-naphthyridin-6(5H)- yl)ethan-1-one | 336 | 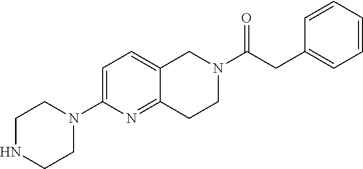 [SMILES: O=C(Cc1ccccc1)N1CCc2nc(N3CCNCC3)ccc2C1] |
| Example 176 | 2-(4-fluorophenyl)-1-(2-(piperazin-1- yl)-7,8-dihydro-1,6-naphthyridin- 6(5H)-yl)ethan-1-one | 354 | 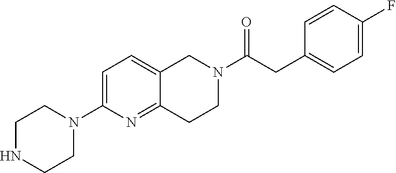 [SMILES: O=C(Cc1ccc(F)cc1)N1CCc2nc(N3CCNCC3)ccc2C1] |
| Example 177 | 1-(3-(3,8-diazabicyclo[3.2.1]octan-8- yl)-5,8-dihydro-1,7-naphthyridin- 7(6H)-yl)-2-cyclopentylethan-1-one | 354 | 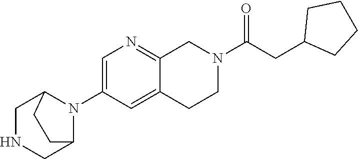 [SMILES: O=C(CC1CCCC1)N1CCc2cc(N3C4CCC3CNC4)cnc2C1] |
| Example 178 | 1-(6-(3,8-diazabicyclo[3.2.1]octan-8- yl)-3,4-dihydro-2,7-naphthyridin- 2(1H)-yl)-2-cyclopentylethan-1-one | 354 | 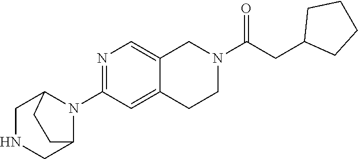 [SMILES: O=C(CC1CCCC1)N1CCc2cc(N3C4CCC3CNC4)ncc2C1] |
| Example 179 | 1-(7-(3,8-diazabicyclo[3.2.1]octan-8- yl)-3,4-dihydro-2,6-naphthyridin- 2(1H)-yl)-2-cyclopentylethan-1-one | 354 | 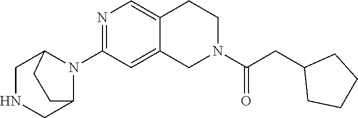 [SMILES: O=C(CC1CCCC1)N1CCc2cnc(N3C4CCC3CNC4)cc2C1] |
| Example 180 | 2-(4-fluorophenoxy)-1 -(2-(piperazin- 1-yl)-7,8-dihydro-1,6-naphthyridin- 6(5H)-yl)ethan-1-one | 370 | 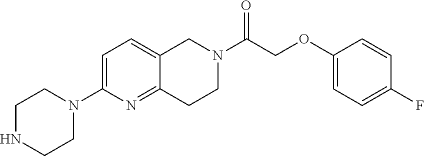 [SMILES: O=C(COc1ccc(F)cc1)N1CCc2nc(N3CCNCC3)ccc2C1] |
| Example 181 | 2-cyclopentyl-1-(2-(2-methylpyridin- 3-yl)-7,8-dihydro-1,6-naphthyridin- 6(5H)-yl)ethan-1-one | 335 | 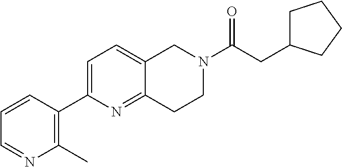 [SMILES: Cc1ncccc1-c1ccc2c(n1)CCN(C(=O)CC1CCCC1)C2] |
| Example 182 | 2-cyclopentyl-1-(2-(5-methylpyridin- 3-yl)-7,8-dihydro-1,6-naphthyridin- 6(5H)-yl)ethan-1-one | 335 | 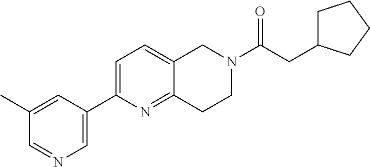 [SMILES: Cc1cncc(-c2ccc3c(n2)CCN(C(=O)CC2CCCC2)C3)c1] |

TABLE 2
Enzyme assay data for PDE4D5, a long form of PDE4 and PDE4B2,
a short form of PDE4
Using the method described in Experiment 1, the following
PDE4 assay data were obtained for exemplary compounds
of the present invention.

| Compound | % increase in PDE4D5 | % increase in PDE4B2 |
|---|---|---|
| Example | (long isoform) activity | (short isoform) activity |
| Number | at 100 μM* (%) | at 100 μM* |
| Example 46 | 94 | 6 |
| Example 48 | 31 | −9 |
| Example 139 | 28 | −1 |
| Example 159 | 35 | 2 |

*Measured as mean % increase in counts over basal activity

TABLE 3
cAMP levels in cultures of m-IMCD3 cells expressing PDE4 long forms
Using the method described in Experiment 2, the following m-IMCD3
cell culture cAMP measurements were obtained for exemplary
compounds of the present invention.

| Compound | | cAMP level (% of |
|---|---|---|
| Example Number | Concentration | PGE2-stimulated)^{§} |
| Example 40 | 10 μM | 43% |
| Example 42 | 3 μM | 28% |
| Example 46 | 3 μM | 42% |
| Example 48 | 3 μM | 57% |
| Example 91 | 3 μM | 31% |
| Example 111 | 10 μM | 38% |
| Example 112 | 3 μM | 54% |
| Example 113 | 3 μM | 63% |
| Example 114 | 3 μM | 47% |
| Example 115 | 3 μM | 32% |
| Example 121 | 3 μM | 44% |
| Example 122 | 3 μM | 66% |
| Example 129 | 3 μM | 44% |
| Example 134 | 3 μM | 55% |
| Example 136 | 3 μM | 38% |
| Example 147 | 3 μM | 40% |
| Example 150 | 3 μM | 49% |
| Example 151 | 3 μM | 51% |
| Example 152 | 3 μM | 41% |
| Example 155 | 3 μM | 63% |
| Example 156 | 3 μM | 49% |
| Example 158 | 3 μM | 46% |
| Example 160 | 3 μM | 35% |
| Example 162 | 3 μM | 38% |
| Example 163 | 3 μM | 39% |
| Example 166 | 3 μM | 35% |

^{§}Compared to 100% for (DMSO + PGE2)

TABLE 4
Inhibition of PGE2-stimulated in vitro cyst formation in m-IMCD3 cells
Using the method described in Experiment 3, the following m-IMCD3
kidney cell cyst suppression data were obtained for exemplary
compounds of the present invention.

| Compound | % Cyst area* after | Compound | % Cyst area* after |
|---|---|---|---|
| Example | incubation with 10 | Example | incubation with 3 |
| Number | μM test compound | Number | μM test compound |
| Example 39 | 5% | Example 120 | 1% |
| Example 40 | 0% | Example 121 | 8% |
| Example 41 | 4% | Example 122 | −1% |
| Example 42 | 0% | Example 123 | −7% |
| Example 43 | −4% | Example 124 | 0% |
| Example 44 | −2% | Example 125 | −3% |
| Example 47 | 5% | Example 126 | 14% |
| Example 48 | −5% | Example 127 | 3% |
| Example 59 | −4% | Example 128 | 3% |
| Example 61 | 3% | Example 133 | −5% |
| Example 63 | 2% | Example 134 | 7% |
| Example 64 | 0% | Example 135 | −5% |
| Example 87 | 0% | Example 136 | 1% |
| Example 89 | 2% | Example 137 | 13% |
| Example 91 | −5% | Example 138 | 0% |
| Example 93 | −3% | Example 139 | 1% |
| Example 95 | −2% | Example 140 | 6% |
| Example 96 | −4% | Example 141 | −2% |
| Example 106 | 1% | Example 142 | 26% |
| Example 111 | −3% | Example 143 | 22% |
| Example 112 | −6% | Example 145 | 15% |
| Example 113 | −4% | Example 146 | 22% |
| Example 114 | −2% | Example 147 | 4% |
| Example 115 | −3% | Example 148 | 7% |
| Example 119 | −4% | Example 149 | 11% |
| Example 129 | 2% | Example 150 | −1% |
| Example 130 | −10% | Example 151 | −8% |
| Example 131 | −4% | Example 152 | −6% |
| Example 132 | −4% | Example 153 | 2% |
| | | Example 154 | −6% |
| | | Example 156 | 4% |
| | | Example 157 | 10% |
| | | Example 158 | 5% |
| | | Example 159 | 1% |
| | | Example 160 | −4% |
| | | Example 161 | 0% |
| | | Example 162 | −3% |
| | | Example 163 | −1% |
| | | Example 164 | 0% |
| | | Example 165 | −1% |
| | | Example 166 | 3% |
| | | Example 167 | −8% |
| | | Example 168 | 4% |
| | | Example 169 | −4% |
| | | Example 170 | 23% |
| | | Example 171 | 4% |
| | | Example 172 | 6% |
| | | Example 173 | 3% |
| | | Example 174 | 1% |
| | | Example 175 | 2% |
| | | Example 176 | −2% |
| | | Example 177 | 10% |
| | | Example 178 | 1% |
| | | Example 179 | 1% |
| | | Example 180 | 11% |
| | | Example 181 | 26% |
| | | Example 182 | 50% |

*Compared to 100% for (DMSO + PGE2) and 0% for DMSO control

It will be appreciated that the above description is made by way of example and not limitation of the scope of the appended claims, including any equivalents as included within the scope of the claims. Various modifications are possible and will be readily apparent to the skilled person in the art. Likewise, features of the described embodiments can be combined with any appropriate aspect described above and optional features of any one aspect can be combined with any other appropriate aspect.

1. A compound of Formula I 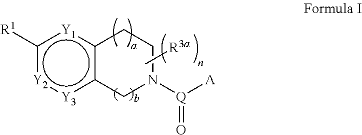
or a pharmaceutically acceptable salt or derivative thereof, wherein:
one or two of Y₁, Y₂ and Y₃ are N and the other(s) are CR^{3b};
Q is C or S(O);
R¹ is a 4- to 10-membered non-aromatic ring that may be monocyclic, bridged or bicyclic containing at least 1 ring N heteroatom and optionally a ring O heteroatom, and wherein R¹ is optionally substituted with 1 or more R⁴;
A is R^{2c}, NR^{2a}R^{2b} or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered carbocyclic ring or heterocyclic ring containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1 and b is 1 or 2, wherein when b is 2, a is 0;
for use in the treatment or prevention of a disease or disorder that can be ameliorated by activation of long isoforms of PDE4 or a disease or disorder mediated by excessive intracellular cyclic AMP signalling.

2. The compound for use of claim 1, where in the compound is a compound of formula: 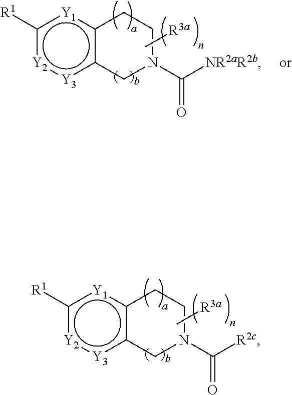
or a pharmaceutically acceptable salt or derivative thereof.

3. The compound, or a pharmaceutically acceptable salt or derivative thereof, for use of claim 1 or 2, wherein R¹ is a 5- to 6-membered saturated, monocyclic ring containing at least 1 ring N heteroatom and optionally a ring O heteroatom; a 7- to 9-membered saturated, bridged ring system containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R¹ is optionally substituted with 1, 2 or 3 R⁴.

4. The compound, or a pharmaceutically acceptable salt or derivative thereof, for use of any preceding claim, wherein R¹ is a 4- to 10-membered non-aromatic ring that may be monocyclic, bridged or bicyclic containing at least 1 ring N heteroatom and optionally a ring O heteroatom; wherein R¹ is optionally substituted with 1 R⁴.

5. The compound, or a pharmaceutically acceptable salt or derivative thereof, for use of any one of claims 1 to 3, wherein R¹ is a 7- to 9-membered saturated, bridged ring system containing 2 ring N heteroatoms, optionally a 7- to 8-membered saturated, bridged ring system containing 2 ring N heteroatoms (for example a bridged piperazine, such as 3,8-diazabicyclo[3.2.1]octanyl), wherein R¹ is optionally substituted with 1 R⁴.

6. The compound, or a pharmaceutically acceptable salt or derivative thereof, for use of any preceding claim, wherein when substituted on an aliphatic group, each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH; and when substituted on an aromatic group, each R⁵ is independently halogen, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH.

7. The compound, or a pharmaceutically acceptable salt or derivative thereof, for use of any preceding claim, wherein:
a) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain, branched or cyclic, or a combination thereof; and wherein R^{2a} is optionally substituted with 1 or more R⁵; and
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵; or
b) R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵; or
c) R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; and wherein R^{2f} is optionally substituted with 1 or more R⁵.

8. The compound, or a pharmaceutically acceptable salt or derivative thereof, for use of any preceding claim, wherein:
a) R^{2a} is a (C5-10)alkyl group comprising a cyclic moiety; and wherein R^{2a} is optionally substituted with 1 or more R⁵; and R^{2b} is H or (C1-6)alkyl, and wherein (C1-6)alkyl is optionally substituted with 1 or more R⁵; or
b) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2a} is substituted with 1 or more R⁵ (optionally wherein R⁵ is halogen); and R^{2b} is H or (C1-6)alkyl, and wherein (C1-6)alkyl is optionally substituted with 1 or more R⁵.

9. The compound, or a pharmaceutically acceptable salt or derivative thereof, for use of any preceding claim, wherein R^{2c} is:
a) a (C3-10)alkyl group comprising a cyclic moeity, wherein a C atom in straight chain or cyclic portion of said (C3-10)alkyl group may be optionally replaced by 1 —O— other than at the point of attachment of R²⁰, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a (C3-10)alkyl group that may be straight chain or branched, wherein a C atom in straight chain portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is substituted with 1 or more R⁵ (optionally wherein R⁵ is halogen).

10. The compound, or a pharmaceutically acceptable salt or derivative thereof, for use of any preceding claim, wherein the compound is of formula: 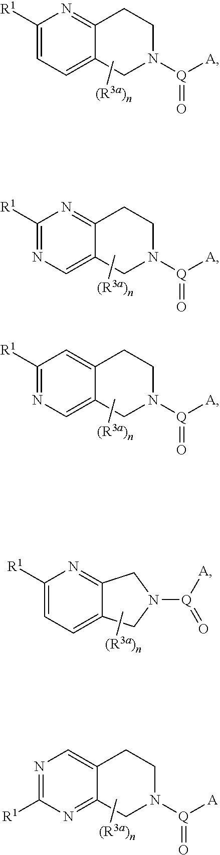
or a pharmaceutically acceptable salt or derivative thereof.

11. The compound, or a pharmaceutically acceptable salt or derivative thereof, for use of any preceding claim, wherein each R^{3a} is-CH₃ or F, or two R^{3a} attached to the same carbon are joined together with the atom to which they are attached to form a cyclopropyl ring.

12. The compound, or a pharmaceutically acceptable salt or derivative thereof, for use of any preceding claim, wherein n is 0, 1 or 2.

12. The compound, or a pharmaceutically acceptable salt or derivative thereof, for use of any preceding claim, wherein n is 0.

13. The compound, or a pharmaceutically acceptable salt or derivative thereof, for use of any preceding claim, wherein Q is C and/or A is R^{2c}.

14. The compound, or a pharmaceutically acceptable salt or derivative thereof, for use of any preceding claim, wherein
Q is C;
R¹ is a a 7- to 9-membered saturated, bridged ring system containing 2 ring N heteroatoms, wherein R¹ is optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2.

15. The compound, or a pharmaceutically acceptable salt or derivative thereof, for use of any preceding claim, wherein one of Y₁, Y₂ and Y₃ is N and the others are each CR^{3b}.

16. The compound, or a pharmaceutically acceptable salt or derivative thereof, for use of any preceding claim, wherein a is 1 and b is 1.

17. The compound, or a pharmaceutically acceptable salt or derivative thereof, for use of any preceding claim, wherein the compound is of formula: 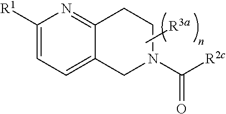
or a pharmaceutically acceptable salt or derivative thereof, optionally wherein:
R¹ is a 6-membered saturated monocyclic ring containing 2 ring N heteroatoms or a 7- to 9-membered saturated, bridged ring system containing 2 ring N heteroatoms, optionally substituted with 1 or more R⁴; and/or
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵.

18. A compound of Formula II: 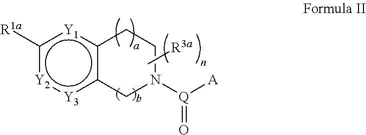
or a pharmaceutically acceptable salt or derivative thereof, wherein:
one or two of Y₁, Y₂ and Y₃ are N and the others are each CR^{3b};
Q is C or S(O);
R^{1a} is a 7- to 9-membered saturated, bridged ring system containing 2 ring N heteroatoms, and wherein R^{1a} is optionally substituted with 1 or more R⁴;
A is R^{2c}, NR^{2a}R^{2b} or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; and wherein R^{2c} is optionally substituted with 1 or more R⁵;
R^{2d} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered carbocylic ring or heterocyclic ring containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1 and b is 1 or 2, wherein when b is 2, a is 0.

19. The compound, or a pharmaceutically acceptable salt or derivative thereof, of claim 18, wherein R^{1a} is a 7- to 8-membered saturated, bridged ring system containing 2 ring N heteroatoms (for example a bridged piperazine, such as 3,8-diazabicyclo[3.2.1]octanyl), wherein R¹ is optionally substituted with 1 R⁴.

20. The compound, or a pharmaceutically acceptable salt or derivative thereof, of any claim 18 or 19, wherein the compound is of formula: 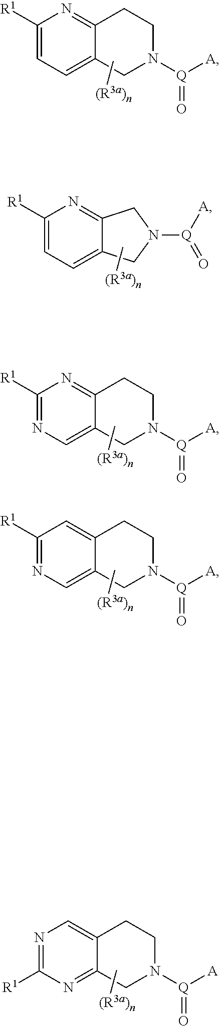
or a pharmaceutically acceptable salt or derivative thereof.

21. The compound, or a pharmaceutically acceptable salt or derivative thereof, of any of claims 18 to 20, wherein one of Y₁, Y₂ and Ya is N and the others are each CR^{3b}.

22. A compound of Formula III: 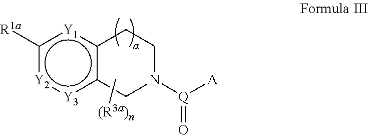
or a pharmaceutically acceptable salt or derivative thereof, wherein:
one of Y₁, Y₂ and Y₃ is N and the others are each CR^{3b};
Q is C or S(O);
R^{1b} is a 4- to 10-membered non-aromatic ring that may be monocyclic, bridged or bicyclic containing at least 1 ring N heteroatom and optionally a ring O heteroatom, wherein at least 1 ring N heteroatom is not at the point of attachment of R^{1b}, and wherein R^{1b} is optionally substituted with 1 or more R⁴;
A is R^{2c} or NR^{2a}R^{2b};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered carbocylic ring or heterocyclic ring containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

23. The compound or a pharmaceutically acceptable salt or derivative thereof of claim 22, wherein R^{1b} is a 5- to 6-membered saturated, monocyclic ring containing at least 1 ring N heteroatom and optionally a ring O heteroatom; or a 7- to 9-membered saturated, bridged ring system containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R^{1b} is optionally substituted with 1, 2 or 3 R⁴.

24. The compound, or a pharmaceutically acceptable salt or derivative thereof, of claim 22 or 23, wherein R^{1b} is a 6-membered saturated monocyclic ring containing 2 ring N heteroatoms or a 7- to 9-membered saturated, bridged ring system containing 2 ring N heteroatoms; wherein R^{1a} is optionally substituted with 1 R⁴, optionally wherein R^{1a} is a 7- to 8-membered saturated, bridged ring system containing 2 ring N heteroatoms (for example a bridged piperazine, such as 3,8-diazabicyclo[3.2.1]octanyl), wherein R^{1b} is optionally substituted with 1 R⁴.

25. The compound, or a pharmaceutically acceptable salt or derivative thereof, of any of claims 18-24, wherein a is 1 and, where present, b is 1.

26. The compound, or a pharmaceutically acceptable salt or derivative thereof, of any of claims 18-25, wherein the compound is of formula: 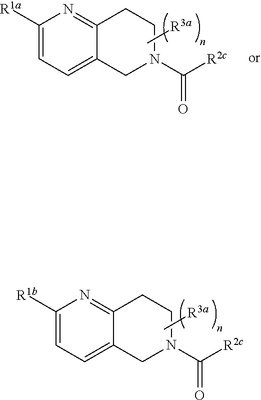
or a pharmaceutically acceptable salt or derivative thereof, optionally wherein the compound is a compound of Formula III and wherein:
R^{1b} is a 6-membered saturated monocyclic ring containing 2 ring N heteroatoms or a 7- to 9-membered saturated, bridged ring system containing 2 ring N heteroatoms, optionally substituted with 1 or more R⁴; and/or
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵.

27. The compound, or a pharmaceutically acceptable salt or derivative thereof, of any of claims 18-25, wherein:
a) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain, branched or cyclic, or a combination thereof; and wherein R^{2a} is optionally substituted with 1 or more R⁵; and
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵; or
b) R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵.

28. The compound, or a pharmaceutically acceptable salt or derivative thereof, of any of claims 18-27, wherein R^{2c} is:
a) a (C3-10)alkyl group comprising a cyclic moeity, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵;
b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or
a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is substituted with 1 or more R⁵ (optionally wherein R⁵ is halogen).

29. The compound, or a pharmaceutically acceptable salt or derivative thereof, of any of claims 18-28, wherein the compound is of formula: 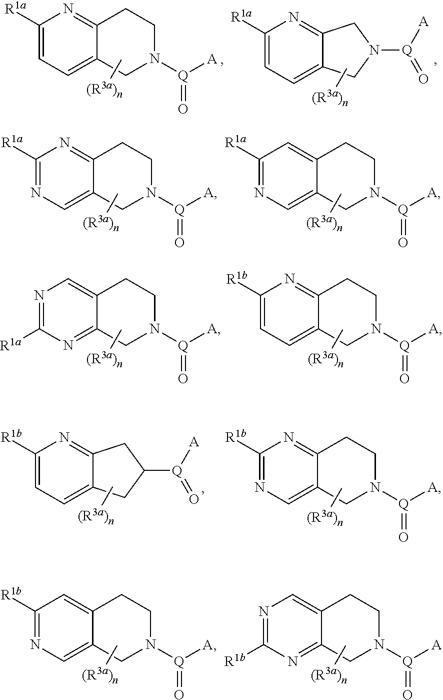
or a pharmaceutically acceptable salt or derivative thereof.

30. The compound, or a pharmaceutically acceptable salt or derivative thereof, of any of claims 18-29, wherein Q is C and/or A is R^{2c}.

31. The compound, or a pharmaceutically acceptable salt or derivative thereof, of any of claims 18-29, wherein
Q is C;
R^{1a} or R^{1b} is a 7- to 9-membered saturated, bridged ring system containing 2 ring N heteroatoms, wherein R^{1a} and R^{1b} are optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2.

32. The compound, or a pharmaceutically acceptable salt or derivative thereof, of any of claims 18-31, wherein each R^{3a} is —CH₃, or two R^{3a} attached to the same carbon are joined together with the atoms to which they are attached to form a cyclopropyl ring.

33. The compound, or a pharmaceutically acceptable salt or derivative thereof, of any of claims 18-32, wherein n is 0, 1 or 2.

34. The compound, or a pharmaceutically acceptable salt or derivative thereof, of any of claims 18-33, wherein n is 0.

35. A compound selected from:
1-(2-(3,8-diazabicyclo[3.2.1]octan-8-yl)-7,8-dihydro-1,6-naphthyridin-6(5H)-yl)-2-cyclopentylethan-1-one;
1-(2-(3,8-diazabicyclo[3.2.1]octan-8-yl)-7,8-dihydro-1,6-naphthyridin-6(5H)-yl)-2-(4,4-difluorocyclohexyl)ethan-1-one;
1-(2-(3,8-diazabicyclo[3.2.1]octan-8-yl)-7,8-dihydro-1,6-naphthyridin-6(5H)-yl)-2-phenoxyethan-1-one;
1-(2-(3,8-diazabicyclo[3.2.1]octan-8-yl)-7,8-dihydro-1,6-naphthyridin-6(5H)-yl)-3,3-dimethylbutan-1-one;
1-(2-(3,8-diazabicyclo[3.2.1]octan-8-yl)-7,8-dihydro-1,6-naphthyridin-6(5H)-yl)-3-methylbutan-1-one;
1-(2-(3,8-diazabicyclo[3.2.1]octan-8-yl)-7,8-dihydro-1,6-naphthyridin-6(5H)-yl)-2,3-dimethylbutan-1-one;
1-(2-(3,8-diazabicyclo[3.2.1]octan-8-yl)-7,8-dihydro-1,6-naphthyridin-6(5H)-yl)-2-phenylethan-1-one;
1-(2-(3,8-diazabicyclo[3.2.1]octan-8-yl)-7,8-dihydro-1,6-naphthyridin-6(5H)-yl)-2-(4-fluorophenoxy)ethan-1-one;
(2-(3,8-diazabicyclo[3.2.1]octan-8-yl)-7,8-dihydro-1,6-naphthyridin-6(5H)-yl)(cyclopentyl)methanone;
1-(2-(3,8-diazabicyclo[3.2.1]octan-8-yl)-7,8-dihydro-1,6-naphthyridin-6(5H)-yl)-2-(4-fluorophenyl)ethan-1-one;
1-(2-(3,8-diazabicyclo[3.2.1]octan-8-yl)-7,8-dihydro-1,6-naphthyridin-6(5H)-yl)-2,2-difluoro-2-phenylethan-1-one;
2-(3,8-diazabicyclo[3.2.1]octan-8-yl)-N-(4,4-difluorocyclohexyl)-7,8-dihydro-1,6-naphthyridine-6(5H)-carboxamide;
(2-(3,8-diazabicyclo[3.2.1]octan-8-yl)-7,8-dihydro-1,6-naphthyridin-6(5H)-yl)(pyrrolidin-1-yl)methanone;
2-(3,8-diazabicyclo[3.2.1]octan-8-yl)-N-cyclopentyl-N-methyl-7,8-dihydro-1,6-naphthyridine-6(5H)-carboxamide;
2-(3,8-diazabicyclo[3.2.1]octan-8-yl)-N-cyclopentyl-N-ethyl-7,8-dihydro-1,6-naphthyridine-6(5H)-carboxamide;
1-(2-(3,8-diazabicyclo[3.2.1]octan-8-yl)-5,7-dihydro-6H-pyrrolo[3,4-b]pyridin-6-yl)-2-(4,4-difluorocyclohexyl)ethan-1-one;
1-(2-(3,8-diazabicyclo[3.2.1]octan-8-yl)-5,7-dihydro-6H-pyrrolo[3,4-b]pyridin-6-yl)-2-cyclopentylethan-1-one;
2-(3,8-diazabicyclo[3.2.1]octan-8-yl)-N-(4,4-difluorocyclohexyl)-5,7-dihydro-6H-pyrrolo[3,4-b]pyridine-6-carboxamide;
2-(3,8-diazabicyclo[3.2.1]octan-8-yl)-N-cyclopentyl-5,7-dihydro-6H-pyrrolo[3,4-b]pyridine-6-carboxamide;
N-(4,4-difluorocyclohexyl)-2-(3-methyl-3,8-diazabicyclo[3.2.1]octan-8-yl)-5,7-dihydro-6H-pyrrolo[3,4-b]pyridine-6-carboxamide;
N-cyclopentyl-2-(3-methyl-3,8-diazabicyclo[3.2.1]octan-8-yl)-5,7-dihydro-6H-pyrrolo[3,4-b]pyridine-6-carboxamide;
2-(4,4-difluorocyclohexyl)-1-(2-(3-methyl-3,8-diazabicyclo[3.2.1]octan-8-yl)-5,7-dihydro-6H-pyrrolo[3,4-b]pyridin-6-yl)ethan-1-one;
2-cyclopentyl-1-(2-(3-methyl-3,8-diazabicyclo[3.2.1]octan-8-yl)-5,7-dihydro-6H-pyrrolo[3,4-b]pyridin-6-yl)ethan-1-one;
(2-(3,8-diazabicyclo[3.2.1]octan-8-yl)-5,7-dihydro-6H-pyrrolo[3,4-b]pyridin-6-yl)(pyrrolidin-1-yl)methanone;
2-(3,8-diazabicyclo[3.2.1]octan-8-yl)-N,N-diethyl-5,7-dihydro-6H-pyrrolo[3,4-b]pyridine-6-carboxamide;
N-(4,4-difluorocyclohexyl)-2-(3-methyl-3,8-diazabicyclo[3.2.1]octan-8-yl)-7,8-dihydro-1,6-naphthyridine-6(5H)-carboxamide;
2-(4,4-difluorocyclohexyl)-1-(2-(3-methyl-3,8-diazabicyclo[3.2.1]octan-8-yl)-7,8-dihydro-1,6-naphthyridin-6(5H)-yl)ethan-1-one;
2-(4,4-difluorocyclohexyl)-1-(2-(piperazin-1-yl)-7,8-dihydro-1,6-naphthyridin-6(5H)-yl)ethan-1-one;
(R)-2-cyclopentyl-1-(2-(hexahydropyrrolo[1,2-a]pyrazin-2 (1H)-yl)-7,8-dihydro-1,6-naphthyridin-6(5H)-yl)ethan-1-one;
(S)-2-cyclopentyl-1-(2-(hexahydropyrrolo[1,2-a]pyrazin-2 (1H)-yl)-7,8-dihydro-1,6-naphthyridin-6(5H)-yl)ethan-1-one;
2-cyclopentyl-1-(2-(piperidin-4-yl)-7,8-dihydro-1,6-naphthyridin-6(5H)-yl)ethan-1-one;
2-cyclopentyl-1-(2-(1-(2-hydroxyethyl)piperidin-4-yl)-7,8-dihydro-1,6-naphthyridin-6(5H)-yl)ethan-1-one;
1-(2-(3,8-diazabicyclo[3.2.1]octan-8-yl)-7,8-dihydro-1,6-naphthyridin-6(5H)-yl)-2-(1-methylcyclopentyl)ethan-1-one;
(2-(3,8-diazabicyclo[3.2.1]octan-8-yl)-7,8-dihydro-1,6-naphthyridin-6(5H)-yl)(phenyl)methanone;
(2-(3,8-diazabicyclo[3.2.1]octan-8-yl)-7,8-dihydro-1,6-naphthyridin-6(5H)-yl)(2-methoxyphenyl)methanone;
(2-(3,8-diazabicyclo[3.2.1]octan-8-yl)-7,8-dihydro-1,6-naphthyridin-6(5H)-yl)(o-tolyl)methanone;
(2-(3,8-diazabicyclo[3.2.1]octan-8-yl)-7,8-dihydro-1,6-naphthyridin-6(5H)-yl)(4-fluorophenyl)methanone;
(2R)-1-(2-(3,8-diazabicyclo[3.2.1]octan-8-yl)-7,8-dihydro-1,6-naphthyridin-6(5H)-yl)-2-cyclopentyl-2-hydroxyethan-1-one;
cyclopentyl 2-(3,8-diazabicyclo[3.2.1]octan-8-yl)-7,8-dihydro-1,6-naphthyridine-6(5H)-carboxylate;
isopropyl 2-(3,8-diazabicyclo[3.2.1]octan-8-yl)-7,8-dihydro-1,6-naphthyridine-6(5H)-carboxylate;
benzyl 2-(3,8-diazabicyclo[3.2.1]octan-8-yl)-7,8-dihydro-1,6-naphthyridine-6(5H)-carboxylate;
(2S)-1-(2-(3,8-diazabicyclo[3.2.1]octan-8-yl)-5,7-dihydro-6H-pyrrolo[3,4-b]pyridin-6-yl)-2,3-dimethylbutan-1-one;
1-(2-(3,8-diazabicyclo[3.2.1]octan-8-yl)-5,7-dihydro-6H-pyrrolo[3,4-b]pyridin-6-yl)-2-(1-methylcyclopentyl)ethan-1-one;
1-(2-(3,8-diazabicyclo[3.2.1]octan-8-yl)-5,7-dihydro-6H-pyrrolo[3,4-b]pyridin-6-yl)-2,2-difluoro-2-phenylethan-1-one;
(R)-2-cyclopentyl-1-(2-(3-(hydroxymethyl) piperazin-1-yl)-7,8-dihydro-1,6-naphthyridin-6(5H)-yl)ethan-1-one;
(S)-2-cyclopentyl-1-(2-(3-(hydroxymethyl) piperazin-1-yl)-7,8-dihydro-1,6-naphthyridin-6(5H)-yl)ethan-1-one;
1-(2-(3,8-diazabicyclo[3.2.1]octan-8-yl)-7,8-dihydropyrido[4,3-d]pyrimidin-6(5H)-yl)-2-phenylethan-1-one;
2-phenyl-1-(2-(piperazin-1-yl)-7,8-dihydropyrido[4,3-d]pyrimidin-6(5H)-yl)ethan-1-one;
1-(2-(3,8-diazabicyclo[3.2.1]octan-3-yl)-7,8-dihydropyrido[4,3-d]pyrimidin-6(5H)-yl)-2-phenylethan-1-one;
4-(2-(2-(3,8-diazabicyclo[3.2.1]octan-8-yl)-7,8-dihydropyrido[4,3-d]pyrimidin-6(5H)-yl)-2-oxoethyl)benzonitrile;
4-(2-oxo-2-(2-(piperazin-1-yl)-7,8-dihydropyrido[4,3-d]pyrimidin-6(5H)-yl)ethyl)benzonitrile;
4-(2-(2-(3,8-diazabicyclo[3.2.1]octan-3-yl)-7,8-dihydropyrido[4,3-d]pyrimidin-6(5H)-yl)-2-oxoethyl)benzonitrile;
1-(2-(3,8-diazabicyclo[3.2.1]octan-8-yl)-7,8-dihydropyrido[4,3-d]pyrimidin-6(5H)-yl)-2-(4-fluorophenyl)ethan-1-one;
2-(4-fluorophenyl)-1-(2-(piperazin-1-yl)-7,8-dihydropyrido[4,3-d]pyrimidin-6(5H)-yl)ethan-1-one;
1-(2-(3,8-diazabicyclo[3.2.1]octan-3-yl)-7,8-dihydropyrido[4,3-d]pyrimidin-6(5H)-yl)-2-(4-fluorophenyl)ethan-1-one;
1-(2-(3,8-diazabicyclo[3.2.1]octan-8-yl)-7,8-dihydropyrido[4,3-d]pyrimidin-6(5H)-yl)-2-cyclopentylethan-1-one;
2-cyclopentyl-1-(2-(piperazin-1-yl)-7,8-dihydropyrido[4,3-d]pyrimidin-6(5H)-yl)ethan-1-one;
1-(2-(3,8-diazabicyclo[3.2.1]octan-8-yl)-7,8-dihydropyrido[4,3-d]pyrimidin-6(5H)-yl)-2-cyclohexylethan-1-one;
2-cyclohexyl-1-(2-(piperazin-1-yl)-7,8-dihydropyrido[4,3-d]pyrimidin-6(5H)-yl)ethan-1-one;
1-(2-(3,8-diazabicyclo[3.2.1]octan-8-yl)-7,8-dihydropyrido[4,3-d]pyrimidin-6(5H)-yl)-3,3-dimethylbutan-1-one;
(4-fluorophenyl)(2-(piperazin-1-yl)-7,8-dihydropyrido[4,3-d]pyrimidin-6(5H)-yl)methanone;
1-(2-(3,8-diazabicyclo[3.2.1]octan-8-yl)-5,8-dihydropyrido[3,4-d]pyrimidin-7 (6H)-yl)-2-cyclopentylethan-1-one;
3,3-dimethyl-1-(2-(piperazin-1-yl)-7,8-dihydro-1,6-naphthyridin-6(5H)-yl) butan-1-one;
1-(2-(4-ethylpiperazin-1-yl)-7,8-dihydro-1,6-naphthyridin-6(5H)-yl)-3,3-dimethylbutan-1-one;
1-(2-(3,9-diazabicyclo[3.3.1]nonan-9-yl)-7,8-dihydro-1,6-naphthyridin-6(5H)-yl)-2-cyclopentylethan-1-one;
1-(2-(3,9-diazabicyclo[3.3.1]nonan-3-yl)-7,8-dihydro-1,6-naphthyridin-6(5H)-yl)-2-cyclopentylethan-1-one;
1-(2-(2,5-diazabicyclo[2.2.2]octan-2-yl)-7,8-dihydro-1,6-naphthyridin-6(5H)-yl)-2-cyclopentylethan-1-one;
1-(2-(3,8-diazabicyclo[3.2.1]octan-3-yl)-7,8-dihydro-1,6-naphthyridin-6(5H)-yl)-2-cyclopentylethan-1-one;
1-(2-((1S,4S)-2,5-diazabicyclo[2.2.1]heptan-2-yl)-7,8-dihydro-1,6-naphthyridin-6(5H)-yl)-2-cyclopentylethan-1-one;
1-(2-((1R,4R)-2,5-diazabicyclo[2.2.1]heptan-2-yl)-7,8-dihydro-1,6-naphthyridin-6(5H)-yl)-2-cyclopentylethan-1-one;
2-cyclopentyl-1-(2-(7-hydroxy-3,9-diazabicyclo[3.3.1]nonan-9-yl)-7,8-dihydro-1,6-naphthyridin-6(5H)-yl)ethan-1-one;
2-(3,8-diazabicyclo[3.2.1]octan-8-yl)-6-((4-fluorobenzyl) sulfonyl)-5,6,7,8-tetrahydro-1,6-naphthyridine;
2-(3-ethyl-3,8-diazabicyclo[3.2.1]octan-8-yl)-6-((4-fluorobenzyl) sulfonyl)-5,6,7,8-tetrahydro-1,6-naphthyridine;
6-((4-fluorobenzyl) sulfonyl)-2-(piperazin-1-yl)-5,6,7,8-tetrahydro-1,6-naphthyridine;
2-(4-ethylpiperazin-1-yl)-6-((4-fluorobenzyl) sulfonyl)-5,6,7,8-tetrahydro-1,6-naphthyridine;
6-(benzylsulfonyl)-2-(3,8-diazabicyclo[3.2.1]octan-8-yl)-5,6,7,8-tetrahydro-1,6-naphthyridine;
6-(benzylsulfonyl)-2-(3-ethyl-3,8-diazabicyclo[3.2.1]octan-8-yl)-5,6,7,8-tetrahydro-1,6-naphthyridine;
6-(benzylsulfonyl)-2-(piperazin-1-yl)-5,6,7,8-tetrahydro-1,6-naphthyridine;
6-(benzylsulfonyl)-2-(4-ethylpiperazin-1-yl)-5,6,7,8-tetrahydro-1,6-naphthyridine;
2-(3,8-diazabicyclo[3.2.1]octan-8-yl)-N—((S)-chroman-4-yl)-7,8-dihydro-1,6-naphthyridine-6(5H)-carboxamide;
N—((S)-chroman-4-yl)-2-(3-ethyl-3,8-diazabicyclo[3.2.1]octan-8-yl)-7,8-dihydro-1,6-naphthyridine-6(5H)-carboxamide;
(S)—N-(chroman-4-yl)-2-(piperazin-1-yl)-7,8-dihydro-1,6-naphthyridine-6(5H)-carboxamide;
(S)—N-(chroman-4-yl)-2-(4-ethylpiperazin-1-yl)-7,8-dihydro-1,6-naphthyridine-6(5H)—N—((R)-chroman-4-yl)-2-(3-ethyl-3,8-diazabicyclo[3.2.1]octan-8-yl)-7,8-dihydro-1,6-naphthyridine-6(5H)-carboxamide;
1-(2-(3-ethyl-3,8-diazabicyclo[3.2.1]octan-8-yl)-7,8-dihydro-1,6-naphthyridin-6(5H)-yl)-2-(4-fluorophenoxy)ethan-1-one;
2-(4-fluorophenoxy)-1-(2-(3-propyl-3,8-diazabicyclo[3.2.1]octan-8-yl)-7,8-dihydro-1,6-naphthyridin-6(5H)-yl)ethan-1-one;
2-(4-fluorophenoxy)-1-(2-(3-(2-methoxyethyl)-3,8-diazabicyclo[3.2.1]octan-8-yl)-7,8-dihydro-1,6-naphthyridin-6(5H)-yl)ethan-1-one;
2-(4-fluorophenoxy)-1-(2-(3-(3-methoxypropyl)-3,8-diazabicyclo[3.2.1]octan-8-yl)-7,8-dihydro-1,6-naphthyridin-6(5H)-yl)ethan-1-one;
2-phenyl-1-(2-(piperazin-1-yl)-7,8-dihydro-1,6-naphthyridin-6(5H)-yl)ethan-1-one;
2-(4-fluorophenyl)-1-(2-(piperazin-1-yl)-7,8-dihydro-1,6-naphthyridin-6(5H)-yl)ethan-1-one;
1-(3-(3,8-diazabicyclo[3.2.1]octan-8-yl)-5,8-dihydro-1,7-naphthyridin-7 (6H)-yl)-2-cyclopentylethan-1-one;
1-(6-(3,8-diazabicyclo[3.2.1]octan-8-yl)-3,4-dihydro-2,7-naphthyridin-2 (1H)-yl)-2-cyclopentylethan-1-one;
1-(7-(3,8-diazabicyclo[3.2.1]octan-8-yl)-3,4-dihydro-2,6-naphthyridin-2 (1H)-yl)-2-cyclopentylethan-1-one;
2-(4-fluorophenoxy)-1-(2-(piperazin-1-yl)-7,8-dihydro-1,6-naphthyridin-6(5H)-yl)ethan-1-one;
or a pharmaceutically acceptable salt or derivative thereof.

36. A pharmaceutical composition comprising a compound or a pharmaceutically acceptable salt or derivative as defined in any of claims 1-35, and a pharmaceutically acceptable excipient.

37. A compound or pharmaceutically acceptable salt or derivative of any of claims 18-35 for use in therapy.

38. A compound or pharmaceutically acceptable salt or derivative of any of claims 18-35 or a pharmaceutical composition of claim 36 for use in the treatment or prevention of a disease or disorder that can be ameliorated by activation of long isoforms of PDE4 or a disease or disorder mediated by excessive intracellular cyclic AMP signalling.

39. The compound or pharmaceutically acceptable salt or derivative for use of any of claims 1 to 17 or the compound or pharmaceutically acceptable salt or derivative or pharmaceutical composition for use of claim 38 in the treatment or prevention of a disease or disorder mediated by excessive intracellular cyclic AMP signalling.

40. A method of treating or preventing a disease or disorder that can be ameliorated by activation of long isoforms of PDE4 or a disease or disorder mediated by excessive intracellular cyclic AMP signalling, comprising administering to a patient in need thereof a therapeutically effective amount of a compound or a pharmaceutically acceptable salt or derivative as defined in any of claims 1 to 35.

41. Use of a compound or a pharmaceutically acceptable salt or derivative as defined in any of claims 1 to 35, in the manufacture of a medicament for treatment or prevention of a disease or disorder that can be ameliorated by activation of long isoforms of PDE4 or a disease or disorder mediated by excessive intracellular cyclic AMP signalling.

42. The method of claim 40 or the use of claim 41, wherein the disease or disorder that can be ameliorated by activation of long isoforms of PDE4 is a disease or disorder mediated by excessive intracellular cyclic AMP signalling.

43. The compound or pharmaceutically acceptable salt or derivative or pharmaceutical composition for use of claim 39, or the method or use of any of claims 40-42, wherein the excessive intracellular cyclic AMP signalling is caused by:
a. excessive hormone levels produced by an adenoma.
b. a gain-of-function gene mutation in a G-protein coupled receptor (GPCR);
c. an activating mutation in the GNAS1 gene, which encodes the α-subunit of the G-protein Gₛ; or
d. a bacterial toxin.

44. The compound or pharmaceutically acceptable salt or derivative or pharmaceutical composition for use of any of claim 1-17 or 38, 39 or 43, or the method or use of any of claims 40-43, wherein the disease is cancer, optionally wherein the cancer is prostate cancer.

45. The compound or pharmaceutically acceptable salt or derivative or pharmaceutical composition for use of any of claim 1-17 or 38, 39 or 43, or the method or use of any of claims 40-43, wherein the disease is:
a. pituitary adenoma, Cushing's disease, polycystic kidney disease or polycystic liver disease;
b. hyperthyroidism, Jansens's metaphyseal chondrodysplasia, hyperparathyroidism, or familial male-limited precocious puberty;
c. McCune-Albright syndrome;
d. cholera, whooping cough, anthrax, or tuberculosis;
e. HIV, AIDS, or Common Variable Immunodeficiency (CVID);
f. melanoma, pancreatic cancer, leukaemia, prostate cancer, adrenocortical tumours, testicular cancer, primary pigmented nodular adrenocortical diseases (PPNAD), or Carney Complex;
g. autosomal dominant polycystic kidney disease (ADPKD) or autosomal recessive polycystic kidney disease (ARPKD); or
h. maturity onset diabetes of young type 5 (MODY5); or
i. cardiac hypertrophy.

46. The compound or pharmaceutically acceptable salt or derivative or pharmaceutical composition for use, method or use of claim 45, wherein the disease is:
a. autosomal dominant polycystic kidney disease (ADPKD) or autosomal recessive polycystic kidney disease (ARPKD); or
b. hyperparathyroidism.
